(19) United States
(12) Patent Application Publication
(10) Pub. No.: US 2024/0400542 A1
(43) Pub. Date: Dec. 5, 2024

(54) SULFONYL-TRIAZOLES USEFUL AS COVALENT KINASE LIGANDS

(71) Applicant: University of Virginia Patent Foundation, Charlottesville, VA (US)

(72) Inventors: Ku-Lung Hsu, Austin, TX (US); Kun Yuan, Charlottesville, VA (US); Minhaj Shaikh, Charlottesville, VA (US); Xiaoding Jiang, Charlottesville, VA (US); Tao Huang, Charlottesville, VA (US); Adam Libby, Charlottesville, VA (US)

(73) Assignee: University of Virginia Patent Foundation, Charlottesville, VA (US)

(21) Appl. No.: 18/685,194

(22) PCT Filed: Aug. 19, 2022
(86) PCT No.: PCT/US2022/075243

§ 371 Date: Feb. 20, 2024

Related U.S. Application Data
(60) Provisional application No. 63/234,977, filed on Aug. 19, 2021.

(51) Int. Cl. C07D 403/14; A61K 31/454; A61K 31/4545; A61K 31/496; A61K 31/506; A61K 31/519; C07D 401/06; C07D 403/06; C07D 405/14; C07D 417/14; C07D 487/04

(52) U.S. Cl. CPC C07D 403/14; A61K 31/454; A61K 31/4545; A61K 31/496; A61K 31/506; A61K 31/519; C07D 401/06; C07D 403/06; C07D 405/14; C07D 417/14; C07D 487/04

(57) ABSTRACT

Sulfonyl-triazole compounds and related sulfonyl-heterocycle compounds are described. The compounds can form covalent adducts with reactive nucleophilic amino acid residues, e.g., reactive tyrosines and reactive lysines, in kinases to form modified kinases and/or alter the biological activity of the kinases. Kinases targetable by the compounds include cyclin-dependent kinase 2 (CDK2), diacylglycerol kinases (DGKs), and phosphofructokinase (PFK). Pharmaceutical compositions including the compounds and methods of inhibiting kinases are also described.

### CROSS-REFERENCE TO RELATED APPLICATION

This application claims priority to and the benefit of U.S. Provisional Patent Application Ser. No. 63/234,977, filed Aug. 19, 2021, the disclosure of which is incorporated herein by referenced in its entirety.

### GRANT STATEMENT

This invention was made with government support under Grant No. DA043571 awarded by the National Institutes of Health. The government has certain rights in the invention.

### REFERENCE TO SEQUENCE LISTING XML SUBMITTED ELECTRONICALLY

The content of the Sequence Listing XML filed using Patent Center as an XML file (Name: 3062_166_PCT.xml; Size: 9,075 bytes; and Date of Creation: Aug. 19, 2022) is incorporated herein by reference in its entirety.

### TECHNICAL FIELD

The presently disclosed subject matter relates to sulfonyl-triazole compounds and their use as targeted covalent ligands to modulate kinase function.

### BACKGROUND

Kinases constitute a large and diverse class of proteins with greater than 500 members in the human proteome¹. Kinases catalyze the adenosine triphosphate (ATP)-dependent transfer of a phosphate group to protein or small molecule substrates². These enzymes are important mediators of signal transduction to regulate cell metabolism, growth, and survival in response to external stimuli³. The reversible phosphorylation of substrate proteins on serine, threonine, and tyrosine residues can alter protein conformation and activation, subcellular localization, and protein-protein interactions⁴⁻⁵. Thus, kinases act as molecular switches to regulate cell biology through post-translational modification of signaling proteins. Given their role in cancer, inflammatory, and neurodegenerative diseases, kinases are prominent drug targets⁶.

Accordingly, there is an ongoing need in the art for additional kinase inhibitors, such as inhibitors that are cell permeable and/or that covalent bond to kinases.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a compound having a structure of formula (I), (II), or (III):

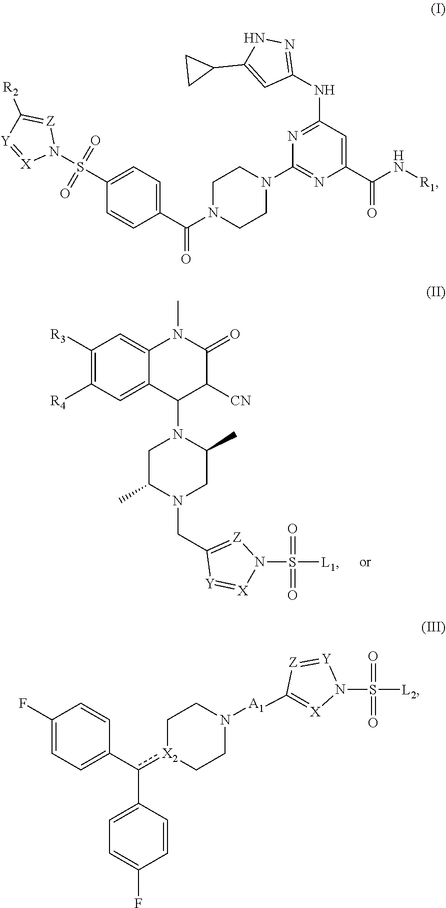

wherein: [symbol] is a double or single bond; X, Y, and Z are independently C or N, subject to the proviso that at least one of X, Y, and Z is N; X₂ is C or N, subject to the proviso that when [symbol] is a single bond, X₂ is N and when [symbol] is a double bond, X₂ is C; R₁ is alkyl, cycloalkyl, aralkyl, aryl, or heteroaryl, which alkyl, cycloalkyl, aralkyl, aryl or heteroaryl is optionally substituted with one or more alkyl or aryl group substituent selected from the group comprising halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene, and subject to the proviso that R₁ does not comprise an alkyne group; R₂ is alkyl, cycloalkyl, aralkyl, or aryl, which alkyl, cycloalkyl, aralkyl, or aryl is optionally substituted with one or more alkyl or aryl group substituent selected from the group comprising halo, cyano, alkyl, alkoxy, perhaloalkyl, perhaloalkoxy, cycloalkyl, aralkyl, aryl, and amido; R₃ and R₄ are independently selected from the group comprising H, halo, alkyl, perhaloalkyl, and alkoxy; L₁ and L₂ are alkyl, cycloalkyl, aralkyl, aryl, or heteroaryl, which alkyl, cycloalkyl, aralkyl, aryl or heteroaryl is optionally substituted with one or more alkyl or aryl group substituent selected from the group comprising halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene, and subject to the proviso that L₁ and L₂ do not comprise an alkyne group; and A₁ is selected from the group consisting of ethylene,

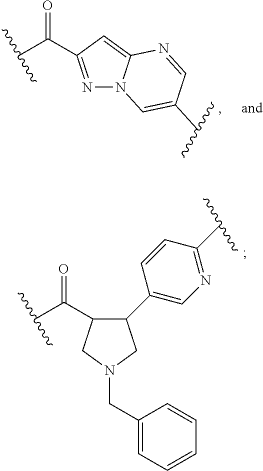 [SMILES: *C(=O)c1cc2ncc(*)cn2n1 ; *C(=O)C1CN(Cc2ccccc2)CC1c1ccc(*)nc1]

or a pharmaceutically acceptable salt or solvate thereof.

In some embodiments, the compound has a structure of formula (I):

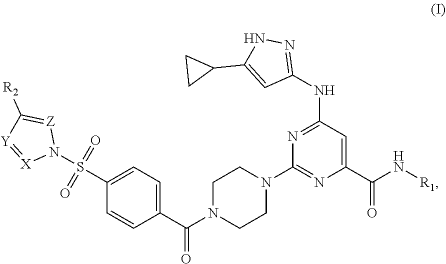

wherein: X, Y, and Z are independently C or N, subject to the proviso that two of X, Y, and Z are N; R₁ is alkyl, cycloalkyl, aralkyl, aryl, or heteroaryl, which alkyl, cycloalkyl, aralkyl, aryl or heteroaryl is optionally substituted with one or more alkyl or aryl group substituent selected from the group comprising halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene, and subject to the proviso that R₁ does not comprise an alkyne group; and R₂ is alkyl, cycloalkyl, aralkyl, or aryl, which alkyl, cycloalkyl, aralkyl, or aryl is optionally substituted with one or more alkyl or aryl group substituent selected from the group comprising halo, cyano, alkyl, alkoxy, perhaloalkyl, perhaloalkoxy, cycloalkyl, aralkyl, aryl, and amido; or a pharmaceutically acceptable salt or solvate thereof. In some embodiments, Y and Z are each N and X is C.

In some embodiments, R₁ is alkyl. In some embodiments, R₁ is n-propyl. In some embodiments, R₂ is aryl. In some embodiments, R₂ is phenyl.

In some embodiments, the compound is 6-((5-cycloproypyl-1H-pyrazol-3-yl)amino)-2-(4-(4-((3-phenyl-1H-1,2,4-triazol-1-yl)sulfonyl)-benzoyl)piperaz-in-1-yl)-N-propylpyrim-idine-4-carboxamide) (KY-424), or a pharmaceutically acceptable salt or solvate thereof.

In some embodiments, the compound has a structure of formula (II):

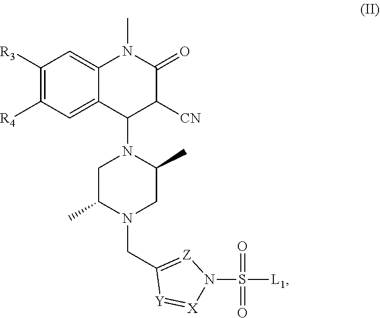

wherein: X, Y, and Z are independently C or N, subject to the proviso that two of X, Y, and Z are N; R₃ and R₄ are independently selected from the group comprising H, halo, alkyl, perhaloalkyl, and alkoxy; and L₁ is alkyl, cycloalkyl, aralkyl, aryl, or heteroaryl, which alkyl, cycloalkyl, aralkyl, aryl or heteroaryl is optionally substituted with one or more alkyl or aryl group substituent selected from the group comprising halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene, and subject to the proviso that L₁ does not contain an alkyne group; or a pharmaceutically acceptable salt or solvate thereof. In some embodiments, X and Y are N and Z is C.

In some embodiments, R₃ and R₄ are independently selected from the group comprising H, halo, and alkoxy. In some embodiments, R₃ is H or methoxy and R₄ is H, Br, or F.

In some embodiments, L₁ is selected from the group comprising alkyl, substituted alkyl, cycloalkyl, aralkyl, phenyl, substituted phenyl, thiazole, and substituted thiazole. In some embodiments, L₁ is selected from the group comprising isopropyl, isobutyl, cyclopropyl, 2-methoxyethyl, 3,3,3-trifluoropropyl, benzyl, phenyl, p-fluorophenyl, p-bromophenyl, p-cyanophenyl, and dimethylthiazole.

In some embodiments, the compound is selected from the group comprising: 4-((2S,5R)-2,5-dimethyl-4-((1-phenylsulfonyl)-1H-1,2,3-triazol-4-yl) methyl)piper-azin-1-yl)-1-methyl-2-oxo-1,2-dihydroquinoline-3-carbonitrile (SMS-55); 4-((2S,5R)-4-((cyclopropylsulfonyl)-1H-1,2,3-triazol-4-yl) methyl) 2,5-dimethyl piperazin-1-yl)-1-methyl-2-oxo-1,2-dihydroquinoline-3-carbonitrile (SMS-59); 4-((2S,5R)-4-((isopropylsulfonyl)-1H-1,2,3-triazol-4-yl)methyl)-2,5-dimethyl piperazin-1-yl)-1-methyl-2-oxo-1,2-dihydroquinoline-3-carbonitrile (SMS-63); 4-((2S,5R)-4-((1-((4-bromophenyl)sulfonyl)-1H-1,2,3-triazol-4-yl)methyl)-2,5-dimethylpiperazin-1-yl)-1-methyl-2-oxo-1,2-dihydroquinoline-3-carbonitrile (SMS-65); 4-((2S,5R)-4-((1-((4-fluorophenyl)sulfonyl)-1H-1,2,3-triazol-4-yl)methyl)-2,5-dimethylpiperazin-1-yl)-1-methyl-2-oxo-1,2-dihydroquinoline-3-carbonitrile (SMS-67); 4-((2S,5R)-4-((1-((4-cyanophenyl)sulfonyl)-1H-1,2,3-triazol-4-yl)methyl)-2,5-dimethylpip-erazin-1-yl)-1-methyl-2-oxo-1,2-dihydroquinoline-3-carbonitrile (SMS-69); 4-((2S,5R)-4-((1-((2,4-dimethylthiazol-5-yl)sulfonyl)-1H-1,2,3-triazol-4-yl) methyl)-2,5-dimethylpiperazin-1-yl)-1-methyl-2-oxo-1,2-dihydroquinoline-3-carbonitrile (SMS-71); 4-((2S,5R)-4-((1-benzylsulfonyl)-1H-1,2,3-triazol-4-yl)methyl)-2,5-dimethyl piperazin-1-yl)-1-methyl-2-oxo-1,2-dihydroquinoline-3-carbonitrile (SMS-73); 4-((2S,5R)-4-((isobutylsulfonyl)-1H-1,2,3-triazol-4-yl)methyl)-2,5-dimethyl piperazin-1-yl)-1-methyl-2-oxo-1,2-dihydroquinoline-3-carbonitrile (SMS-75); 4-((2S,5R)-4-((2-methoxyethyl)sulfonyl)-1H-1,2,3-triazol-4-yl)methyl)-2,5-dimethyl piperazin-1-yl)-1-methyl-2-oxo-1,2-dihydroquinoline-3-carbonitrile (SMS-77); 4-((2S,5R)-2,5-dimethyl-4-((1-((3,3,3-trifluoropropyl)sulfonyl)-1H-1,2,3-triazol-4-yl) methyl)-piperazin-1-yl)-1-methyl-2-oxo-1,2-dihydroquinoline-3-carbonitrile (SMS-79); 6-bromo-4-((2S,5R)-2,5-dimethyl-4-((1-phenylsulfonyl)-1H-1,2,3-triazol-4-yl) methyl)-piperazin-1-yl)-1-methyl-2-oxo-1,2-dihydroquinoline-3-carbonitrile (SMS-81); 4-((2S,5R)-2,5-dimethyl-4-((1-phenylsulfonyl)-1H-1,2,3-triazol-4-yl)methyl) piperazin-1-yl)-6-fluoro-1-methyl-2-oxo-1,2-dihydroquinoline-3-carbonitrile (SMS-83); 6-fluoro-4-((2S,5R)-4-((isopropylsulfonyl)-1H-1,2,3-triazol-4-yl)methyl)-2,5-dimethylpiperazin-1-yl)-1-methyl-2-oxo-1,2-dihydroquinoline-3-carbonitrile (SMS-85); 4-((2S,5R)-2,5-dimethyl-4-((1-phenyl-sulfonyl)-1H-1,2,3-triazol-4-yl)methyl) piperazin-1-yl)-7-methoxy-1-methyl-2-oxo-1,2-dihydroquinoline-3-carbonitrile (SMS-87); and pharmaceutically acceptable salts or solvates thereof.

In some embodiments, the compound has a structure of formula (III):

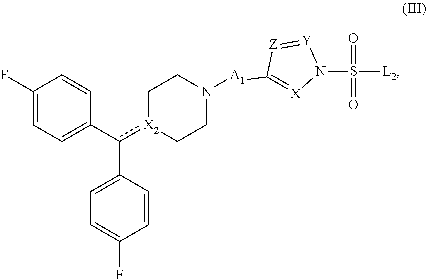

wherein: [symbol] is a double or single bond; X, Y, and Z are independently C or N, subject to the proviso that two of X, Y, and Z are N; X₂ is C or N, subject to the proviso that when [symbol] is a single bond, X₂ is N and when [symbol] is a double bond, X₂ is C; L₂ is selected from alkyl, cycloalkyl, aralkyl, aryl, or heteroaryl, which alkyl, cycloalkyl, aralkyl, aryl or heteroaryl is optionally substituted with one or more alkyl or aryl group substituent selected from the group comprising halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene, and subject to the proviso that L₂ does not comprise an alkyne group; and A₁ is selected from the group consisting of ethylene,

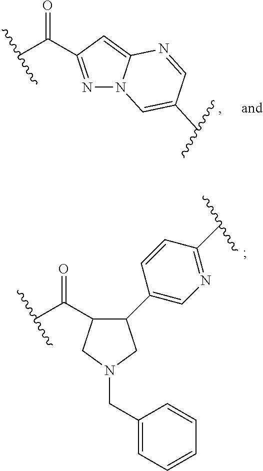 [SMILES: *C(=O)c1cc2ncc(*)cn2n1 ; *C(=O)C1CN(Cc2ccccc2)CC1c1ccc(*)nc1]

or a pharmaceutically acceptable salt or solvate thereof. In some embodiments, Y and Z are each N and X is C.

In some embodiments, L₂ is selected from the group comprising cyclopropyl, phenyl, substituted phenyl, thiazole, and dimethylthiazole. In some embodiments, L₂ is phenyl substituted with one or two substituents selected from the group comprising alkyl, alkoxy, halo, and amido, or L₂ is phenyl substituted with two substituents that together form an alkylene or substituted alkylene. In some embodiments, A₁ is ethylene.

In some embodiments, the compound is selected from the group comprising: 4-((4-(2-(4-(Bis(4-fluorophenyl)methylene)piperidin-1-yl)ethyl)-1H-1,2,3-triazol-1-yl)sulfonyl)-N-propylbenzamide (TH225); 4-(Bis(4-fluorophenyl)methylene)-1-(2-(1-tosyl-1H-1,2,3-triazol-4-yl)ethyl)-piperidine (TH207); 4-(bis(4-fluorophenyl)methylene)-1-(2-(1-(cyclopropylsulfonyl)-1H-1,2,3-triazol-4-yl)ethyl)piperidine (TH223); 1-(Bis(4-fluorophenyl)methyl)-4-(2-(1-tosyl-1H-1,2,3-triazol-4-yl)ethyl)piperazine (TH208); 4-(Bis(4-fluorophenyl)methylene)-1-(2-(1-tosyl-1H-1,2,3-triazol-4-yl)ethyl)-piperidine (TH220); 4-(Bis(4-fluorophenyl)methylene)-1-(2-(1-((4-fluorophenyl)sulfonyl)-1H-1,2,3-triazol-4-yl)ethyl)piperidine (TH221); (4-(Bis(4-fluorophenyl)methylene)piperidin-1-yl)(6-(1-((4-methoxyphenylsulfonyl)-1H-1,2,3-triazol-4-yl)pyrazolo[1,5-a]pyrimidin-2-yl)methanone (XJ-2-47); (1-Benzyl-4-(6-(1-((2,4-dimethylthiazol-5-yl)sulfonyl)-1H-1,2,3-triazol-4-yl)pyridin-3-yl)pyrrolidin-3-yl)(4-(bis(4-fluorophenyl)methylene)piperidin-1-yl)methanone (XJ-2-65); 4-(Bis(4-fluorophenyl)methylene)-1-(2-(1-((2,3-dihydrobenzo[b][1,4]dioxin-6-yl)sulfonyl)-1H-1,2,3-triazol-4-yl)ethyl)piperidine (XJ-2-77); 1-(2-(1-(Benzo[d][1,3]dioxol-5-ylsulfonyl)-1H-1,2,3-triazol-4-yl)ethyl)-4-(bis(4-fluorophenyl)methylene)piperidine (XJ-2-87); 5-((4-(2-(4-(Bis(4-fluorophenyl)methylene)piperidin-1-yl)ethyl)-1H-1,2,3-triazol-1-yl)sulfonyl)-2,4-dimethylthiazole (XJ-2-105); 4-(Bis(4-fluorophenyl)methylene)-1-(2-(1-((2,3-dihydrobenzofuran-6-yl)sulfonyl)-1H-1,2,3-triazol-4-yl)ethyl)piperidine (XJ-2-111); 4-(Bis(4-fluorophenyl)methylene)-1-(2-(1-((2,2-difluorobenzo[d][1,3]dioxol-5-yl)sulfonyl)-1H-1,2,3-triazol-4-yl)ethyl)piperidine (XJ-2-115); 4-(Bis(4-fluorophenyl)methylene)-1-(2-(1-((2,4-dimethoxyphenyl)sulfonyl)-1H-1,2,3-triazol-4-yl)ethyl)piperidine (XJ-2-139); 4-(Bis(4-fluorophenyl)methylene)-1-(2-(1-((2-methoxyphenyl)sulfonyl)-1H-1,2,3-triazol-4-yl)ethyl)-piperidine (XJ-2-141); and pharmaceutically acceptable salts and solvates thereof.

In some embodiments, the presently disclosed subject matter provides a pharmaceutical composition comprising a compound having a structure of formula (I), (II), or (III), or a pharmaceutically acceptable salt or solvate thereof, and a pharmaceutically acceptable carrier.

In some embodiments, the presently disclosed subject matter provides a method of inhibiting a kinase, the method comprising contacting a sample comprising the kinase with a compound having a structure of formula (I), (II), or (III), or a pharmaceutically acceptable salt or solvate thereof, or a pharmaceutical composition thereof. In some embodiments, the sample is selected from the group comprising a biological fluid, a cell culture, a cell extract, a tissue, a tissue extract, an organ, and an organism. In some embodiments, the kinase is selected from the group comprising Cyclin-dependent kinase 1 (CDK1), Cyclin-dependent kinase 2 (CDK2), Cyclin-dependent-like kinase 5 (CDK5), Dual specificity mitogen-activated protein kinase kinase 1, eIF-2-alpha kinase GCN2, Interleukin-1 receptor-associated kinase 4, MAP/microtubule affinity-regulating kinase 4, Mitogen-activated protein kinase kinase kinase kinase 1, Mitogen-activated protein kinase kinase kinase kinase 2, Mitogen-activated protein kinase kinase kinase kinase 5, Phosphatidylinositol 4,5-bisphosphate 3-kinase catalytic subunit delta, Phosphoglycerate kinase 1, Protein-tyrosine kinase 2-beta, Pyruvate kinase PKM, Receptor-interacting serine/threonine-protein kinase 1, Serine/threonine-protein kinase 4, Serine/threonine-protein kinase MARK2, Serine/threonine-protein kinase tousled-like 2, Thymidylate kinase, Tyrosine-protein kinase Fer, Tyrosine-protein kinase Lck, 5′-AMP-activated protein kinase catalytic subunit alpha-1, Cyclin-dependent-like kinase 6, Dual specificity mitogen-activated protein kinase kinase 2, Interferon-induced, double-stranded RNA-activated protein kinase, Nucleoside diphosphate kinase B, Serine/threonine-protein kinase tousled-like 1,Tyrosine-protein kinase CSK, a diacylglycerol kinase (DGK), and phosphofructokinase, liver type (PFKL).

Accordingly, it is an object of the presently disclosed subject matter to provide compounds of formula (I), (II) and (III), as well as to provide related pharmaceutical compositions and methods. In some embodiments, the compounds are useful in inhibiting kinases. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and Examples.

### BRIEF DESCRIPTIONS OF THE FIGURES

FIG. 1: Schematic diagram showing chemical structures of XO44 and KY-26 probes. The sulfur-fluoride exchange (SuFEx) molecule, XO44, has been used to enrich for kinases in live cells by modifying catalytic lysine residues. The intact modification is difficult to detect with a purified protein, which, without being bound to any one theory, may be due to the stability of the molecule. Modifications were made to the compound by synthesizing the sulfur-triazole exchange (SuTEx) probe analog KY-26, predicted to modify both lysine and tyrosine residues found within kinase active sites. The triazole replaced the fluorine as the leaving group, and an amide bond para to the sulfonyl group was added based on previous studies for SuTEx structure activity relationships²⁶.

FIGS. 2A-2C: Comparison of solution reactivity of XO44 and KY-26 against nucleophiles. Graphs of reaction progress of XO44 (FIG. 2A) and KY-26 (FIG. 2B) with n-butylamine and p-cresol as a function of time. The addition of 1,1,3,3-tetramethylguanidine (TMG) base catalyzed the covalent reaction. The ultraviolet (UV) signals from each compound at the measured time point compared with t=0 is used to quantify the percentage of substrate consumed (percent conversion). Overall, the results indicate KY-26 reacts more rapidly than XO44 with nucleophiles in solution. (FIG. 2C) Image of in-gel fluorescence results for XO44 (sulfur-fluoride exchange (SuFEx)) and KY-26 (sulfur-triazole exchange (SuTEx)) labeled proteins from in situ treatments of Jurkat cells (5 micromolar (μM) probe, 30 minutes (min)). Rhodamine azide tags were appended to probe-modified proteomes by copper-catalyzed azide-alkyne cycloaddition (CuAAC) to detect modified proteins from treated cells. High performance liquid chromatography (HPLC) data (FIGS. 2A and 2B) are representative of n=3 independent experiments.

FIGS. 3A and 3B: KY-26 labeling activity is dependent on molecular recognition. Pair of images of competition of KY-26 labeling of proteins in Jurkat proteomes as assessed by gel-based chemical proteomics. Pretreatment with free adenosine triphosphate (ATP; 10-0.5 millimolar (mM), 30 minutes (min), 37° C.; FIG. 3A) or a non-clickable version of KY-26 (KY-424, at 1 and 0.5 mM, 30 min, 37° C.; FIG. 3B) resulted in concentration-dependent blockade of KY-26 probe labeling (5 micromolar (μM), 30 min). Rhodamine-azide tags were appended to probe-modified proteomes by copper-catalyzed azide-alkyne cycloaddition (CuAAC) to detect modified proteins from KY-26 labeled lysates.

FIG. 4: Image of gel analysis of KY-26 in situ dose response assay. After cell treatment, cells were lysed, and the cytosol fractions were collected. A rhodamine azide tag was added to probe-modified proteins by copper-catalyzed azide-alkyne cycloaddition (CuAAC), which was then detected by in-gel fluorescence scanning. There was no identifiable difference between 12.5 micromolar (μM) or 25 μM conditions.

FIG. 5: Schematic diagram showing reaction of KY-26 probe with a tyrosine-containing synthetic peptide for optimization of liquid chromatography tandem mass spectrometry (LC-MS/MS) conditions for LC-MS/MS identification. The N- and C-termini of a synthetic peptide (SEQ ID NO: 1, indicated by wavy line) were acetylated and amidated, respectively, to minimize side reactions. Containing both a lysine and a tyrosine, the reaction could yield a mixture of products with either modification. The predicted added mass after reaction of KY-26 with tyrosine or lysine was 532.1641 Dalton (Da). After probe labeling, a desthiobiotin tag was appended to the alkyne handle via copper-catalyzed azide-alkyne cycloaddition (CuAAC) for a final added mass of 946.4232 Da. Both reactions were monitored by separating products on a reversed phase analytical column interfaced with an ultraviolet (UV) detector. TMG: 1,1,3,3-Tetramethylguanidine; TCEP: Tris(2-carboxyethyl)phosphine; TBTA: Tris[(1-benzyl-1H-1,2,3-triazol-4-yl)methyl]amine.

FIGS. 6A and 6B: PLRP-S chromatographic separation of a KY-26-modified peptide product. (FIG. 6A) Chromatograms that contain the base-peak chromatogram (BPC), extracted ion chromatograms (EIC) for the vasoactive standard peptide, angiotensin standard peptide, unmodified synthetic peptide (SEQ ID NO: 1) and KY-26 modified synthetic peptide (modification on tyrosine). By using a PLRP-S column, typically used for whole protein separation, KY-26-modified peptide could be separated from the unmodified peptide. An added benefit of PLRP-S columns was that gradients could be shortened from 2-3 hours (hr) to 1 hr, improving the throughput for chemical proteomic experiments. (FIG. 6B) Schematic diagram of chemical structures and observed m/z of probe+1,1,3,3-tetramethylguanidine (TMG) and hydrolyzed probe side products ([M+2H]⁺²) of the peptide modification reactions.

FIGS. 7A and 7B: Collisionally-activated dissociation (CAD) second stage mass spectrometer (MS2) spectrum of KY-26 tyrosine modification of a synthetic peptide (SEQ ID NO: 1). (FIG. 7A) The +4 ion (679.35 m/z) of the peptide containing a KY-26 modification was selected for an MS2 scan in the ion trap. With CAD, predominant fragment ions from the fragmentation of the desthiobiotin tag at 240 and 197 m/z are present in the tandem mass spectrometry (MS/MS) spectra. The peptide and site localization of the KY-26 modification on the tyrosine residue (highlighted in box) is identified but the b and y-ion series are incomplete. (FIG. 7B) Zoomed image of the CAD spectrum with peaks normalized to the largest peak in each subsection.

FIGS. 8A-8C: Electron-transfer dissociation (ETD) second stage mass spectrometer (MS2) spectrum of KY-26-tyrosine modification of a synthetic peptide (SEQ ID NO: 1). (FIG. 8A) The +4 ion (679.35 m/z) of the peptide containing a KY-26 modification was selected for an MS2 scan in the ion trap. Using ETD, instead of desthiobiotin fragments, the loss of the entire KY-26 side chain is observed but not in high abundance. The benefit of ETD is that labile bonds are preserved, and the intact modification can be easily localized, as seen in this spectrum containing a near complete c and z-ion series. (FIG. 8B) Zoomed image of the same ETD MS2 spectrum with peaks normalized to the largest peak in each subsection. (FIG. 8C) Schematic diagram showing origin of MS2 fragment ions arising from a KY-26 modified peptide. A desthiobiotin tag was conjugated to KY-26 after peptide probe labeling. Fragment ions detected with m/z 197 and 240 in collisionally-activated dissociation (CAD) spectra originate from the desthiobiotin affinity tag.

FIG. 9: Exemplary workflow for proteomic identification of KY-26 site of binding on target proteins.

FIGS. 10A and 10B: KY-26 modified proteins and binding sites using higher energy collisional dissociation/electron-transfer dissociation (HCD/ETD) compared with HCD alone. Proteins (FIG. 10A) and peptides (FIG. 10B) were identified using HCD and ETD compared with HCD alone. Five proteins unique to HCD analysis (stress-induced phosphoprotein 1, GTP-binding nuclear protein, septin-7, heat shock protein 90-beta, and MAP/microtubule affinity-regulating kinase 4) were identified. Akin to the total number of proteins identified, additional probe-modified peptides were identified when ETD was included in analyses. These results indicate that ETD analysis substantially improves protein and peptide identification from KY-26-modified peptides. All results presented in the above diagrams are peptides analyzed from tryptic digests.

FIG. 11: Gel image analysis of gateway cloning to generate recombinant human cyclin-dependent kinase 2 (CDK2, containing a FLAG tag) overexpression plasmid. Restriction digest with EcoRI and KpnI (1 hour at 37° C. and 20 minutes at 65° C.) to assess the success of gateway cloning experiments. Digest was followed by plasmid confirmation via sequencing using SP6 and T3 primers (samples CDK2-1-3 and CDK2-2-4).

FIG. 12: Composite gel image showing confirmation of recombinant human cyclin-dependent kinase 2 (CDK2) overexpression in human embryonic kidney (HEK293T) cells. Western blot verifying recombinant human CDK2 (containing FLAG tag) overexpression in HEK239T cell lysates (soluble fractions) using rabbit anti-Flag and goat anti-rabbit DYLIGHT™ 550 antibodies. Transfections were performed for 24- and 48-hour time points. Pyruvate kinase 2 (PKM2) was included as a positive control for the Western blot assay.

FIG. 13: Gel image of KY-26 and TH211 activity-based probe (ABP) labeling of recombinant cyclin-dependent kinase 2 (CDK2, human). Recombinant human CDK2 overexpressed human embryonic kidney (HEK293T) lysates incubated for 30 minutes at 37° C. with various concentrations of TH211 (broad-spectrum kinase ABP) or KY-26 (targeted covalent kinase ABP) for 30- and 60-minute probe labeling condition.

FIG. 14: Gel mage showing confirmation of recombinant cyclin-dependent kinase (CDK2) overexpression in gel-based activity-based probe (ABP) studies. Western blot confirming CDK2 overexpression with rabbit anti-FLAG and goat anti-rabbit 650 antibodies for samples incubated with KY-26 or TH211 probes.

FIG. 15: Gel image showing that target covalent inhibitor KY-424 potently competes KY-26 activity-based probe (ABP) labeling of recombinant human cyclin-dependent kinase 2 (CDK2). Recombinant human CDK2 overexpressed human embryonic kidney (HEK293T) cell lysates were incubated with KY-424 or free adenosine triphosphate (ATP) at indicated concentrations (1, 2, 5, or 10 micromolar (μM)) for 30 minutes at 37° C. Subsequently, samples were incubated with 2.5 μM KY-26 for 30 minutes at 37° C.

FIG. 16: Gel image showing that KY-424 competes KY-26 activity-based probe (ABP) labeling of recombinant human cyclin-dependent kinase (CDK2) in a concentration-dependent manner. Recombinant human CDK2 overexpressed human embryonic kidney (HEK293T) cell lysates were incubated with KY-424 or free adenosine triphosphate (ATP) at the indicated concentrations (40 nanomolar (nM)-1 micromolar (μM KY-424 or 400 μM to 10 millimolar (mM) ATP) for 30 minutes at 37° C. Subsequently, samples were incubated with 2.5 μM KY-26 for 30 minutes at 37° C. Under these treatment conditions, KY-424 shows approximately 50% blockade of KY-26 labeling at 40 nM concentration. Lysates from 24-hour (KY-424 competition) and 48-hour transfections (ATP competition) were used for the depicted studies.

FIG. 17: Gel image showing activation of human T cell lines with sulfur-triazole exchange (SuTEx) ligands. Western blotting for ERK and pERK on SuTEx compounds. Jurkat cell lines were treated with anti-CD3/28 antibody 0.01 milligrams per milliliter (mg/ml; 6.7 nanomolar (nM)) for 15 minutes. The cell lysates were analyzed by Western blotting with ERK and pERK antibodies as described in the Examples. Data are representative of three biological experiments.

FIGS. 18A-18C: In situ activity-based protein profiling (ABPP) analysis of human embryonic kidney (HEK293T) cells expressing recombinant phosphofructokinase, liver type (PFKL) treated with sulfur-triazole exchange (SuTEx) ligands. (FIG. 18A) Image of gel-based ABPP analysis showing inhibitory activity of SuTEx ligand series for blockade of TH211 SuTEx probe labeling, which measures covalent binding of SuTEx ligands to PFKL. (FIG. 18B) Image of gel-based analysis of concentration dependent activity of SuTEx ligand TH220 (5 to 100 nanomolar (nM)) for covalent binding to PFKL. (FIG. 18C) Graph of determination of 50% inhibitory (IC₅₀) concentration for TH220 against PFKL as determined by in situ TH211 chemical proteomic assay. The IC₅₀ was determined to be 305.6 nM.

FIGS. 19A-19C: In vitro activity-based protein profiling (ABPP) analysis of cell lysate of human embryonic kidney (HEK293T) cells expressing recombinant phosphofructokinase, liver type (PFKL); phosphofructokinase, platelet (PFKP); or phosphofructokinase, muscle (PFKM) treated with sulfur-triazole exchange (SuTEx) ligands. (FIG. 19A) Image of gel-based in vitro ABPP analysis showing activity of various SuTEx ligands for competing TH211 probe labeling in cell lysate of cells expressing PFKP. (FIG. 19B) Image of gel-based in vitro analysis of concentration dependence of binding of SuTEx probe TH221 (1 to 200 micromolar (μM)) in cell lysate of cells expressing PFKL (left) and PFKM (right). (FIG. 19C) Image of gel-based in situ ABPP analysis of human embryonic kidney (HEK293T) cells treated with sulfur-triazole exchange (SuTEx) ligands showing inhibitory activity of SuTEx ligand series for blockade of TH211 SuTEx probe labeling.

### DETAILED DESCRIPTION

The presently disclosed subject matter relates to the use of sulfur-heterocycle exchange chemistry for investigating tyrosine and/or lysine reactivity, function and post-translational modification state in proteomes and live cells, as well as for use in preparing pharmaceuticals that target druggable tyrosines and/or lysines. For example, sulfonyl-triazoles have emerged as a new reactive group for covalent modification of tyrosine sites on proteins through sulfur-triazole exchange (SuTEx) chemistry. See PCT International Publication No. WO 2020/214336 to Hsu et al., published Oct. 22, 2020, the disclosure of which is incorporated by reference in its entirety. The presently disclosed subject matter relates, in one aspect, to the further development of this sulfur electrophile and related sulfur-heterocycles as ligands with cellular activity and to the use of SuTEx chemistry in chemical proteomics.

Chemical proteomics is widely used for the global investigation of protein activity and binding of small molecule ligands. Covalent probe binding and inhibition are assessed using liquid chromatography-tandem mass spectrometry (LC-MS/MS) to gain molecular information on targeted proteins and probe-modified sites. The identification of amino acid sites modified by large complex probes, however, is particularly challenging because of the increased size, hydrophobicity, and charge state of peptides derived from modified proteins. These studies are important for direct evaluation of proteome-wide selectivity of inhibitor scaffolds used to develop targeted covalent inhibitors. Hereinbelow are disclosed reverse-phase chromatography and MS dissociation conditions tailored for binding site identification using a clickable covalent kinase inhibitor containing a sulfonyl-triazole reactive group (KY-26). This LC-MS/MS strategy was applied to identify tyrosine and lysine sites modified by KY-26 in functional sites of kinases and other ATP-/NAD-binding proteins (>65 in total) in live cells. The presently disclosed studies provide bioanalytical conditions to guide chemical proteomic workflows for direct target site identification of complex irreversible probes and inhibitors.

In addition, a KY-26 analog, i.e., KY-424, is described. In KY-424, the alkyne group of KY-26 is replaced by an alkyl group. As disclosed herein, KY-424 is a potent ligand and inhibitor for human kinase CDK2. Additional SuTEx ligands with kinase inhibitory activity (e.g., TH-207, TH-208, TH-220, TH-221, TH-223, TH-225, XJ-2-47, XJ-2-65, XJ-2-77, XJ-2-87, XJ-2-105, XJ-2-105, XJ-2-111, XJ-2-115, XJ-2-139, XJ-2-141, SMS-55, SMS-59, SMS-63, SMS-65, SMS-67, SMS-69, SMS-71, SMS-73, SMS-75, SMS-77, SMS-79, SMS-81, SMS-83, SMS-85, and SMS-87) are also described.

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

Throughout the specification and claims, a given chemical formula or name shall encompass all optical and stereoisomers, as well as racemic mixtures where such isomers and mixtures exist, unless as otherwise specifically indicated.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a” “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is leukemia, which in some embodiments is Acute Myeloid Leukemia (AML).

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

In some embodiments, the terms “fragment”, “segment”, or “subsequence” refer to a portion of an amino acid sequence, comprising at least one amino acid, or a portion of a nucleic acid sequence comprising at least one nucleotide. The terms “fragment”, “segment”, and “subsequence” can be used interchangeably herein. In some embodiments, the term “fragment” refers to a compound (e.g., a small molecule compound) that can react with a reactive amino acid residue (e.g., a reactive tyrosine or a reactive lysine) to form an adduct comprising a modified amino acid (e.g., tyrosine or lysine) residue. In some embodiments, the term “fragment” and “ligand” can be used interchangeable. In some embodiments, the term “fragment” refers to that portion of a ligand that remains covalently attached to the reactive amino acid residue.

As used herein, a “functional” biological molecule is a biological molecule in a form in which it exhibits a property by which it can be characterized. A functional enzyme, for example, is one that exhibits the characteristic catalytic activity by which the enzyme can be characterized.

The terms “high throughput protein identification,” “proteomics” and other related terms are used herein to refer to the processes of identification of a large number or (in some cases, all) proteins in a certain protein complement. Post-translational protein modifications and quantitative information can also be assessed by such methods. One example of “high throughput protein identification” is a gel-based process that includes the pre-fractionation and purification of proteins by one-dimensional protein gel electrophoresis. The gel can then be fractionated into several molecular weight fractions to reduce sample complexity, and proteins can be in-gel digested with trypsin. The tryptic peptides are extracted from the gel, further fractionated by liquid chromatography and analyzed by mass spectrometry. In another approach, a sample can be fractionated without using the gels, for example, by protein extraction followed by liquid chromatography. The proteins can then be digested in-solution, and the proteolytic fragments further fractionated by liquid chromatography and analyzed by mass spectrometry.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds. In some embodiments, a ligand can modulate (increase or decrease) a biological activity of biological target, e.g. a protein or peptide. In some embodiments, the ligand can act as an inhibitor.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

As used herein, the term “mass spectrometry” (MS) refers to a technique for the identification and/or quantitation of molecules in a sample. MS includes ionizing the molecules in a sample, forming charged molecules; separating the charged molecules according to their mass-to-charge ratio; and detecting the charged molecules. MS allows for both the qualitative and quantitative detection of molecules in a sample. The molecules can be ionized and detected by any suitable approach known to one of skill in the art. Some examples of mass spectrometry are “tandem mass spectrometry” or “MS/MS,” which are the techniques wherein multiple rounds of mass spectrometry occur, either simultaneously using more than one mass analyzer or sequentially using a single mass analyzer. The term “mass spectrometry” can refer to the application of mass spectrometry to protein analysis. In some embodiments, electrospray ionization (ESI) and matrix-assisted laser desorption/ionization (MALDI) can be used in this context. In some embodiments, intact protein molecules can be ionized by the above techniques, and then introduced to a mass analyzer. Alternatively, protein molecules can be broken down into smaller peptides, for example, by enzymatic digestion by a protease, such as trypsin. Subsequently, the peptides are introduced into the mass spectrometer and identified by peptide mass fingerprinting or tandem mass spectrometry.

As used herein, the term “mass spectrometer” is used to refer an apparatus for performing mass spectrometry that includes a component for ionizing molecules and detecting charged molecules. Various types of mass spectrometers can be employed in the methods of the presently disclosed subject matter. For example, whole protein mass spectroscopy analysis can be conducted using time-of-flight (TOF) or Fourier transform ion cyclotron resonance (FT-ICR) instruments. For peptide mass analysis, MALDI time-of-flight instruments can be employed, as they permit the acquisition of peptide mass fingerprints (PMFs) at high pace. Multiple stage quadrupole-time-of-flight and the quadrupole ion trap instruments can also be used.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The expression “stable isotope labeling by amino acids in cell culture” (SILAC) is used herein to refer to an approach for incorporation of a label into proteins for mass spectrometry (MS)-based quantitative proteomics. SILAC comprises metabolic incorporation of a given “light” or “heavy” form of the amino acid into the proteins. For example, SILAC comprises the incorporation of amino acids with substituted stable isotopic nuclei (e.g. deuterium, ¹³C, ¹⁵N). In an illustrative SILAC experiment, two cell populations are grown in culture media that are identical, except that one of them contains a “light” and the other a “heavy” form of a particular amino acid (for example, ¹²C and ¹³C labeled L-lysine, respectively). When the labeled analog of an amino acid is supplied to cells in culture instead of the natural amino acid, it is incorporated into all newly synthesized proteins. After a number of cell divisions, each instance of the amino acid is replaced by its isotope-labeled analog. Since there is little chemical difference between the labeled amino acid and the natural amino acid isotopes, the cells behave substantially similar to the control cell population grown in the presence of a normal amino acid.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

The term “subject” as used herein can refer to a member of a species for whom analysis, diagnosis, and/or treatment of a disease or disorder using the compositions and methods of the presently disclosed subject matter can be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

As used herein, the term “Western blot,” which can be also referred to as “immunoblot”, and related terms refer to an analytical technique used to detect specific proteins in a sample. The technique uses gel electrophoresis to separate the proteins, which are then transferred from the gel to a membrane (typically nitrocellulose or PVDF) and stained, in membrane, with antibodies specific to the target protein.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. In some embodiments, the alkyl group is “lower alkyl.” “Lower alkyl” refers to an alkyl group having 1 to about 8 carbon atoms (i.e., a C₁₋₈ alkyl), e.g., 1, 2, 3, 4, 5, 6, 7, or 8 carbon atoms. In some embodiments, the alkyl is “higher alkyl.” “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic moiety that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In some embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, carbonyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, triazole, pyrazine, triazine, tetrazole, pyrimidine, quinoline, isoquinoline, indole, carbazole, and the like.

The term “heteroaryl” refers to aryl groups wherein at least one atom of the backbone of the aromatic ring or rings is an atom other than carbon. Thus, heteroaryl groups have one or more non-carbon atoms selected from the group including, but not limited to, nitrogen, oxygen, and sulfur. The term “N-heteroaryl” refers to heteroaryl groups comprising one or more nitrogen atoms, such as, but not limited to, pyrazole, imidazole, tetrazole, and triazole.

As used herein, the term “acyl” refers to an organic carboxylic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The terms “heterocycle” or “heterocyclic” refer to cycloalkyl groups (i.e., non-aromatic, cyclic groups as described hereinabove) wherein one or more of the backbone carbon atoms of a cyclic ring is replaced by a heteroatom (e.g., nitrogen, sulfur, or oxygen). Examples of heterocycles include, but are not limited to, tetrahydrofuran, tetrahydropyran, morpholine, dioxane, piperidine, piperazine, and pyrrolidine.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Alkoxyl” or “alkoxy” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangably with “alkoxyl”.

“Aryloxy” or “aryloxyl” refer to an aryl-O— group, where aryl is as previously described. Exemplary aryloxy groups include phenoxy.

“Aralkyl” refers to an aryl-alkyl- group wherein aryl and alkyl are as previously described and include substituted aryl and substituted alkyl. Exemplary aralkyl groups include benzyl, phenylethyl, and naphthylmethyl.

The term “amino” refers to the —NR′R″ group, wherein R′ and R″ are each independently selected from the group including H and substituted and unsubstituted alkyl, cycloalkyl, aralkyl, and aryl. In some embodiments, the amino group is —NH₂. In some embodiments, R′ and R″, taken together with the nitrogen atom to which they are attached, form a heterocyclic ring having from 4 to 8 atoms (i.e., R′ and R″ together form an alkylene group, wherein optionally one or more carbon atoms of the alkylene group are replaced by an oxygen, sulfur or NH group). Amino groups can be primary (where R′ and R″ are each H), secondary (where one of R′ and R″ is H and the other is substituted or unsubstituted alkyl, cycloalkyl, aralkyl, or aryl), or tertiary (where both R′ and R″ are independently substituted or unsubstituted alkyl, cycloalkyl, aralkyl, or aryl), and in cationic form, may be quaternary (—⁺NH¹(R′)(R″)). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

The term “carbonyl” refers to the —(C═O)— or a double bonded oxygen substituent attached to a carbon atom of a previously named parent group.

The terms “carboxyl” and “carboxylic acid” refer to the —COOH group. The term “carboxylate” can refer to the —COO⁻ group, i.e., to a deprotonated carboxylic acid group.

The terms “halo”, “halide”, or “halogen” as used herein refer to fluoro, chloro, bromo, and iodo groups.

The term “haloalkyl” can be used to refer to an alkyl group wherein one or more hydrogen atoms have been replaced by halo groups.

The term “perhaloalkyl” refers to an alkyl group wherein all of the hydrogen atoms are replaced by halo. Thus, for example, perhaloalkyl can refer to a “perfluroalkyl” group wherein all of the hydrogen atoms of the alkyl group are replaced by fluoro. Perhaloalkyl groups include, but are not limited to, —CF₃.

The terms “hydroxyl” and “hydroxy” refer to the —OH group.

The term “oxo” refers to a compound described previously herein wherein a carbon atom is replaced by an oxygen atom.

The term “cyano” refers to the —CN group.

The term “nitro” refers to the —NO₂ group.

The term “azido” refers to the —N₃ group.

The term ester refers to the —C(═O)OR group, wherein R is selected from alkyl, aralkyl, cycloalkyl, and aryl. Examples of ester groups include, but are not limited to, —C(═O)OCH, —C(═O)OCH₂CH₃, —C(═O)OC(CH₃)₃, and —C(═O)OPh.

The term “amido” as used refer refers to a —C(═O)NR′R″, wherein R′ and R″ are independently selected from H, alkyl, aralkyl, cycloalkyl and aryl, or wherein R′ and R″ together with the nitrogen to which they are attached from a form a heterocyclic ring having from 4 to 8 atoms (i.e., R and R″ together form an alkylene group, wherein optionally one or more carbon atoms of the alkylene group are replaced by an oxygen, sulfur or NH group). Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′ and R″, together with the nitrogen atom to which they are attached, form a heterocyclic structure as in, for example, piperidinocarbonyl, morpholinocarbonyl, thiomorpholinocarbonyl, and piperazinocarbonyl. Amido groups can also be referred to as carbamoyl.

The term “sulfonyl” refers to the —S(═O)₂R group, wherein R is alkyl, substituted alkyl, aralkyl, aryl, or substituted aryl.

A dashed line representing a bond in a chemical formula indicates that the bond can be either present or absent. For example, the chemical structure:

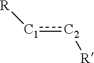

refers to compounds wherein C₁ and C₂ can be joined by either a single or double bond.

A line crossed by a wavy line, e.g., in the structure:

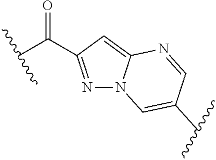 [SMILES: *C(=O)c1cc2ncc(*)cn2n1]

indicates the site where a substituent can bond to another group.

### II. General Considerations

Methods capable of studying endogenous kinase activity and inhibition by small molecules can be useful for the development of potent and selective kinase inhibitors. Several chemical proteomic methods including ATP acyl phosphate activity-based probes⁷⁻⁹ and bead-immobilized kinase inhibitors (kinobeads¹⁰⁻¹¹) can be used for functional profiling of kinases by liquid chromatography-tandem mass spectrometry (LC-MS/MS). However, although widely adopted for parallel analysis of hundreds of kinases, the reagents used for the aforementioned methods are not cell permeable, which precludes their use in live cell studies. Cell permeable affinity-based kinase probes containing a photoreactive diazirine group have been developed, but can show a reduced target scope (˜20 intracellular kinases) in proteomic analyses¹²⁻¹³.

Recently, a cell-permeable pan-kinase probe (XO44) was shown to be effective for chemical proteomic evaluation of the kinome¹⁴. The XO44 kinase probe contains a pyrimidine 3-aminopyrazole group for binding recognition and a sulfonyl-fluoride reactive group¹⁵⁻²⁰ for facilitating covalent modification with lysine residues in kinase active sites. XO44 was capable of profiling dasatinib binding against ˜130 endogenous kinases in intact cells. Target deconvolution using XO44 was accomplished by LC-MS/MS detection of tryptic peptides generated from probe-modified proteins enriched by affinity chromatography. While effective for protein-level identification, the exact amino acid site(s) modified by XO44 on these kinase targets could not be ascertained from tryptic peptide digest analyses. Site of binding analyses using XO44 were pursued but yielded LC-MS/MS data that provided for identification of only a single binding site on SRC kinase (K295)¹⁴. In this regard, it can be noted that probe reaction, especially larger probes such as XO44, at an amino acid site increases the molecular mass, hydrophobicity, and charge state of resulting probe-modified peptides, which can complicate LC-MS/MS identification. To address these structurally complex probe adducts, custom proteomic workflows have been developed to understand LC-MS/MS fragmentation mechanisms and increase the ability for binding site identifications of covalent kinase inhibitors that target cysteines²¹⁻²².

The presently disclosed subject matter relates in one aspect to the identification of chromatography and LC-MS/MS fragmentation conditions tailored for chemical proteomic evaluation of covalent kinase probes that produce large complex adducts with a target site. According to one aspect, a sulfonyl-triazole²³ analog of XO44 (referred to as KY-26) was synthesized that contains a more reactive triazole leaving group in order to modify tyrosine and lysine residues, and thereby increase capability to capture peptides that contain binding site residues. The ability to identify KY-26-modified sites on kinases and other target proteins was greatly improved by (1) replacing the conventional C18 stationary phase in analytical HPLC columns with PLRP-S (polystyrene/divinylbenzene) media, (2) including electron transfer dissociation (ETD) in the MS data acquisition methods and (3) utilizing hydrophilic interaction liquid chromatography (HILIC) to reduce contaminant ions for LC-MS/MS analyses. These modifications provided for the identification of nearly 70 protein targets and corresponding binding sites of KY-26 as proof of concept for improved LC-MS/MS methodology for chemical proteomics.

The presently disclosed subject matter further relates, in some aspects, to the development of additional kinase ligands (e.g., covalent kinase inhibitors) that can be used to modulate kinase biological activity. For example, in some aspects, the presently disclosed subject matter relates to KY-424, an analog of KY-26, and its use as a ligand in chemical proteomics and as an inhibitor for human kinase CDK2.

The presently disclosed subject matter further provides for additional kinase ligands (e.g., kinase inhibitors) that comprise sulfonyl-N-heteroaryl groups (e.g., sulfonyl-triazole groups). These compounds include, for example, TH-207, TH-208, TH-220, TH-221, TH-223, TH-225, XJ-2-47, XJ-2-65, XJ-2-77, XJ-2-87, XJ-2-105, XJ-2-105, XJ-2-111, XJ-2-115, XJ-2-139, XJ-2-141, SMS-55, SMS-59, SMS-63, SMS-65, SMS-67, SMS-69, SMS-71, SMS-73, SMS-75, SMS-77, SMS-79, SMS-81, SMS-83, SMS-85, and SMS-87. In some embodiments, the compound has use as a ligand (e.g., an inhibitor) for diacylglycerol kinase (DGK), which catalyzes the phosphorylation of diacylglycerol to form phosphatidic acid. DGK inhibitors can be used to activate T cells, promote T cell proliferation and can have anti-tumor activity. In some embodiments, the compound has use as a ligand (e.g., an inhibitor) of a phosphofructokinase (e.g., ATP-dependent 6-phosphofructokinase, liver type (PFKL)). Such inhibitors also have potential as cancer treatments.

### III. Ligands

Small molecules can serve as versatile tools for perturbing the functions of proteins in biological systems. Many human proteins currently lack selective chemical ligands; and there are several classes of proteins that are currently considered as undruggable. Covalent ligands (also referred to herein as “fragments”) offer a strategy to expand the landscape of proteins amenable to targeting by small molecules. In some instances, covalent ligands combine features of recognition and reactivity, thereby providing for the targeting of sites on proteins that are difficult to address by reversible binding interactions alone.

As noted hereinabove, sulfonyl-triazoles have emerged as a new class of reactive compounds for covalent modification of tyrosine and/or lysine sites on proteins through sulfur-triazole exchange (SuTEx) chemistry. See PCT International Publication No. WO 2020/214336 to Hsu et al., published on Oct. 22, 2020, the disclosure of which is incorporated by reference in its entirety. For example, Scheme 1, below, shows the reaction of a SuTEx compound (e.g., a SuTEx ligand or a SuTEx probe) with a protein having a reactive tyrosine (Y) or lysine (K). The SuTEx compound comprises a sulfur electrophile, i.e., a sulfonyl group directed attached to a nitrogen atom of a nitrogen-containing heteroaryl group. The nitrogen-containing heteroaryl group acts as a leaving group in the reaction of the compound with the nucleophilic phenol or amine of the tyrosine or lysine, resulting in a modified protein where a modified tyrosine or lysine residue is covalently attached to the SuTEx compound sulfonyl group, which is itself directly attached to an adduct group (AG) or “fragment” from the original SuTEx compound. AGs of SuTEx ligands can include a variety of optionally substituted alkyl, cycloalkyl (including heterocyclic), aryl (including heteroaryl), and aralkyl groups, while SuTEx “probes” can contain AG groups that comprise an alkyne group, a fluorophore moiety, a detectable moiety, or a combination thereof. For instance, the alkyne group of a SuTEx probe can be used as the site of reaction of a protein modified by the probe with a detectable moiety. While the nitrogen-containing heteroaryl group shown in the SuTEx compound of Scheme 1 is a 1,2,4-triazole or a 1,2,3-triazole substituted by an R group (i.e., H or an aryl group substituent), SuTEx compounds can also include other nitrogen-containing heteroaryl groups as the leaving group, e.g., pyrazole, imidazole, or tetrazole, each of which can be optionally substituted by one or more aryl group substituents.

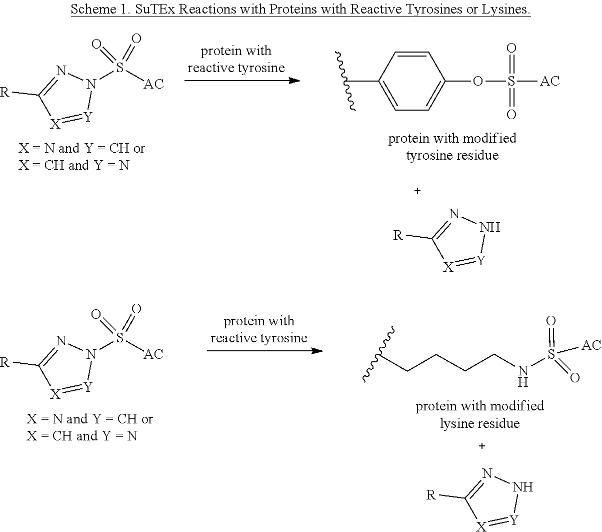

In some embodiments, a ligand of the presently disclosed subject matter can compete with a probe compound described herein for binding with a reactive tyrosine and/or lysine residue. In some embodiments, the ligand molecule comprises a fragment moiety that facilitates interaction of the compound with a reactive tyrosine and/or lysine residue. In some cases, the ligand comprises a fragment moiety that facilitates hydrophobic interaction, hydrogen bonding, or a combination thereof. The presently disclosed ligands are typically non-naturally occurring and/or form non-naturally occurring products after reaction with the phenol group of a tyrosine residue of a tyrosine-containing protein or an amino group of a lysine residue of a lysine containing protein.

The presently disclosed subject matter relates, in one aspect, to the further development of SuTEx ligands. In some embodiments, the presently disclosed subject matter provides a compound (e.g., a tyrosine-reactive and/or lysine-reactive ligand compound) having a structure of formula (I), (II), or (III):

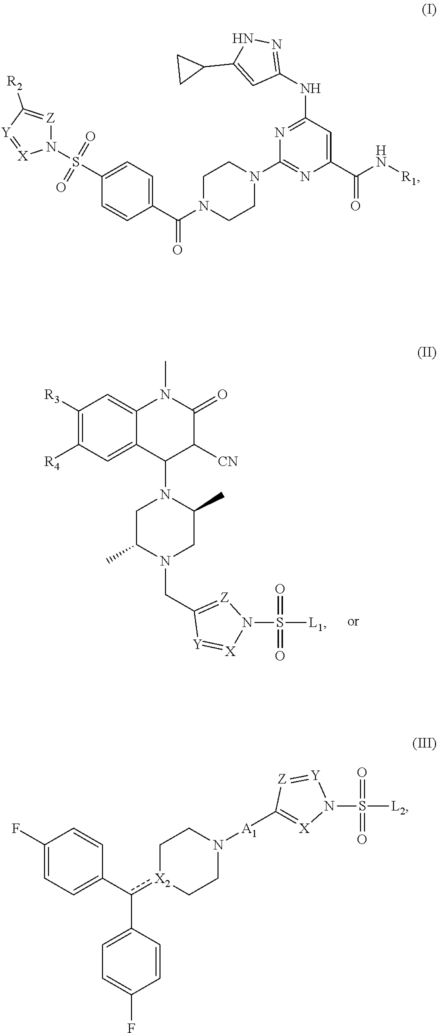

wherein: [symbol] is a double or single bond; X, Y, and Z are independently C or N, subject to the proviso that at least one of X, Y, and Z is N (e.g., where the ring comprising X, Y, and Z is an imidazole, a triazole, a pyrazole, or a tetrazole); X₂ is C or N, subject to the proviso that when [symbol] is a single bond, X₂ is N and when [symbol] is a double bond, X₂ is C; R₁ is selected from alkyl, cycloalkyl, aralkyl, aryl, or heteroaryl, which alkyl, cycloalkyl, aralkyl, aryl or heteroaryl is optionally substituted with one or more alkyl or aryl group substituent (e.g., selected from the group comprising halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene) and subject to the proviso that R₁ does not comprise an alkyne group; R₂ is alkyl, cycloalkyl, aralkyl, or aryl, which alkyl, cycloalkyl, aralkyl, or aryl is optionally substituted with one or more alkyl or aryl group substituent (e.g., selected from the group comprising halo, cyano, alkyl, alkoxy, perhaloalkyl, perhaloalkoxy, cycloalkyl, aralkyl, aryl, and amido); R₃ and R₄ are independently selected from H, halo, alkyl, perhaloalkyl, and alkoxy; L₁ and L₂ are each alkyl, cycloalkyl, aralkyl, aryl, or heteroaryl, which alkyl, cycloalkyl, aralkyl, aryl or heteroaryl is optionally substituted with one or more alkyl or aryl group substituent (e.g., selected from the group comprising halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene), and subject to the proviso that L₁ and L₂ do not comprise an alkyne group; and A₁ is selected from the group consisting of ethylene,

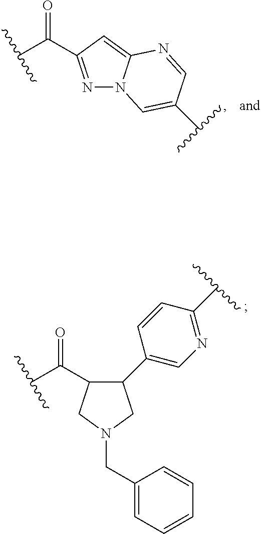 [SMILES: *C(=O)c1cc2ncc(*)cn2n1 ; *C(=O)C1CN(Cc2ccccc2)CC1c1ccc(*)nc1]

or a pharmaceutically acceptable salt or solvate thereof.

In some embodiments, the compound having a structure of formula (I), (II), or (III) binds to one or more kinase. In some embodiments, the compound covalently modifies the kinase. In some embodiments, the compound modulates the activity of the kinase, e.g., inhibits one or more biological activity of the kinase. Accordingly, in some embodiments, the compound having a structure of formula (I), (II), or (III) is a kinase inhibitor.

In some embodiments, the compound has a structure of formula (I):

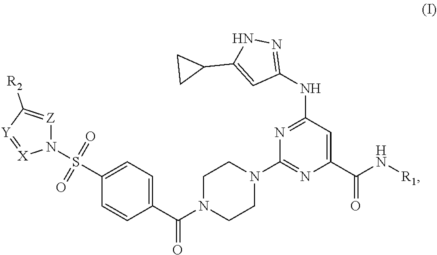

wherein: X, Y, and Z are independently C or N, subject to the proviso that at least one of X, Y, and Z are N (e.g., wherein two of X, Y, and Z are N); R₁ is alkyl, cycloalkyl, aralkyl, aryl, or heteroaryl, which alkyl, cycloalkyl, aralkyl, aryl or heteroaryl is optionally substituted with one or more alkyl or aryl group substituent (e.g., selected from the group comprising halo, cyano, alkyl, alkoxy, perhaloalkyl (e.g., perfluoroalkyl, such as —CF₃), perhaloalkoxy, cycloalkyl (e.g., cyclopropyl), aralkyl, aryl, and amido, or wherein two alkyl or aryl group substituents together form alkylene or substituted alkylene), and subject to the proviso that R₁ does not comprise an alkyne group; and R₂ is alkyl, cycloalkyl, aralkyl, or aryl, which alkyl, cycloalkyl, aralkyl, or aryl is optionally substituted with one or more alkyl or aryl group substituent (e.g., selected from the group comprising halo, cyano, alkyl, alkoxy, perhaloalkyl, perhaloalkoxy, cycloalkyl, aralkyl, aryl, and amido); or a pharmaceutically acceptable salt or solvate thereof.

In some embodiments, at least two of X, Y, and Z are N. In some embodiments, two of X, Y, and Z are N (and the compound of formula (I) comprises a triazole). In some embodiments, the compound comprises a 1,2,3-triazole. In some embodiments, the compound comprises a 1,2,4-triazole. In some embodiments, Y and Z are each N and X is C.

In some embodiments, R₁ is alkyl (e.g., C₁-C₁₂ alkyl). In some embodiments, R₁ is C₁-C₆ alkyl. In some embodiments, R₁ is propyl. In some embodiments, R₁ is n-propyl (i.e., —CH₂CH₂CH₃).

In some embodiments, R₂ is aryl or substituted aryl. In some embodiments, R₂ is phenyl or substituted phenyl. In some embodiments, R₂ is phenyl.

In some embodiments, the compound of formula (I) is 6-((5-cycloproypyl-1H-pyrazol-3-yl)amino)-2-(4-(4-((3-phenyl-1H-1,2,4-triazol-1-yl)sulfonyl)-benzoyl)piperazin-1-yl)-N-propylpyrimidine-4-carboxamide) (also referred to herein as “KY-424”), or a pharmaceutically acceptable salt or solvate thereof. In some embodiments, the compound of formula (I) inhibits a cyclin-dependent kinase (CDK). In some embodiments, the CDK is CDK2.

In some embodiments, the compound is a compound having a structure of formula (II).

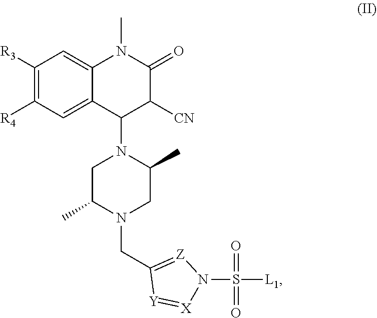

wherein: X, Y, and Z are independently C or N, subject to the proviso that at least one of X, Y, and Z are N (e.g., wherein two of X, Y, and Z are N); R₃ and R₄ are independently selected from the group comprising H, halo, alkyl, perhaloalkyl, and alkoxy; and L₁ is alkyl, cycloalkyl, aralkyl, aryl, or heteroaryl, which alkyl, cycloalkyl, aralkyl, aryl or heteroaryl is optionally substituted with one or more alkyl or aryl group substituent (e.g., selected from the group comprising halo, cyano, alkyl, alkoxy, perhaloalkyl (e.g., perfluoroalkyl), perhaloalkoxy (e.g., perfluoroalkoxy), cycloalkyl (e.g., cyclopropyl), aralkyl (e.g., benzyl), aryl, and amido, or wherein two alkyl or aryl group substituents together form alkylene or substituted alkylene), and subject to the proviso that L₁ does not contain an alkyne group; or a pharmaceutically acceptable salt or solvate thereof.

In some embodiments, two of X, Y, and Z are N and the compound of formula (II) comprises a triazole. In some embodiments, the triazole is a 1,2,3-triazole. In some embodiments, the triazole is a 1,2,4-triazole. In some embodiments, X and Y are N and Z is C.

In some embodiments, R₃ and R₄ are independently selected from the group comprising H, halo (e.g., F, Cl, Br, or I) and alkoxy (e.g., C₁-C₆ alkoxy). In some embodiments, at least one of R₃ and R₄ is H. In some embodiments, R₃ is H or alkoxy and R₄ is H or halo. In some embodiments, R₃ is H or methoxy and R₄ is H, Br, or F.

In some embodiments, L₁ is selected from the group comprising alkyl, substituted alkyl, cycloalkyl, aralkyl, phenyl, substituted phenyl, thiazole, and substituted thiazole. In some embodiments, L₁ is selected from the group comprising isopropyl, isobutyl, cyclopropyl, 2-methoxyethyl, 3,3,3-trifluoropropyl, benzyl, phenyl, p-fluorophenyl, p-bromophenyl, p-cyanophenyl, and dimethylthiazole.

In some embodiments, the compound of formula (II) is selected from the group comprising: 4-((2S,5R)-2,5-dimethyl-4-((1-phenylsulfonyl)-1H-1,2,3-triazol-4-yl) methyl)piperazin-1-yl)-1-methyl-2-oxo-1,2-dihydroquinoline-3-carbonitrile (also referred to herein as “SMS-55”); 4-((2S,5R)-4-((cyclopropylsulfonyl)-1H-1,2,3-triazol-4-yl) methyl) 2,5-dimethyl piperazin-1-yl)-1-methyl-2-oxo-1,2-dihydroquinoline-3-carbonitrile (also referred to herein as “SMS-59”); 4-((2S,5R)-4-((isopropylsulfonyl)-1H-1,2,3-triazol-4-yl)methyl)-2,5-dimethyl piperazin-1-yl)-1-methyl-2-oxo-1,2-dihydroquinoline-3-carbonitrile (also referred to herein as “SMS-63”); 4-((2S,5R)-4-((1-((4-bromophenyl)sulfonyl)-1H-1,2,3-triazol-4-yl)methyl)-2,5-dimethylpiperazin-1-yl)-1-methyl-2-oxo-1,2-dihydroquinoline-3-carbonitrile (also referred to herein as “SMS-65”); 4-((2S,5R)-4-((1-((4-fluorophenyl)sulfonyl)-1H-1,2,3-triazol-4-yl)methyl)-2,5-dimethylpiper-azin-1-yl)-1-methyl-2-oxo-1,2-dihydroquinoline-3-carbonitrile (also referred to herein as “SMS-67”); 4-((2S,5R)-4-((1-((4-cyanophenyl)sulfonyl)-1H-1,2,3-triazol-4-yl)methyl)-2,5-dimethylpiperazin-1-yl)-1-methyl-2-oxo-1,2-dihydro-quinoline-3-carbonitrile (also referred to herein as “SMS-69”); 4-((2S,5R)-4-((1-((2,4-dimethylthiazol-5-yl)sulfonyl)-1H-1,2,3-triazol-4-yl) methyl)-2,5-dimethylpiperazin-1-yl)-1-methyl-2-oxo-1,2-dihydroquinoline-3-carbo-nitrile (also referred to herein as “SMS-71”); 4-((2S,5R)-4-((1-benzylsulfonyl)-1H-1,2,3-triazol-4-yl)methyl)-2,5-dimethyl piperazin-1-yl)-1-methyl-2-oxo-1,2-dihydroquinoline-3-carbonitrile (also referred to herein as “SMS-73”); 4-((2S,5R)-4-((isobutylsulfonyl)-1H-1,2,3-triazol-4-yl)methyl)-2,5-dimethyl piperazin-1-yl)-1-methyl-2-oxo-1,2-dihydroquinoline-3-carbonitrile (also referred to herein as “SMS-75”); 4-((2S,5R)-4-((2-methoxyethyl)sulfonyl)-1H-1,2,3-triazol-4-yl)methyl)-2,5-dimethyl piperazin-1-yl)-1-methyl-2-oxo-1,2-dihydroquin-oline-3-carbonitrile (also referred to herein as “SMS-77”); 4-((2S,5R)-2,5-dimethyl-4-((1-((3,3,3-trifluoropropyl)sulfonyl)-1H-1,2,3-triazol-4-yl) methyl)-piperazin-1-yl)-1-methyl-2-oxo-1,2-dihydroquinoline-3-carbonitrile (also referred to herein as “SMS-79”); 6-bromo-4-((2S,5R)-2,5-dimethyl-4-((1-phenylsulfonyl)-1H-1,2,3-triazol-4-yl) methyl)piperazin-1-yl)-1-methyl-2-oxo-1,2-dihydroquinoline-3-carbonitrile (also referred to herein as “SMS-81”); 4-((2S,5R)-2,5-dimethyl-4-((1-phenylsulfonyl)-1H-1,2,3-triazol-4-yl)methyl) piperazin-1-yl)-6-fluoro-1-methyl-2-oxo-1,2-dihydroquinoline-3-carbonitrile (also referred to herein as “SMS-83”); 6-fluoro-4-((2S,5R)-4-((isopropylsulfonyl)-1H-1,2,3-triazol-4-yl)methyl)-2,5-dimethyl-piperazin-1-yl)-1-methyl-2-oxo-1,2-dihydro-quinoline-3-carbonitrile (also referred to herein as “SMS-85”); 4-((2S,5R)-2,5-dimethyl-4-((1-phenylsulfonyl)-1H-1,2,3-triazol-4-yl)methyl) piperazin-1-yl)-7-methoxy-1-methyl-2-oxo-1,2-dihydroquinoline-3-carbonitrile (also referred to herein as “SMS-87”); and pharmaceutically acceptable salts and solvates thereof. In some embodiments, the compound of formula (II) inhibits a DGK (e.g., DGK alpha (DGKA) or DKG zeta (DGKZ).

In some embodiments, the compound has a structure of formula (III):

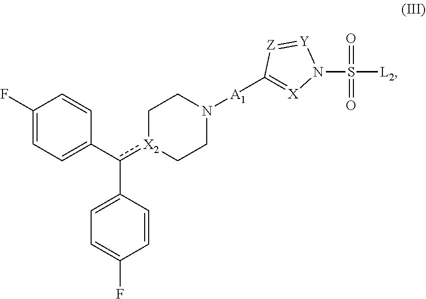

wherein: [symbol] is a double or single bond; X, Y, and Z are independently C or N, subject to the proviso that at least one of X, Y, and Z is N (e.g., where two of X, Y, and Z are N); X₂ is C or N, subject to the proviso that when [symbol] is a single bond, X₂ is N and when [symbol] is a double bond, X₂ is C; L₂ is alkyl, cycloalkyl, aralkyl, aryl, or heteroaryl, which alkyl, cycloalkyl, aralkyl, aryl or heteroaryl is optionally substituted with one or more alkyl or aryl group substituent selected from the group comprising halo, cyano, alkyl, alkoxy, perhaloalkyl (e.g., perfluoroalkyl), perhaloalkoxy (e.g., perfluoroalkoxy), cycloalkyl, aralkyl, aryl, and amido, or wherein two alkyl or aryl group substituents together form alkylene or substituted alkylene, and subject to the proviso that L₂ does not comprise an alkyne group; and A₁ is selected from the group consisting of ethylene,

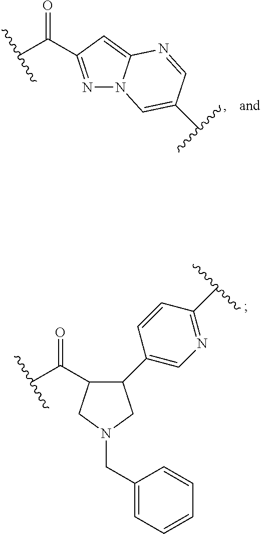 [SMILES: *C(=O)c1cc2ncc(*)cn2n1 ; *C(=O)C1CN(Cc2ccccc2)CC1c1ccc(*)nc1]

or a pharmaceutically acceptable salt or solvate thereof.

In some embodiments, at least two of X, Y, and Z are N. In some embodiments, two of X, Y, and Z are N and the compound of formula (III) comprises a triazole. In some embodiments, the triazole is a 1,2,3-triazole. In some embodiments, the triazole is a 1,2,4-triazole. In some embodiments, Y and Z are each N and X is C.

In some embodiments, L₂ is selected from the group comprising cycloalkyl, aryl, substituted aryl, thiazole, and substituted thiazole. In some embodiments, L₂ is selected from the group comprising cyclopropyl, phenyl, substituted phenyl, thiazole, and dimethylthiazole. In some embodiments, L₂ is substituted phenyl. In some embodiments, L₂ is phenyl substituted by one or two substituents selected from the group comprising alkyl, alkoxy, halo, and amido. In some embodiments, L₂ is phenyl substituted with two substituents that together form an alkylene or substituted alkylene (e.g., substituted or unsubstituted propylene or butylene, that together with the phenyl to which they are attached form a fused ring structure). In some embodiments, the alkylene is alkylene where one or two carbon atoms are replaced by oxygen. In some embodiments, the alkylene is substituted by one or more alkyl (e.g., methyl) or halo (e.g., F) groups.

In some embodiments, A₁ is ethylene.

In some embodiments, the compound of formula (III) is selected from the group comprising 4-((4-(2-(4-(Bis(4-fluorophenyl)methylene)piperidin-1-yl)ethyl)-1H-1,2,3-triazol-1-yl)sulfonyl)-N-propylbenzamide (also referred to herein as “TH225”); 4-(Bis(4-fluorophenyl)methylene)-1-(2-(1-tosyl-1H-1,2,3-triazol-4-yl)ethyl)-piperidine (also referred to herein as “TH207”); 4-(bis(4-fluorophenyl)methylene)-1-(2-(1-(cyclopropylsulfonyl)-1H-1,2,3-triazol-4-yl)ethyl)-piperidine (also referred to herein as “TH223”); 1-(Bis(4-fluorophenyl)methyl)-4-(2-(1-tosyl-1H-1,2,3-triazol-4-yl)ethyl)piperazine (also referred to herein as “TH208”); 4-(Bis(4-fluorophenyl)methylene)-1-(2-(1-tosyl-1H-1,2,3-triazol-4-yl)ethyl)-piperidine (also referred to herein as “TH220”); 4-(Bis(4-fluorophenyl)methylene)-1-(2-(1-((4-fluorophenyl)sulfonyl)-1H-1,2,3-triazol-4-yl)ethyl)-piperidine (also referred to herein as “TH221”); (4-(Bis(4-fluorophenyl)methylene)piperidin-1-yl)(6-(1-((4-methoxyphenylsulfonyl)-1H-1,2,3-triazol-4-yl)pyrazolo[1,5-a]pyrimidin-2-yl)methanone (also referred to herein as “XJ-2-47”); (1-Benzyl-4-(6-(1-((2,4-dimethylthiazol-5-yl)sulfonyl)-1H-1,2,3-triazol-4-yl)pyridin-3-yl)pyrrolidin-3-yl)(4-(bis(4-fluorophenyl)-methylene)piperidin-1-yl)methanone (also referred to herein as “XJ-2-65”); 4-(Bis(4-fluorophenyl)methylene)-1-(2-(1-((2,3-dihydrobenzo[b][1,4]dioxin-6-yl)sulfonyl)-1H-1,2,3-triazol-4-yl)ethyl)piperidine (also referred to herein as “XJ-2-77”); 1-(2-(1-(Benzo[d][1,3]dioxol-5-ylsulfonyl)-1H-1,2,3-triazol-4-yl)ethyl)-4-(bis(4-fluorophenyl)-methylene)piperidine (also referred to herein as “XJ-2-87”); 5-((4-(2-(4-(Bis(4-fluorophenyl)methylene)piperidin-1-yl)ethyl)-1H-1,2,3-triazol-1-yl)sulfonyl)-2,4-dimethyl-thiazole (also referred to herein as “XJ-2-105”); 4-(Bis(4-fluorophenyl)methylene)-1-(2-(1-((2,3-dihydrobenzofuran-6-yl)sulfonyl)-1H-1,2,3-triazol-4-yl)ethyl)piperidine (also referred to herein as “XJ-2-111”); 4-(Bis(4-fluorophenyl)methylene)-1-(2-(1-((2,2-difluorobenzo-[d][1,3]dioxol-5-yl)sulfonyl)-1H-1,2,3-triazol-4-yl)ethyl)piperidine (also referred to herein as “XJ-2-115”); 4-(Bis(4-fluorophenyl)methylene)-1-(2-(1-((2,4-dimethoxyphenyl)sulfonyl)-1H-1,2,3-triazol-4-yl)ethyl)piperidine (also referred to herein as “XJ-2-139”); 4-(Bis(4-fluorophenyl)methylene)-1-(2-(1-((2-methoxyphenyl)sulfonyl)-1H-1,2,3-triazol-4-yl)ethyl)-piperidine (also referred to herein as “XJ-2-141”); and pharmaceutically acceptable salts and solvates thereof. In some embodiments, the compound is TH220. In some embodiments, the compound is TH207. In some embodiments, the compound of formula (III) inhibits a phosphofructokinase (e.g., PFKL).

As noted above, in some embodiments, the presently disclosed compounds can be provided as a pharmaceutically acceptable salt. As used herein, the term “physiologically acceptable salt” means a salt form of the recited compound which is compatible with any other ingredients of a pharmaceutical composition and/or which is not deleterious to a subject to which the composition is to be administered (e.g., a human or other mammalian subject).

Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N, N′-dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like.

Examples of metal salts include lithium, sodium, potassium, and magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

In some embodiments, the presently disclosed compounds can further be provided as a solvate.

In some embodiments, the presently disclosed subject matter encompasses the preparation and use of pharmaceutical compositions comprising a ligand compound as described herein. The pharmaceutical compositions can be useful for treatment of diseases and disorders as would be apparent upon review of the instant disclosure as an active ingredient. Such a pharmaceutical composition can comprise, consist essentially of, or consist of the active ingredient alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art. Thus, in some embodiments, the presently disclosed subject matter provides a pharmaceutical composition comprising (a) a compound of formula (I), (II), or (III), or a pharmaceutical salt and/or solvate thereof; and (b) a pharmaceutically acceptable carrier.

The compositions of the presently disclosed subject matter can comprise at least one active ingredient (e.g., at least one compound of formula (I), (II), or (III) or a pharmaceutically acceptable salt or solvate thereof), one or more acceptable carriers, and optionally other active ingredients or therapeutic agents.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions can also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter or pharmaceutical compositions comprising these compositions can be administered so that the compositions can have a physiological effect. Administration can occur enterally or parenterally; for example, orally, rectally, intracisternally, intravaginally, intraperitoneally, locally (e.g., with powders, ointments or drops), or as a buccal or nasal spray or aerosol. Parenteral administration is an approach. Particular parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, and direct application to the target area, e.g., intratumoral injection, for example by a catheter or other placement device.

Where the administration of the composition is by injection or direct application, the injection or direct application can be in a single dose or in multiple doses. Where the administration of the compound is by infusion, the infusion can be a single sustained dose over a prolonged period of time or multiple infusions.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially and/or socially relevant mammals such as cattle, pigs, horses, sheep, cats, and dogs, birds including commercially and/or socially relevant birds such as chickens, ducks, geese, parrots, and turkeys.

A pharmaceutical composition of the presently disclosed subject matter can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition can comprise between 0.1% and 100% (w/w) active ingredient.

In addition to the active ingredient, a pharmaceutical composition of the presently disclosed subject matter can further comprise one or more additional pharmaceutically active agents.

Controlled- or sustained-release formulations of a pharmaceutical composition of the presently disclosed subject matter can be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro (1990) Remington's Pharmaceutical Sciences, 18th ed., Mack Pub. Co., Easton, Pennsylvania, United States of America and/or Gennaro (ed.) (2003) Remington: The Science and Practice of Pharmacy, 20th edition Lippincott, Williams & Wilkins, Philadelphia, Pennsylvania, United States of America, each of which is incorporated herein by reference.

The compositions may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Other approaches include but are not limited to nanosizing the composition comprising a ligand compound as described herein to be delivered as a nanoparticle intravenously, intraperitoneal injection, or implanted beads with time release of a ligand compound as described herein.

Suitable preparations include injectables, either as liquid solutions or suspensions, however, solid forms suitable for solution in, suspension in, liquid prior to injection, may also be prepared. The preparation may also be emulsified, or the compositions encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a cell or a tissue of a subject. In some embodiments, this kit comprises a (in some embodiments sterile) solvent suitable for dissolving or suspending the composition of the presently disclosed subject matter prior to administering the compound to the subject and/or a device suitable for administering the composition such as a syringe, injector, or the like or other device as would be apparent to one of ordinary skill in the art upon a review of the instant disclosure.

As used herein, an “instructional material” includes a publication, a recording, a diagram, or any other medium of expression which can be used to communicate the usefulness of the composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter can, for example, be affixed to a container which contains a composition of the presently disclosed subject matter or be shipped together with a container which contains the composition. Alternatively, the instructional material can be shipped separately from the container with the intention that the instructional material and the composition be used cooperatively by the recipient.

### IV. Probes

In some embodiments, the presently disclosed subject matter provides a probe compound or provides for the use of a probe compound (e.g., a small molecule probe compound) that comprises a reactive moiety (i.e., a reactive electrophilic moiety) which can interact with the phenol group of a tyrosine residue of a tyrosine-containing protein and/or a nucleophilic group of the side chain of another amino acid residue, such as the primary amino group of a lysine residue of a lysine-containing protein. In some instances, the probe reacts with a tyrosine and/or lysine residue to form a covalent bond. Typically, the probe is a non-naturally occurring molecule, or forms a non-naturally occurring product (i.e., a “modified” protein or adduct) after reaction with the phenol group of a tyrosine residue of a tyrosine containing protein or other nucleophilic group of an amino acid, e.g., the primary amino group of a lysine residue. In some instances, the phenol group of a reactive tyrosine in the tyrosine-containing protein is connected to the small molecule fragment moiety via an —O—S(═O)₂— bond. In some instances, the primary amino group of a reactive lysine in a lysine-containing protein is connected to a small molecule fragment moiety via an —NH—S(═O)₂— bond.

For example, in some embodiments, the presently disclosed subject matter provides a probe compound that has a structure of formula:

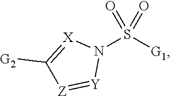

wherein: G₁ is a monovalent moiety comprising an alkyne moiety, a fluorophore moiety, a detectable labeling group, or a combination thereof, X, Y, and Z are independently selected from N and C, subject to the proviso that at least one of X, Y, and Z is N; and G₂ is an aryl group substituent, e.g., alkyl, substituted alkyl, aralkyl, substituted aralkyl, aryl, or substituted aryl. In some embodiments, two of X, Y, and Z are N and the probe comprises a sulfonyl-triazole group.

Thus, in some embodiments, the probe compound of can form a protein or peptide comprising at least one modified reactive tyrosine residue, wherein the modified reactive tyrosine comprises a structure:

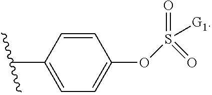

In some embodiments, the probe compound can form a protein or peptide comprising at least one modified reactive lysine residue, wherein the modified reactive lysine residue comprises a structure:

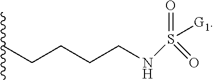

The fluorophore of G₁ can be any suitable fluorophore. In some embodiments, the fluorophore is selected from the group including, but not limited to, rhodamine, rhodol, fluorescein, thiofluorescein, aminofluorescein, carboxyfluorescein, chlorofluorescein, methylfluorescein, sulfofluorescein, aminorhodol, carboxyrhodol, chlororhodol, methylrhodol, sulforhodol; aminorhodamine, carboxyrhodamine, chlororhodamine, methylrhodamine, sulforhodamine, thiorhodamine, cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiazole derivatives, pyridyloxazole, nitrobenzoxadiazole, benzoxadiazole, pyren derivatives, cascade blue, oxazine derivatives, Nile red, Nile blue, cresyl violet, oxazine 170, acridine derivatives, proflavin, acridine orange, acridine yellow, arylmethine derivatives, auramine, crystal violet, malachite green, tetrapyrrole derivatives, porphin, phtalocyanine, bilirubin 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl)maleimide, stilbenes, pyrenes, 6-FAM (Fluorescein), 6-FAM (NHS Ester), 5(6)-FAM, 5-FAM, Fluorescein dT, 5-TAMRA-cadavarine, 2-aminoacridone, HEX, JOE (NHS Ester), MAX, TET, ROX, and TAMRA.

In some embodiments, G₁ comprises a fluorophore moiety. In some cases, G₁ is obtained from a compound library. In some cases, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library.

In some embodiments, the detectable labeling moiety is selected from the group comprising a member of a specific binding pair (e.g., biotin:streptavidin, antigen-antibody, nucleic acid:nucleic acid), a bead, a resin, a solid support, or a combination thereof. In some embodiments, the detectable labeling group is a biotin moiety, a streptavidin moiety, bead, resin, a solid support, or a combination thereof. In some embodiments, the detectable labeling moiety comprises biotin or a derivative thereof (e.g., desthiobiotin). In some embodiments, the detectable labeling moiety comprises a heavy isotope (i.e., ¹³C).

In some embodiments, G₁ comprises an aryl group directly attached to the sulfur atom of the sulfonyl group. Thus, in some embodiments, G₁ has a structure —Ar₂-G₃, wherein Ar₂ is aryl and G₃ is a monovalent moiety comprising an alkyne moiety, a fluorophore moiety, a detectable labeling group, or a combination thereof. In some embodiments, Ar₂ is selected from the group comprising phenyl, naphthyl, and pyridyl. In some embodiments, Ar₂ is phenyl. In some embodiments, G₃ comprises or consists of —C≡CH, -alkylene-C≡CH, —O-alkylene-C≡CH (e.g., —O—CH₂—C≡CH), or —C(═O)—NH-alkylene-C≡CH (e.g., C(═O)—NH—CH₂—C≡CH). In some embodiments, the alkylene group is a C1-C5 alkylene group. In some embodiments, the alkylene group is methylene.

In some embodiments, the probe compound is a compound of one of formula (I), (II), or (III), except where R₁, L₁, or L₂ comprises an alkyne group. Exemplary probe compounds used in the Examples hereinbelow include 6-((5-cyclopropyl-1H-pyrazol-3-yl)amino-2-(4-(4-((3-phenyl-1H-1,2,4-triazol-1-yl)sulfonyl)-benzoyl)piperazin-1-yl)-N-prop-2-yn-1-yl)pyram-idine-4-carboxamide (also referred to herein as “KY-26”) and 4-((4-(2-(4-(bis(4-fluorophenyl)methylene)piperidin-1-yl)ethyl)-1H-1,2,3-triazol-1-yl)sulfonyl)-N-(prop-2-yn-1-yl)benzamide (also referred to herein as “TH211”).

### V. Synthesis

The probes and ligands of the presently disclosed subject matter can be prepared using organic group transformations known in the art of organic synthesis, as further described in the Examples below, and via methods analogous to those described in PCT International Publication No. WO 2020/214336 to Hsu et al., published Oct. 22, 2020, the disclosure of which is incorporated herein by reference in its entirety.

By way of example, SuTEx probes and ligands comprising a substituted 1,2,4-triazole group can be prepared by reacting sulfonyl chlorides with N-heteroaryl compounds. For example, Scheme 2, below shows an exemplary synthetic route to a sulfonyl-triazole compound starting from an amide reagent precursor of a substituted triazole. Thus, as shown in Scheme 2, an amide starting material (compound A in Scheme 2, where J represents the triazole substituent in the final SuTEx compound) can be coupled with DMF-DMA to produce an amidine intermediate (B). The amidine intermediate can undergo cyclization in acetic acid with hydrazine hydrate to form the corresponding 1,2,4-triazole⁵³, i.e., compound C in Scheme 2. The 1,2,4-triazole can then be reacted with a suitable sulfonyl chloride to provide the final SuTEx probe or ligand or a compound that can be further reacted to provide the SuTEx probe or ligand. J′ in Scheme 2 represents the AG of a SuTEx compound or a moiety that can be further reacted to provide the AG. Additional compounds for sulfur heterocycle exchange chemistry can be prepared by reacting the sulfonyl chlorides of with other N-heteroaryl compounds, e.g., imidazole, a substituted imidazole, pyrazole, a substituted pyrazole, tetrazole, or a substituted pyrazole.

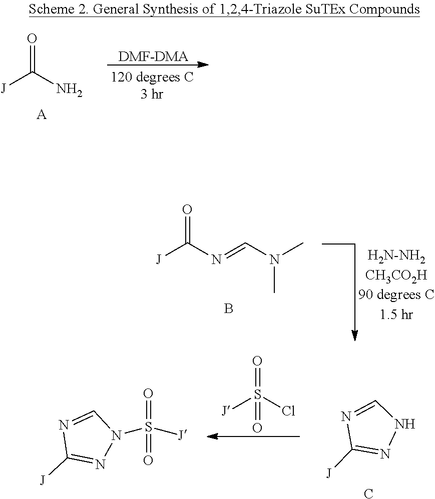

SuTEx probes comprising a 1,2,3-triazole group can be prepared as using a previously reported procedure⁵⁴, involving a copper catalyzed azide-alkyne cycloaddition using copper(I) thiophene-2-carboxylate (CuTC) in toluene. See Scheme 3, below. This initial cycloaddition provides a 1,4-regioisomer of the 1,2,3-triazole (compound D in Scheme 3), which can be converted to the 2,4-regioisomer⁵⁵ (compound E) using dimethylaminopyridine (DMAP) in acetonitrile.

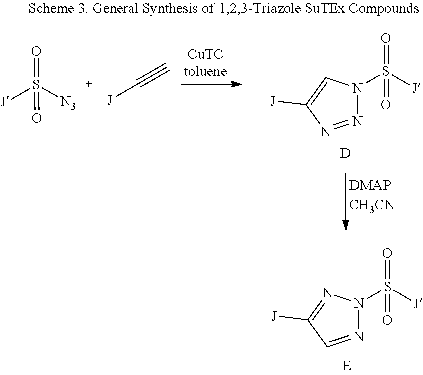

Alternatively, sulfonyl-triazole compounds can be prepared by synthetic routes involving a sulfide intermediate. Scheme 4, below, shows the synthesis of a sulfonyl-triazole compound by a route involving a benzyl sulfide intermediate.

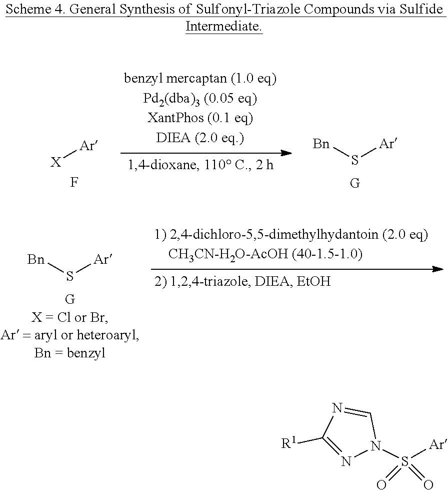

For example, as shown in Scheme 4, halo-substituted arene or heteroarene F can be reacted with benzyl mercaptan to provide benzyl sulfide intermediate G. Treatment of benzyl sulfide G with 1,2-dichloro-5,5-dimethylhydantoin in acetonitrile/water/acetic acid, followed by reaction with a 1,2,4-triazole provides the sulfonyl-triazole product. Other sulfonyl-heteroaryl compounds can be prepared by analogous routes using other nitrogen-containing heteroaryl compounds (e.g., imidazole) in place of the 1,2,4-triazole.

### VI. Methods of Identifying Reactive Amino Acid Residues

Covalent probes can serve as valuable tools for the global investigation of protein function and ligand binding capacity. Despite efforts to expand coverage of residues available for chemical proteomics (e.g. cysteine and lysine), a large fraction of the proteome remains inaccessible with current activity-based probes. According to one aspect of the presently disclosed subject matter is described sulfur-heterocycle exchange chemistry (e.g., sulfur-triazole exchange (SuTEx) chemistry) as a tunable platform for developing covalent probes and ligands with broad applications for chemical proteomics. Sulfur-heterocycle probes and ligands can act as electrophiles for reactive nucleophilic amino acid side chains of proteins, where reaction of the nucleophilic group of the nucleophilic amino acid side chain with the sulfur-heterocycle probe results in formation of a covalent bond between the nucleophilic group and the sulfur atom of a sulfonyl group in the probe and the breaking of a bond between the sulfonyl group and the heterocycle.

As example of the tunability of this platform, in SuTEx probes, modifications to the triazole leaving group can furnish sulfonyl probes with ˜5-fold enhanced chemoselectivity for tyrosines over other nucleophilic amino acids to investigate, for the first time, more than 10,000 tyrosine sites in lysates and live cells. Tyrosines with enhanced nucleophilicity have been found to be enriched in enzymatic, protein-protein interaction, and nucleotide recognition domains. In addition, SuTEx can be used as a chemical phosphoproteomics strategy to monitor activation of phosphotyrosine sites. Accordingly, collectively, SuTEx and related sulfur-heterocycle exchange chemistry compounds provide a biocompatible chemistry for chemical biology investigations of the human proteome.

In some embodiments, the presently disclosed subject matter provides small molecule probes that interact with reactive nucleophilic residues on proteins or peptides, such as a reactive tyrosine residue of a tyrosine-containing protein and/or a reactive lysine residue of a lysine-containing protein, as well as methods of identifying a protein or peptide that contains such a reactive residue (e.g., a druggable tyrosine residue and/or a druggable lysine residue). In some instances, also described herein are methods of profiling a ligand that interacts with one or more tyrosine- and/or lysine-containing protein comprising one or more reactive tyrosines and/or lysines.

In some embodiments, the presently disclosed subject matter provides a method of identifying a reactive tyrosine of a protein, the method comprising: (a) providing a protein sample comprising isolated proteins, living cells, or a cell lysate; (b) contacting the protein sample with a probe compound as described hereinabove for a period of time sufficient for the probe compound to react with at least one reactive tyrosine in a protein in the protein sample, thereby forming at least one modified reactive tyrosine residue; and (c) analyzing proteins in the protein sample to identify at least one modified tyrosine residue, thereby identifying at least one reactive tyrosine of a protein.

In some embodiments, the at least one modified reactive tyrosine residue comprises a modified tyrosine residue comprising a structure:

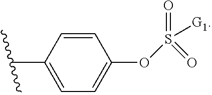

In some embodiments, G₁ comprises a fluorophore or detectable labeling moiety as described hereinbelow. In some embodiments, G₁ comprises an aryl group substituted by an alkyne-substituted alkyl group, an alkyne-substituted alkoxy group, or a group having the formula —C(═O)—NH-alkylene-C≡CH.

In some embodiments, the presently disclosed methods can alternatively or additionally provide for identifying reactive lysine residues in a protein. For example, during the contacting step (b) of the method described hereinabove, the probe compound can react with at least one reactive lysine in a protein in the protein sample, thereby forming at least one modified reactive lysine residue, and during the analyzing step (c), the method can further comprise analyzing the proteins in the protein sample to identify the at least one modified lysine residue, thereby identifying at least one reactive lysine of a protein.

Thus, in some embodiments, the presently disclosed subject matter provides a method of identifying a reactive lysine of a protein, the method comprising: (a) providing a protein sample comprising isolated proteins, living cells, or a cell lysate; (b) contacting the protein sample with a probe compound for a period of time sufficient for the probe compound to react with at least one reactive lysine in a protein in the protein sample, thereby forming at least one modified reactive lysine residue; and (c) analyzing proteins in the protein sample to identify at least one modified lysine residue, thereby identifying at least one reactive lysine of a protein. In some embodiments, the at least one modified reactive lysine residue comprises a modified lysine residue comprising a structure:

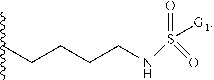

In some embodiments, the at least one modified reactive lysine residue is in a kinase.

In some embodiments, the presently disclosed subject matter provides a method of identifying a reactive tyrosine and/or a reactive lysine of a protein, the method comprising: (a) providing a protein sample comprising isolated proteins, living cells, or a cell lysate; (b) contacting the protein sample with a probe compound for a period of time sufficient for the probe compound to react with at least one reactive tyrosine and/or at least one reactive lysine in a protein in the protein sample, thereby forming at least one modified reactive tyrosine residue and/or at least one modified reactive lysine residue; and (c) analyzing proteins in the protein sample to identify at least one modified tyrosine residue and/or at least one modified lysine residue, thereby identifying at least one reactive tyrosine and/or at least one reactive lysine of a protein; wherein the at least one modified reactive tyrosine residue and/or one modified reactive lysine residue comprise a terminal alkyne. In some embodiments, the probe compound is KY-26 or TH211.

In some embodiments, the analyzing of step (c) further comprises tagging the at least one modified reactive tyrosine residue and/or at least one reactive lysine residue with a compound comprising a detectable labeling group, thereby forming at least one tagged reactive tyrosine residue comprising said detectable labeling group and/or at least one tagged reactive lysine residue comprising said detectable labeling group. In some embodiments, the detectable labeling group comprises biotin or a biotin derivative. In some embodiments, the biotin derivative is desthiobiotin.

In some embodiments, the tagging comprises reacting a terminal alkyne group of at least one tagged reactive tyrosine residue and/or at least one tagged reactive lysine residue with a compound comprising both an azide moiety (or other alkyne-reactive group) and a detectable labeling group (e.g., biotin or a biotin derivative. In some embodiments, the compound comprising the azide moiety and the detectable labeling group further comprises an alkylene linker, which in some embodiments, can comprise a polyether group, such as an oligomer of methylene glycol, ethylene glycol or propylene glycol (e.g., a group having the formula —(O—C₂H₄—)ₓ—). In some embodiments, the tagging comprises performing a copper-catalyzed azide-alkyne cycloaddition (CuAAC) coupling reaction.

In some embodiments, the analyzing further comprises digesting the protein sample to provide a digested protein sample comprising a protein fragment comprising the at least one tagged reactive tyrosine moiety comprising the detectable group and/or the at least one tagged reactive lysine residue comprising the detectable group. In some embodiments, the digesting is performed with a peptidase. In some embodiments, the digesting is performed with trypsin. In some embodiments, the digesting is performed with chymotrypsin. In some embodiments, the digesting is performed with both trypsin and chymotrypsin.

In some embodiments, the analyzing further comprises enriching the digested protein sample for the detectable labeling group. For example, in some embodiments, the enriching comprises contacting the digested protein sample with a solid support comprising a binding partner of the detectable labeling group. In some embodiments, when the detectable labeling group comprises biotin or a derivative thereof, the solid support comprises streptavidin. In some embodiments, the analyzing further comprises analyzing the digested protein sample (e.g., the enriched digested protein sample) via liquid chromatography-mass spectrometry or via a gel-based assay.

In some embodiments, providing the protein sample further comprises separating the protein sample into a first protein sample and a second protein sample. Then, in the contacting step, the first protein sample can be contacted with a first probe compound at a first probe concentration for a first period of time and the second protein sample can be contacted with a second probe compound (e.g., a probe compound having a different structure than that of the first probe compound) at the same probe concentration (i.e., at the first probe concentration) for the same time period (i.e., for the first period of time). Alternatively, the second protein sample can be contacted with the same probe compound as the first protein sample, but at a different probe concentration (i.e., a second probe concentration) or for a different period of time. In some embodiments, analyzing proteins comprises analyzing the first and second protein samples to determine the presence and/or identity of a modified reactive tyrosine and/or lysine residue in the first sample and the presence and/or identity of a modified reactive tyrosine and/or lysine residue in the second sample. In some embodiments, the identities and/or amounts of identified modified reactive tyrosine and/or lysine residues from the first and second protein samples are compared.

In some embodiments, the protein sample comprises living cells. In some embodiments, providing the protein sample further comprises separating the protein sample into a first protein sample and a second protein sample and culturing the first protein sample in a first cell culture medium comprising heavy isotopes prior to the contacting of step (b) and culturing the second protein sample in a second cell culture medium, wherein the second culture medium comprises a naturally occurring isotope distribution prior to the contacting of step (b). In some embodiments, the first cell culture medium comprises ¹³C- and/or ¹⁵N-labeled amino acids. In some embodiments, the first cell culture medium comprises ¹³C-¹⁵N-labeled lysine and arginine.

In some embodiments, e.g., if the protein sample does not comprise living cells, the probe compound can comprise a detectable labeling group comprising a heavy isotope (e.g., a ¹³C label) or the method can comprise tagging the at least one modified tyrosine residue and/or at least one modified lysine residue with a detectable labeling group comprising a heavy isotope.

In some embodiments, the protein sample is separated into a first and a second protein sample and one of the first and the second protein sample is cultured in the presences of a tyrosine phosphatase inhibitor (e.g., pervanadate). Thus, in some embodiments, the presently disclosed methods can be used in phosphoproteomics.

### VII. Modified Proteins

In some embodiments, the presently disclosed subject matter provides a modified tyrosine- and/or lysine-containing protein. The modified protein can be a protein comprising the adduct (e.g., the covalent adduct) formed between a tyrosine phenol group or a lysine primary amino group and a probe or ligand of the presently disclosed subject matter. The modified protein can have a different biological activity than the unmodified protein.

In some embodiments, the presently disclosed subject matter provides a modified tyrosine-containing protein comprising a modified tyrosine residue wherein the modified tyrosine residue is formed by the reaction of a tyrosine residue with a non-naturally occurring compound having a structure of formula (I), (II), or (III) or a pharmaceutically acceptable salt or solvate thereof. In some embodiments, the presently disclosed subject matter provides a modified lysine-containing protein comprising a modified lysine reside wherein the modified lysine residue is formed by the reaction of a lysine residue with a non-naturally occurring compound having a structure of formula (I), (II), or (III) or a pharmaceutically acceptable salt or solvate thereof.

The modified tyrosine and/or lysine-containing protein can be a protein that comprises a tyrosine or lysine residue as denoted in Tables 1-3, 5 or 6. For example, the proteins that are targeted by the KY-26 probe can be also be targeted by corresponding inhibitor compounds, e.g., KY-424 and other compounds of formula (I). In some embodiments, the modified tyrosine and/or lysine-containing protein is a kinase selected from the group including, but not limited to, Cyclin-dependent kinase 1 (CDK1), Cyclin-dependent kinase 2 (CDK2), Cyclin-dependent-like kinase 5 (CDK5), Dual specificity mitogen-activated protein kinase kinase 1, eIF-2-alpha kinase GCN2, Interleukin-1 receptor-associated kinase 4, MAP/microtubule affinity-regulating kinase 4, Mitogen-activated protein kinase kinase kinase kinase 1, Mitogen-activated protein kinase kinase kinase kinase 2, Mitogen-activated protein kinase kinase kinase kinase 5, Phosphatidylinositol 4,5-bisphosphate 3-kinase catalytic subunit delta, Phosphoglycerate kinase 1, Protein-tyrosine kinase 2-beta, Pyruvate kinase PKM, Receptor-interacting serine/threonine-protein kinase 1, Serine/threonine-protein kinase 4, Serine/threonine-protein kinase MARK2, Serine/threonine-protein kinase tousled-like 2, Thymidylate kinase, Tyrosine-protein kinase Fer, Tyrosine-protein kinase Lck, 5′-AMP-activated protein kinase catalytic subunit alpha-1, Cyclin-dependent-like kinase 6, Dual specificity mitogen-activated protein kinase kinase 2, Interferon-induced, double-stranded RNA-activated protein kinase, Nucleoside diphosphate kinase B, Serine/threonine-protein kinase tousled-like 1,Tyrosine-protein kinase CSK, PFKL, and a DGK.

In some embodiments, the modified tyrosine-containing protein is modified at a tyrosine residue in CDK2, PFKL, or a DGK. In some embodiments, the modified tyrosine residue is a tyrosine modified by a compound selected from the group comprising KY-424, TH-207, TH-208, TH-220, TH-221, TH-223, TH-225, XJ-2-47, XJ-2-65, XJ-2-77, XJ-2-87, XJ-2-105, XJ-2-105, XJ-2-111, XJ-2-115, XJ-2-139, XJ-2-141, SMS-55, SMS-59, SMS-63, SMS-65, SMS-67, SMS-69, SMS-71, SMS-73, SMS-75, SMS-77, SMS-79, SMS-81, SMS-83, SMS-85, SMS-87, and pharmaceutically acceptable salts and solvates thereof. In some embodiments, the compound is KY-424. In some embodiments, the compound is TH207 or TH220. In some embodiments, the compound is XJ-2-87, XJ-2-115, or XJ-2-141.

In some embodiments, the presently disclosed subject matter provides a modified lysine-containing protein comprising a modified lysine residue wherein the modified lysine residue is formed by the reaction of a lysine residue with a non-naturally occurring compound having a structure of formula (I), (II), or (III), or a pharmaceutically acceptable salt or solvate thereof. In some embodiments, the modified lysine residue is a lysine modified by a compound selected from the group comprising KY-424, TH-207, TH-208, TH-220, TH-221, TH-223, TH-225, XJ-2-47, XJ-2-65, XJ-2-77, XJ-2-87, XJ-2-105, XJ-2-105, XJ-2-111, XJ-2-115, XJ-2-139, XJ-2-141, SMS-55, SMS-59, SMS-63, SMS-65, SMS-67, SMS-69, SMS-71, SMS-73, SMS-75, SMS-77, SMS-79, SMS-81, SMS-83, SMS-85, SMS-87, and pharmaceutically acceptable salts and solvates thereof. In some embodiments, the compound is KY-424. In some embodiments, the compound is TH207 or TH220. In some embodiments, the compound is XJ-2-87, XJ-2-115, or XJ-2-141.

The amino acid sequence of human CDK2 (UniProt ID P24941.2; Accession No. NP_001789.2 of the GENBANK® biosequence database) is: MENFQKVEKIGEGTYGVVYKARNKLTGEVVALKKIRLDTETEGVPSTAIREISLLK ELNHPNIVKLLDVIHTENKLYLVFEFLHQDLKKFMDASALTGIPLPLIKSYLFQLLQ GLAFCHSHRVLHRDLKPQNLLINTEGAIKLADFGLARAFGVPVRTYTHEVVTLWY RAPEILLGCKYYSTAVDIWSLGCIFAEMVTRRALFPGDSEIDQLFRIFRTLGTPDEV VWPGVTSMPDYKPSFPKWARQDFSKVVPPLDEDGRSLLSQMLHYDPNKRISAKA ALAHPFFQDVTKPVPHLRL (SEQ ID NO: 2). In some embodiments, the modified protein is human CDK2 modified at the lysine at residue 33 of SEQ ID NO: 2 (i.e., K33).

The amino acid sequence of human DGK alpha (DGKA; UniProt ID P23743.3; Accession No. NP_001336.2 of the GENBANK® biosequence database) is: MAKERGLISPSDFAQLQKYMEYSTKKVSDVLKLFEDGEMAKYVQGDAIGYEGFQ QFLKIYLEVDNVPRHLSLALFQSFETGHCLNETNVTKDVVCLNDVSCYFSLLEGG RPEDKLEFTFKLYDTDRNGILDSSEVDKIILQMMRVAEYLDWDVSELRPILQEMM KEIDYDGSGSVSQAEWVRAGATTVPLLVLLGLEMTLKDDGQHMWRPKRFPRPV YCNLCESSIGLGKQGLSCNLCKYTVHDQCAMKALPCEVSTYAKSRKDIGVQSHV WVRGGCESGRCDRCQKKIRIYHSLTGLHCVWCHLEIHDDCLQAVGHECDCGLLR DHILPPSSIYPSVLASGPDRKNSKTSQKTMDDLNLSTSEALRIDPVPNTHPLLVFVN PKSGGKQGQRVLWKFQYILNPRQVFNLLKDGPEIGLRLFKDVPDSRILVCGGDGT VGWILETIDKANLPVLPPVAVLPLGTGNDLARCLRWGGGYEGQNLAKILKDLEM SKVVHMDRWSVEVIPQQTEEKSDPVPFQIINNYFSIGVDASIAHRFHIMREKYPEKF NSRMKNKLWYFEFATSESIFSTCKKLEESLTVEICGKPLDLSNLSLEGIAVLNIPSM HGGSNLWGDTRRPHGDIYGINQALGATAKVITDPDILKTCVPDLSDKRLEVVGLE GAIEMGQIYTKLKNAGRRLAKCSEITFHTTKTLPMQIDGEPWMQTPCTIKITHKNQ MPMLMGPPPRSTNFFGFLS (SEQ ID NO: 3). In some embodiments, the modified protein is human DGKA modified at one or more tyrosine of residues 19, 42, 50, 169, 240, 335, 399, 544, and 669 of SEQ ID NO: 3 and/or one or more lysine of residues 18, 25, 26, 32, 260, 353, 384, 411, 543, and 547 of SEQ ID NO: 3 (i.e., at one or more of Y19, Y42, Y50, Y169, Y240, Y258, Y335, Y399, Y544, Y669, K18, K25, K26, K32, K260, K353, K384, K411, K543, and K547 of SEQ ID NO: 3).

The amino acid sequence of human DGK zeta (DGKZ; UniProt ID Q13574.4; Accession No. NP_001186196.1 of the GENBANK® biosequence database) is: MEPRDGSPEARSSDSESASASSSGSERDAGPEPDKAPRRLNKRRFPGLRLFGHRKA ITKSGLQHLAPPPPTPGAPCSESERQIRSTVDWSESATYGEHIWFETNVSGDFCYVG EQYCVARMLKSVSRRKCAACKIVVHTPCIEQLEKINFRCKPSFRESGSRNVREPTF VRHHWVHRRRQDGKCRHCGKGFQQKFTFHSKEIVAISCSWCKQAYHSKVSCFM LQQIEEPCSLGVHAAVVIPPTWILRARRPQNTLKASKKKKRASFKRKSSKKGPEEG RWRPFIIRPTPSPLMKPLLVFVNPKSGGNQGAKIIQSFLWYLNPRQVFDLSQGGPK EALEMYRKVHNLRILACGGDGTVGWILSTLDQLRLKPPPPVAILPLGTGNDLART LNWGGGYTDEPVSKILSHVEEGNVVQLDRWDLHAEPNPEAGPEDRDEGATDRLP LDVFNNYFSLGFDAHVTLEFHESREANPEKFNSRFRNKMFYAGTAFSDFLMGSSK DLAKHIRVVCDGMDLTPKIQDLKPQCVVFLNIPRYCAGTMPWGGHPGEHHDFEPQ RHDDGYLEVIGFTMTSLAALQVGGHGERLTQCREVVLTTSKAIPVQVDGEPCKLA ASRIRIALRNQATMVQKAKRRSAAPLHSDQQPVPEQLRIQVSRVSMHDYEALHYD KEQLKEASVPLGTVVVPGDSDLELCRAHIERLQQEPDGAGAKSPTCQKLSPKWCF LDATTASRFYRIDRAQEHLNYVTEIAQDEIYILDPELLGASARPDLPTPTSPLPTSPC SPTPRSLQGDAAPPQGEELIEAAKRNDFCKLQELHRAGGDLMHRDEQSRTLLHHA VSTGSKDVVRYLLDHAPPEILDAVEENGETCLHQAAALGQRTICHYIVEAGASLM KTDQQGDTPRQRAEKAQDTELAAYLENRQHYQMIQREDQETAV (SEQ ID NO: 4). In some embodiments, the modified protein is human DGKZ modified at one or more tyrosine of residues 319, 340, 484, 656, 661, 841, 876, and 909 of SEQ ID NO: 4 and/or at one or more lysine of residues 59, 123, 134, 147, 189, 194, 211, 256, 311, 342, 370, 403, 473, 481, 498, 502, 516, 521, 593, 605, 624, 663, 667, 704, 714, 836, 886, and 900 of SEQ ID NO: 4 (i.e., at one or more of Y319, Y340, Y484, Y656, Y661, Y841, Y876, Y909, K59, K123, K134, K147, K189, K194, K211, K256, K311, K342, K370, K403, K473, K481, K498, K502, K516, K521, K593, K605, K624, K663, K667, K704, K714, K836, K886, and K900 of SEQ ID NO: 4).

The amino acid sequence of human PFKL (UniProt ID P17858.6; Accession No. NP_002617.3 of the GENBANK (RO biosequence database) is: MAAVDLEKLRASGAGKAIGVLTSGGDAQGMNAAVRAVTRMGIYVGAKVFLIYE GYEGLVEGGENIKQANWLSVSNIIQLGGTIIGSARCKAFTTREGRRAAAYNLVQH GITNLCVIGGDGSLTGANIFRSEWGSLLEELVAEGKISETTARTYSHLNIAGLVGSI DNDFCGTDMTIGTDSALHRIMEVIDAITTTAQSHQRTFVLEVMGRHCGYLALVSA LASGADWLFIPEAPPEDGWENFMCERLGETRSRGSRLNIIIIAEGAIDRNGKPISSSY VKDLVVQRLGFDTRVTVLGHVQRGGTPSAFDRILSSKMGMEAVMALLEATPDTP ACVVTLSGNQSVRLPLMECVQMTKEVQKAMDDKRFDEATQLRGGSFENNWNIY KLLAHQKPPKEKSNFSLAILNVGAPAAGMNAAVRSAVRTGISHGHTVYVVHDGF EGLAKGQVQEVGWHDVAGWLGRGGSMLGTKRTLPKGQLESIVENIRIYGIHALL VVGGFEAYEGVLQLVEARGRYEELCIVMCVIPATISNNVPGTDFSLGSDTAVNAA MESCDRIKQSASGTKRRVFIVETMGGYCGYLATVTGIAVGADAAYVFEDPFNIHD LKVNVEHMTEKMKTDIQRGLVLRNEKCHDYYTTEFLYNLYSSEGKGVFDCRTNV LGHLQQGGAPTPFDRNYGTKLGVKAMLWLSEKLREVYRKGRVFANAPDSACVI GLKKKAVAFSPVTELKKDTDFEHRMPREQWWLSLRLMLKMLAQYRISMAAYVS GELEHVTRRTLSMDKGF (SEQ ID NO: 5). In some embodiments, the modified protein is human PFKL modified at the tyrosine of residue 674 of SEQ ID NO: 5 and/or the lysine of residue 677 of SEQ ID NO: 5 (i.e., at Y674 and/or K677 of SEQ ID NO: 5).

In some embodiments, the modified protein is CDK2 (or a tyrosine- and/or lysine-containing fragment thereof) modified by a compound of formula (I) (e.g., KY-424) or a pharmaceutically acceptable salt or solvate thereof. In some embodiments, said modified protein is human CDK2 modified by a compound of formula (I) at K33 of SEQ ID NO: 2. In some embodiments, the modified protein is a DGK (or a tyrosine- and/or lysine-containing fragment thereof) modified by a compound of formula (II) or a pharmaceutically acceptable salt or solvate thereof. In some embodiments, the modified protein is human DGKA modified by a compound of formula (II) at one or more of Y19, Y42, Y50, Y169, Y240, Y258, Y335, Y399, Y544, Y669, K18, K25, K26, K32, K260, K353, K384, K411, K543, and K547 of SEQ ID NO: 3. In some embodiments, the modified protein is human DGKZ modified by a compound of formula (II) at one or more of Y319, Y340, Y484, Y656, Y661, Y841, Y876, Y909, K59, K123, K134, K147, K189, K194, K211, K256, K311, K342, K370, K403, K473, K481, K498, K502, K516, K521, K593, K605, K624, K663, K667, K704, K714, K836, K886, and K900 of SEQ ID NO: 4. In some embodiments, the modified protein is a PFKL (or a tyrosine- and/or lysine-containing fragment thereof) modified by a compound of formula (III) or a pharmaceutically acceptable salt or solvate thereof. In some embodiments, the modified protein is human PFKL modified by a compound of formula (III) at Y674 and/or K677 of SEQ ID NO: 5.

### VIII. Therapeutic Uses and Pharmaceutical Compositions

Small molecules, such as the presently disclosed ligands and probes, present an alternative method to selectively modulate proteins and to serve as leads for the development of novel therapeutics.

Dysregulated expression of a tyrosine-containing protein (e.g., a tyrosine-containing kinase), in many cases, is associated with or modulates a disease, such as an inflammatory related disease, a neurodegenerative disease, or cancer. As such, identification of a potential agonist/antagonist to a tyrosine-containing protein aids in improving the disease condition in a patient.

Thus, in some embodiments, disclosed herein are tyrosine-containing proteins that comprise one or more ligandable tyrosines. In some instances, the tyrosine-containing protein is a soluble protein or a membrane protein. In some instances, the tyrosine-containing protein is involved in one or more of a biological process such as protein transport, lipid metabolism, apoptosis, transcription, electron transport, mRNA processing, or host-virus interaction. In some instances, the tyrosine-containing protein is associated with one or more of diseases such as cancer or one or more disorders or conditions such as immune, metabolic, developmental, reproductive, neurological, psychiatric, renal, cardiovascular, or hematological disorders or conditions.

In some embodiments, disclosed herein are lysine-containing proteins that comprise one or more ligandable lysines. In some instances, the lysine-containing protein is a soluble protein. In other instances, the lysine-containing protein is a membrane protein. In some cases, the lysine-containing protein is involved in one or more of a biological process such as protein transport, lipid metabolism, apoptosis, transcription, electron transport, mRNA processing, or host-virus interaction. In additional cases, the lysine-containing protein is associated with one or more of diseases such as cancer or one or more disorders or conditions such as immune, metabolic, developmental, reproductive, neurological, psychiatric, renal, cardiovascular, or hematological disorders or conditions.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C ¹⁵N¹⁸O, ¹⁷O, ³⁵, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the presently disclosed subject matter provides pharmaceutical compositions comprising one or more of the presently disclosed ligands. The pharmaceutical compositions comprise at least one ligand compound, e.g. selected from compounds of formula (I), (II), or (III), described herein, or a pharmaceutically acceptable salt or solvate thereof, in combination with a pharmaceutically acceptable carrier, vehicle, or diluent, such as an aqueous buffer at a physiologically acceptable pH (e.g., pH 7 to 8.5), a non-aqueous liquid, a polymer-based nanoparticle vehicle, a liposome, and the like. The pharmaceutical compositions can be delivered in any suitable dosage form, such as a liquid, gel, solid, cream, or paste dosage form. In one embodiment, the compositions can be adapted to give sustained release of the active compound.

In some embodiments, the pharmaceutical compositions include, but are not limited to, those forms suitable for oral, rectal, nasal, topical, (including buccal and sublingual), transdermal, vaginal, parenteral (including intramuscular, subcutaneous, and intravenous), spinal (epidural, intrathecal), central (intracerebroventricular) administration, in a form suitable for administration by inhalation or insufflation. The compositions can, where appropriate, be provided in discrete dosage units. The pharmaceutical compositions of the presently disclosed subject matter can be prepared by any of the methods well known in the pharmaceutical arts. Some preferred modes of administration include intravenous (i.v.), intraperitoneal (i.p.), topical, subcutaneous, and oral.

Pharmaceutical formulations suitable for oral administration include capsules, cachets, or tablets, each containing a predetermined amount of one or more of the ligands, as a powder or granules. In another embodiment, the oral composition is a solution, a suspension, or an emulsion. Alternatively, the ligands can be provided as a bolus, electuary, or paste. Tablets and capsules for oral administration can contain conventional excipients such as binding agents, fillers, lubricants, disintegrants, colorants, flavoring agents, preservatives, or wetting agents. The tablets can be coated according to methods well known in the art, if desired. Oral liquid preparations include, for example, aqueous or oily suspensions, solutions, emulsions, syrups, or elixirs. Alternatively, the compositions can be provided as a dry product for constitution with water or another suitable vehicle before use. Such liquid preparations can contain conventional additives such as suspending agents, emulsifying agents, non-aqueous vehicles (which may include edible oils), preservatives, and the like. The additives, excipients, and the like typically will be included in the compositions for oral administration within a range of concentrations suitable for their intended use or function in the composition, and which are well known in the pharmaceutical formulation art. The presently disclosed ligands will be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

Pharmaceutical compositions for parenteral, spinal, or central administration (e.g. by bolus injection or continuous infusion) or injection into amniotic fluid can be provided in unit dose form in ampoules, pre-filled syringes, small volume infusion, or in multi-dose containers, and preferably include an added preservative. The compositions for parenteral administration can be suspensions, solutions, or emulsions, and can contain excipients such as suspending agents, stabilizing agents, and dispersing agents. Alternatively, the ligands can be provided in powder form, obtained by aseptic isolation of sterile solid or by lyophilization from solution, for constitution with a suitable vehicle, e.g. sterile, pyrogen-free water, before use. The additives, excipients, and the like typically will be included in the compositions for parenteral administration within a range of concentrations suitable for their intended use or function in the composition, and which are well known in the pharmaceutical formulation art. The ligands of the presently disclosed subject matter can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 100 millimolar, preferably at least about 1 nanomolar to about 10 millimolar.

Pharmaceutical compositions for topical administration of the ligands to the epidermis (mucosal or cutaneous surfaces) can be formulated as ointments, creams, lotions, gels, or as a transdermal patch. Such transdermal patches can contain penetration enhancers such as linalool, carvacrol, thymol, citral, menthol, t-anethole, and the like. Ointments and creams can, for example, include an aqueous or oily base with the addition of suitable thickening agents, gelling agents, colorants, and the like. Lotions and creams can include an aqueous or oily base and typically also contain one or more emulsifying agents, stabilizing agents, dispersing agents, suspending agents, thickening agents, coloring agents, and the like. Gels preferably include an aqueous carrier base and include a gelling agent such as cross-linked polyacrylic acid polymer, a derivatized polysaccharide (e.g., carboxymethyl cellulose), and the like. The additives, excipients, and the like typically will be included in the compositions for topical administration to the epidermis within a range of concentrations suitable for their intended use or function in the composition, and which are well known in the pharmaceutical formulation art. The ligands of the presently disclosed subject matter can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

Pharmaceutical compositions suitable for topical administration in the mouth (e.g., buccal or sublingual administration) include lozenges comprising the ligand in a flavored base, such as sucrose, acacia, or tragacanth; pastilles comprising the ligand in an inert base such as gelatin and glycerin or sucrose and acacia; and mouthwashes comprising the active ingredient in a suitable liquid carrier. The pharmaceutical compositions for topical administration in the mouth can include penetration enhancing agents, if desired. The additives, excipients, and the like typically will be included in the compositions of topical oral administration within a range of concentrations suitable for their intended use or function in the composition, and which are well known in the pharmaceutical formulation art. The ligands of the presently disclosed subject matter can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

A pharmaceutical composition suitable for rectal administration comprises a ligand of the presently disclosed subject matter in combination with a solid or semisolid (e.g., cream or paste) carrier or vehicle. For example, such rectal compositions can be provided as unit dose suppositories. Suitable carriers or vehicles include cocoa butter and other materials commonly used in the art. The additives, excipients, and the like typically will be included in the compositions of rectal administration within a range of concentrations suitable for their intended use or function in the composition, and which are well known in the pharmaceutical formulation art. The ligands of the presently disclosed subject matter can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

According to one embodiment, pharmaceutical compositions of the presently disclosed subject matter suitable for vaginal administration are provided as pessaries, tampons, creams, gels, pastes, foams, or sprays containing a ligand of the presently disclosed subject matter in combination with a carrier as are known in the art. Alternatively, compositions suitable for vaginal administration can be delivered in a liquid or solid dosage form. The additives, excipients, and the like typically will be included in the compositions of vaginal administration within a range of concentrations suitable for their intended use or function in the composition, and which are well known in the pharmaceutical formulation art. The ligands of the presently disclosed subject matter will be included in the compositions within a therapeutically useful and effective concentration range, as determined by routine methods that are well known in the medical and pharmaceutical arts. For example, a typical composition can include one or more of the presently disclosed ligands at a concentration in the range of at least about 0.01 nanomolar to about 1 molar, preferably at least about 1 nanomolar to about 100 millimolar.

Pharmaceutical compositions suitable for intra-nasal administration are also encompassed by the presently disclosed subject matter. Such intra-nasal compositions comprise a ligand of the presently disclosed subject matter in a vehicle and suitable administration device to deliver a liquid spray, dispersible powder, or drops. Drops may be formulated with an aqueous or non-aqueous base also comprising one or more dispersing agents, solubilizing agents, or suspending agents. Liquid sprays are conveniently delivered from a pressurized pack, an insufflator, a nebulizer, or other convenient approach of delivering an aerosol comprising the ligand. Pressurized packs comprise a suitable propellant such as dichlorodifluoromethane, trichlorofluoromethane, dichlorotetrafluoroethane, carbon dioxide, or other suitable gas as is well known in the art. Aerosol dosages can be controlled by providing a valve to deliver a metered amount of the ligand. Alternatively, pharmaceutical compositions for administration by inhalation or insufflation can be provided in the form of a dry powder composition, for example, a powder mix of the ligand and a suitable powder base such as lactose or starch. Such powder composition can be provided in unit dosage form, for example, in capsules, cartridges, gelatin packs, or blister packs, from which the powder can be administered with the aid of an inhalator or insufflator. The additives, excipients, and the like typically will be included in the compositions of intra-nasal administration within a range of concentrations suitable for their intended use or function in the composition, and which are well known in the pharmaceutical formulation art. The ligand of the presently disclosed subject matter will be included in the compositions within a therapeutically useful and effective concentration range, as determined by routine methods that are well known in the medical and pharmaceutical arts. For example, a typical composition can include one or more ligand at a concentration in the range of at least about 0.01 nanomolar to about 1 molar, preferably at least about 1 nanomolar to about 100 millimolar.

Optionally, the pharmaceutical compositions of the presently disclosed subject matter can include one or more other therapeutic agent, e.g., as a combination therapy. The additional therapeutic agent will be included in the compositions within a therapeutically useful and effective concentration range, as determined by routine methods that are well known in the medical and pharmaceutical arts. The concentration of any particular additional therapeutic agent may be in the same range as is typical for use of that agent as a monotherapy, or the concentration can be lower than a typical monotherapy concentration if there is a synergy when combined with a ligand of the presently disclosed subject matter.

In some embodiments, the presently disclosed subject matter provides a method of inhibiting a kinase, wherein the method comprises contacting a sample comprising a kinase with an effective amount of a ligand compound as described hereinabove, i.e., a compound of formula (I), (II), or (III), or a pharmaceutically acceptable salt or solvate thereof, and/or a pharmaceutical composition thereof. In some embodiments, the kinase is selected from the group comprising Cyclin-dependent kinase 1 (CDK1), Cyclin-dependent kinase 2 (CDK2), Cyclin-dependent-like kinase 5 (CDK5), Dual specificity mitogen-activated protein kinase kinase 1, eIF-2-alpha kinase GCN2, Interleukin-1 receptor-associated kinase 4, MAP/microtubule affinity-regulating kinase 4, Mitogen-activated protein kinase kinase kinase kinase 1, Mitogen-activated protein kinase kinase kinase kinase 2, Mitogen-activated protein kinase kinase kinase kinase 5, Phosphatidylinositol 4,5-bisphosphate 3-kinase catalytic subunit delta, Phosphoglycerate kinase 1, Protein-tyrosine kinase 2-beta, Pyruvate kinase PKM, Receptor-interacting serine/threonine-protein kinase 1, Serine/threonine-protein kinase 4, Serine/threonine-protein kinase MARK2, Serine/threonine-protein kinase tousled-like 2, Thymidylate kinase, Tyrosine-protein kinase Fer, Tyrosine-protein kinase Lck, 5′-AMP-activated protein kinase catalytic subunit alpha-1, Cyclin-dependent-like kinase 6, Dual specificity mitogen-activated protein kinase kinase 2, Interferon-induced, double-stranded RNA-activated protein kinase, Nucleoside diphosphate kinase B, Serine/threonine-protein kinase tousled-like 1,Tyrosine-protein kinase CSK, PFKL, and a DGK.

In some embodiments, the presently disclosed compounds can act as cyclin-dependent kinase (e.g., CDK2) inhibitors, phosphofructokinase (e.g., PFKL) inhibitors, and/or DGK inhibitors. For instance, in some embodiments, a compound of formula (I) can be used as a CDK2 inhibitor. In some embodiments, a compound of formula (II) can be used as a DGK inhibitor (e.g., a DGK alpha or DGK zeta inhibitor). In some embodiments, a compound of formula (III) can be used as a phosphofructokinase (e.g., PFKL) inhibitor.

The sample comprising the kinase can be, for example, a biological sample, such as, but not limited to, a biological fluid, a cell, a cell culture, a cell extract, a tissue, a tissue extract, an organ or an organism (e.g., a living organism, such as a human or other mammal). In some embodiments, inhibiting the kinase can treat and/or prevent a disease or disorder, e.g., associated with kinase activity. In some embodiments, the disease or disorder treatable with the presently disclosed kinase inhibitors include, but are not limited to, cancer, inflammatory diseases, and neurodegenerative diseases. In some embodiments, the disease is cancer. For instance, DGKA AND DGKZ can be of use in treating cancer by activating the immune system (e.g., in immuno-oncology and immunotherapy). PFKL is a glycolytic enzyme that can be used as a targeted therapy for oncology. CDK2 is a cell cycle protein that can be used as a targeted therapy for oncology.

Thus, in some embodiments, the presently disclosed subject matter presents a method of treating a disease or disorder in a subject in need thereof, wherein the method comprises administering to the subject a compound of formula (I), (II), or (III), or a pharmaceutically acceptable salt and/or solvate and/or pharmaceutical composition thereof. In some embodiments, the compound is selected from the group comprising KY-424, TH-207, TH-208, TH-220, TH-221, TH-223, TH-225, XJ-2-47, XJ-2-65, XJ-2-77, XJ-2-87, XJ-2-105, XJ-2-105, XJ-2-111, XJ-2-115, XJ-2-139, XJ-2-141, SMS-55, SMS-59, SMS-63, SMS-65, SMS-67, SMS-69, SMS-71, SMS-73, SMS-75, SMS-77, SMS-79, SMS-81, SMS-83, SMS-85, SMS-87, and pharmaceutically acceptable salts or solvates thereof. In some embodiments, the compound is KY-424. In some embodiments, the compound is TH207 or TH220. In some embodiments, the compound is XJ-2-87, XJ-2-115, or XJ-2-141.

In some embodiments, the presently disclosed subject matter provides a pharmaceutical composition for use in inhibiting a kinase in a subject, wherein the pharmaceutical composition comprises a compound of formula (I), (II), or (III), or a pharmaceutically acceptable salt, solvate and/or pharmaceutical composition thereof. In some embodiments, the presently disclosed subject matter provides a pharmaceutical composition for use in treating a disease or disorder treatable by inhibiting CDK2, a DGK, or a PFK (e.g., cancer, an inflammatory disorder, or a neurodegenerative disorder) in a subject, wherein the pharmaceutical composition comprises a compound of formula (I), (II), or (III) or a pharmaceutically acceptable salt, solvate, and/or pharmaceutical composition thereof. In some embodiments, the compound is selected from the group comprising KY-424, TH-207, TH-208, TH-220, TH-221, TH-223, TH-225, XJ-2-47, XJ-2-65, XJ-2-77, XJ-2-87, XJ-2-105, XJ-2-105, XJ-2-111, XJ-2-115, XJ-2-139, XJ-2-141, SMS-55, SMS-59, SMS-63, SMS-65, SMS-67, SMS-69, SMS-71, SMS-73, SMS-75, SMS-77, SMS-79, SMS-81, SMS-83, SMS-85, and SMS-87, and pharmaceutically acceptable salts and solvates thereof. In some embodiments, the compound is KY-424. In some embodiments, the compound is TH220 or TH207. In some embodiments, the compound is XJ-2-87, XJ-2-115, or XJ-2-141.

### IX. Cells, Analytical Techniques and Instrumentation

In some embodiments, one or more of the methods disclosed herein comprise a sample (e.g., a cell sample, or a cell lysate sample). In some embodiments, the sample for use with the methods described herein is obtained from cells of an animal. In some instances, the animal cell includes a cell from a marine invertebrate, fish, insects, amphibian, reptile, or mammal. In some instances, the mammalian cell is a primate, ape, equine, bovine, porcine, canine, feline, or rodent. In some instances, the mammal is a primate, ape, dog, cat, rabbit, ferret, or the like. In some cases, the rodent is a mouse, rat, hamster, gerbil, hamster, chinchilla, or guinea pig. In some embodiments, the bird cell is from a canary, parakeet or parrots. In some embodiments, the reptile cell is from a turtles, lizard or snake. In some cases, the fish cell is from a tropical fish. In some cases, the fish cell is from a zebrafish (e.g. Danino rerio). In some cases, the worm cell is from a nematode (e.g. C. elegans). In some cases, the amphibian cell is from a frog. In some embodiments, the arthropod cell is from a tarantula or hermit crab.

In some embodiments, the sample for use with the methods described herein is obtained from a mammalian cell. In some instances, the mammalian cell is an epithelial cell, connective tissue cell, hormone secreting cell, a nerve cell, a skeletal muscle cell, a blood cell, or an immune system cell. Exemplary mammalian cell lines include, but are not limited to, 293A cells, 293FT cells, 293F cells, 293H cells, HEK 293 cells, CHO DG44 cells, CHO—S cells, CHO-K1 cells, and PC12 cells.

In some embodiments, the sample for use with the methods described herein is obtained from cells of a tumor cell line. In some instances, the sample is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, and telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the sample is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some instances, the hematologic malignant cell line is obtained from a B-cell cell line of: acute lymphoblastic leukemia (ALL), acute myelogenous leukemia (AML), chronic myelogenous leukemia (CMIL), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the sample for use with the methods described herein is obtained from a tumor cell line. Exemplary tumor cell lines include, but are not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, the sample for use in the methods is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, the sample is a tissue sample, such as a sample obtained from a biopsy or a tumor tissue sample. In some embodiments, the sample is a blood serum sample. In some embodiments, the sample is a blood cell sample containing one or more peripheral blood mononuclear cells (PBMCs). In some embodiments, the sample contains one or more circulating tumor cells (CTCs). In some embodiments, the sample contains one or more disseminated tumor cells (DTC, e.g., in a bone marrow aspirate sample).

In some embodiments, the samples are obtained from the individual by any suitable approach of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, will be examined under a microscope.

### X. Sample Preparation and Analysis

In some embodiments, the sample (e.g., cell sample, cell lysate sample, or comprising isolated proteins) is a sample solution. In some instances, the sample solution comprises a solution such as a buffer (e.g. phosphate buffered saline) or a media. In some embodiments, the media is an isotopically labeled media. In some instances, the sample solution is a cell solution.

In some embodiments, the sample (e.g., cell sample, cell lysate sample, or comprising isolated proteins) is incubated with one or more compound probes for analysis of protein-probe interactions. In some instances, the sample (e.g., cell sample, cell lysate sample, or comprising isolated proteins) is further incubated in the presence of an additional compound probe prior to addition of the one or more probes. In other instances, the sample (e.g., cell sample, cell lysate sample, or comprising isolated proteins) is further incubated with a non-probe small molecule ligand, in which the non-probe small molecule ligand does not contain a photoreactive moiety and/or an alkyne group. In such instances, the sample is incubated with a probe and non-probe small molecule ligand for competitive protein profiling analysis.

In some cases, the sample is compared with a control. In some cases, a difference is observed between a set of probe protein interactions between the sample and the control. In some instances, the difference correlates to the interaction between the small molecule fragment and the proteins.

In some embodiments, one or more methods are utilized for labeling a sample (e.g. cell sample, cell lysate sample, or comprising isolated proteins) for analysis of probe protein interactions. In some instances, a method comprises labeling the sample (e.g. cell sample, cell lysate sample, or comprising isolated proteins) with an enriched media. In some cases, the sample (e.g. cell sample, cell lysate sample, or comprising isolated proteins) is labeled with isotope-labeled amino acids, such as ¹³C or ¹⁵N-labeled amino acids. In some cases, the labeled sample is further compared with a non-labeled sample to detect differences in probe protein interactions between the two samples. In some instances, this difference is a difference of a target protein and its interaction with a small molecule ligand in the labeled sample versus the non-labeled sample. In some instances, the difference is an increase, decrease or a lack of protein-probe interaction in the two samples. In some instances, the isotope-labeled method is termed SILAC, stable isotope labeling using amino acids in cell culture.

In some embodiments, a method comprises incubating a sample (e.g. cell sample, cell lysate sample, or comprising isolated proteins) with a labeling group (e.g., an isotopically labeled labeling group) to tag one or more proteins of interest for further analysis. In such cases, the detectable labeling group comprises a biotin, a streptavidin, bead, resin, a solid support, or a combination thereof, and further comprises a linker that is optionally isotopically labeled. As described above, the linker can be about 5, 6, 7, 8, 9, 10, 11, 12, 13, 14, 15 or more residues in length and might further comprise a cleavage site, such as a protease cleavage site (e.g., TEV cleavage site). In some cases, the labeling group is a biotin-linker moiety, which is optionally isotopically labeled with ¹³C and ¹⁵N atoms at one or more amino acid residue positions within the linker. In some cases, the biotin-linker moiety is a isotopically-labeled TEV-tag.

In some embodiments, an isotopic reductive dimethylation (ReDi) method is utilized for processing a sample. In some cases, the ReDi labeling method involves reacting peptides with formaldehyde to form a Schiff base, which is then reduced by cyanoborohydride. This reaction dimethylates free amino groups on N-termini and lysine side chains and monomethylates N-terminal prolines. In some cases, the ReDi labeling method comprises methylating peptides from a first processed sample with a “light” label using reagents with hydrogen atoms in their natural isotopic distribution and peptides from a second processed sample with a “heavy” label using deuterated formaldehyde and cyanoborohydride. Subsequent proteomic analysis (e.g., mass spectrometry analysis) based on a relative peptide abundance between the heavy and light peptide version might be used for analysis of probe-protein interactions.

In some embodiments, isobaric tags for relative and absolute quantitation (iTRAQ) method is utilized for processing a sample. In some cases, the iTRAQ method is based on the covalent labeling of the N-terminus and side chain amines of peptides from a processed sample. In some cases, reagent such as 4-plex or 8-plex is used for labeling the peptides.

In some embodiments, the probe-protein complex is further conjugated to a chromophore, such as a fluorophore. In some instances, the probe-protein complex is separated and visualized utilizing an electrophoresis system, such as through a gel electrophoresis, or a capillary electrophoresis. Exemplary gel electrophoresis includes agarose based gels, polyacrylamide based gels, or starch based gels. In some instances, the probe-protein is subjected to a native electrophoresis condition. In some instances, the probe-protein is subjected to a denaturing electrophoresis condition.

In some instances, the probe-protein after harvesting is further fragmentized to generate protein fragments. In some instances, fragmentation is generated through mechanical stress, pressure, or chemical approach. In some instances, the protein from the probe-protein complexes is fragmented by a chemical approach. In some embodiments, the chemical approach is a protease. Exemplary proteases include, but are not limited to, serine proteases such as chymotrypsin A, penicillin G acylase precursor, dipeptidase E, DmpA aminopeptidase, subtilisin, prolyl oligopeptidase, D-Ala-D-Ala peptidase C, signal peptidase I, cytomegalovirus assemblin, Lon-A peptidase, peptidase Clp, Escherichia coli phage KIF endosialidase CIMCD self-cleaving protein, nucleoporin 145, lactoferrin, murein tetrapeptidase LD-carboxypeptidase, or rhomboid-1; threonine proteases such as ornithine acetyltransferase; cysteine proteases such as TEV protease, amidophosphoribosyltransferase precursor, gamma-glutamyl hydrolase (Rattus norvegicus), hedgehog protein, DmpA aminopeptidase, papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, or DeSI-1 peptidase; aspartate proteases such as beta-secretase 1 (BACE1), beta-secretase 2 (BACE2), cathepsin D, cathepsin E, chymosin, napsin-A, nepenthesin, pepsin, plasmepsin, presenilin, or renin; glutamic acid proteases such as AfuGprA; and metalloproteases such as peptidase_M48.

In some instances, the fragmentation is a random fragmentation. In some instances, the fragmentation generates specific lengths of protein fragments, or the shearing occurs at particular sequence of amino acid regions.

In some instances, the protein fragments are further analyzed by a proteomic method such as by liquid chromatography (LC) (e.g. high performance liquid chromatography), liquid chromatography-mass spectrometry (LC-MS), matrix-assisted laser desorption/ionization (MALDI-TOF), gas chromatography-mass spectrometry (GC-MS), capillary electrophoresis-mass spectrometry (CE-MS), or nuclear magnetic resonance imaging (NMR).

In some embodiments, the LC method is any suitable LC methods well known in the art, for separation of a sample into its individual parts. This separation occurs based on the interaction of the sample with the mobile and stationary phases. Since there are many stationary/mobile phase combinations that are employed when separating a mixture, there are several different types of chromatography that are classified based on the physical states of those phases. In some embodiments, the LC is further classified as normal-phase chromatography, reverse-phase chromatography, size-exclusion chromatography, ion-exchange chromatography, affinity chromatography, displacement chromatography, partition chromatography, flash chromatography, chiral chromatography, and aqueous normal-phase chromatography.

In some embodiments, the LC method is a high performance liquid chromatography (HPLC) method. In some embodiments, the HPLC method is further categorized as normal-phase chromatography, reverse-phase chromatography, size-exclusion chromatography, ion-exchange chromatography, affinity chromatography, displacement chromatography, partition chromatography, chiral chromatography, and aqueous normal-phase chromatography.

In some embodiments, the HPLC method of the present disclosure is performed by any standard techniques well known in the art. Exemplary HPLC methods include hydrophilic interaction liquid chromatography (HILIC), electrostatic repulsion-hydrophilic interaction liquid chromatography (ERLIC) and reverse phase liquid chromatography (RPLC).

In some embodiments, the LC is coupled to a mass spectroscopy as a LC-MS method. In some embodiments, the LC-MS method includes ultra-performance liquid chromatography-electrospray ionization quadrupole time-of-flight mass spectrometry (UPLC-ESI-QTOF-MS), ultra-performance liquid chromatography-electro spray ionization tandem mass spectrometry (UPLC-ESI-MS/MS), reverse phase liquid chromatography-mass spectrometry (RPLC-MS), hydrophilic interaction liquid chromatography-mass spectrometry (HILIC-MS), hydrophilic interaction liquid chromatography-triple quadrupole tandem mass spectrometry (HILIC-QQQ), electrostatic repulsion-hydrophilic interaction liquid chromatography-mass spectrometry (ERLIC-MS), liquid chromatography time-of-flight mass spectrometry (LC-QTOF-MS), liquid chromatography-tandem mass spectrometry (LC-MS/MS), multidimensional liquid chromatography coupled with tandem mass spectrometry (LC/LC-MS/MS). In some instances, the LC-MS method is LC/LC-MS/MS. In some embodiments, the LC-MS methods of the present disclosure are performed by standard techniques well known in the art.

In some embodiments, the GC is coupled to a mass spectroscopy as a GC-MS method. In some embodiments, the GC-MS method includes two-dimensional gas chromatography time-of-flight mass spectrometry (GC*GC-TOFMS), gas chromatography time-of-flight mass spectrometry (GC-QTOF-MS) and gas chromatography-tandem mass spectrometry (GC-MS/MS).

In some embodiments, CE is coupled to a mass spectroscopy as a CE-MS method. In some embodiments, the CE-MS method includes capillary electrophoresis-negative electrospray ionization-mass spectrometry (CE-ESI-MS), capillary electrophoresis-negative electrospray ionization-quadrupole time of flight-mass spectrometry (CE-ESI-QTOF-MS) and capillary electrophoresis-quadrupole time of flight-mass spectrometry (CE-QTOF-MS).

In some embodiments, the nuclear magnetic resonance (NMR) method is any suitable method well known in the art for the detection of one or more cysteine binding proteins or protein fragments disclosed herein. In some embodiments, the NMR method includes one dimensional (1D) NMR methods, two dimensional (2D) NMR methods, solid state NMR methods and NMR chromatography. Exemplary 1D NMR methods include ¹Hydrogen, ¹³Carbon, ¹⁵Nitrogen, ¹⁷Oxygen, ¹⁹Fluorine, ³¹Phosphorus, ³⁹Potassium, ²³Sodium, ³³Sulfur, ⁸⁷Strontium, ²⁷Aluminium, ⁴³Calcium, ³⁵Chlorine, ³⁷Chlorine, ⁶³Copper, ⁶⁵Copper, ⁵⁷Iron, ²⁵Magnesium, ¹⁹⁹Mercury or ⁶⁷Zinc NMR method, distortionless enhancement by polarization transfer (DEPT) method, attached proton test (APT) method and 1D-incredible natural abundance double quantum transition experiment (INADEQUATE) method. Exemplary 2D NMR methods include correlation spectroscopy (COSY), total correlation spectroscopy (TOCSY), 2D-INADEQUATE, 2D-adequate double quantum transfer experiment (ADEQUATE), nuclear overhauser effect spectroscopy (NOSEY), rotating-frame NOE spectroscopy (ROESY), heteronuclear multiple-quantum correlation spectroscopy (HMQC), heteronuclear single quantum coherence spectroscopy (HSQC), short range coupling and long range coupling methods. Exemplary solid state NMR method include solid state ¹³Carbon NMR, high resolution magic angle spinning (HR-MAS) and cross polarization magic angle spinning (CP-MAS) NMR methods. Exemplary NMR techniques include diffusion ordered spectroscopy (DOSY), DOSY-TOCSY and DOSY-HSQC.

In some embodiments, the protein fragments are analyzed by a method as previously described. See PCT International Publication No. WO 2020/214336 to Hsu et al., published Oct. 22, 2020, the disclosure of which is incorporated herein by reference in its entirety.

In some embodiments, the results from the mass spectroscopy method are analyzed by an algorithm for protein identification. In some embodiments, the algorithm combines the results from the mass spectroscopy method with a protein sequence database for protein identification. In some embodiments, the algorithm comprises ProLuCID algorithm, Probity, Scaffold, SEQUEST, or Mascot.

In accordance with the presently disclosed subject matter, as described above or as discussed in the EXAMPLES below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al. (1989) Molecular Cloning: A Laboratory Manual, Cold Spring Harbor Laboratory Publications, Cold Spring Harbor, New York, United States of America; Glover (1985) DNA Cloning: A Practical Approach. Oxford Press, Oxford; Gait (1984) Oligonucleotide Synthesis: A Practical Approach, IRL Press, Oxford, England; Harlow & Lane, 1988, Antibodies, A Laboratory Manual, Cold Spring Harbor Publications, New York; Roe et al. (1996) DNA Isolation and Sequencing: Essential Techniques, John Wiley, New York, New York, United States of America; and Ausubel et al. (1995) Current Protocols in Molecular Biology, Greene Publishing.

### XI. Kits/Articles of Manufacture

Disclosed herein, in certain embodiments, are kits and articles of manufacture for use with one or more methods described herein. In some embodiments, described herein is a kit for generating a protein comprising a detectable group and/or a fragment of a ligand compound described herein. In some embodiments, such kit includes a probe or ligand as described herein, small molecule fragments or libraries, and/or controls, and reagents suitable for carrying out one or more of the methods described herein. In some instances, the kit further comprises samples, such as a cell sample, and suitable solutions such as buffers or media. In some embodiments, the kit further comprises recombinant proteins for use in one or more of the methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use. For example, the container(s) include probes, ligands, control compounds, and one or more reagents for use in a method disclosed herein.

The presently disclosed kits and articles of manufacture optionally include an identifying description or label or instructions relating to its use in the methods described herein. For example, a kit typically includes labels listing contents and/or instructions for use, and package inserts with instructions for use. A set of instructions will also typically be included. In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. The label also indicates directions for use of the contents, such as in the methods described herein.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Example 1

### Synthesis of KY-26 and KY-424

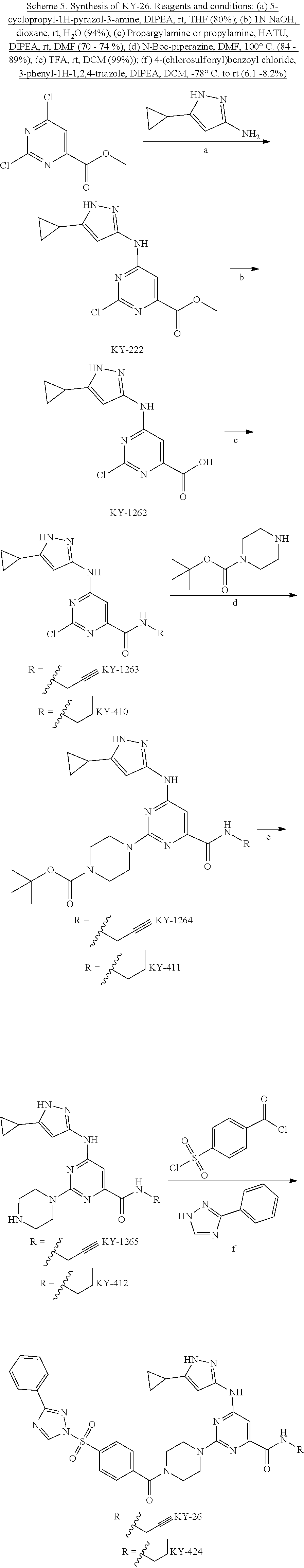

Compounds KY-1262, KY-1263, KY-410, KY-1264, KY-411, KY-1265, KY-412, KY-26 and KY-424 were prepared as shown in Scheme 5, above. KY-222 (i.e., the methyl ester of 2-chloro-6-[(5-cyclopropyl-1H-pyrazol-3-yl)amino]pyrimidinepyrimidine-carboxylic acid) can be prepared as shown in Scheme 5, above, from the methyl ester of 2,6-dichloropyrimidine-4-carboxylic acid or purchased from a commercial source (e.g., Combi-Blocks, Inc., San Diego, California, United States of America). Additional details regarding the synthesis of KY-410, KY-411, KY-412, KY-26 and KY-424 are described below.

### 2-chloro-6-((5-cyclopropyl-1H-pyrazol-3-yl)amino)-N-propylpyrimidine-4-carboxamide (KY-410)

As shown in Scheme 5, above, to a solution of KY-222 (883.5 mg, 3.2 mmol), propylamine (205.1 mg, 3.5 mmol), and DIPEA (1628.2 mg, 12.6 mmol) in DMF (20 mL) was added HATU (1558.1 mg, 4.1 mmol) over 15 min. The reaction was left overnight under nitrogen at room temperature. The reaction was concentrated in vacuo and the residue was partitioned between 50 mL of ethyl acetate and 50 mL of water. The aqueous phase was extracted 2 additional times with 50 mL of ethyl acetate. The organic phase was combined and washed with 150 mL of brine and dried over MgSO₄. The organic phase was concentrated in vacuo and purified via silica gel chromatography (1:1 ethyl acetate:hexanes to 100% ethyl acetate). White solid (750.2 mg, 74% yield). ¹H NMR (600 MHz, DMSO-d₆) δ 12.32 (s, 0.5H), 12.23 (s, 0.5H), 10.75 (s, 0.5H), 10.53 (s, 0.5H), 8.64 (s, 1H), 8.18 (s, OH), 7.31 (s, 1H), 6.39 (s, 1H), 5.66 (s, OH), 3.20 (dt, J=8.0, 6.3 Hz, 2H), 1.96-1.84 (m, 1H), 1.51 (h, J=7.4 Hz, 2H), 0.98-0.90 (m, 2H), 0.85 (t, J=7.4 Hz, 3H), 0.73-0.64 (m, 2H).ESI-TOF (HRMS) m/z [M+H]⁺ calculated for chemical formula: C₁₄H₁₈ClN₆O 321.1225, found 321.1224.

### tert-butyl 4-(4-((5-cyclopropyl-1H-pyrazol-3-yl)amino)-6-(propylcarbamoyl)pyrimidin-2-yl)piperazine-1-carboxylate (KY-411)

KY-410 (662.1 mg, 2.1 mmol) and N-Boc-piperazine (768.8 mg, 4.1 mmol) was dissolved in 20 mL DMF and heated to 100° C. for 2 hrs. The reaction progress was monitored via TLC (100% ethyl acetate as the solvent system). The reaction was concentrated in vacuo and the crude residue dissolved in ethyl acetate (30 mL). The organic layer was washed with saturated ammonium chloride (30 mL), saturated sodium bicarbonate (30 mL), and brine (30 mL). The organic phase was dried over MgSO₄, concentrated in vacuo and purified via silica gel chromatography (ethyl acetate:hexanes=1:1 to 2:1). (White solid 863.1 mg, 89%). ¹H NMR (600 MHz, DMSO-d₆) δ 12.02 (s, 1H), 9.84 (s, 1H), 8.56 (t, J=6.2 Hz, 1H), 7.95 (s, 1H), 6.73 (s, 0.5H), 6.24 (s, 0.5H), 3.81-3.73 (m, 4H), 3.43-3.38 (m, 4H), 3.23-3.15 (m, 2H), 1.94-1.84 (m, 1H), 1.51 (h, J=7.5 Hz, 2H), 1.43 (s, 9H), 0.93 (d, J=7.8 Hz, 2H), 0.86 (t, J=7.4 Hz, 3H), 0.72-0.62 (m, 2H). ESI-TOF (HRMS) m/z [M+H]⁺ calculated for chemical formula: C₂₃H₃₅N₈O₃ 471.2827, found 471.2830.

### 6-((5-cyclopropyl-1H-pyrazol-3-yl)amino)-2-(piperazin-1-yl)-N-propylpyrimidine-4-carboxamide 2,2,2-trifluoroacetate (KY-412)

12 mL of trifluoroacetic acid (TFA) was added to a solution of KY-411 (809.2 mg, 1.7 mmol) in DCM (20 mL). The mixture was stirred at room temperature for 1 hr before concentrating the reaction mixture in vacuo. The TFA was removed azeotropically. Light yellow solid (825.3 mg, 99% yield). ¹H NMR (600 MHz, DMSO-d₆) δ 10.02 (s, 1H), 9.03 (s, 2H), 8.65 (t, J=6.3 Hz, 1H), 6.93 (s, 1H), 6.12 (s, 1H), 4.00 (s, 4H), 3.27-3.12 (m, 6H), 1.91 (tt, J=8.3, 5.0 Hz, 1H), 1.51 (h, J=7.4 Hz, 2H), 0.94 (dt, J=8.5, 3.2 Hz, 2H), 0.86 (td, J=7.4, 1.9 Hz, 3H), 0.77-0.65 (m, 2H). ESI-TOF (HRMS) m/z [M+Na]⁺ calculated for chemical formula: C₁₈H₂₆N₈ONa 393.2122, found 393.2120.

### 6-((5-cyclopropyl-1H-pyrazol-3-yl)amino)-2-(4-(4-((3-phenyl-1H-1,2,4-triazol-1-yl)sulfonyl)benzoyl)piperazin-1-yl)-N-(prop-2-yn-1-yl)pyrimidine-4-carboxamide (KY-26)

To a solution of 4-(chlorosulfonyl)benzoyl chloride (392 mg, 1.64 mmol) in anhydrous DCM (10 mL) was added DIPEA (789 μL, 4.52 mmol) and KY-1265 (500 mg, 1.37 mmol) in anhydrous DCM (20 mL) over the course of 15 min at −78° C. The reaction temperature was maintained at −78° C. for 1 hr. 3-phenyl-1H-1,2,4-triazole (198 mg, 1.37 mmol) and DIPEA (263 μL, 1.51 mmol) in DCM (10 mL) were added to the reaction mixture and the reaction was stirred at room temperature for 2 hrs. The reaction was concentrated in vacuo and was purified via silica gel flash chromatography (hexanes:ethyl acetate=1:4 to 1:10). KY-26 (76 mg, 0.11 mmol, 8.2%) was obtained as a white solid. KY-26 purity was estimated to be 95.6% based on HPLC analysis (see data in section VI). ¹H NMR (600 MHz, DMSO-d₆) δ 12.00 (s, 1H), 9.86 (s, 1H), 9.52 (s, 1H), 8.90 (s, 1H), 8.27-8.23 (m, 2H), 8.06-7.99 (m, 2H), 7.84-7.79 (m, 2H), 7.55-7.48 (m, 3H), 6.76 (s, 1H), 6.14 (s, 1H), 4.14-3.52 (m, 1 OH), 3.08 (t, J=2.5 Hz, 1H), 1.84 (s, 1H), 0.98-0.72 (m, 2H), 0.63 (m, 2H). ESI-TOF (HRMS) m/z [M+H]⁺ calculated for chemical formula: C₃₃H₃₂N₁₁O₄S 678.2354, found 678.2352.

### 6-((5-cyclopropyl-1H-pyrazol-3-yl)amino)-2-(4-(4-((3-phenyl-1H-1,2,4-triazol-1-yl)sulfonyl)benzoyl)piperazin-1-yl)-N-propylpyrimidine-4-carboxamide (KY-424)

To a solution of 4-(chlorosulfonyl)benzoyl chloride (52.8 mg, 0.2 mmol) in anhydrous DCM (10 mL) was added DIPEA (64.3 μL, 0.4 mmol) and KY-412 (89.2 mg, 0.18 mmol) in anhydrous DCM (20 mL) over the course of 15 min at −78° C. for 1 hr before adding 3-phenyl-1H-1,2,4-triazole (31.8 mg, 0.2 mmol) and DIPEA (38.6 μL, 0.2 mmol) in DCM (10 mL). The reaction was kept at room temperature overnight. The reaction was concentrated in vacuo and was purified via silica gel flash chromatography (hexanes:ethyl acetate=1:4 to 0:100). KY-424 (7.6 mg, 6.1%) was obtained as a beige solid. ¹H NMR (600 MHz, DMSO-d₆) δ 12.01 (s, 1H), 9.83 (s, 1H), 9.53 (d, J=2.0 Hz, 1H), 8.52 (s, 1H), 8.27-8.21 (m, 2H), 8.05-7.98 (m, 2H), 7.85-7.77 (m, 2H), 7.55-7.47 (m, 3H), 6.74 (s, 1H), 6.15 (s, 1H), 4.00-3.85 (m, 2H), 3.80-3.67 (m, 4H), 3.30-3.14 (m, 4H), 1.90-1.78 (m, 1H), 1.58-1.37 (m, 2H), 0.92-0.76 (m, 5H), 0.69-0.55 (m, 2H). ESI-TOF (HRMS) m/z [M+H]⁺ calculated for chemical formula: C₃₃H₃₆N₁₁O₄S 682.2667, found 682.2666.

### Example 2

### Methods for Examples 3-6

HPLC Analysis of KY-26 Reactions with Amino Acid Mimetics and Synthetic Peptides:

Briefly, reaction progress of KY-26 or XO44 with p-cresol or n-butylamine (16.5 μmol, 3.3 eq.) in the presence of TMG base (1,1,3,3-tetramethylguanidine, 1.1 eq) was evaluated by monitoring probe consumption and quantified based on the signal from the caffeine standard using HPLC. Synthetic peptide reactions were conducted by mixing the peptide (Ac-RLNERHYGGLTGLNK-NH₂, 50 nmol, 1.0 eq.) with 1.1 eq of TMG. KY-26 (550 nmol, 11.0 eq) was added to the mixture and the reaction was kept at 37° C. until the reaction achieved at least 50% conversion.

More particularly, an acetonitrile solution of p-cresol or n-butylamine (16.5 μmol, 3.3 eq.) was mixed with 1.1 eq of TMG. KY-26 or XO44 in ACN:DMF=6:4 (5 μmol, 1.0 eq.) was added to the mixture and the reaction was kept at 0° C. The final molarity for KY-26 or XO44 is 10 mM. Aliquots (50 μl) of the reaction were taken and quenched by adding acetic acid (0.5 M final, 5.0 μmol) and caffeine standard (0.05 M final, 0.5 μmol) at ten-minute intervals for 1.5 hrs. 1.0 μL sample was injected and analyzed by reverse-phase HPLC with mobile phases A (H₂O, 0.1% AcOH) and B (CH₃CN, 0.1% AcOH) with a gradient of 0-15-85-100% B in 0-0.5-6.5-7 min. UV measurements taken at 254 nm. Reaction progress was evaluated by monitoring KY-26 or XO44 consumption and quantified based on the signal from the caffeine standard.

Synthetic peptide reactions were conducted by mixing the peptide (Ac-RLNERHYGGLTGLNK-NH₂, 50 nmol, 1.0 eq.) with 1.1 eq of TMG. KY-26 (550 nmol, 11.0 eq) was added to the mixture and the reaction was kept at 37° C. The reaction progress was monitored via Shimadzu Prominent Series HPLC (Shimadzu Corporation, Kyoto, Japan) and SPD-20A series UV-vis spectrometer at 220 nm using a Thermo Fisher Scientific C30 column (sold under the tradename ACCLAIM™, Thermo Fisher Scientific, Waltham, Massachusetts, United States of America; 3 μm, 2.3×150 mm). The mobile phases A and B were composed 0.1% AcOH in H₂O and 0.1% AcOH in CH₃CN, respectively. Using a constant flow rate of 0.3 mL/min, the gradient was as follows: 0-1 min, 5% B; 1-16 min 5-36% B (linear gradient); 16-19 min 36-100% B (linear gradient); 19-24 min 100% B; 24-25 min 100-5% B (linear gradient); 25-30 min 15% B. Once the reaction achieved at least 50% conversion, a desthiobiotin tag was appended to the product by the addition of TCEP (550 nmol, 11.0 eq.), TBTA (1.1 μmol, 22.0 eq.), desthiobiotin-PEG₃-azide (550 nmol, 11.0 eq.), and CuSO₄ (50 nmol, 1.0 eq.). The mixture was stirred for 24 hrs and lyophilized prior to LC-MS analysis.

### Gel-Based Chemical Proteomics:

Briefly, Jurkat cells were grown to 80% confluency and treated with either DMSO or probe at the designated final concentration (KY-26 or XO44, 1,000× stock in serum-free media (SFM)) and incubated at 37° C. with 5% CO₂ for 30 min. Cells were harvested and lysed in PBS buffer containing EDTA-free protease inhibitors. Addition of the rhodamine fluorescent tag was accomplished by CuAAC and fluorescently labeled proteins visualized by SDS-PAGE and in-gel fluorescence scanning. For KY-26 and XO44 live cell treated samples, 50 μL aliquots of proteome were used for gel experiments. For KY-26 lysate labeling, 49 μL aliquots of cell lysate were used for each dose and time point before adding 1 μL of 50× KY-26 stock (5 μM final) and incubated for 30 min at 37° C. For competition experiments, ATP or KY-424 (1 μL of a 50× stock) was added to a proteome sample (48 μL) and incubated for 30 min at 37° C. before adding KY-26 (1 μL of a 50× stock, 5 μM final).

More particularly, Jurkat cells were cultured at 37° C. with 5% CO₂ in RPMI with 10% fetal bovine serum and 1% L-glutamine in 10 cm² tissue culture dishes. Cells were grown to 80% confluency for experimental use or to passage. For treatments, Jurkat cells were grown to 80% confluency and treated with either DMSO or probe at a final concentration of 5 μM of KY-26 or XO44 from a 1,000× stock in serum-free media. Cells were subsequently incubated at 37° C. with 5% CO₂ for 30 min. Cells were harvested and pelleted at 400×g for 5 min and the supernatant was decanted. Cells were re-suspended in cold PBS and centrifuged at 400×g for 5 min and the supernatant was decanted once more. The PBS wash was repeated for a second time before cells were snap frozen and stored at −80° C. for future experiments. Dose-response assays were performed to optimize treatment conditions for KY-26 in a similar manner. Jurkat cells were treated with increasing concentrations of KY-26 (5 μM-25 μM) and were harvested at 0, 30, 60, 90, and 120 min.

Cell pellets were lysed in PBS buffer (PBS+a protease inhibitor (sold under the tradename PIERCE™, Thermo Fisher Scientific, Waltham, Massachusetts, United States of America), EDTA free) by sonicating 3 times for 1 second×20% amplitude. The lysate was fractionated by centrifuging at 100,000×g for 25 min at 4° C., separating membrane and soluble fractions. Protein concentrations were measured using the Bio-Rad DC protein assay (Bio-Rad Laboratories, Hercules, California, United States of America), and fractions were diluted to a concentration of 1 mg/mL in PBS. For KY-26 and XO44 live cell treated samples, 50 μL aliquots of proteome were used for gel experiments. For KY-26 lysate labeling, 49 μL aliquots of cell lysate were used for each dose and time point before adding 1 μL of 50×KY-26 stock (5 μM final) and incubated for 30 min at 37 C. For ATP and KY-424 competition experiment, 1 μL of the 50× stock of ATP or KY-424 was added to the 48 μL of proteome aliquot and incubated for 30 min at 37° C. before adding 1 μL of 50×KY-26 stock (5 μM final). Addition of the rhodamine fluorescent tag was accomplished by adding CuAAC reagents in the following manner: 1 μl of 1.25 mM stock of rhodamine-azide in DMSO (25 μM final), 1 μl of freshly prepared 50 mM TCEP stock in water (1 mM final), 3 μl of a 1.7 mM TCEP stock in 4:1 t-butanol/DMSO (100 μM final), and 1 μl of a 50 mM CuSO₄ stock (1 mM final concentration). Samples were immediately vortexed, and the reaction proceeded for 1 hr at room temperature. Reactions were quenched with 17 μL of 4×SDS-PAGE loading buffer and beta-mercaptoethanol. 30 μL of each sample were separated by SDS-PAGE and analyzed by in-gel fluorescence scanning for the rhodamine azide tag. Coomassie staining was used to control for equivalent protein loading across lanes.

### Enrichment of KY-26 Modified Peptides for LC-MS/MS Analysis:

Soluble proteomes (0.5 mg) were diluted to 432 μL in kinase buffer (PBS, 50 mM MgCl₂, & protease inhibitor (sold under the tradename PIERCE™ (Thermo Fisher Scientific, Waltham, Massachusetts, United States of America), EDTA free) in a low-bind microfuge tube. Addition of the desthiobiotin affinity tag was accomplished by adding CuAAC reagents in the following manner: 10 μl of 2.5 mM stock in of desthiobiotin-PEG₃-azide in DMSO (50 μM final), 10 μl of freshly prepared 50 mM TCEP stock in water (1 mM final), 33 μl of a 1.7 mM TBTA stock in 4:1 t-butanol/DMSO (100 μM final), and 10 μl of a 50 mM CuSO₄ stock (1 mM final concentration). Samples were quickly vortexed and incubated for 2 hrs under constant rotation at room temperature (25° C.). Click chemistry reagents were subsequently removed by adding 500 μL of MeOH, 125 μL CHCl₃, vortexing, and centrifuging at 1,300×g for 3 min to precipitate the protein between the organic and aqueous interface. The solvents were removed, and the protein precipitate was re-suspended in 400 μL of 6 M urea in 50 mM ammonium bicarbonate. DTT (10 mM) was added to the solution, which was then incubated at 50° C. for 15 min for reduction of disulfide bonds. Samples were cooled to room temperature before addition of IAA (20 mM) to alkylate cysteines at room temperature, in the dark, for 30 min. Excess IAA and DTT were removed by repeating the chloroform:methanol precipitation step once more. Samples were diluted with 50 mM ammonium bicarbonate and split into two low-bind tubes. For proteolytic digestion, a 1:200 ratio of trypsin or chymotrypsin to total protein was incubated overnight at 37° C. or room temperature, respectively. Digests were combined in 15 mL conicals containing 50 μL of avidin beads and diluted to 5.5 mL in PBS, KY-26 modified peptides were enriched by rotating conicals for 2 hrs at room temperature. Beads were washed 3 times with 50 mM ammonium bicarbonate and 3 times with LC-MS grade water. Bound peptides were eluted by incubating beads with avidin elution buffer (50% acetonitrile:50% water:0.1% formic acid) for 3 min. Beads were centrifuged at 1,300×g for 3 min, and the supernatant was transferred to a new low-bind microfuge tube. The elution was repeated twice more (two more times), and samples were dried down and stored in a −80° C. freezer.

### Sample Cleanup by Hydrophilic Interaction Liquid Chromatography (HILIC)⁴⁹

A PHEA slurry was prepared with 200 mM ammonium formate (pH 3) and added to a fritted 200 μL pipette tip to a bed length of 5 mm. The media was washed once more with 200 mM ammonium formate, twice with water, and twice with loading buffer (90% acetonitrile:10% water:10 mM ammonium formate, pH 3). Peptide standards (50 fmol/μL final concentration) were added to the dried sample, reconstituted in loading buffer, and added to the PHEA column. The flow through was collected in a low-bind microfuge tube. The column was washed once more in loading buffer, and the flow through was collected in the same tube. Peptides were eluted from the column by the addition of elution buffer (50% acetonitrile:50% water:10 mM ammonium formate, pH 3 followed by 20% acetonitrile:80% water:10 mM ammonium formate, pH 3), and the flow through was collected into a second low-bind microfuge tube. A final wash was performed using 0.2% formic acid and collected into a third low-bind tube. All fractions were dried down and either analyzed immediately or stored at −80° C. Before analysis by LC-MS, peptides were reconstituted in 1 μL acetic acid, vortexed vigorously, and diluted with 15 μL of LC-MS grade water.

Common methods to de-salt and reduce contaminant ions for LC-MS analysis typically employ C18 stationary phase^{50,51}. However, C18 was incompatible with KY-26 modified peptides and resulted in significant loss of peptide products. Further, C18 cleanup would not remove polymer contaminants, only ionic salts, and can potentially concentrate polymer contaminants⁴. Thus, hydrophilic interaction liquid chromatography (HILIC) was used to remove most of these contaminants. The added benefit of an offline HILIC cleanup was that the stationary phase does not retain polymer contaminants and has been used to improve fractionation of PTM peptides (e.g. glycosylation⁴). With a hydrophobic modification such as KY-26, HILIC was predicted to elute modified peptides without significant loss of desired analytes.

### LC-MS/MS Analysis of KY-26-Modified Peptides

Probe-modified synthetic peptide was reconstituted in 5% AcOH and diluted to 5 pmol/μL concentration and analyzed using C18 (3 μm) or PLRP-S (3 μm) in a fused silica capillary (360 μm O.D.×75 μm I.D.) on an Agilent 1100 Series Binary HPLC (Agilent Technologies, Santa Clara, California, United States of America) interfaced with a Thermo Scientific (Waltham, Massachusetts, United States of America) mass spectrometer sold under the tradename LTQ-XL™. Samples loaded onto C18 columns were washed with solvent A (0.3% formic acid in water) for 30 min and eluted with a gradient of 0-100% solvent B (72% ACN, 18% IPA, 10% water, 0.3% formic acid). Additional attempts to elute the modified peptide used solvent B consisting of 90% ACN, 10% IPA, and 0.3% formic acid with the same gradient. Samples loaded onto PLRP-S columns were washed with solvent A (0.3% formic acid in water) for 30 min, then eluted from the column with increasing solvent B (72% ACN, 18% IPA, 10% water, 0.3% formic acid) from 0-30-70-100% in 0-5-25-30 min. A top 3 data dependent MS2 method was used, where the top 3 ions were selected from an MS1 scan of m/z 300-2000 for dissociation by CAD and ETD.

Probe-modified peptides (1 μl samples) from live cell studies (subjected to offline HILIC cleanup, as described below) were pressure loaded into a nanocapillary analytical column (10 cm, 3 μm 1000 Å PLRP-S packing material in 360 μm o.d.×75 μm i.d. fused silica), with an integrated electrospray tip. Samples were washed with solvent A (0.3% formic acid in water) for 15 min before peptide elution with 0-30-50-100% solvent B (72% ACN, 18% IPA, 10% water, 0.3% formic acid) in 0-5-60-65 min. Samples were initially electrosprayed into an in-house modified LTQ Velos Orbitrap mass spectrometer (Thermo Fisher Scientific, Waltham, Massachusetts, United States of America) operating with a data-dependent acquisition method that consisted of one full MS1 scan (300-2000 m/z, 120,000 resolution) followed by HCD and ETD MS2 scans for the top 5 most abundant ions recorded in the MS1 scan. Samples confirmed to contain KY-26 modified peptides were then analyzed on a mass spectrometer (sold under the tradename Orbitrap FUSION™ TRIBRID™ (Thermo Fisher Scientific, Waltham, Massachusetts, United States of America). Peptides were eluted from the same analytical column with a gradient of 0-30-50-100% solvent B in 0-5-60-65 min. All ions in the MS1 (300-2000 m/z) with charges 2-9 were initially fragmented by HCD (25% NCE, 80,000 resolution). A targeted mass trigger from HCD MS2 scans was used to detect ions arising from the desthiobiotin tag (240.1712 and 197.1290 m/z) for subsequent low-resolution ETD fragmentation of precursors (charges 3-9), in the ion trap. Comparison samples were analyzed as previously described.

### Data Analysis:

Briefly, identification of KY-26 modified peptides was accomplished by searching data files using Byonic version 3.3.4. Data were searched against the SwissProt human protein database (Jan. 6, 2020). Search parameters were set with a 10-ppm mass tolerance for precursor ions. High-resolution HCD MS/MS spectra were searched with a 50-ppm fragment mass tolerance and low-resolution ETD spectra with a 0.5 Da mass tolerance. Too low (wide) precursor isotope off by x, precursor and charge assignment computed from MS1. Modifications included a variable methionine oxidation (+15.9949 Da) and static cysteine alkylation (Iodoacetamide, +57.021464 Da). The addition of KY-26 was included as a variable (common) modification of one lysine, tyrosine, serine, and threonine residues with an added mass of +946.4232 Da. Tryptic searches allowed for 3 missed cleavages and were set to specific protease activity. Chymotryptic searches allowed for 10 missed cleavages and were set for non-specific protease activity. All searches included a 1% false discovery rate. Byonic results were exported to a spreadsheet and unmodified peptides and peptides with a score lower than 250 were filtered out.

### Example 3

### KY-26 Shows Enhanced Solution and Proteome Reactivity Compared with XO44

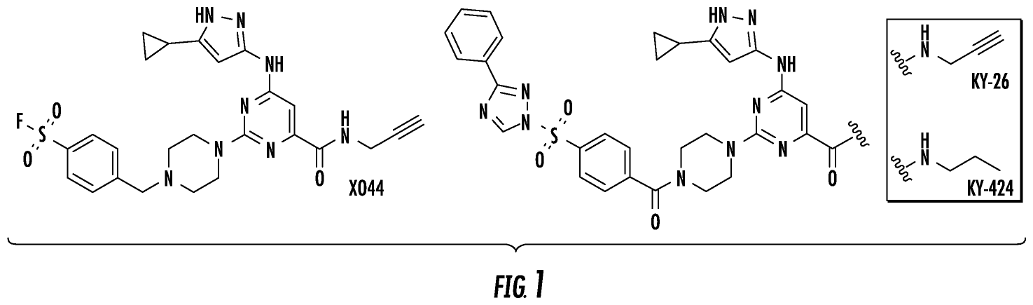

A sulfonyl-triazole analog of XO44 named KY-26 was synthesized for chemical proteomic studies. See FIG. 1. A rationale for selecting the triazolide in place of the fluoride as a leaving group is based on studies that demonstrated enhanced reactivity at protein sites for sulfur-triazole exchange (SuTEx) compared with sulfur-fluoride exchange (SuFEx) chemistry²⁴⁻²⁵. The sulfonyl-triazole reactive group was connected to the 2-aminopyrazole kinase-recognition unit through an amide linkage to increase the electron-withdrawing character of the adduct group²⁶ for enhanced reactivity of KY-26. Details of the synthesis and characterization of KY-26 and analogs can be found in Example 1, above.

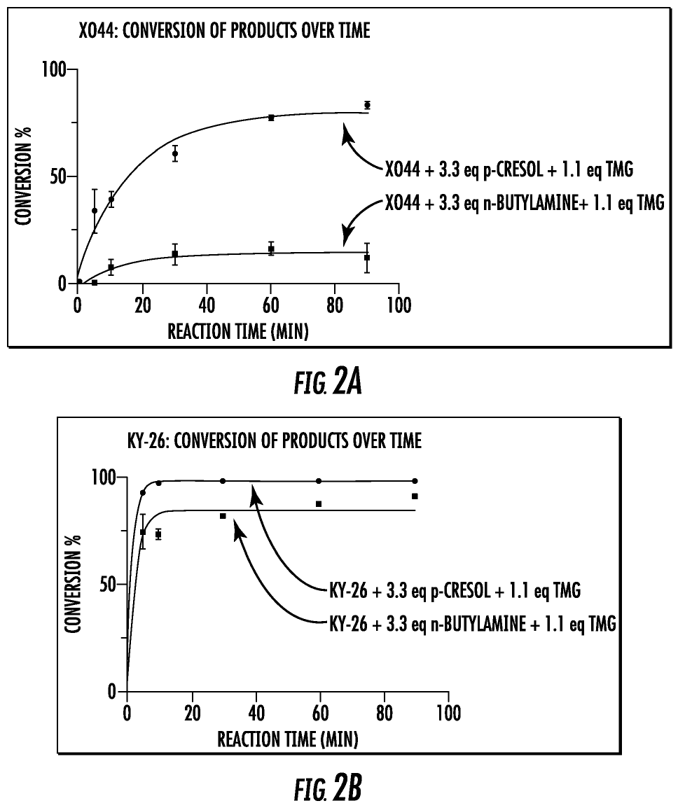

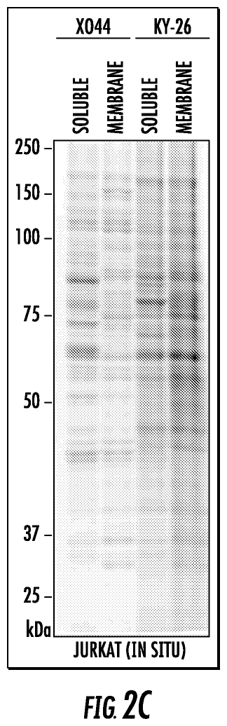

Initially, solution reactivity of KY-26 and XO44 against nucleophiles that mimic tyrosine (p-cresol) and lysine (n-butylamine) side-chains was compared by high-performance liquid chromatography (HPLC) as previously reported²⁵ and described in Example 2. In these assays, the reactivity of electrophilic compound is evaluated by its depletion, resulting in reduced UV signals, upon reaction with nucleophile as a function of time. Covalent reaction in solution was facilitated by addition of 1,1,3,3-tetramethylguanidine (TMG) base. Although both probes showed time-dependent reaction with p-cresol, KY-26 was more reactive as evidenced by near-complete depletion by 20 minutes (min), while XO44 reaction was incomplete at the longest time point tested (90 min). See FIGS. 2A and 2B. Interestingly, although XO44 was reported as a lysine-targeted kinase probe, minimal reaction with n-butylamine was observed in the HPLC studies. In agreement with the tyrosine chemoselectivity reported for SuTEx probes²⁴⁻²⁵ reduced activity of KY-26 against n-butylamine compared with p-cresol was observed. See FIG. 2B.

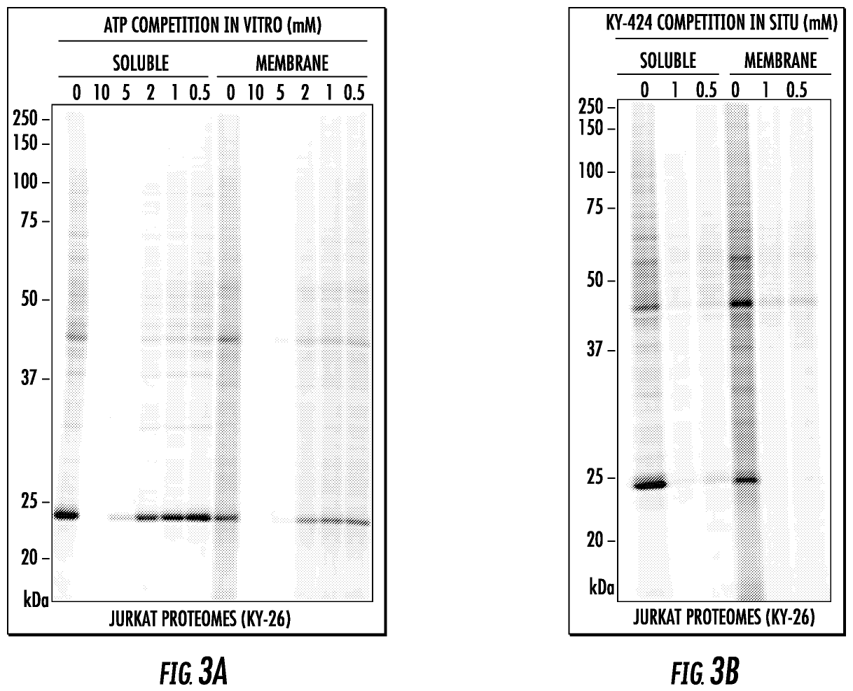

In addition to solution reactivity, activity of KY-26 and XO44 in proteomes was compared using gel-based chemical proteomics. Considering that both SuFEx¹⁴ and SuTEx²⁶ probes are cell permeable, chemical proteomic studies were performed under live cell treatment conditions as described in Example 2. In brief, Jurkat cells were treated with KY-26 or XO44 (5 μM, 30 min, 37° C.) followed by cell lysis, separation of soluble and membrane lysates, and conjugation of a rhodamine tag to the alkyne handle of probe-modified proteins by copper-catalyzed azide-alkyne cycloaddition (CuAAC²⁷). Probe-modified proteins were visualized by SDS-PAGE and in-gel fluorescence scanning. In agreement with the HPLC findings, substantially increased reactivity of KY-26 was observed compared with XO44 in proteomes from probe-treated cells as evidenced by increased fluorescence intensity across the entire molecular weight range in the gel-based studies. See FIG. 2C. Although KY-26 was generally more reactive, the gel-based analyses revealed shared and distinct proteome-wide targets for XO44 and KY-26. Specifically, several protein bands were labeled more prominently in proteomes from XO44-compared with KY-26-treated cells, which supports differences in selectivity between these probes. To test specificity of KY-26 labeling activity, in vitro competition studies were performed with both free ATP (10-0.5 mM) and a non-clickable analog of KY-26 (1 and 0.5 mM KY-424). See FIG. 1. A concentration-dependent blockade of KY-26 labeling of proteomes was observed with pretreatments from both competitors. See FIGS. 3A and 3B. These findings support detection of probe labeling events within the ATP-binding site of target proteins that is dependent on the KY-26 inhibitor scaffold.

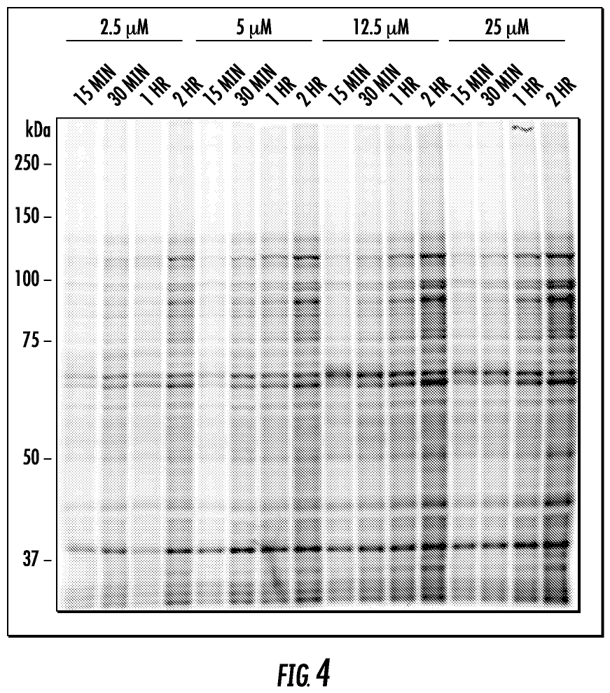

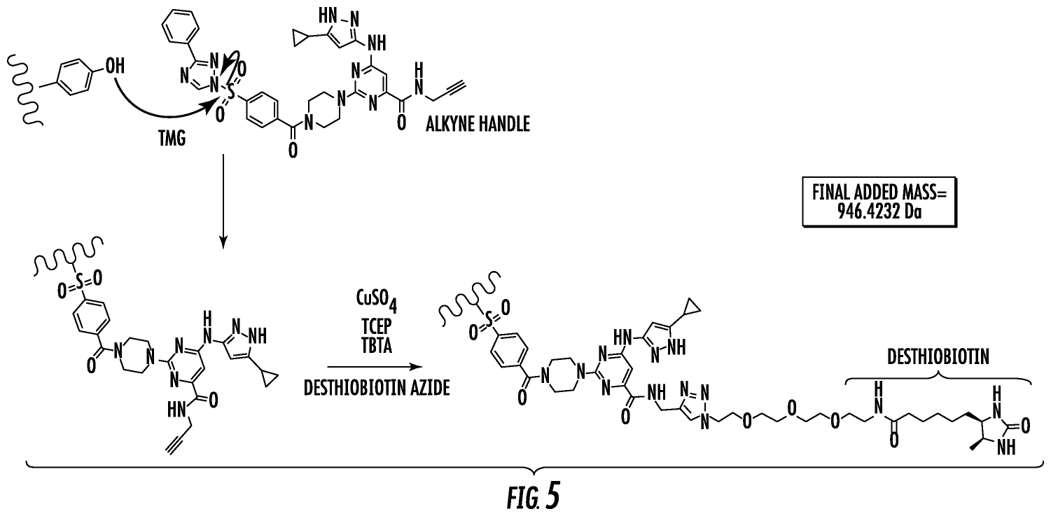

Next, concentration- and time-dependent labeling studies were performed with KY-26 in Jurkat cells to identify optimal probe labeling conditions. Cells were treated at varying concentrations (2.5-25 μM) and harvested cells for gel-based chemical proteomic analyses at different time points (15-120 min). Both concentration- and time-dependent probe labeling was observed. See FIG. 4. The latter finding further supports a covalent mode of action for KY-26 activity. Based on these findings, it was concluded that a live cell-treatment condition of 12.5 μM of KY-26 for 2 hours was optimal for probe labeling in situ.

### Example 4

### Improving Reverse-Phase Chromatography of KY-26 Probe-Modified Peptides Using PLRP-S Media

The ability to accurately identify binding sites from covalent probe modification is dependent on chromatographic separation of tryptic peptide digests of the proteome for MS identification. Probe-modified peptides generated from target proteins by protease digestion are conjugated to (desthio)biotin by CuAAC and enriched by avidin chromatography. Reverse-phase chromatography using C18 media separate these probe-modified peptides for site of binding identification using LC-MS/MS. While suitable for small covalent probes, larger and more structurally complex versions such as KY-26 are not likely to be efficiently eluted using standard reverse-phase LC conditions. This hypothesis was tested by using modifying synthetic peptides with KY-26 and comparing retention, elution, and MS detection of resulting probe-modified peptides under different LC conditions.

A synthetic peptide with the sequence Ac-RLNERHYGGLTGLNK-NH₂ (SEQ ID NO: 1) was reacted with KY-26 in solution. The progress of reaction was tracked by HPLC (UV detection) to confirm at least 50% conversion before subjecting to LC-MS/MS analyses. The N- and C-termini of the peptide were acetylated and amidated, respectively, to prevent reactions at the peptide termini. The substrate peptide contained a tyrosine and a lysine to provide multiple sites for KY-26 modification that was facilitated by the addition of TMG base. Prior to LC-MS/MS analyses, a desthiobiotin tag was conjugated by CuAAC in order to model a probe-modified peptide detected by chemical proteomics. See FIG. 5. Under typical C18 reverse-phase conditions, the KY-26-modified peptide was not detected in the reaction mixture, while the unmodified peptide was detected. Even with increased concentration of organic solvents in the mobile phase, the KY-26-modified peptide was not detected using the C18 analytical column. KY-26 is a large probe molecule that adds 946.4232 Da to a peptide after covalent reaction. Consequently, the present findings show that C18 media, while appropriate for standard tryptic peptide analysis, was not suitable for reverse-phase LC of peptides modified with bulky probe adducts.

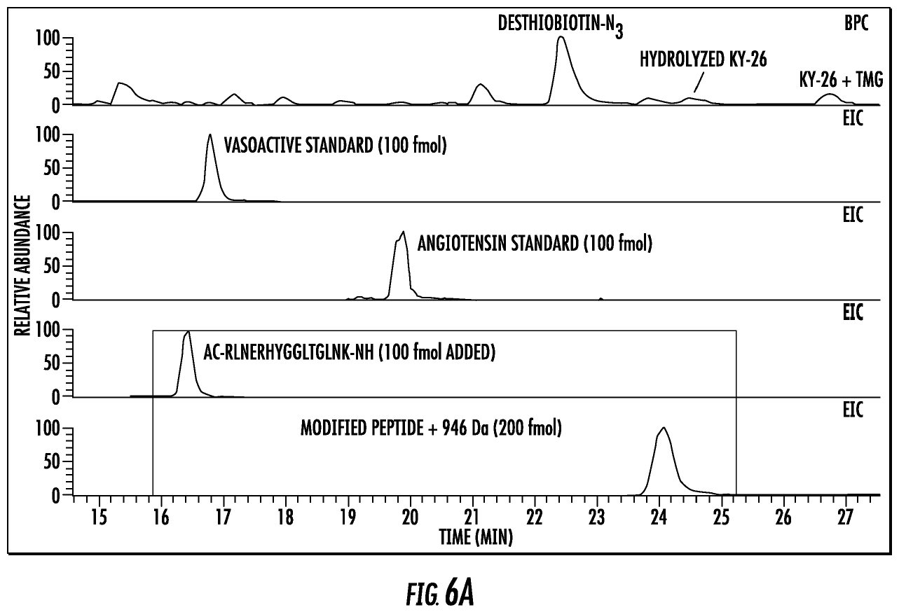

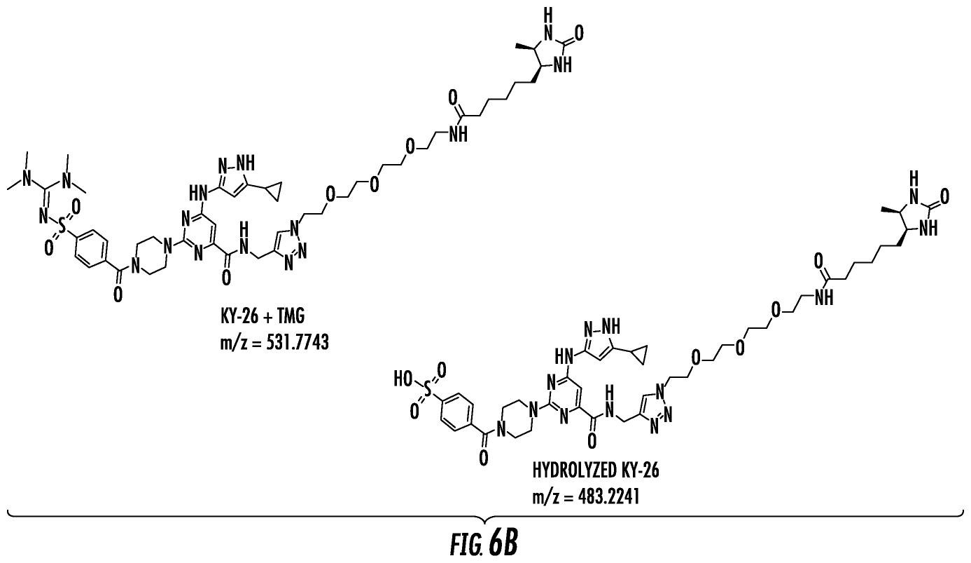

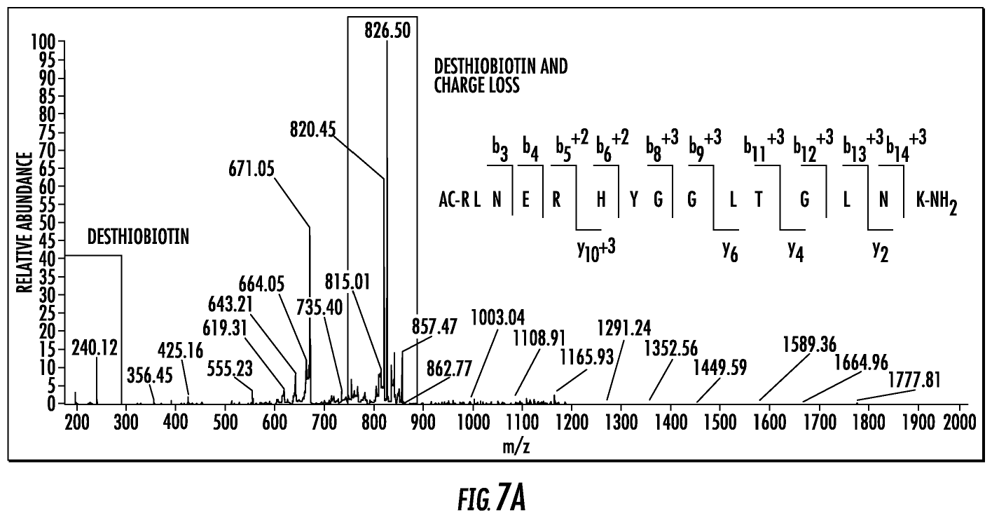

PLRP-S media has been used to elute hydrophobic molecules such as vancomycin, and in the chromatographic separation of proteins, including monoclonal antibodies²⁸⁻³⁰ Thus, it was surmised that PLRP-S could be a suitable alternative for chromatographic separation of KY-26-modified peptides. Analysis of reaction mixtures using a PLRP-S analytical column yielded detection of the KY-26-modified peptide. See FIGS. 6A and 6B. In support of the hypothesis, a ˜8 min retention time difference between the modified and unmodified peptides was observed, which indicates a substantial increase in hydrophobicity following KY-26 modification. See FIG. 6A. In summary, the presently disclosed findings support the PLRP-S stationary phase as an effective alternative to C18 for reverse-phase LC-MS/MS detection of KY-26 and potentially other covalent probes that produce bulky adducts on amino acid sites to increase hydrophobicity of resulting peptides.

### Example 5

### Electron-Transfer Dissociation Improved Sequence Coverage of a KY-26 Modified Synthetic Peptide

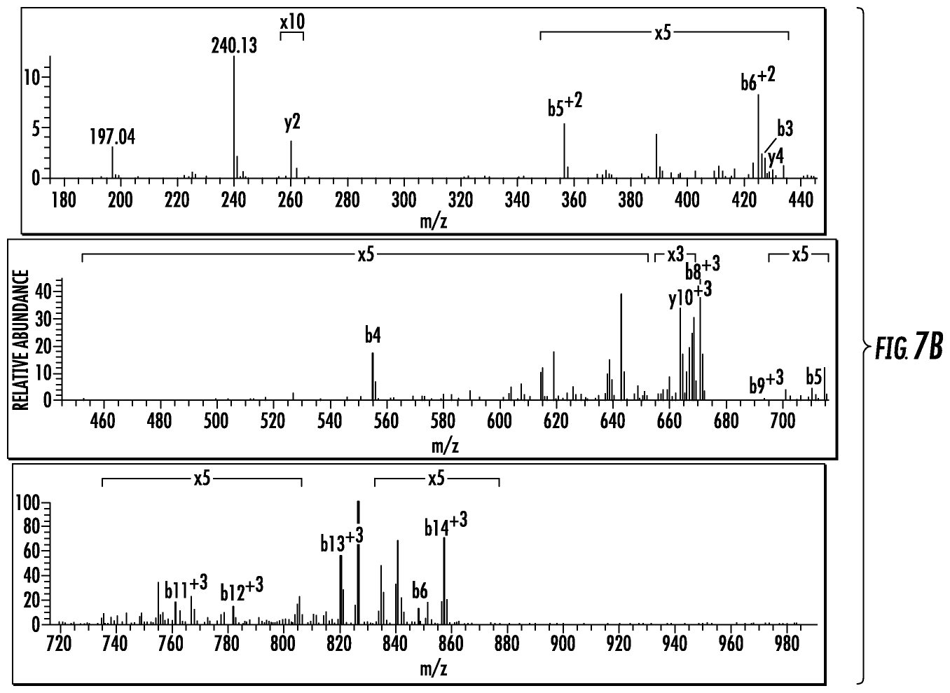

After identifying suitable LC conditions, the KY-26 modified synthetic peptide was sequenced by MS analysis to identify the site of KY-26 modification. Initially, fragmentation was performed by collisionally-activated dissociation (CAD), which yielded reasonable sequence coverage including the identity of the tyrosine residue modified by KY-26. See FIG. 7A. In addition to the standard b- and y-ion series, additional fragment ions that are derived from the desthiobiotin affinity tag (240 and 197 m/z; see FIG. 7B) were observed. These diagnostic fragment ions from KY-26 modification are consistent with findings from previous SuTEx probe studies using similar fragmentation (higher-energy C-trap dissociation or HCD)³¹. The present CAD studies also revealed that KY-26 modification results in increased peptide charge state (+4 ion; 679.35 m/z versus +3 because of an additional proton on the probe moiety). This result was expected as kinase inhibitors such as the binding element of KY-26 contain heterocycles that increase gas-phase basicity and impact the charge state of resulting peptides subjected to LC-MS/MS analysis²².

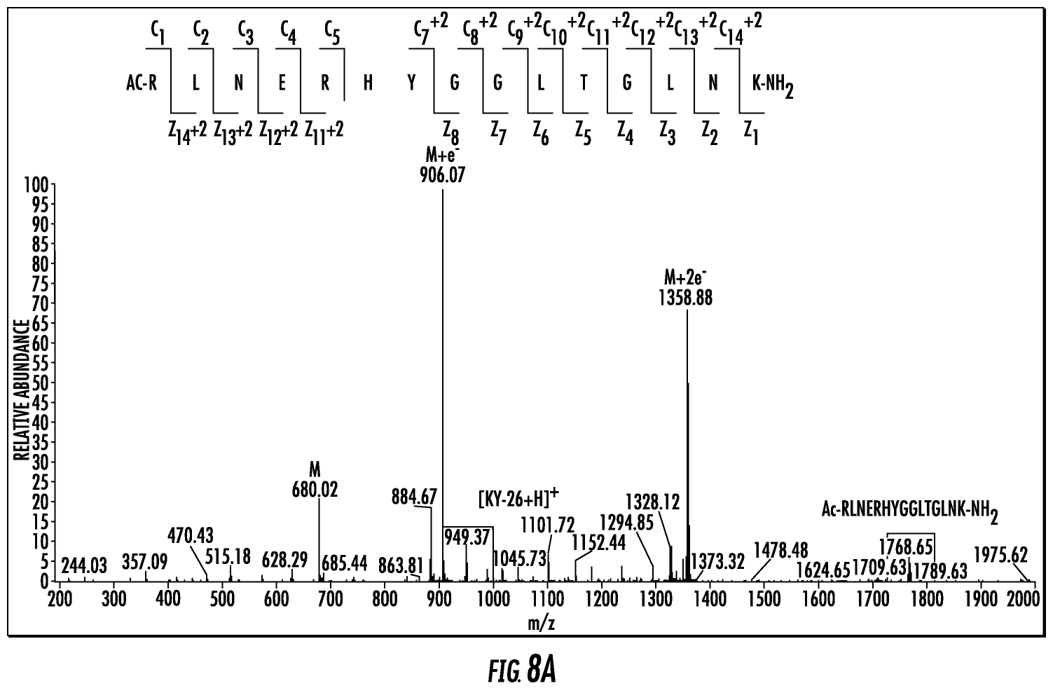

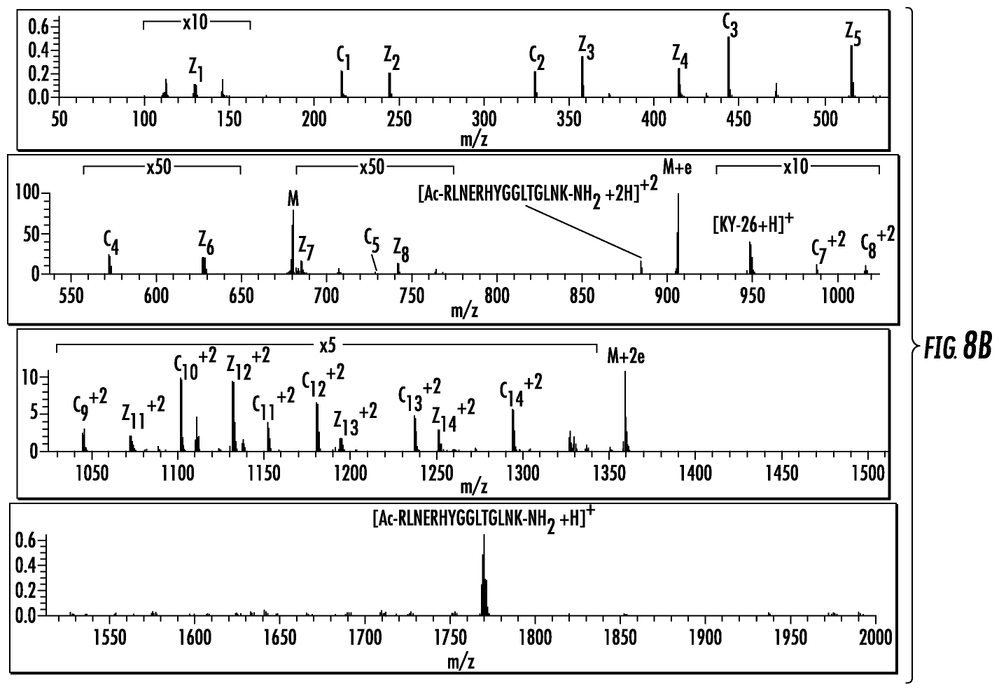

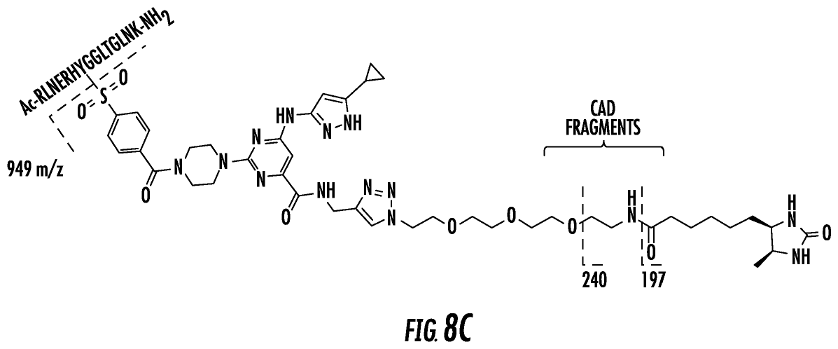

Higher charge state peptides yield complicated product ion spectra that contain multiply charged fragment ions that reduce the accuracy of search algorithms used for peptide identifications. The incomplete sequencing of the KY-26-modified peptide by CAD is likely a result of its higher charge state. This hypothesis was tested by sequencing the KY-26-modified peptide using electron-transfer dissociation (ETD), which is well-suited for analysis of higher charge state peptides (z ≥+3)³². Another beneficial feature of ETD is the ability to preserve labile modifications (e.g. phosphorylation³³) that would be predicted to reduce fragments generated from the desthiobiotin tag. Using ETD, near-complete sequencing (c- and z-ion series) of the KY-26-modified peptide including the site of probe modification (see FIG. 8A) was achieved. The 240 or 197 m/z fragment ions were not detected, indicative of preservation of the desthiobiotin tag. See FIG. 8B. A 949 m/z fragment ion that corresponded to loss of the KY-26 modification, which could be used to confirm the probe modification on peptides, was observed. See FIG. 8C.

### Example 6

### Increasing the Number of KY-26 Target Site Identifications in Live Cell Chemical Proteomic Studies

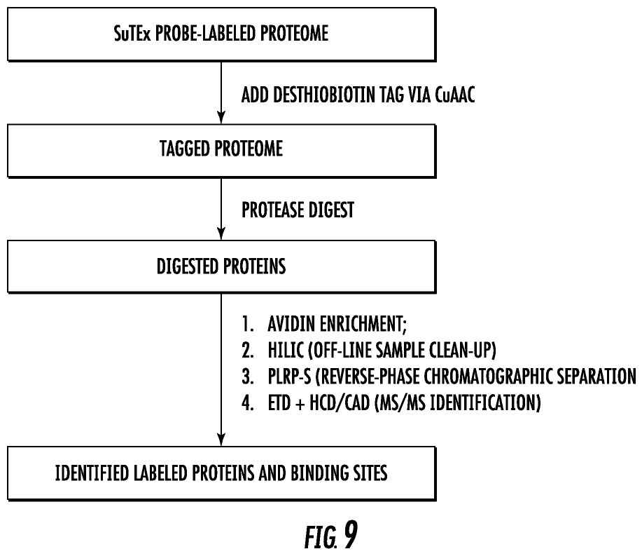

Next, Jurkat cells were treated with KY-26 using optimized treatment conditions (12.5 μM, 2 hr) followed by cell lysis and chemical proteomics analysis using a tailored experimental workflow. See FIG. 9. A combination of HCD and ETD were employed to take advantage of the benefits of both MS dissociation methods. Poly(ethylene glycol) (PEG)-related species are common polymer contaminants in MS samples that can be introduced into samples from plastics, pharmaceuticals, and personal care products³⁴. Desalting of samples by reverse-phased C18 resin (e.g. StageTips³⁵), while effective for removing salts, are not able to remove polymers that bind to these resins³⁶. Thus, hydrophilic interaction liquid chromatography (HILIC), which has been previously shown to be effective for removing PEG polymers³⁷, was used to reduce contaminant ions in the LC-MS/MS analyses. Probe-modified peptides derived from KY-26-targeted proteins from live cell treatments were analyzed on an Orbitrap FUSION™ TRIBRID™ (Thermo Fisher Scientific, Waltham, Massachusetts, United States of America). capable of high-resolution data acquisition employing both HCD and ETD fragmentation. Additional details of the HILIC cleanup, chemical proteomics, and LC-MS/MS analysis can be found in Example 2, above.

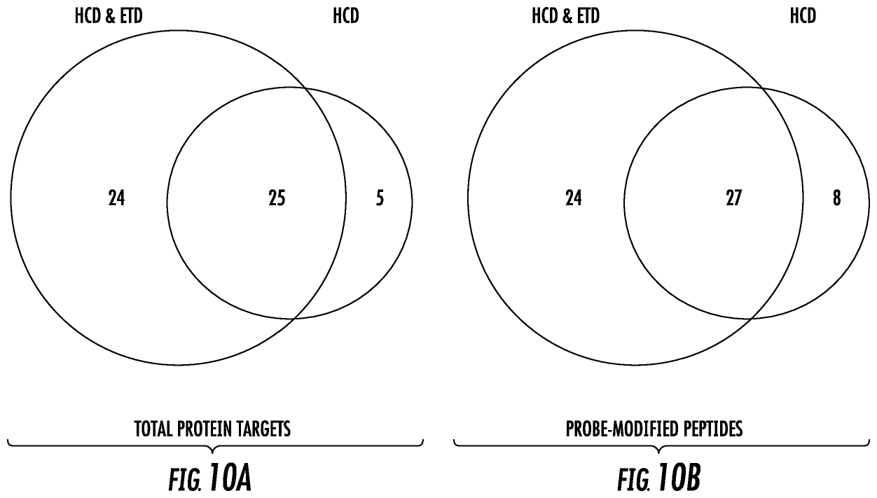

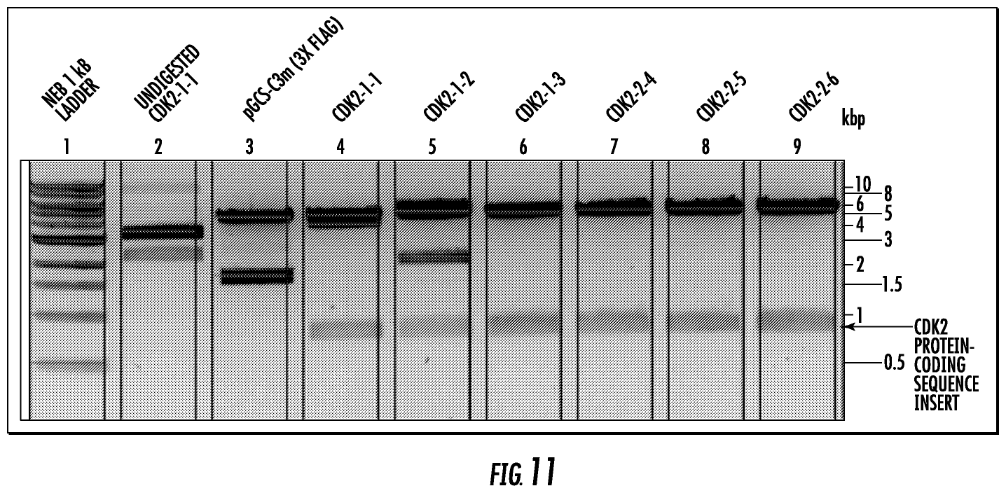

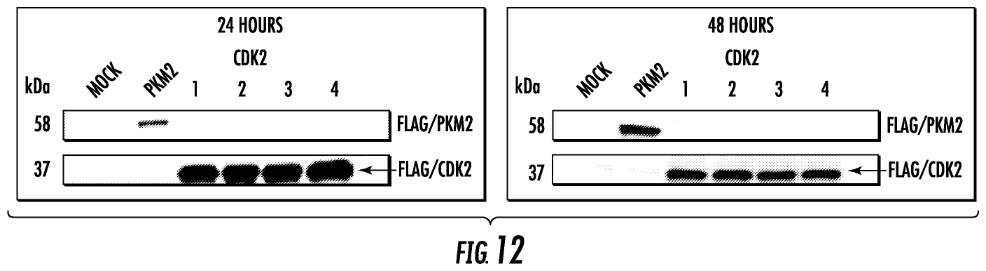

KY-26-modified tyrosine and lysine sites were identified on probe-modified peptides from kinases and other target proteins. See Tables 1-3, below. Importantly, KY-26-modified lysines were catalytic residues that resided in kinase active sites. These findings support the initial rationale for choosing the pyrimidine 3-aminopyrazole for mediating binding recognition of XO44¹⁴. It was also confirmed that KY-26 would modify tyrosine residues in kinase hydrophobic binding pockets and specifically, within the nucleotide binding domain. See Table 1, below. These findings combined with a recent report using SuTEx probes²⁶ support tyrosines as ligandable sites for future development of covalent kinase inhibitors. The use of HCD/ETD compared with HCD fragmentation alone increased the number of detected probe-modified proteins and peptides containing the KY-26 modified site. See FIGS. 10A and 10B. See also Table 2, below.

TABLE 1
List of KY-26 Modified Peptides from HCD MS2 Analysis.

| Uniprot | Modified | |
|---|---|---|
| ID | Residue | Protein |
| P06733.2 | 60 | Alpha-enolase |
| P53396.3 | 384 | ATP-citrate synthase |
| P06493.3 | 33 | Cyclin-dependent kinase 1 |
| P24941.2 | 33 | Cyclin-dependent kinase 2 |
| Q00535.3 | 33 | Cyclin-dependent-like kinase 5 |
| Q02750.2 | 97 | Dual specificity mitogen-activated protein |
| | | kinase kinase 1 |
| Q9P2K8.3 | 616 | eIF-2-alpha kinase GCN2 |
| P04075.2 | 328 | Fructose-bisphosphate aldolase A |
| P62826.3 | 147 | GTP-binding nuclear protein Ran |
| P07900.5 | 61 | Heat shock protein HSP 90-beta |
| Q9NWZ3.1 | 213 | Interleukin-1 receptor-associated kinase 4 |
| Q96L34.1 | 88 | MAP/microtubule affinity-regulating kinase 4 |
| Q92918.1 | 46 | Mitogen-activated protein kinase kinase kinase |
| | | kinase 1 |
| Q12851.2 | 27 | Mitogen-activated protein kinase kinase kinase |
| | | kinase 2 |
| Q9Y4K4.2 | 31 | Mitogen-activated protein kinase kinase kinase |
| | | kinase 5 |
| O00329.2 | 708 | Phosphatidylinositol 4,5-bisphosphate 3-kinase |
| | | catalytic subunit delta |
| P00558.3 | 324 | Phosphoglycerate kinase 1 |
| P00558.3 | 343 | Phosphoglycerate kinase 1 |
| P00558.3 | 76 | Phosphoglycerate kinase 1 |
| P28074.3 | 150 | Proteasome subunit beta type-5 |
| Q14289.2 | 457, 459 | Protein-tyrosine kinase 2-beta |
| Q5NVN0.3 | 370 | Pyruvate kinase PKM |
| Q13546.3 | 308 | Receptor-interacting serine/threonine-protein |
| | | kinase 1 |
| Q16181.2 | 319 | Septin-7 |
| Q13043.2 | 41 | Serine/threonine-protein kinase 4 |
| Q7KZI7.2 | 82 | Serine/threonine-protein kinase MARK2 |
| Q86UE8.2 | 491 | Serine/threonine-protein kinase tousled-like 2 |
| P31948.1 | 442 | Stress-induced-phosphoprotein 1 |
| P31948.1 | 461 | Stress-induced-phosphoprotein 1 |
| P31948.1 | 404 | Stress-induced-phosphoprotein 1 |
| Q99832.2 | 157 | T-complex protein 1 subunit eta |
| P23919.4 | 151 | Thymidylate kinase |
| P16591.2 | 501 | Tyrosine-protein kinase Fer |
| P16591.2 | 714 | Tyrosine-protein kinase Fer |
| P06239.6 | 273 | Tyrosine-protein kinase Lck |

TABLE 2
List of KY-26 Modified Peptides when both
HCD and ETD were used for MS2 analysis.

| Uniprot | Modified | |
|---|---|---|
| ID | Residue | Protein |
| Q13131.4 | 55 | 5′-AMP-activated protein kinase catalytic |
| | | subunit alpha-1 |
| P10809.2 | 227 | 60 kDa heat shock protein, mitochondrial |
| P06733.2 | 60 | Alpha-enolase |
| P53396.3 | 384 | ATP-citrate synthase |
| Q16740.1 | 73 | ATP-dependent Clp protease proteolytic |
| | | subunit, mitochondrial |
| P11586.4 | 240 | C-1-tetrahydrofolate synthase, cytoplasmic |
| P06493.3 | 33 | Cyclin-dependent kinase 1 |
| P24941.2 | 33 | Cyclin-dependent kinase 2 |
| Q00534.1 | 43 | Cyclin-dependent kinase 6 |
| Q00535.3 | 33 | Cyclin-dependent-like kinase 5 |
| O00154.3 | 286 | Cytosolic acyl coenzyme A thioester hydrolase |
| Q9NY33.2 | 417 | Dipeptidyl peptidase 3 |
| P36507.1 | 101 | Dual specificity mitogen-activated protein |
| | | kinase kinase 1/2 |
| Q9P2K8.3 | 616 | eIF-2-alpha kinase GCN2 |
| Q01469.3 | 131 | Fatty acid-binding protein 5 |
| P04075.2 | 328 | Fructose-bisphosphate aldolase A |
| P16930.2 | 244 | Fumarylacetoacetase |
| P28161.2 | 50 | Glutathione S-transferase Mu 2 |
| P07900.5 | 61 | Heat shock protein HSP 90-beta |
| Q16836.3 | 264 | Hydroxyacyl-coenzyme A dehydrogenase, |
| | | mitochondrial |
| P12268.2 | 430 | Inosine-5′-monophosphate dehydrogenase 2 |
| P19525.2 | 297 | Interferon-induced, double-stranded RNA- |
| | | activated protein kinase |
| Q9NWZ3.1 | 213 | Interleukin-1 receptor-associated kinase 4 |
| P40926.3 | 105 | Malate dehydrogenase, mitochondrial |
| Q92918.1 | 46 | Mitogen-activated protein kinase kinase |
| | | kinase kinase 1 |
| Q12851.2 | 27 | Mitogen-activated protein kinase kinase |
| | | kinase kinase 2 |
| Q9Y4K4.2 | 31 | Mitogen-activated protein kinase kinase |
| | | kinase kinase 5 |
| P23368.1 | 84 | NAD-dependent malic enzyme, mitochondrial |
| P22392.1 | 12 | Nucleoside diphosphate kinase B |
| Q15067.3 | 232 | Peroxisomal acyl-coenzyme A oxidase 1 |
| O00329.2 | 708 | Phosphatidylinositol 4,5-bisphosphate 3-kinase |
| | | catalytic subunit delta |
| P00558.3 | 324 | Phosphoglycerate kinase 1 |
| P00558.3 | 76 | Phosphoglycerate kinase 1 |
| O14818.1 | 118 | Proteasome subunit alpha type-7 |
| P28074.3 | 150 | Proteasome subunit beta type-5 |
| P49354.1 | 166 | Protein farnesyltransferase/geranylgera- |
| | | nyltransferase type-1 subunit alpha |
| Q14289.2 | 457 | Protein-tyrosine kinase 2-beta |
| Q5NVN0.3 | 370 | Pyruvate kinase PKM |
| Q13546.3 | 308 | Receptor-interacting serine/threonine-protein |
| | | kinase 1 |
| P34896.1 | 82 | Serine hydroxymethyltransferase, cytosolic |
| Q13043.2 | 41 | Serine/threonine-protein kinase 4 |
| Q7KZI7.2 | 82 | Serine/threonine-protein kinase MARK2 |
| Q9UKI8.2 | 485 | Serine/threonine-protein kinase tousled-like 1 |
| Q86UE8.2 | 491 | Serine/threonine-protein kinase tousled-like 2 |
| Q99832.2 | 157 | T-complex protein 1 subunit eta |
| P23919.4 | 151 | Thymidylate kinase |
| Q14166.2 | 452 | Tubulin--tyrosine ligase-like protein 12 |
| P41240.1 | 222 | Tyrosine-protein kinase Csk |
| P16591.2 | 501 | Tyrosine-protein kinase Fer |
| P16591.2 | 714 | Tyrosine-protein kinase Fer |
| P06239.6 | 273 | Tyrosine-protein kinase Lck |

TABLE 3
List of KY-26 Protein Targets from Chymotryptic Peptides.

| | | |
|---|---|---|
| Uniprot | Modified | |
| ID | Residue | Protein |

Kinases

| | | |
|---|---|---|
| P17858.6 | 674 | ATP-dependent 6-phosphofructokinase, |
| | | liver type |
| Q01813.2 | 223 | ATP-dependent 6-phosphofructokinase, |
| | | platelet type |
| P27707.1 | 86 | Deoxycytidine kinase |
| Q8TD19.2 | 81 | Serine/threonine-protein kinase Nek9 |

Additional Proteins

| | | |
|---|---|---|
| Q9NVE7.1 | 258 | 4′-phosphopantetheine phosphatase |
| Q9BWD1.2 | 235 | Acetyl-CoA acetyltransferase, cytosolic |
| Q16836.3 | 264 | Hydroxyacyl-coenzyme A dehydrogenase, |
| | | mitochondrial |
| P49354.1 | 164 | Protein farnesyltransferase/geranylgera- |
| | | nyltransferase type-1 subunit alpha |
| P48643.1 | 12 | T-complex protein 1 subunit epsilon |
| P17516.3 | 24 | Aldo-keto reductase family 1 member C4 |

Next, alternative proteases were explored in order to improve LC-MS/MS identification. Trypsin is a widely used protease for LC-MS/MS analysis and generates peptides in the range of 700-1500 Da³⁸. While predictable and ideal for CAD and HCD MS/MS analysis, tryptic peptides are not always well-suited for ETD analysis, which is more useful for peptides with higher charge density³⁸. Furthermore, high sequence homology within kinase active sites can reduce the ability to differentiate peptides from related members including, for example, different kinase isoforms. Chymotrypsin was chosen as a second protease for the LC-MS/MS studies with the goal of producing larger peptides for ETD analysis and improving kinase identification from enriched probe-modified peptides. The addition of chymotrypsin aided in the detection of 4 new kinase targets and 6 non-kinase targets of KY-26. See Table 3.

In summary, it was found that the combination of HCD and ETD along with both trypsin and chymotrypsin protease allowed assignment of KY-26 modification across >65 probe-modified peptides in proteomes from live cell treatments. The target proteins were enriched for kinases with modifications occurring at the expected catalytic lysine and novel tyrosine sites. several non-kinase protein targets were also identified, including probe modifications in the nucleotide binding domains of ATP- and NAD-binding proteins. Collectively, these data support the ability of KY-26 to target nucleotide binding domains in live cells.

### Example 7

### Discussion of Example 1-6

Targeted covalent inhibitors are emerging as enabling probe molecules³⁹⁻⁴¹ and effective drug compounds⁴²⁻⁴⁴. Methods capable of direct identification of site of binding (i.e. covalent adduct of probe with a target protein amino acid site) are advantageous to understand mode of action of larger probe scaffolds and guide development of targeted covalent inhibitors with improved selectivity. In contrast with smaller covalent probes and ligands, targeted covalent inhibitors are generally larger in molecular mass, which complicates binding site identifications by increasing the hydrophobicity and charge state of resulting probe-modified peptides analyzed by chemical proteomics. Consequently, a common alternative approach is the LC-MS/MS detection of tryptic peptides generated from probe-modified proteins enriched by affinity chromatography for protein-level identification.

To facilitate site of binding analyses for targeted covalent inhibitors, herein presented are LC-MS/MS conditions tailored for chemical proteomic evaluation of a SuTEx probe based on a kinase inhibitor scaffold (KY-26). Akin to other targeted covalent inhibitors, KY-26 modification increases the molecular weight (+946 Da), hydrophobicity, and charge imparted onto peptides from modified proteins. Chromatography conditions and dissociation strategies were tested and identified to guide LC-MS/MS analysis of bulky probe adducts introduced by KY-26. These optimized LC-MS/MS conditions were applied to identify tyrosine and lysine sites modified by KY-26 in functional sites of kinases and other ATP-/NAD-binding proteins (>65 in total) in live cell chemical proteomic studies. Competition of KY-26 labeling with free ATP and a non-clickable analog supports molecular recognition as an important feature of KY-26 labeling activity. See FIGS. 3A and 3B.

As disclosed herein, it was found that C18 media can be ineffective for reverse-phase separation of KY-26-modified peptides due to the increased hydrophobicity of peptides. PLRP-S was identified as an alternative medium for analytical columns used for nanoflow LC, which enabled the retention and elution of KY-26-modified peptides. See FIGS. 6A and 6B. PLRP-S is advantageous due to its chemical and mechanical stability, and unlike C18, does not contain surface silanols which result in analyte tailing²⁸. It was also found that KY-26 modification changed the chromatography of probe-modified peptides substantially when compared to the unmodified peptide; the difference in elution times (˜8 min) is indicative of increased hydrophobicity from KY-26 modification.

MS dissociation strategies were identified to increase coverage of identified KY-26-modified proteins and corresponding sites. Particularly, the benefits of including ETD fragmentation in chemical proteomic workflows were demonstrated, including increased sequence coverage on high charge state peptides that result from KY-26 modification. ETD was first described for sequencing phosphopeptides and has since been deployed for LC-MS/MS analysis of various post-translational modifications (glycosylation, palmitoylation, etc.)^{32-33,45-46}. The ability to preserve labile bonds with ETD was also important for reducing fragment ions from the desthiobiotin tag to reduce complexity of MS/MS spectra and increase sequence coverage. See FIGS. 8A-8C. The combination of HCD and ETD in the presently disclosed LC-MS/MS studies facilitated increased identification of KY-26 modified sites in proteomes from probe treated cells. Analyses of probe-modified peptides with HCD/ETD yielded 51 target site assignments compared with 35 sites from HCD alone. See FIGS. 10A and 10 and Table 2. Peptides were also found that were too small for differentiating between kinase members in the analyses of tryptic samples (<6 amino acids, e.g., K[+KY-26]K). The use of chymotrypsin as an alternative protease to generate larger peptides for LC-MS/MS protein identification yielded 4 new KY-26 kinase targets. See Table 3.

The number of probe-enriched kinases identified using KY-26 is lower than reported for XO44 despite higher reactivity for KY-26. The present approach enriches for and detects probe-modified peptides derived from KY-26 labeled proteins and thus measures probe-bound proteins exclusively. In contrast, protein-level identification strategies for assigning targets to XO44 and additional targeted covalent inhibitors measure tryptic peptides derived from proteins bound to affinity resin. While some proteins are enriched by affinity chromatography through direct probe binding, indirect mechanisms (e.g. protein-protein interactions with probe-bound proteins) can artificially inflate reported protein targets. Additional reversed phase or ion exchange resins can be tested, as well as incorporating reactive groups with specificity for other amino acids⁴⁷. The present LC-MS/MS workflow can also prove useful for detecting peptides modified by photoreactive probes⁴⁸ through improved chromatography and sequence coverage.

In summary, KY-26 along with additional covalent kinase probes/inhibitors^{14,21,41} are among a collection of activity-based probes used for chemical proteomic evaluation of kinase function and inhibitor binding. The development of additional LC-MS/MS methodology, including those described herein, can support these chemical proteomic efforts to advance basic and translational investigations of the human kinome.

### Example 8

### Inhibition of Human CDK2 with KY-424

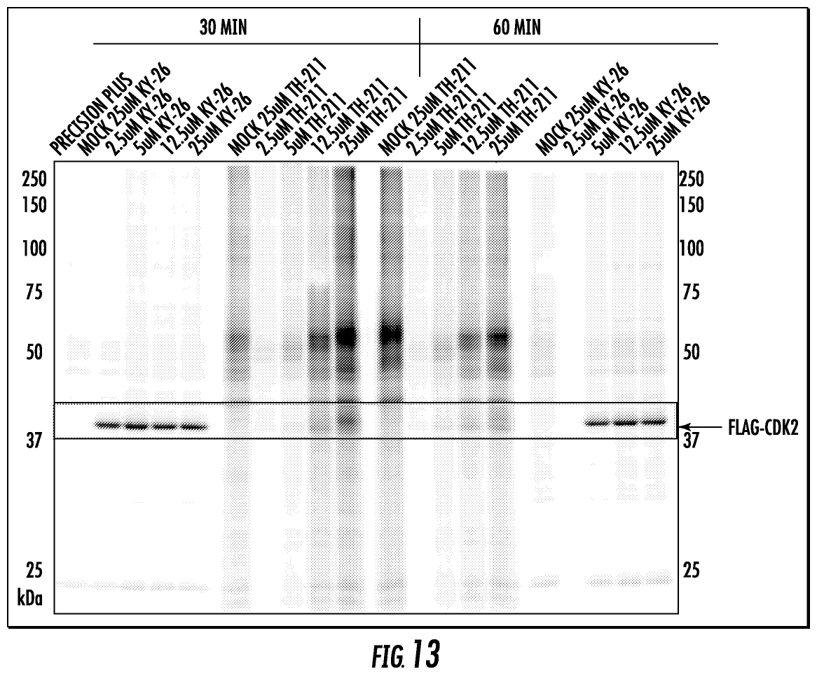

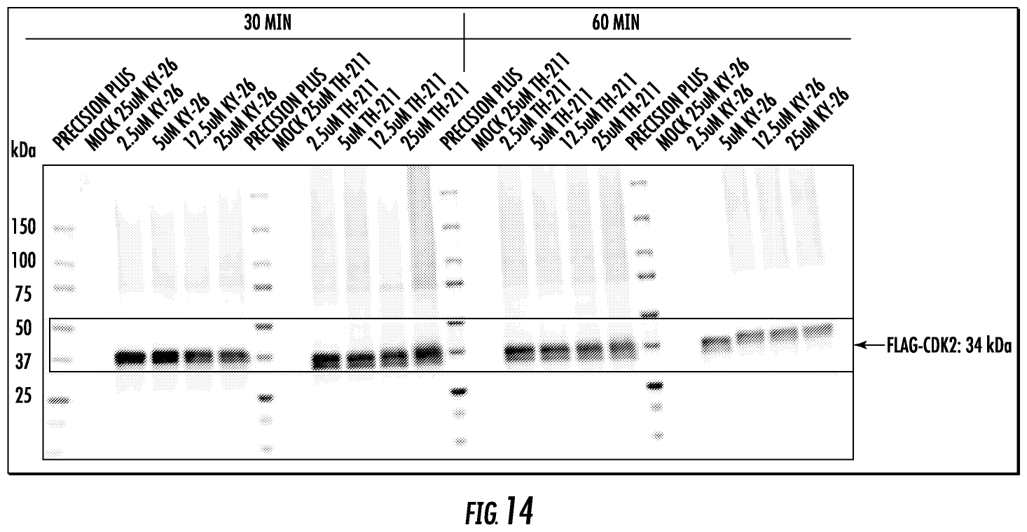

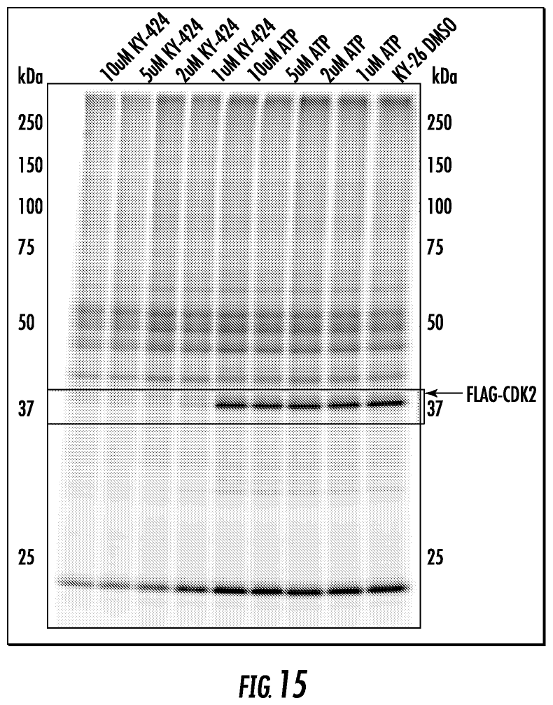

Gateway cloning was performed to generate recombinant human CDK2 (containing a FLAG tag) overexpression plasmid (see FIG. 11) and recombinant human CDK2 was overexpressed in HEK293T mammalian cells. See FIG. 12. KY-26 and TH211 activity-based probe (ABP) labeling of the recombinant human CDK2 was performed. Briefly, recombinant human CDK2 overexpressed HEK293T lysates were incubated for 30 minutes at 37° C. with various concentrations of TH211 (a broad-spectrum kinase ABP) or KY-26 (a targeted covalent kinase ABP) for 30- and 60-minutes. See FIG. 13. Western blots confirmed CDK2 overexpression with rabbit anti-FLAG and goat anti-rabbit 650 antibodies for samples incubated with KY-26 or TH211 probes. See FIG. 14.

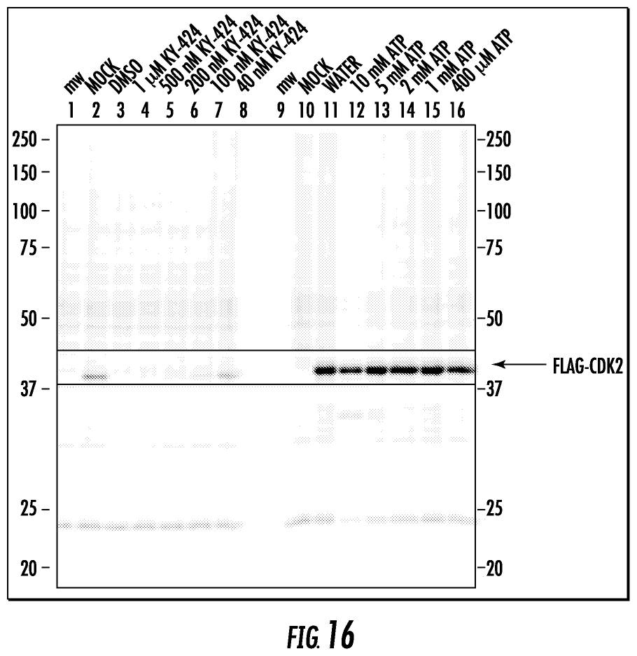

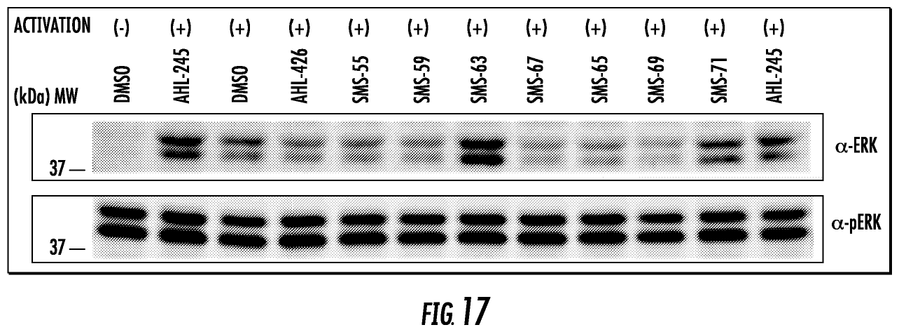

Recombinant human CDK2 overexpressed HEK293T lysates were incubated with KY-424 (1 μM-10 μM) or free ATP (1 μM-10 μM) for 30 minutes at 37° C. Subsequently, samples were incubated with 2.5 μM KY-26 for 30 minutes at 37° C. Covalent inhibitor KY-424 potently competes KY-26 ABP labeling of recombinant human CDK2. See FIG. 15. Based on this finding, the study was performed using additional concentrations of KY-424 (40 nM-μM) or free ATP (400 μM-10 mM) for 30 minutes at 37° C. Again, samples were then incubated with 2.5 μM KY-26 for 30 minutes at 37° C. Under these treatment conditions, KY-424 showed approximately 50% blockade of KY-26 labeling at a 40 nM concentration. See FIG. 16. Lysates from 24- (KY-424 competition) and 48-hour transfections (ATP competition) were used.

### Example 9

### Synthesis of Covalent Kinase Inhibitors SMS-55 to SMS-87

All chemicals used were all reagent grade and used as supplied, except where noted. ¹H and ¹³C spectra were recorded on a Varian Inova 600 (600 MHz) spectrometer (Varian, Inc., Palo Alto, California, United States of America) in CDCl₃ with chemical shifts referenced to internal standards (CDCl₃: 7.26 ppm ¹H, 77.16 ppm ¹³C) unless stated otherwise. Splitting patterns are indicated as follows: s, singlet; d, doublet; t, triplet; q, quartet; m, multiplet; br, broad singlet for ¹H-NMR data. NMR chemical shifts (δ) are reported in ppm and coupling constants (J) are reported in Hz. All reactions were monitored by thin-layer chromatography (TLC) carried out on precoated Merck silica gel 60 F254 plates (0.25 mm thickness); compounds were visualized by UV light and different stains of a TLC plate. All reactions were carried out under nitrogen or argon atmosphere with dried solvents under anhydrous conditions and yields refer to chromatographically homogenous materials unless otherwise stated. Flash chromatography refers to automated flash chromatography (Biotage, Uppsala, Sweden) unless otherwise specified. All evaporations were carried out under reduced pressure on a rotary evaporator below 40° C. unless otherwise specified.

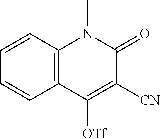 [SMILES: Cn1c(=O)c(C#N)c(OS(=O)(=O)C(F)(F)F)c2ccccc21]

### 3-cyano-1-methyl2-oxo-1,2-dihydroquinolin-4-yl trifluoromethanesulfonate (SMS-1)

As shown in Scheme 6, below, to a stirred solution of 4-hydroxy-1-methyl-2-oxo-1,2-dihydroquinoline-3-carbonitrile (2.5 g, 12.5 mmol) in 25 mL DCM was added trifluoromethanesulfonic anhydride (2.3 mL, 13.74 mmol), DMAP (0.160 g, 1.2 mmol) and triethylamine (2.5 mL, 18.73 mmol). The resulting mixture was stirred at room temperature for 3 h, followed by addition of water (30 mL) and then extracted with dichloromethane (3×30 mL). The organic layers were combined and dried over anhydrous Na₂SO₄. The residue was purified by flash chromatography using 35% EtOAc in n-hexane as an eluent to give the desired product SMS-1 (2.2 g, 54% yield) as a yellow solid. ¹H NMR (600 MHz, CDCl₃) δ 7.96-7.94 (m, 1H), 7.88-7.85 (m, 1H), 7.53-7.51 (m, 1H), 7.49-7.46 (m, 1H). 3.80 (s, 3H).

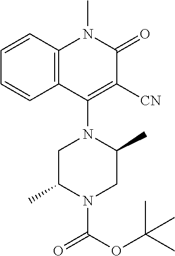 [SMILES: C[C@@H]1CN(c2c(C#N)c(=O)n(C)c3ccccc23)[C@@H](C)CN1C(=O)OC(C)(C)C]

### tert-butyl (2R,5S)-4-(3-cyano-1-methyl-2-oxo-1,2-dihydroquinolin-4-yl)-2,5-dimethylpiperazine-1-carboxylate (SMS-2)

The compound SMS-1 (2.2 g, 6.62 mmol) and commercially available tert-butyl (2R, 5S)-2,5-dimethylpiperyzine-1-caroboxylate (1.4 g, 6.62 mmol) were dissolved in anhydrous dimethylformamide (15 mL), and Hunig's base (1.8 mL, 13.24 mmol) was added at room temperature. The reaction mixture was stirred at 85° C. for 16 h, after which it was diluted with brine (30 mL) and then extracted with Et₂O (30 mL), and the aqueous layer was separated and Et₂O (3×30 mL). The combined organic layer was dried over Na₂SO₄. The residue was purified by flash chromatography using 40% EtOAc in n-hexane as an eluent to give the desired product SMS-2 (2.5 g, 96% yield) as a yellow solid. ¹H NMR (600 MHz, CDCl₃) δ 7.84-7.83 (m, 1H), 7.67-7.65 (m, 1H), 7.40-738 (m, 1H), 7.28-7.27 (m, 1H), 4.49 (s, 1H), 4.35-4.33 (m, 1H), 4.13-4.09 (m, 1H), 3.88-3.86 (m, 1H), 3.74 (m, 1H), 3.68 (s, 3H), 3.07-3.05 (m, 1H), 1.49 (brs, 9H), 1.28 (t, J=6.8 Hz, 6H). ¹³C NMR (151 MHz, CDCl₃) δ 163.7, 160.8, 155.3, 141.4, 133.5, 126.4, 122.5, 117.9, 116.5, 115.6, 80.4, 66.0, 30.1, 28.6, 15.7, 15.1.

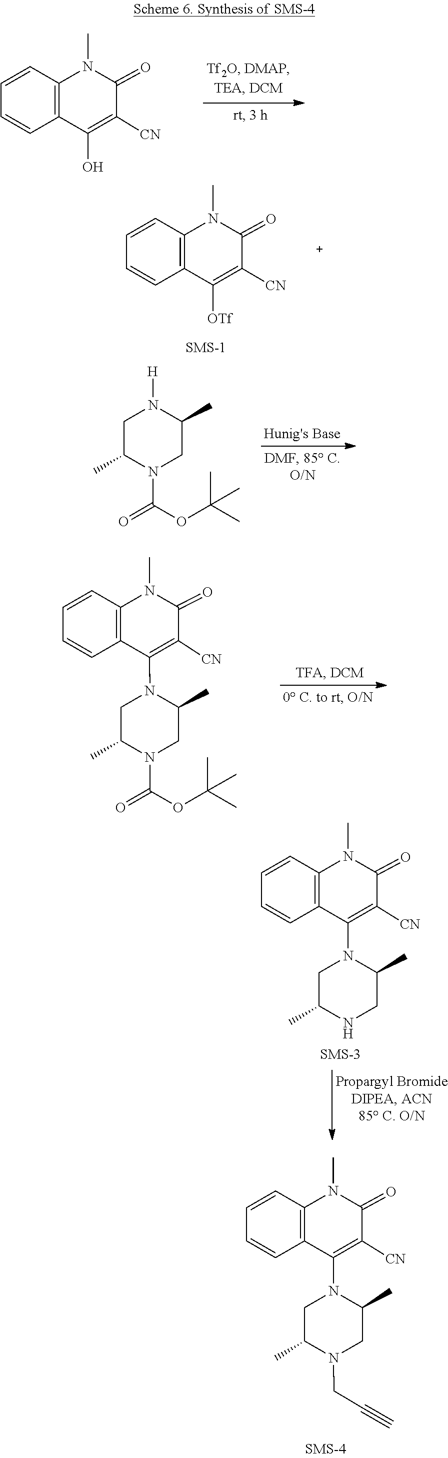

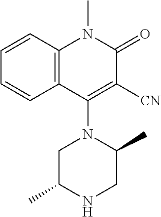 [SMILES: C[C@@H]1CN(c2c(C#N)c(=O)n(C)c3ccccc23)[C@@H](C)CN1]

### 4-((2S,5R)-2,5-dimethylpiperazine-1-yl)-1-methyl-2-oxo-1,2-dihydroquinoline-3-carbonitrile (SMS-3)

The compound SMS-2 (2.5 g, 6.30 mmol) was dissolved in anhydrous dichloromethane (30 mL), and trifluoroacetic acid (5.1 mL, 63.09 mmol) was dropwise added at 0° C. under nitrogen (N₂) atmosphere for 10-15 min. The reaction mixture was stirred at room temperature for 16 h, The mixture was cooled down to room temperature and concentrated under reduced pressure. The resultant residue was washed three times with n-Pentane:Et₂O (3:1), and then dried under high vacuum to afford the desired final product SMS-3 (2.8 g, 100% yield) as a white solid. ¹H NMR (600 MHz, CDCl₃) δ 8.14 (m, 1H), 7.80 (t, J=7.7 Hz, 1H).), 7.50-7.43 (m, 2H), 4.38 (s, 1H), 3.85 (s, 1H), 3.78 (s, 31H), 3.66-3.57 (m, 2H), 3.34-3.20 (m, 1H), 1.47 (m, 3H), 1.12 (m, 3H). ¹³C NMR (151 MHz, CDCl₃) δ 188.5, 160.4, 135.5, 1264, 124.3, 119.8, 115.6, 54.9, 52.3, 50.1, 30.7, 16.3, 15.7.

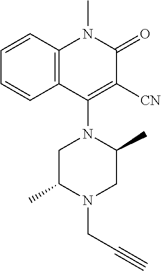 [SMILES: C#CCN1C[C@H](C)N(c2c(C#N)c(=O)n(C)c3ccccc23)C[C@H]1C]

### 4-((2S,5R)-2,5-dimethyl-4-(prop-2-yn-1-yl) piperazine-yl)-1-methyl-2-oxo-1,2-dihydroquinoline-3-carbonitrile (SMS-4)

The compound SMS-3 (2.8 g, 9.45 mmol) and propargyl bromide (1 mL, 11.81 mmol) was dissolved in anhydrous acetonitrile (30 mL), and N, N-diisopropylethylamine base (5 mL, 28.35 mmol) was added at room temperature. The reaction mixture was stirred at 85° C. for 16 h, after which it was diluted with water (30 mL) and then extracted with ethyl acetate (30 mL), and the aqueous layer was separated and ethyl acetate (3×30 mL). The combined organic layer was dried over Na₂SO₄. The residue was purified by flash chromatography using 65% EtOAc in n-hexane as an eluent to give the desired product SMS-4 (1.4 g, 45% yield) as a yellow solid. ¹H NMR (600 MHz, CDCl₃) δ 8.14-8.13 (m, 1H), 7.70-7.67 (m, 1H), 7.39-7.38 (m, 1H), 7.33-7.30 (m, 1H), 4.20 (s, 1H), 3.72 (s, 3H). 3.70 (s, 1H), 3.44-3.41 (m, 1H), 3.18 (s, 1H), 3.02-2.99 (m, 1H), 2.90-2.88 (m, 1H), 2.62-2.58 (m, 1H), 2.31 (t, J=2.4 Hz, 1H), 1.06 (t, J−6.9 Hz, 3H). ¹³C NMR (151 MHz, CDCl₃) δ 163.0, 160.1, 1411, 133.8, 126.4, 122.9, 119.6, 115.0, 115.0, 101.3, 77.6, 73.7, 59.4, 58.9, 53.4, 52.2, 42.5, 30.0, 16.9, 15.8.

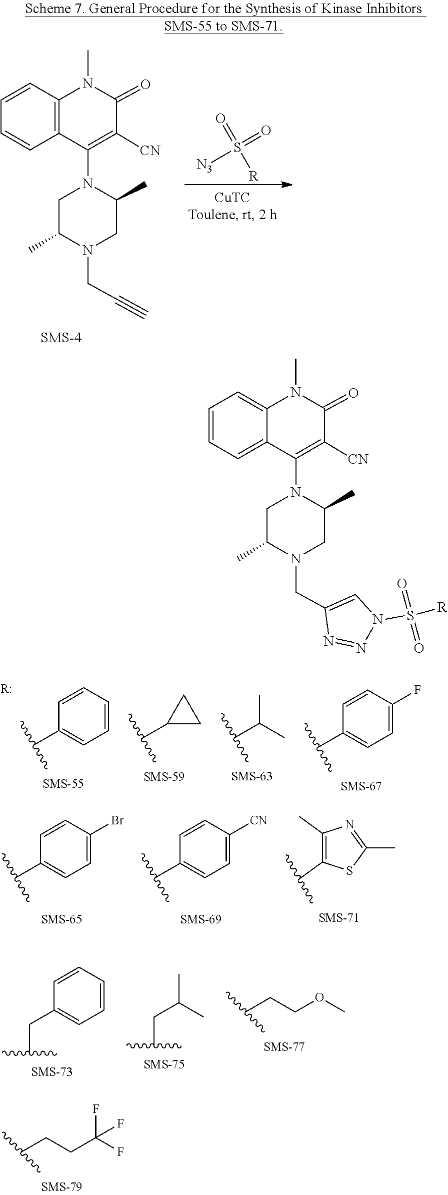

The intermediate SMS-4 and substituted sulphonyl azide and Copper (i)-thiophene-2-carboxylate in anhydrous toluene was charged into a 25 mL round bottom flask, which was equipped with N₂ balloon. The reaction mixture was stirred at room temperature for 2 h, after which it was diluted with water (10 mL) and then extracted with ethyl acetate (10 mL), and the aqueous layer was separated and ethyl acetate (3×10 mL). The combined organic layer was dried over Na₂SO₄. The residue was purified by flash chromatography using 90% EtOAc in n-hexane as an eluent to give the desired final product as a yellow solid.

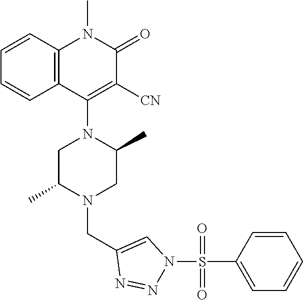 [SMILES: C[C@@H]1CN(c2c(C#N)c(=O)n(C)c3ccccc23)[C@@H](C)CN1Cc1cn(S(=O)(=O)c2ccccc2)nn1]

### 4-((2S,5R)-2,5-dimethyl-4-((1-phenylsulfonyl)-1H-1,2,3-triazol-4-yl) methyl)piperazin-1-yl)-1-methyl-2-oxo-1,2-dihydroquinoline-3-carbonitrile (SMS-55)

As shown in Scheme 7, above, SMS-4 (200 mg, 0.598 mmol), benzene sulfonyl azide (160 mg, 0.718 mmol), Copper (t)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-55 (0.300 g, 97% yield) as a yellow solid. ¹H NMR (600 MHz, CDCl₃) δ 8.14-8.13 (m, 2H), 8.11 (s, 1H), 7.99-7.98 (m, 1H), 7.75-7.72 (m, 1H), 7.68-7.65 (m, 1H), 7.63-7.60 (m, 2H), 7.38-7.36 (m, 1H), 7.28-7.27 (m, 1H), 4.11-4.08 (m, 1H), 3.97-3.94 (m, 1H), 3.85-3.81 (m, 1H), 3.69 (s, 3H), 3.02-2.98 (m, 2H), 2.78 (s, 2H), 2.29-2.26 (m, 1H), 1.23 (t, J−7.1 Hz, 1H), 1.14 (d, J−6.2 Hz, 1H), 1.04-1.03 (m, 3H). ¹³C NMR (151 MHz, CDCl₃) δ 163.3, 160.3, 144.8, 141.2, 136.2, 135.8, 133.8, 130.0, 128.7, 126.5, 122.9, 122.5, 115.4, 115.1, 60.5, 54.6, 48.4, 301, 21.1, 16.8, 14.3.

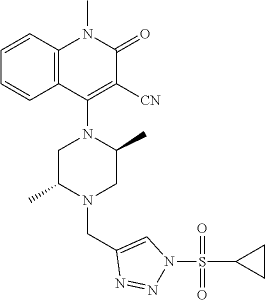 [SMILES: C[C@@H]1CN(c2c(C#N)c(=O)n(C)c3ccccc23)[C@@H](C)CN1Cc1cn(S(=O)(=O)C2CC2)nn1]

### 4-((2S,5R)-4-((cyclopropyl sulfonyl)-1H-1,2,3-triazol-4-yl) methyl) 2,5-dimethyl piperazin-1-yl)-1-methyl-2-oxo-1,2-dihydroquinoline-3-carbonitrile (SMS-59)

As shown in Scheme 7, above, SMS-4 (200 mg, 0.598 mmol), cyclopropyl sulfonyl azide (105 mg, 0.718 mmol), Copper (t)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-59 (0.250 g, 96% yield) as a yellow solid. ¹H NMR (600 MHz, CDCl₃) δ 8.04 (s, 1H), 8.01-7.99 (m, 1H) 7.67-7.61 (m, 1H), 738-7.36 (m, 1H) 7.29-727 (m, 1H), 4.12-4.08 (m, 1H), 4.02-4.00 (m, 1H), 3.92-3.89 (m, 1H), 3.68 (s, 3H), 3.06-3.00 (m, 2H), 2.95-2.91 (m, 1H), 2.81 (s, 1H), 2.34-2.31 (m, 1H), 1.63-1.61 (m, 2H), 1.37-1.29 (m, 2H), 1.24-1.21 (m, 1H), 1.15 (d, J−6.2 Hz, 1H), 1.06-1.05 (m, 3H), ¹³C NMR (151 MHz, CDCl₃) δ 188.4, 163.3, 160.3, 1443, 141.2, 133.8, 126.4, 122.8, 122.8, 1193, 115.4, 115.1, 60.4, 54.5, 48.2, 32.3, 30.1, 29.7, 21.1, 16.7, 14.2, 8.0, 8.0.

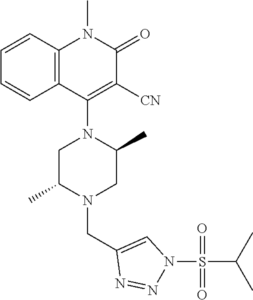 [SMILES: CC(C)S(=O)(=O)n1cc(CN2C[C@H](C)N(c3c(C#N)c(=O)n(C)c4ccccc34)C[C@H]2C)nn1]

### 4-((2S,5R)-4-((Isopropyl sulfonyl)-1H-1,2,3-triazol-4-yl) methyl)-2,5-dimethyl piperazin-1-yl)-1-methyl-2-oxo-1,2-dihydroquinoline-3-carbonitrile (SMS-63)

As shown in Scheme 7, above, SMS-4 (200 mg, 0.598 mmol), isopropyl sulfonyl azide (106 mg, 0.718 mmol), Copper (t)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-63 (0.275 g, 95% yield) as a yellow solid. ¹H NMR (600 MHz, CDCl₃) δ 8.05 (s, 1H), 7.99-7.97 (m, 1H), 7.67-7.64 (m, 1H), 7.37-7.36 (m, 1H), 7.28-7.25 (m, 1H), 4.11-4.06 (m, 1H), 4.02-3.99 (m, 1H), 3.93-3.90 (m, 1H), 3.87-3.83 (m, 1H), 3.68 (s, 3H), 3.02-2.99 (m, 2H), 2.79 (s, 1H), 2.36-2.27 (m, 1H), 2.00 (s, 1H), 1.42 (t, J −6.8 Hz, 6H), 1.22 (t, J−7.1 Hz, 1H), 1.15 (d, J−6.2 Hz, 1H), 1.05-1.04 (m, 3H). ¹³C NMR (151 MHz, CDCl₃) δ 188.4, 163.2, 160.2, 144.2, 141.1, 133.8, 126.4, 123.9, 122.8, 119.3, 115.4, 115.1, 60.4, 57.5, 54.5, 48.2, 30.0, 21.1, 16.7, 16.0, 16.0, 14.2.

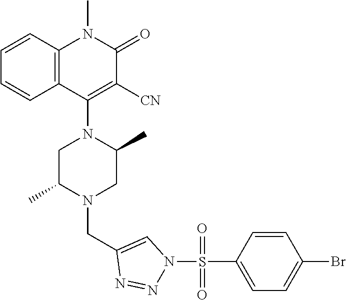 [SMILES: C[C@@H]1CN(c2c(C#N)c(=O)n(C)c3ccccc23)[C@@H](C)CN1Cc1cn(S(=O)(=O)c2ccc(Br)cc2)nn1]

### 4-((2S,5R)-4-((1-((4-bromophenyl) sulfonyl)-1H-1,2,3-triazol-4-yl) methyl)-2,5-dimethyl piperazin-1-yl)-1-methyl-2-oxo-1,2-dihydroquinoline-3-carbonitrile (SMS-65)

As shown in Scheme 7, above, SMS-4 (200 mg, 0.598 mmol), 4-bromobenzene sulfonyl azide (187 mg, 0.718 mmol), Copper (i)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-65 (0.300 g, 84% yield) as a yellow solid. ¹H NMR (600 MHz, CDCl₃) δ 8.11 (s, 1H), 7.99-7.98 (m, 2H), 7.98-7.97 (m, 1H), 7.75-7.73 (m, 2H), 7.67-7.64 (m, 1H), 7.38-7.36 (m, 1H), 7.27-7.25 (m, 1H), 4.10-4.06 (m, 1H), 3.97-3.94 (m, 1H), 3.83-3.81 (m, 1H), 3.68 (s, 3H), 3.01-2.97 (m, 2H), 2.78 (s, 1H), 2.29-2.26 (m, 1H), 2.20 (s, 1H), 1.22 (t, J−7.1 Hz, 1H), 1.12 (d, J−6.2 Hz, 1H), 1.03 (m, 3H). ¹³C NMR (151 MHz, CDCl₃) δ 171.1, 163.2, 160.2, 144.9, 141.1, 135.0, 133.8, 133.3, 131.5, 130.0, 126.4, 122.8, 122.5, 115.4, 115.1, 60.4, 54.5, 48.2, 30.0, 21.1, 16.7, 14.2.

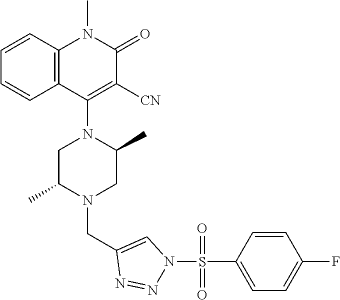 [SMILES: C[C@@H]1CN(c2c(C#N)c(=O)n(C)c3ccccc23)[C@@H](C)CN1Cc1cn(S(=O)(=O)c2ccc(F)cc2)nn1]

### 4-((2S,5R)-4-((1-((4-fluorophenyl) sulfonyl)-1H-1,2,3-triazol-4-yl) methyl)-2,5-dimethyl piperazin-1-yl)-1-methyl-2-oxo-1,2-dihydroquinoline-3-carbonitrile (SMS-67)

As shown in Scheme 7, above, SMS-4 (200 mg, 0.598 mmol), 4-fluorobenzene sulfonyl azide (144 mg, 0.718 mmol), Copper (i)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-67 (0.290 g, 91% yield) as a yellow solid. ¹H NMR (600 MHz, CDCl₃) δ 8.20-8.17 (m, 2H), 8.11 (s, 1H), 8.00-7.98 (m, 1H), 7.68-7.65 (m, 1H), 7.38-7.37 (m, 1H), 7.31-7.28 (m, 2H), 7.27-7.26 (m, 1H), 4.11-4.07 (m, 1H), 3.98-3.95 (m, 1H), 3.84-3.82 (m, 1H), 3.69 (s, 3H), 3.02-2.99 (m, 2H), 2.78 (s, 1H), 2.32-2.2.6 (m, 1H), 2.02 (s, 1H), 1.23 (t, J−7.1 Hz, 1H), 1.13 (t, J−6.2 Hz, 1H), 1.03 (m, 3H). ¹³C NMR (151 MHz, CDCl₃) δ 188.4, 167.8, 166.1, 163.3, 160.3, 141.2, 133.8, 132.1, 132.1, 132.0, 131.9, 130.0, 126.4, 122.8, 122.4, 119.3, 117.6, 117.4, 115.4, 1151, 60.4, 54.6, 48.3, 30.1, 21.1, 16.7, 14.3

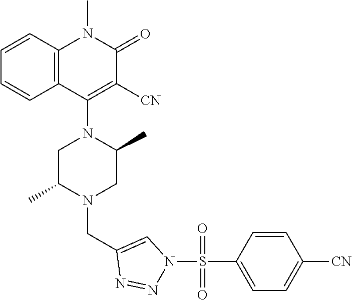 [SMILES: C[C@@H]1CN(c2c(C#N)c(=O)n(C)c3ccccc23)[C@@H](C)CN1Cc1cn(S(=O)(=O)c2ccc(C#N)cc2)nn1]

### 4-((2S,5R)-4-((1-((4-cyanophenyl) sulfonyl)-1H-1, 2, 3-triazol-4-yl) methyl)-2,5-dimethyl piperazin-1-yl)-1-methyl-2-oxo-1,2-dihydroquinoline-3-carbonitrile (SMS-69)

As shown in Scheme 7, above, SMS-4 (200 mg, 0.598 mmol), 4-cynobenzene sulfonyl azide (150 mg, 0.718 mmol), Copper (t)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-69 (0.280 g, 86% yield) as a yellow solid. ¹H NMR (600 MHz, CDCl₃) δ 8.30 (d, J−8.4 Hz, 2H), 8.14 (s, 1H), 8.01-8.00 (m, 1H), 7.94-7.92 (d, J−8.4 Hz, 2H), 7.69-7.66 (m, 1H), 7.39-7.38 (m, 1H), 7.30-7.27 (m, 1H), 4.13-4.09 (m, 1H), 4.01-3.98 (m, 1H), 3.84-3.81 (m, 1H), 3.71 (s, 3H), 3.05-3.02 (m, 2H), 2.81 (s, 1H), 2.31-2.30 (m, 1H), 2.04 (s, 1H), 1.26-1.24 (m, 1H), 1.14 (d, J−6.1 Hz, 3H), 1.06 (m, 3H). ¹³C NMR (151 MHz, CDCl₃) δ 188.5, 163.3, 160.3, 141.3, 140.2, 133.9, 133.7, 129.5, 126.5, 122.9, 122.7, 119.5, 116.6, 115.2, 60.5, 54.8, 48.4, 30.2, 29.8, 21.2, 16.8, 14.3.

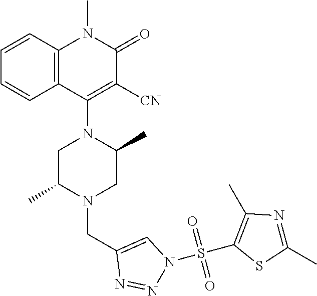 [SMILES: Cc1nc(C)c(S(=O)(=O)n2cc(CN3C[C@H](C)N(c4c(C#N)c(=O)n(C)c5ccccc45)C[C@H]3C)nn2)s1]

### 4-((2S,5R)-4-((1-((2,4-dimethylthiazol-5-yl) sulfonyl)-1H-1,2,3-triazol-4-yl) methyl)-2,5-dimethylpiperazin-1-yl)-1-methyl-2-oxo-1,2-dihydroquinoline-3-carbonitrile (SMS-71)

As shown in Scheme 7, above, SMS-4 (200 mg, 0.598 mmol), 2,4-dimethyltiazol sulfonyl azide (15 mg, 0.718 mmol), Copper (t)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-71 (0.280 g, 84% yield) as a yellow solid. ¹H NMR (600 MHz, CDCl₃) δ 8.09 (s, 1H), 7.99-7.98 (m, 1H), 7.67-7.64 (m, 1H), 7.37 (d, J−8.1 Hz, 1H), 7.28-7.25 (m, 1H), 4.11-4.07 (m, 1H), 3.99-3.96 (m, 1H), 3.88-3.86 (m, 1H), 3.68 (s, 3H), 3.02-2.99 (m, 2H), 2.75 (s, 3H), 2.71 (s, 3H), 2.31-2.27 (m, 1H), 2.01 (s, 1H), 1.22 (t, J−2H), 1.25 (t, J−7.1 Hz, 1H), 1.14 (d, J−6.2 Hz, 3H), 1.05 (m, 3H). ¹³C NMR (151 MHz, CDCl₃) δ 173.8, 163.2 162.5, 160.3, 141.2, 133.8, 126.4, 124.7, 122.8, 122.2, 1 19.3, 115.4, 115.1, 60.4, 54.6, 48.3, 30.1, 29.7, 19.9, 17.1, 16.8.

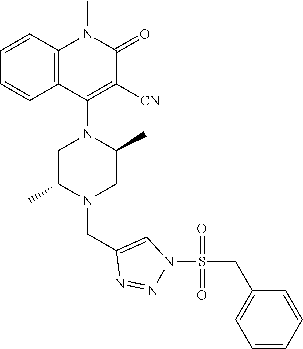 [SMILES: C[C@@H]1CN(c2c(C#N)c(=O)n(C)c3ccccc23)[C@@H](C)CN1Cc1cn(S(=O)(=O)Cc2ccccc2)nn1]

### 4-((2S,5R)-4-((1-benzylsulfonyl)-1H-1,2,3-triazol-4-yl) methyl) 2,5-dimethylpiperazin-1-yl)-1-methyl-2-oxo-1,2-dihydroquinoline-3-carbonitrile (SMS-73)

As shown in Scheme 7, above, SMS-4 (200 mg, 0.598 mmol), phenylmethane sulfonyl azide (141 mg, 0.718 mmol), Copper (i)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-73 (0.210 g, 67% yield) as a yellow solid. ¹H NMR (600 MHz, CDCl₃) δ 8.00-7.99 (m, 1H), 7.69-7.66 (m, 1H) 7.59 (s, 1H) 7.39-7.36 (m, 1H), 7.33-7.30 (m, 2H), 7.29-7.27 (m, 1H), 7.13-7.12 (m, 2H), 4.87 (m, 2H), 4.12-4.07 (m, 1H), 3.91-3.88 (m, 1H), 3.82-3.78 (m, 1H), 3.70 (s, 31H), 2.99-2.90 (m, 2H), 2.71 (s, 1H), 2.21-2.18 (m, 1H), 2.03 (s, 1H), 1.24 (t, J−7.1 Hz, 2H), 1.10 (d, J−6.2 Hz, 3H), 1.04 (m, 31H). ¹³C NMR (151 MHz, CDCl₃) δ 188.4, 163.3, 160.3, 144.4, 141.4, 133.8, 133.7, 130.8, 130.7, 130.1, 130.0, 129.4, 129.2, 126.5, 126.5, 125.7, 124.1, 124.0, 122.8, 122.7, 119.5, 115.3, 115.2, 115.0, 61.6, 61.5, 61.3, 54.5, 53.2, 48.2, 30.3, 30.2, 30.1, 16.8, 16.7.

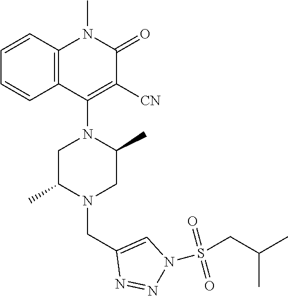 [SMILES: CC(C)CS(=O)(=O)n1cc(CN2C[C@H](C)N(c3c(C#N)c(=O)n(C)c4ccccc34)C[C@H]2C)nn1]

### 4-((2S,5R)-4-((Isobutylsulfonyl)-1H-1, 2, 3-triazol-4-yl) methyl)-2,5-dimethyl piperazin-1-yl)-1-methyl-2-oxo-1,2-dihydroquinoline-3-carbonitrile (SMS-75)

As shown in Scheme 7, above, SMS-4 (200 mg, 0.598 mmol), 2-methylpropane1-sulfonyl azide (117 mg, 0.718 mmol), Copper (i)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-75 (0.300 g, 98% yield) as a yellow solid. ¹H NMR (600 MHz, CDCl₃) δ 8.07 (s, 1H), 7.99-7.98 (m, 1H), 7.67-7.64 (m, 1H). 7.38-7.36 (m, 1H), 7.28-7.25 (m, 1H), 4.11-4.06 (m, 1H), 4.03-3.99 (m, 1H), 3.91-3.88 (m, 1H), 3.68 (s, 3H), 3.55-3.54 (m, 1H), 3.02-2.99 (m, 2H), 2.79 (s, 1H), 2.33-2.26 (m, 2H), 2.00 (s, 1H), 1.22 (t, J−7.1 Hz, 1H), 1.15 (d, J−6.2 Hz, 3H), 1.10 (d, J−2.4 Hz, 3H), 1.09 (d, J−2.4 Hz, 3H), 105-1.04 (m, 3H). ¹³C NMR (151 MHz, CDCl₃) δ 1632, 160.3, 144.4, 141.2, 133.8, 126.4, 122.8, 122.8, 119.3, 115.4, 115.1, 62.9, 60.4, 54.5, 48.2, 30.1, 24.8, 22.2, 22.1, 21.1, 16.7, 14.2.

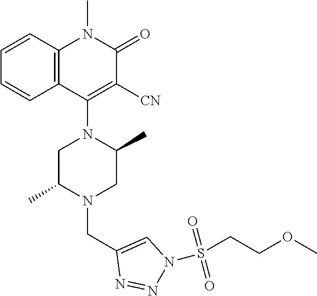 [SMILES: COCCS(=O)(=O)n1cc(CN2C[C@H](C)N(c3c(C#N)c(=O)n(C)c4ccccc34)C[C@H]2C)nn1]

### 4-((2S,5R)-4-((2-methoxyethyl) sulfonyl)-1H-1,2,3-triazol-4-yl) methyl)-2,5-dimethyl piperazin-1-yl)-1-methyl-2-oxo-1,2-dihydroquinoline-3-carbonitrile (SMS-77)

As shown in Scheme 7, above, SMS-4 (200 mg, 0.598 mmol), 2-methylpropane1-sulfonyl azide (120 mg, 0.718 mmol), Copper (i)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-77 (0.250 g, 83% yield) as a yellow solid. ¹H NMR (600 MHz, CDCl₃) δ 8.09 (s, 1H), 8.04-8.00 (m, 1H), 7.68-7.65 (m, 1H), 7.38-7.37 (m, 1H), 7.29-7.27 (m, 1H), 4.14-4.09 (m, 1H), 4.05-4.01 (m, 1H), 3.93-3.88 (m, 2H), 3.88-3.87 (m, 1H), 3.85-3.82 (m, 2H), 3.70 (s, 3H), 3.24 (s, 3H), 3.05-3.01 (m, 2H), 2.81 (s, 1H), 2.34-2.31 (m, 1H), 2.03-1.23 (m, 1H), 1.19 (d, J−6.2 Hz, 3H), 1.07-1.06 (m, 3H). ¹³C NMR (151 MHz, CDCl₃) δ 163.2, 160.3, 144.0, 141.2, 133.9, 126.5, 123.5, 122.9, 119.4, 115.4, 115.1, 68.4, 65.3, 59.1, 55.5, 54.7, 48.2, 30.2, 16.8.

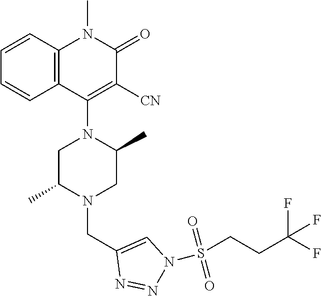 [SMILES: C[C@@H]1CN(c2c(C#N)c(=O)n(C)c3ccccc23)[C@@H](C)CN1Cc1cn(S(=O)(=O)CCC(F)(F)F)nn1]

### 4-((2S,5R)-2,5-dimethyl-4-((1-((3,3,3-trifluoropropyl) sulfonyl)-1H-1, 2, 3-triazol-4-yl) methyl)-piperazin-1-yl)-1-methyl-2-oxo-1,2-dihydroquinoline-3-carbonitrile (SMS-79)

As shown in Scheme 7, above, SMS-4 (200 mg, 0.598 mmol), 3,3,3-trifloropropane-1-sulfonyl azide (118 mg, 0.718 mmol), Copper (i)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-79 (0.250 g, 78% yield) as a yellow solid. 1H NMR (600 MHz, CDCl₃) δ 7.77-7.76 (m, 1H), 7.70-7.66 (m, 1H), 7.42-7.39 (m, 1H), 7.32-7.28 (m, 1H), 6.62 (s, 1H), 5.86-5.84 (m, 1H) 5.78-5.75 (m, 1H), 4.38-4.07 (m, 2H), 3.67 (s, 3H), 3.24-3.19 (m, 2H), 3.19-3.17 (m, 1H), 2.69-2.63 (m, 2H), 2.02 (s, 1H), 1.45-1.40 (m, 3H), 1.32-1.29 (m, 3H), 1.23 (t, J−7.1 Hz, 1H). ¹³C NMR (151 MHz, CDCl₃) δ 188.1, 171.2, 163.0, 160.4, 141.4, 133.9, 128.2, 127.0, 126.9, 126.0, 125.9, 125.1, 122.7, 117.5, 116.3, 115.8, 60.5, 55.0, 50.6, 48.1, 30.2, 30.0, 29.8, 29.6, 29.4, 21.1, 15.0, 14.3.

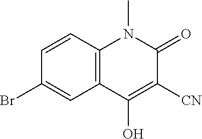 [SMILES: Cn1c(=O)c(C#N)c(O)c2cc(Br)ccc21]

### 6-bromo-4-hydroxy-1-methyl-2-oxo-1,2-dihydroquinoline-3-carbonitrile (SMS-5)

As described in Scheme 8, above, to a stirred solution of 6-bromo-1-methyl-2H-benzo (1,3) oxazine-2,4(1H)-dione (2.5 g, 9.77 mmol) and ethyl cyanoacetate (2.0 mL, 19.54 mmol), in 20 mL THE was added triethylamine (5.4 mL, 39.04 mmol). The resulting mixture was stirred at 90° C. for 72 h, followed by addition of water (50 mL) and then extracted with ethyl acetate (3×50 mL). The mixture was acidified to a pH of 1 by addition of hydrochloric acid (2N). The resulting precipitate was collected by filtration and dried over anhydrous Na₂SO₄. The residue was purified by flash chromatography using 25% EtOAc in n-hexane as an eluent to give the desired product SMS-5 (2.0 g, 91% yield) as a yellow solid. ¹H NMR (600 MHz, CDCl₃) δ 8.06 (d, J−2.4 Hz, 1H), 7.49-7.47 (m, 1H), 6.60 (d, J−9 Hz, 1H), 2.92 (s, 3H).

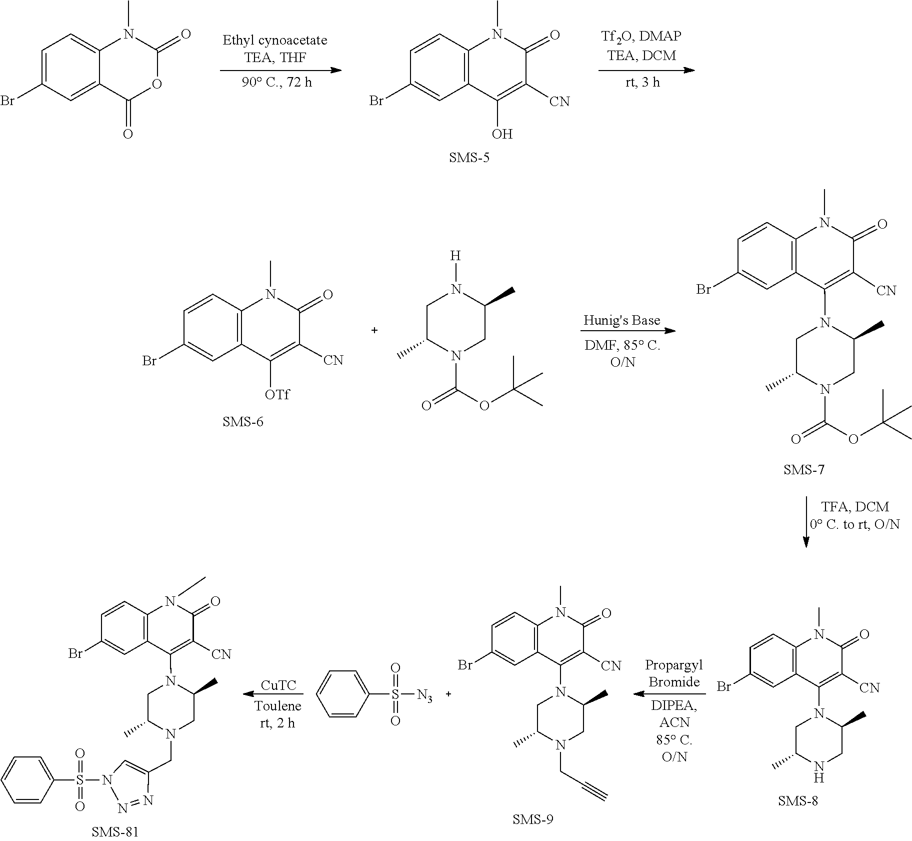

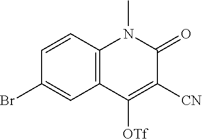 [SMILES: Cn1c(=O)c(C#N)c(OS(=O)(=O)C(F)(F)F)c2cc(Br)ccc21]

### 6-bromo-3-cyano-1-methyl-2-oxo-1,2-dihydroquinolin-4-yl trifluoromethanesulfonate (SMS-6)

Continuing with Scheme 8, above, to a stirred solution of SMS-5 (2.0 g, 7.2 mmol) in 25 mL DCM was added trifluoromethanesulfonic anhydride (1.3 mL, 7.92 mmol), DMAP (0.220 g, 1.8 mmol) and Triethylamine (2.0 mL, 14.4 mmol). The resulting mixture was stirred at room temperature for 3 h, followed by addition of water (30 mL) and then extracted with dichloromethane (3×30 mL). The organic layers were combined and dried over anhydrous Na₂SO₄. The residue was purified by flash chromatography using 45% EtOAc in n-hexane as an eluent to give the desired product SMS-6 (2.0 g, 68% yield) as a yellow solid. ¹H NMR (600 MHz, CDCl₃) δ 8.04 (d, J−2.2 Hz, 1H), 7.93-7.91 (m, 1H), 7.40-7.38 (d, J−9.1 Hz, 1H), 3.77 (s, 3H).

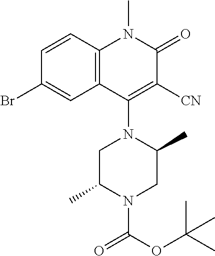 [SMILES: C[C@@H]1CN(c2c(C#N)c(=O)n(C)c3ccc(Br)cc23)[C@@H](C)CN1C(=O)OC(C)(C)C]

### tert-butyl (2R,5S)-4-(6-bromo-3-cyano-1-methyl-2-oxo-1,2-dihydroquinolin-4-yl)-2,5-dimethylpiperazine-1-carboxylate (SMS-7)

The compound SMS-6 (2.0 g, 4.8 mmol) and commercially available tert-butyl (2R, 5S)-2,5-dimethylpiperyzine-1-caroboxylate (1.1 g, 4.8 mmol) was dissolved in anhydrous dimethylformamide (15 mL), and Hunig's base (1.3 mL, 9.6 mmol) was added at room temperature. The reaction mixture was stirred at 85° C. for 16 h, after which it was diluted with brine (30 mL) and then extracted with Et₂O (30 mL), and the aqueous layer was separated and Et₂O (3×30 mL). The combined organic layer was dried over Na₂SO₄. The residue was purified by flash chromatography using 45% EtOAc in n-hexane as an eluent to give the desired product SMS-7 (1.8 g, 78% yield) as a yellow solid. ¹H NMR (600 MHz, CDCl₃) δ 7.91 (m, 1H), 7.73-7.71 (m, 1H), 7.28 (s, 1H), 4.49 (s, 1H), 4.33-4.30 (m, 1H), 4.12-4.06 (m, 1H), 3.90-3.88 (m, 1H), 3.73-3.72 (m, 2H), 3.65 (s, 3H), 304-3.02 (m, 1H), 2.04 (s, 1H), 1.49 (brs, 9H), 1.28 (d, J−6.7 Hz, 6H). ¹³C NMR (151 MHz, CDCl₃) δ 162.5, 160.3, 155.1, 140.2, 136.1, 128.7, 119.3, 117.3, 116.0, 115.5, 80.4, 71.9, 60.5, 55.4, 51.0, 47.0, 42.9, 30.2, 28.5, 15.7, 15.1.

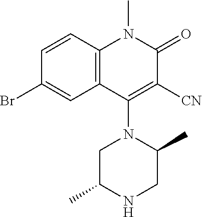 [SMILES: C[C@@H]1CN(c2c(C#N)c(=O)n(C)c3ccc(Br)cc23)[C@@H](C)CN1]

### 6-bromo-4-((2S,5R)-2,5-dimethylpiperazine-1-yl)-1-methyl-2-oxo-1,2-dihydroquinoline-3-carbonitrile (SMS-8)

The compound SMS-7 (1.8 g, 3.7 mmol) was dissolved in anhydrous dichloromethane (30 mL), and trifluoroacetic acid (2.9 mL, 38.0 mmol) was dropwise added at 0° C. under nitrogen (N₂) atmosphere for 10-15 min. The reaction mixture was stirred at room temperature for 16 h, The mixture was cooled down to room temperature and concentrated under reduced pressure. The resultant residue was washed three times with n-Pentane:Et₂O (3:1), and then dried under high vacuum to afford the desired final product SMS-8 (1.2 g, 86% yield) as a white solid. ¹H NMR (600 MHz, CDCl₃) δ 8.16 (m, 1H), 7.84-7.82 (3, 1H), 7.36-7.34 (m, 1H), 4.36 (s, 1H), 3.78 (s, 1H), 3.73 (s, 3H). 3.68-3.63 (m, 1H), 3.54-3.47 (m, 1H), 3.35-3.14 (m, 2H), 1.46 (d, J−6.5 Hz, 3H), 1.12 (d, J−6.2 Hz, 3H).

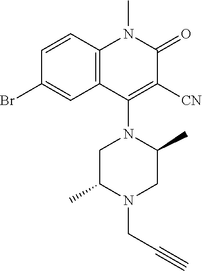 [SMILES: C#CCN1C[C@H](C)N(c2c(C#N)c(=O)n(C)c3ccc(Br)cc23)C[C@H]1C]

### 6-bromo-4-((2S,5R)-2,5-dimethyl-4-(prop-2-yn-1-yl) piperazine-yl)-1-methyl-2-oxo-1,2-dihydroquinoline-3-carbonitrile (SMS-9)

The compound SMS-8 (1.2 g, 3.2 mmol) and propargyl bromide (0.400 mL, 4.0 mmol) was dissolved in anhydrous acetonitrile (25 mL), and N, N-diisopropylethylamine base (1.2 mL, 6.9 mmol) was added at room temperature. The reaction mixture was stirred at 85° C. for 16 h, after which it was diluted with water (30 mL) and then extracted with ethyl acetate (30 mL), and the aqueous layer was separated and ethyl acetate (3×30 mL). The combined organic layer was dried over Na₂SO₄. The residue was purified by flash chromatography using 55% EtOAc in n-hexane as an eluent to give the desired product SMS-9 (1.0 g, 77% yield) as a yellow solid. ¹H NMR (600 MHz, CDCl₃) δ 8.15 (m, 1H), 7.73-7.71 (m, 1H), 7.27-7.25 (m, 1H), 4.14-4.08 (m, 1H), 3.65 (s, 3H). 3.61 (s, 1H), 3.39-3.36 (m, 1H), 3.08 (s, 1H), 2.94-2.90 (m, 1H), 2.86-2.83 (m, 1H) 2.56-2.52 (m, 1H), 2.28 (t, J−2.3 Hz, 1H), 1.02-0.99 (m, 6H.). ¹³C NMR (151 MHz, CDCl₃) δ 161.9, 159.6, 140.0, 136.4, 128.5, 121.2, 116.9, 116.1, 114.5, 102.5, 77.5, 73.7, 58.8, 53.5, 52.3, 42.5, 30.2, 16.8, 15.8.

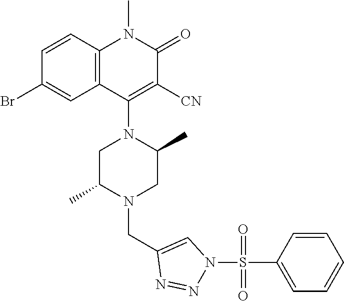 [SMILES: C[C@@H]1CN(c2c(C#N)c(=O)n(C)c3ccc(Br)cc23)[C@@H](C)CN1Cc1cn(S(=O)(=O)c2ccccc2)nn1]

### 6-bromo-4-((2S,5R)-2,5-dimethyl-4-((1-phenylsulfonyl)-1H-1,2,3-triazol-4-yl) methyl)piperazin-1-yl)-1-methyl-2-oxo-1,2-dihydroquinoline-3-carbonitrile (SMS-81)

The compound SMS-9 (200 mg, 0.485 mmol), benzene sulfonyl azide (106 mg, 0.582 mmol), Copper (t)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-81 (0.200 g, 90% yield) as a yellow solid. ¹H NMR (600 MHz, CDCl₃) δ 8.12-8.11 (m, 2H), 8.10 (s, 1H), 8.05 (m, 1H), 7.75-7.70 (m, 2H), 7.62-7.59 (M, 2H), 7.27 (s, 1H), 4.09-3.96 (m, 2H), 3.77-3.75 (m, 1H), 3.65 (s, 3H), 2.99-2.96 (m, 2H), 277 (s, 1H), 2.29-225 (m, 1H), 2.00 (s, 1H), 1.22 (t, J−7.11 Hz, 1H), 1.12 (d, J−6.3 Hz, 3H), 1.02-1.0 (m, 3H). ¹³C NMR (151 MHz, CDCl₃) δ 171.1, 162.1, 159.8, 145.0, 140.0, 136.4, 136.1, 135.8, 129.9, 128.6, 128.5, 122.5, 120.8, 116.9, 116.0, 114.9, 604, 54.7, 48.4, 30.2, 30.2, 29.7, 21.1, 16.6, 14.2.

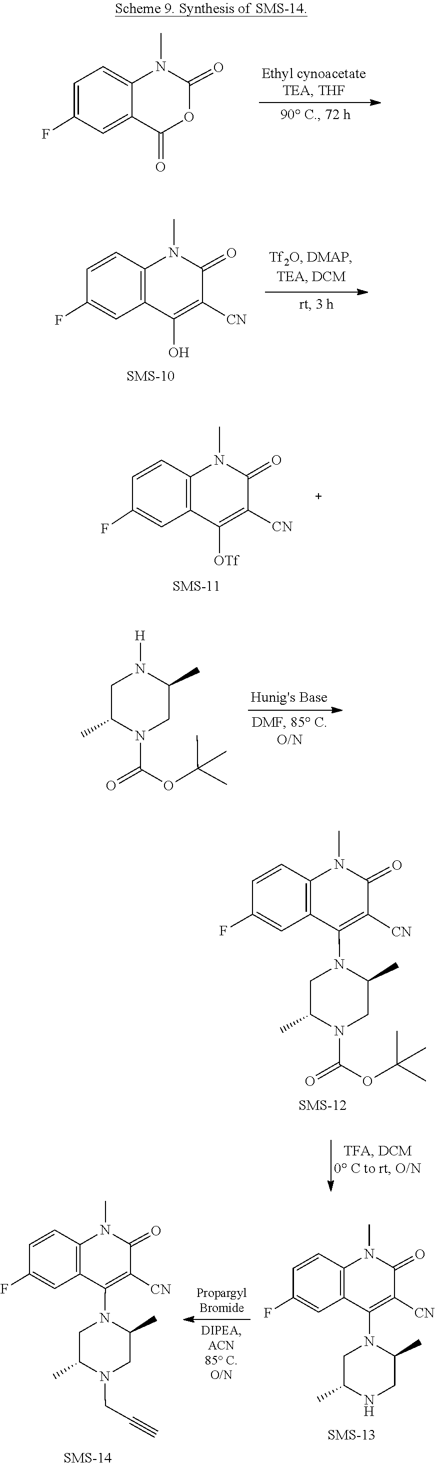

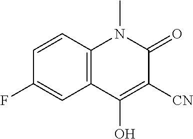 [SMILES: Cn1c(=O)c(C#N)c(O)c2cc(F)ccc21]

### 6-fluoro-4-hydroxy-1-methyl-2-oxo-1,2-dihydroquinoline-3-carbonitrile (SMS-10)

As shown in Scheme 9, above, to a stirred solution of 6-fluoro-1-methyl-2H-benzo (1,3) oxazine-2,4(1H)-dione (3.0 g 15.38 mmol) and ethyl cyanoacetate (3.8 mL, 33.84 mmol), in 25 mL THE was added triethylamine (6.3 mL, 46.14 mmol). The resulting mixture was stirred at 90° C. for 72 h, followed by addition of water (50 mL) and then extracted with ethyl acetate (3×50 mL). The mixture was acidified to pH=1 by addition of hydrochloric acid (2N). The resulting precipitate was collected by filtration and dried over anhydrous Na₂SO₄. The residue was purified by flash chromatography using 25% EtOAc in n-hexane as an eluent to give the desired product SMS-10 (2.8 g, 83% yield) as a yellow solid. ¹H NMR (600 MHz, CDCl₃) δ 7.85-7.83 (m, 1H), 7.52-7.49 (m, 1H), 7.19-7.16 (m, 1H), 3.59 (s, 3H).

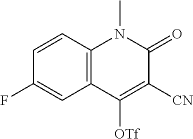 [SMILES: Cn1c(=O)c(C#N)c(OS(=O)(=O)C(F)(F)F)c2cc(F)ccc21]

### 3-cyano-6-fluoro-1-methyl-2-oxo-1,2-dihydroquinolin-4-yl trifluoromethanesulfonate (SMS-11)

To a stirred solution of SMS-10 (2.8 g, 12.84 mmol) in 30 mL DCM was added trifluoromethanesulfonic anhydride (2.3 mL, 14.12 mmol), DMAP (0.390 g, 3.21 mmol) and triethylamine (2.6 mL, 19.26 mmol). The resulting mixture was stirred at room temperature for 3 h, followed by addition of water (30 mL) and then extracted with dichloromethane (3×30 mL). The organic layers were combined and dried over anhydrous Na₂SO₄. The residue was purified by flash chromatography using 65% EtOAc in n-hexane as an eluent to give the desired product SMS-11 (2.2 g, 55% yield) as a yellow solid. ¹H NMR (600 MHz, CDCl₃) δ 7.85-7.83 (m, 1H), 7.52-7.49 (m, 1H), 7.19-7.17 (m, 1H), 3.59 (s, 3H).

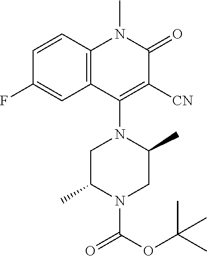 [SMILES: C[C@@H]1CN(c2c(C#N)c(=O)n(C)c3ccc(F)cc23)[C@@H](C)CN1C(=O)OC(C)(C)C]

### tert-butyl (2R,5S)-4-(3-cyano-6-fluoro-1-methyl-2-oxo-1,2-dihydroquinolin-4-yl)-2,5-dimethylpiperazine-1-carboxylate (SMS-12)

The compound SMS-11 (2.2 g, 6.2 mmol) and commercially available tert-butyl (2R, 5S)-2,5-dimethylpiperyzine-1-caroboxylate (1.4 g, 6.2 mmol) was dissolved in anhydrous dimethylformamide (20 mL), and Hunig's base (1.6 mL, 12.4 mmol) was added at room temperature. The reaction mixture was stirred at 85° C. for 16 h, after which it was diluted with brine (30 mL) and then extracted with Et₂O (30 mL), and the aqueous layer was separated and Et₂O (3×30 mL). The combined organic layer was dried over Na₂SO₄. The residue was purified by flash chromatography using 45% EtOAc in n-hexane as an eluent to give the desired product SMS-12 (2.2 g, 84% yield) as a yellow solid. ¹H NMR (600 MHz, CDCl₃) δ 7.96 (m, 1H), 7.45-7.44 (m, 1H), 7.40-7.35 (m, 2H), 4.45 (s, 1H), 4.29-4.27 (m, 1H), 4.04 (s, 1H), 3.85-3.83 (m, 1H), 3.72-3.67 (m, 1H), 3.64 (s, 3H), 1.14 (brs, 9H), 1.24 (t, J−7 Hz, 6H).

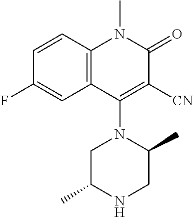 [SMILES: C[C@@H]1CN(c2c(C#N)c(=O)n(C)c3ccc(F)cc23)[C@@H](C)CN1]

### 4-((2S,5R)-2,5-dimethylpiperazine-1-yl)-6-fluoro-1-methyl-2-oxo-1,2-dihydroquinoline-3-carbonitrile (SMS-13)

The compound SMS-12 (2.2 g, 5.3 mmol) was dissolved in anhydrous dichloromethane (30 mL), and trifluoroacetic acid (4.3 mL, 53.50 mmol) was dropwise added at 0° C. under nitrogen (N₂) atmosphere for 10-15 min. The reaction mixture was stirred at room temperature for 16 h. The mixture was concentrated under reduced pressure. The resultant residue was washed three times with n-Pentane:Et₂O (3:1), and then dried under high vacuum to afford the desired final product SMS-13 (1.5 g, 93% yield) as a white solid. ¹H NMR (600 MHz, CDCl₃) δ 7.79 (s, 1H), 7.53-7.39 (m, 1H), 4.36 (s, 1H), 3.76 (s, 3H), 3.70-3.63 (m, 2H), 3.52-3.49 (m, 2H). 3.19-3.16 (m, 1H), 1.46 (d, J−6.4 Hz, 3H), 1.11 (d, J−6.2 Hz, 3H).

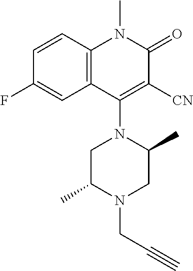 [SMILES: C#CCN1C[C@H](C)N(c2c(C#N)c(=O)n(C)c3ccc(F)cc23)C[C@H]1C]

### 4-((2S,5R)-2,5-dimethyl-4-(prop-2-yn-1-yl) piperazine-yl)-6-fluoro-1-methyl-2-oxo-1,2-dihydroquinoline-3-carbonitrile (SMS-14)

The compound SMS-13 (1.5 g, 4.7 mmol) and propargyl bromide (0.452 mL, 5.9 mmol) was dissolved in anhydrous acetonitrile (25 mL), and N, N-Diisopropylethylamine base (2.4 mL, 14.1 mmol) was added at room temperature. The reaction mixture was stirred at 85° C. for 16 h, after which it was diluted with water (30 mL) and then extracted with ethyl acetate (30 mL), and the aqueous layer was separated and ethyl acetate (3×30 mL). The combined organic layer was dried over Na₂SO₄. The residue was purified by flash chromatography using 50% EtOAc in n-hexane as an eluent to give the desired product SMS-14 (1.4 g, 83% yield) as a yellow solid. ¹H NMR (600 MHz, CDCl₃) δ 7.80-7.79 (m, 1H), 7.44-7.41 (m, 1H), 7.38-7.35 (m, 1H), 4.16 (s, 1H), 3.71 (s, 3H). 3.68 (s, 1H), 3.42-3.39 (m, 1H), 3.10 (s, 1H), 2.99-2.95 (m, 1H), 2.88-2.86 (m, 2H), 2.60-2.56 (m, 1H), 2.30 (t, J−2.2 Hz, 1H), 1.05-1.03 (m, 6H).

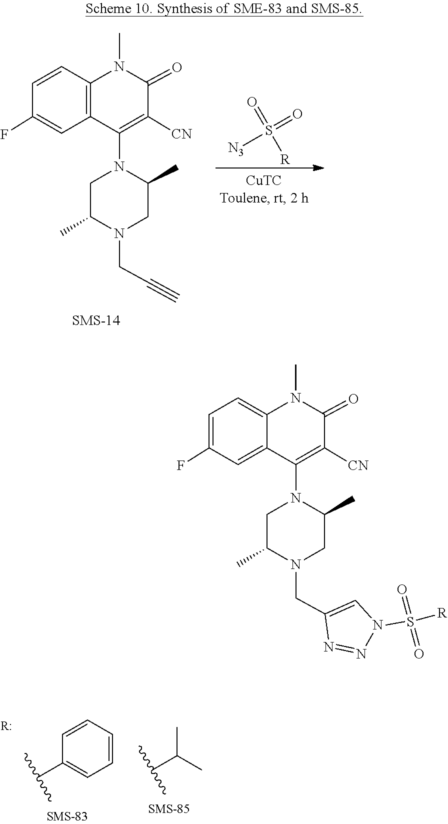

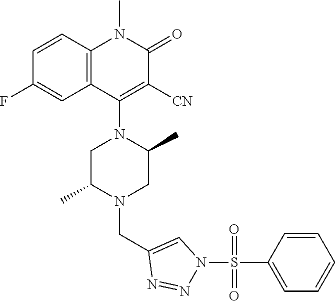 [SMILES: C[C@@H]1CN(c2c(C#N)c(=O)n(C)c3ccc(F)cc23)[C@@H](C)CN1Cc1cn(S(=O)(=O)c2ccccc2)nn1]

### 4-((2S,5R)-2,5-dimethyl-4-((1-phenylsulfonyl)-1H-1, 2, 3-triazol-4-yl) methyl) piperazin-1-yl)-6-fluoro-1-methyl-2-oxo-1,2-dihydroquinoline-3-carbonitrile (SMS-83)

As shown in Scheme 10, above, the compound SMS-14 (200 mg, 0.560 mmol), benzene sulfonyl azide (155 mg, 0.851 mmol), Copper (1)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-83 (0.250 g, 83% yield) as a yellow solid. ¹H NMR (600 MHz, CDCl₃) δ 8.12 (m, 2H), 8.11-8.10 (m, 1H), 8.05 (m, 1H), 7.74-7.71 (m, 1H), 7.63-7.60 (m, 2H), 7.60-7.59 (m, 1H), 7.41-7.35 (m, 2H), 4.09-4.06 (m, 2H), 3.97-3.95 (m, 1H), 3.80-3.75 (m, 1H), 3.68 (s, 31), 2.98-2.94 (m, 2H), 2.73 (s, 1H), 2.27-2.23 (m, 1H), 2.00 (s, 1H), 1.21 (t, J 7.0 Hz, 2H), 1.12 (d, J−6.2 Hz, 3H), 1.0 (m, 3H). ¹³C NMR (151 MHz, CDCl₃) δ 188.1, 171.1, 162.2, 159.8, 159.0, 157.4, 145.7, 137.7, 136.1, 135.8, 129.9, 128.6, 122.5, 121.8, 121.6, 120.5, 117.0, 114.9, 111.5, 111.3, 60.3, 54.6, 48.2, 30.3, 21.0, 16.6, 142.

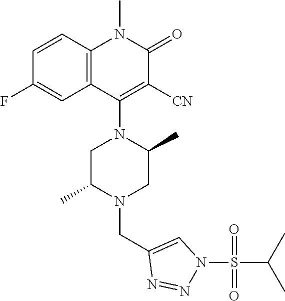 [SMILES: CC(C)S(=O)(=O)n1cc(CN2C[C@H](C)N(c3c(C#N)c(=O)n(C)c4ccc(F)cc34)C[C@H]2C)nn1]

### 6-fluoro-4-((2S,5R)-4-((isopropyl sulfonyl)-1H-1,2,3-triazol-4-yl) methyl)-2,5-dimethyl piperazin-1-yl)-1-methyl-2-oxo-1,2-dihydroquinoline-3-carbonitrile (SMS-85)

The compound SMS-14 (200 mg, 0.549 mmol), isopropyl sulfonyl azide (98 mg, 0.658 mmol), Copper (i)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-85 (0.195 g, 83% yield) as a yellow solid. ¹H NMR (600 MHz, CDCl₃) δ 8.05 (s, 1H), 7.68-7.67 (m, 1H), 7.43-7.40 (m, 1H), 7.37-7.35 (m, 1H), 4.13-4.09 (m, 1H), 4.06-4.03 (m, 1H), 3.92-3.90 (m, 1H), 3.89-3.85 (m, 1H), 3.70 (s, 3H), 3.69 (s, 1H), 3.04-00 (m, 2H), 2.79 (s, 1H), 2.33-2.30 (m, 1H), 2.03 (s, 1H), 1.45 (t, J −6.7 Hz, 6H), 1.26-1.24 (m, 1H), 1.19 (d, J−6.2 Hz, 3H), 1.05 (m, 3H). ¹³C NMR (151 MHz, CDCl₃) δ 188.2, 162.3, 159.9, 159.1, 157.5, 144.6, 137.8, 123.9, 121.8, 121.7, 117.0, 116.9, 111.7, 111.5, 57.5, 54.8, 48.3, 30.5, 16.8, 16.2, 16.1, 14.3.

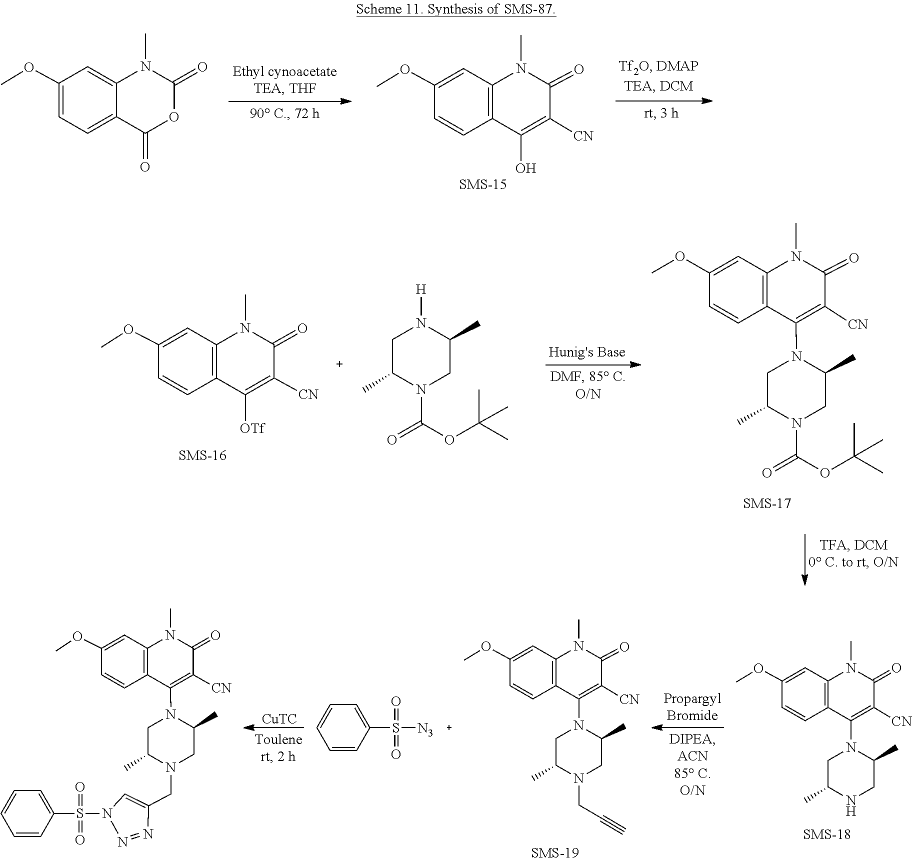

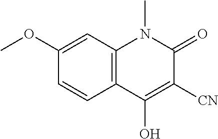 [SMILES: COc1ccc2c(O)c(C#N)c(=O)n(C)c2c1]

### 4-hydroxy-7-methoxy-1-methyl-2-oxo-1,2-dihydroquinoline-3-carbonitrile (SMS-15)

As shown in Scheme 11, above, to a stirred solution of 7-methoxy-1-methyl-2H-benzo (1,3) oxazine-2,4(1H)-dione (3.0 g 14.48 mmol) and ethyl cyanoacetate (3.5 mL, 31.87 mmol), in 30 mL THE was added triethylamine (8 mL, 57.92 mmol). The resulting mixture was stirred at 90° C. for 72 h, followed by addition of water (50 mL) and then extracted with ethyl acetate (3×50 mL). The mixture was acidified to pH=1 by addition of hydrochloric acid (2N). The resulting precipitate was collected by filtration and dried over anhydrous Na₂SO₄. The residue was purified by flash chromatography using 25% EtOAc in n-hexane as an eluent to give the desired product SMS-15 (2.5 g, 67% yield) as a brown solid. ¹H NMR (600 MHz, DMSO-d₆) δ 8.00 (d, J−8.9 Hz, 1H), 7.0-6.9 (m, 1H), 6.9 (m, 1H), 3.9 (s, 3H), 3.5 (s, 3H).

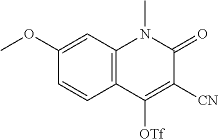 [SMILES: COc1ccc2c(OS(=O)(=O)C(F)(F)F)c(C#N)c(=O)n(C)c2c1]

### 3-cyano-7-methoxy-1-methyl-2-oxo-1,2-dihydroquinolin-4-yl trifluoromethanesulfonate (SMS-16)

To a stirred solution of SMS-15 (2.5 g, 8.99 mmol) in 30 mL DCM was added trifluoromethanesulfonic anhydride (2.2 mL, 13.49 mmol), DMAP (0.110 g, 0.899 mmol) and Triethylamine (2.5 mL, 17.98 mmol). The resulting mixture was stirred at room temperature for 3 h, followed by addition of water (30 mL) and then extracted with dichloromethane (3×30 mL). The organic layers were combined and dried over anhydrous Na₂SO₄. The residue was purified by flash chromatography using 75% EtOAc in n-hexane as an eluent to give the desired product SMS-16 (3.8 g, 93% yield) as a brown yellow solid. ¹H NMR (600 M Hz, CDCl₃) δ 780 (d, J−9.1 Hz, 1H), 7.03-7.02 (m, 1H), 6.87-6.86 (m, 1H), 4.01 (s, 3H), 3.73 (s, 3H).

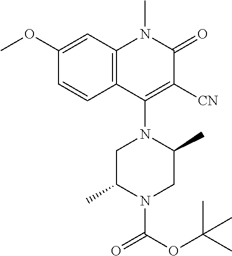 [SMILES: COc1ccc2c(N3C[C@@H](C)N(C(=O)OC(C)(C)C)C[C@@H]3C)c(C#N)c(=O)n(C)c2c1]

### tert-butyl (2R,5S)-4-(3-cyano-7-methoxy-1-methyl-2-oxo-1,2-dihydroquinolin-4-yl)-2,5-dimethylpiperazine-1-carboxylate (SMS-17)

The compound SMS-16 (3.8 g, 10.49 mmol) and commercially available tert-butyl (2R, 5S)-2,5-dimethylpiperyzine-1-caroboxylate (2.5 g, 10.49 mmol) was dissolved in anhydrous dimethylformamide (20 mL), and Hunig's base (2.7 mL, 20.98 mmol) was added at room temperature. The reaction mixture was stirred at 85° C. for 16 h, after which it was diluted with brine (40 mL) and then extracted with Et₂O (40 mL), and the aqueous layer was separated and Et₂O (3×30 mL). The combined organic layer was dried over Na₂SO₄. The residue was purified by flash chromatography using 60% EtOAc in n-hexane as an eluent to give the desired product SMS-17 (2.8 g, 64% yield) as a brown solid. ¹H NMR (600 MHz, CDCl₃) δ 7.74-7.72 (m, 1H), 6.85-6.81 (m, 1H), 6.75 (d, J−0.3 Hz, 1H), 4.46 (s, 1H), 4.29-4.28 (m, 1H), 4.10-4.05 (m, 1H), 3.93 (s, 3H), 3.89-3.81 (m, 1H), 3.76-3.70 (m, 1H), 3.63 (s, 3H), 3.04-3.02 (m, 1H), 1.48 (brs 9H), 1.26 (t, J−6.8 Hz, 6H).

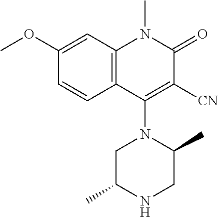 [SMILES: COc1ccc2c(N3C[C@@H](C)NC[C@@H]3C)c(C#N)c(=O)n(C)c2c1]

### 4-((2S,5R)-2,5-dimethylpiperazine-1-yl)-7-methoxy-1-methyl-2-oxo-1,2-dihydroquinoline-3-carbonitrile (SMS-18)

The compound SMS-17 (2.8 g, 6.56 mmol) was dissolved in anhydrous dichloromethane (30 mL), and trifluoroacetic acid (5.5 mL, 65.69 mmol) was dropwise added at 0° C. under nitrogen (N₂) atmosphere for 10-15 min. The reaction mixture was stirred at room temperature for 16 h, The mixture was cooled down to room temperature and concentrated under reduced pressure. The resultant residue was washed three times with n-Pentane:Et₂O (3:1), and then dried under high vacuum to afford the desired final product SMS-18 (2.0 g, 93% yield) as a white solid. ¹H NMR (600 MHz, CDCl₃) δ 9.46 (s, 1H), 8.54 (s, 1H), 7.45-7.43 (m, 1H), 4.75 (m, 1H), 4.40 (s, 3H), 4.22 (m, 1H), 4.14 (s, 3H), 4.10-3.98 (m, 2H), 3.79-3.58 (m, 2H), 1.88 (d, J−5.8 Hz, 3H), 1.50 (d, J−5.7 Hz, 3H).

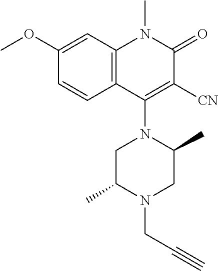 [SMILES: C#CCN1C[C@H](C)N(c2c(C#N)c(=O)n(C)c3cc(OC)ccc23)C[C@H]1C]

### 4-((2S,5R)-2,5-dimethyl-4-(prop-2-yn-1-yl) piperazine-yl)-7-methoxy-1-methyl-2-oxo-1,2-dihydroquinoline-3-carbonitrile (SMS-19)

The compound SMS-18 (2.0 g, 6.13 mmol) and propargyl bromide (0.912 mL, 7.66 mmol) was dissolved in anhydrous acetonitrile (25 mL), and N, N-diisopropylethylamine base (3.2 mL, 18.39 mmol) was added at room temperature. The reaction mixture was stirred at 85° C. for 16 h, after which it was diluted with water (30 mL) and then extracted with ethyl acetate (30 mL), and the aqueous layer was separated and ethyl acetate (3×30 mL). The combined organic layer was dried over Na₂SO₄. The residue was purified by flash chromatography using 50% EtOAc in n-hexane as an eluent to give the desired product SMS-19 (1.6 g, 86% yield) as a yellow solid. ¹H NMR (600 MH-z, CDCl₃) δ 8.04-8.02 (m, 1H), 6.87-6.85 (m, 1H), 6.73 (d, J−2.3 Hz, 1H), 4.10-4.05 (m, 1H), 3.92 (s, 3H), 3.67-3.66 (m, 1H), 3.64 (s, 3H), 3.39-3.35 (m, 1H), 3.07 (s, 1H), 2.98-2.94 (m, 1H), 2.83-2.81 (m, 2H), 2.55-2.52 (m. 1H), 2.28 (t, J−2.31 Hz, 1H), 1.0 (t, J−6.3 Hz, 6H).

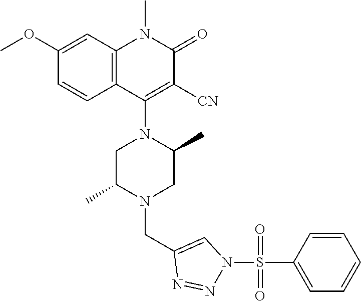 [SMILES: COc1ccc2c(N3C[C@@H](C)N(Cc4cn(S(=O)(=O)c5ccccc5)nn4)C[C@@H]3C)c(C#N)c(=O)n(C)c2c1]

### 4-((2S,5R)-2,5-dimethyl-4-((1-phenylsulfonyl)-1H-1,2,3-triazol-4-yl) methyl) piperazin-1-yl)-7-methoxy-1-methyl-2-oxo-1,2-dihydroquinoline-3-carbonitrile (SMS-87)

The compound SMS-19 (200 mg, 0.549 mmol), benzene sulfonyl azide (122 mg, 0.658 mmol), Copper (t)-thiophene-2-carboxylate (25 mg, 0.150 mmol) and anhydrous toluene (5 mL) were used. The resulting mixture was stirred at room temperature for 2 h, followed by addition of water (30 mL) and then extracted with ethyl acetate (3×30 mL). The organic layers were combined and dried over anhydrous Na₂SO₄. The residue was purified by flash chromatography using 90% EtOAc in n-hexane as an eluent to give the desired product SMS-87 (0.260 g, 87% yield) as a yellow solid. ¹H NMR (600 MHz, CDCl₃) δ 8.15-8.14 (m, 2H), 8.10 (s, 1H), 7.94-7.93 (m, 1H), 7.75-7.72 (m, 1H), 7.63-7.61 (m, 2H), 6.86-6.85 (m, 2H), 6.76-6.74 (m, 1H), 4.12-4.06 (m, 2H), 3.98-3.94 (m, 1H), 3.93 (s, 3H), 3.85-3.82 (m, 1H), 3.66 (s, 3H), 3.03-2.95 (m, 2H), 2.73 (s, 1H), 2.26-2.23 (m, 1H), 2.03-2.02 (m, 1H), 1.26-1.23 (m, 1H), 1.12 (d, J−6.1 Hz, 3H), 1.01 (m, 1H). ¹³C NMR (151 MHz, CDCl₃) δ 188.1, 164.3, 163.3, 161.0, 143.3, 136.2, 135.8, 130.0, 128.7, 1284, 122.5, 115.8, 110.8, 99.0, 60.4, 55.9, 54.7, 483, 301, 211, 168, 14.3.

### Example 10

### Activation of Human T Cell Lines with SuTEx Compounds

### Cell Culture and Jurkat Stimulation:

Jurkat T cell line was maintained in RPMI 1640 (Invitrogen Life Technologies, Carlsbad, California, United States of America) supplemented with 10% FBS (U.S. Source, Omega Scientific, Inc., Tarzana, California, United States of America), 1% L-glutamine (Thermo Fisher Scientific, Waltham, Massachusetts, United States of America), and 1% penicillin/streptomycin in 75-cm³ flasks with a starting density of 3×10⁵/ml at 37° C. under 5% CO₂. Cells were grown for 48 hrs. A 12-well plate treated with a mixture of both CD3 antibodies (anti-human CD3 antibodies sold under the tradename IN VIVO READY™ Clone: UCHT1 40-0038-U500, Tonbo Biosciences (San Diego, California, United States of America); 0.25 ml at 2 mg/ml) and CD28 antibodies (anti-human CD28 antibodies sold under the tradename IN VIVO READY™ Clone: CD28.2, 40-0289-U500, Tonbo Biosciences (San Diego, California, United States of America); 0.25 mg at 2 mg/ml) in 500 μL of PBS for 24 hrs. Cells were switched to serum-free RPMI 1640 prior to inhibitor treatment. Jurkat cells were suspended in serum-free RPMI with a density of 2×10⁷/ml with 300 nM with the assigned inhibitor in Eppendorf tube and incubated for 15 min at 37° C. under 5% CO₂. The treated 12 well-plate was washed 3× with warm PBS to remove excess VD3/CD28 Then, followed by having each sample transferred to one of the wells in the 12-well plate for 15 min at 37° C. under 5% CO₂. Finally, each well was quenching with cold PBS. Transferred pellet in 100 μL PBS with protease/phosphatase (EDTA-free). Sonicated for 1 sec, 20% amp, 3x. Stored at −80° C.

### Western Blot Analysis for TC Stimulation:

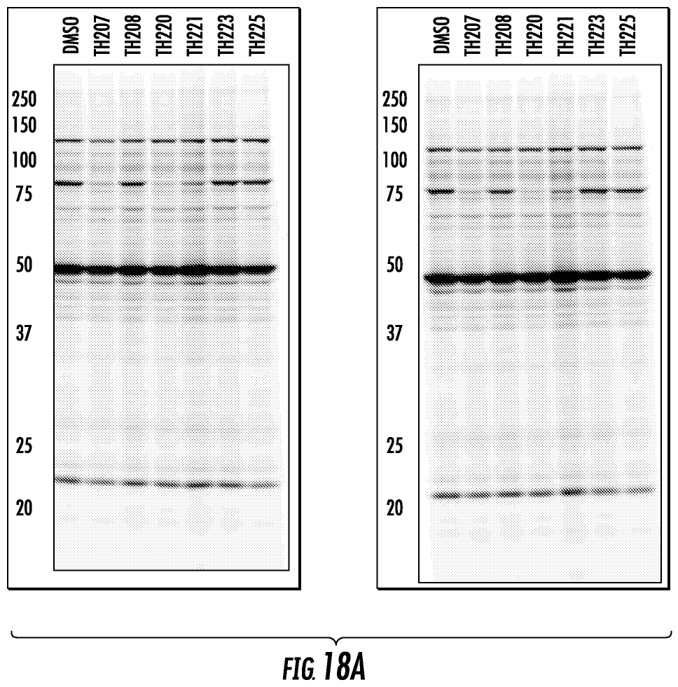

The following antibodies were purchased for western blot studies: Anti-ERK antibody produced in rabbit (Cell Signaling Technology, Inc. (Danvers, Massachusetts, United States of America), p44/42 MAPK (Erk1/2, 137F5) Cat #4695S); Anti-pERK rabbit polyclonal antibody (Cell Signaling Technology, P-p44/42 MAPK (T202/Y204) Cat #9102S) Goat anti-Rabbit antibody sold under the tradename DYLIGHT™ 650 (Thermo Fisher Scientific Cat #84545 (Thermo Fisher Scientific, Waltham, Massachusetts, United States of America)); and Goat anti-Rabbit antibody sold under the tradename DYLIGHT™ 550 (Thermo Fisher Scientific Cat #84541 (Thermo Fisher Scientific, Waltham, Massachusetts, United States of America)). Proteins were separated by SDS-PAGE (7.5% polyacrylamide, TGX Stain-Free Mini Gel) at 150 V for 55 min. Gel transfers were performed using the Bio-Rad Trans-Blot Turbo RTA Midi Nitrocellulose Transfer Kit with a Bio-Rad Trans-Blot Turbo Transfer System (25V, 10 min) (both from Bio-Rad Laboratories, Hercules, California, United States of America). The nitrocellulose blot was then incubated in blocking solution (30 mL, 3% BSA in TBS-T (1.5 M NaCl, 0.25 M Tris pH 7.4 in ddH₂O)) for 1 h at 25° C. with gentle shaking. The blot was then transferred immediately to primary antibody solution (1:1,000 anti-ERK) and incubated overnight at 4° C. with gentle shaking. The blot was then rinsed 5 times for 5 min in TBS-T, transferred immediately into secondary antibody solution (1:10,000 anti-species DYLIGHT™ 550 or DYLIGHT™ 650 in TBS-T), and incubated for 1 h at 25° C. with gentle shaking. The blot was then rinsed 5 times for 5 min in TBS-T, transferred into ddH₂O, and imaged by in-blot fluorescence scanning on an imaging system sold under the tradename CHEMIDOC™ MP (Bio-Rad Laboratories, Hercules, California, United States of America). Each lane displayed in western blots represents an individual biological replicate of that overexpression/treatment condition. Results are shown in FIG. 17.

### Discussion:

T cell receptor (TCR) signaling is mediated by secondary messengers including diacylglycerols (DAGs) that act as ligands to alter subcellular localization and activation of key proteins (e.g. MAPK⁵⁶ and PKC⁵⁷) for T cell activation⁵⁹. DGK-alpha (DGKα) and -zeta (DGKζ) are negative regulators of TCR signaling by phosphorylating the secondary messenger DAG to terminate its signaling activity⁵⁹. Excessive DGK activity and thus attenuated DAG signaling has been linked to defective T cell function⁵⁹⁻⁷². Accordingly, it is believed that compounds that can activate T cells, such as in the assays described herein, act as DGK-alpha and/or zeta inhibitors.

Without being bound to any one theory, it is believed that development of DGK-selective inhibitors can restore deficient DAG signaling to overcome immunosuppression of tumor infiltrating T cell (TIL) activity. The rationale for focusing on DGKα and DGKζ over other DGK isoforms include (i) gene expression data showing the potential for tissue specificity using DGKα/ζ inhibitors because of the enriched expression of these DGKs in T cells, (ii) genetic evidence that DGKα mediates a hyporesponsive T cell state known as anergy^{73,74} and disruption of DGKα restored cytokine production and activation, and promotes resistance to T cell anergy^{64,65}, (iii) clinical evidence showing TILs isolated from renal carcinoma patients exhibit increased expression of DGKα, which correlated with impaired cytotoxic responses that could be reversed with non-selective DGKα inhibitors⁶⁶, and (iv) DGKα inactivation delays the exhaustion of tumor-specific T cells and enhances the efficacy of anti-PD-1 therapy^{75,76}.

### Example 11

### Synthesis of Covalent Inhibitors with a bis((4-fluorophenyl)methylene)piperidine or Structurally Related Leaving Group

Additional SuTEx inhibitor compounds were prepared with bis((4-fluorophenyl)methylene)piperidine-based or bis((4-fluorophenyl)methyl)piperazine-based leaving groups. The inhibitor compounds were prepared using via reactions between alkynes and azides to form sulfonyl-triazoles, similar to methods described in Example 9, above. The alkyne 4-((Bis(4-fluorophenyl)methylene)-1-but-3-yn-1-yl)piperidine was prepared as previously described. See PCT International Patent Application Publication No. WO 2020/214336 to Hsu et al., published Oct. 22, 2020, and U.S. Patent Application Publication No. 2022/0214355 to Hsu et al., published Jul. 7, 2022, the disclosures of which are incorporated herein by reference in their entireties. The azides can be prepared as described by the general procedure below.

### General Procedure for Preparation of Substituted Sulfonyl Azides

Sodium azide (4.8 mmol, 1.2 equiv.) was dissolved in 20 mL of water and added dropwise to a stirred solution of substituted sulfonyl chloride (4 mmol, 1.0 equiv.) in acetonitrile (30 mL) at room temperature. Upon completion, the acetonitrile in reaction solution was removed in vacuo, and the remaining aqueous solution was extracted 3 times with ether. The combined organic layers were dried over anhydrous sodium sulfate, filtered and then concentrated under reduced pressure. The crude product was purified by chromatography with elution system (hexane:ethyl acetate=8:1) to provide the pure product.

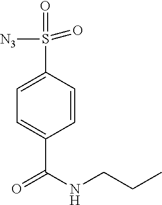 [SMILES: CCCNC(=O)c1ccc(S(=O)(=O)N=[N+]=[N-])cc1]

### 4-(Propylcarbamoyl)benzenesulfonyl azide (XJ-1-67)

¹H NMR (600 MHz, CDCl₃) δ 8.04-7.99 (m, 2H), 7.98-7.95 (m, 2H), 6.21 (s, 1H), 3.57-3.30 (m, 2H), 1.77-1.62 (m, 2H), 1.06-0.94 (m, 3H). ¹³C NMR (200 MHz, CDCl₃) δ 165.46, 140.75, 140.65, 128.27, 127.81, 42.12, 22.82, 11.37. HRMS (ESI) calcd for C₁₀H₁₂N₄O₃S [M+H]⁺: 269.0664, found: 269.0665.

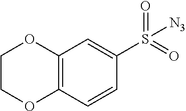 [SMILES: [N-]=[N+]=NS(=O)(=O)c1ccc2c(c1)OCCO2]

### 2,3-Dihydrobenzo[b][1,4]dioxine-6-sulfonyl azide (XJ-2-75)

¹H NMR (800 MHz, CDCl₃) δ 7.56 (d, J=2.3 Hz, 1H), 7.54 (dd, J=8.6, 2.3 Hz, 1H), 7.12 (d, J=8.6 Hz, 1H), 4.46-4.44 (m, 2H), 4.43-4.41 (m, 2H). ¹³C NMR (200 MHz, CDCl₃) δ 148.88, 143.51, 129.99, 120.95, 117.85, 116.73, 64.23, 63.69. HRMS (ESI) calcd for C₈H₇N₃O₄S [M+H]⁺: 242.0157, found: 242.0158.

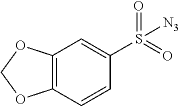 [SMILES: [N-]=[N+]=NS(=O)(=O)c1ccc2c(c1)OCO2]

### Benzo[d][1,3]dioxole-5-sulfonyl azide (XJ-2-85)

¹H NMR (800 MHz, CDCl₃) δ 7.53 (ddd, J=2.0, 0.8 Hz, 1H), 7.31 (dd, J=2.1, 0.8 Hz, 1H), 6.95 (dd, J=8.3, 0.7 Hz, 1H), 6.14 (s, 2H). ¹³C NMR (201 MHz, CDCl₃) δ 152.83, 148.27, 130.85, 123.52, 108.21, 107.00, 102.47. HRMS (ESI) calcd for C₇H₅N₃O₄S [M+H]⁺: 228.0035, found: 228.0036.

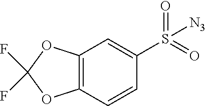 [SMILES: [N-]=[N+]=NS(=O)(=O)c1ccc2c(c1)OC(F)(F)O2]

### 2,2-Difluorobenzo[d][1,3]dioxole-5-sulfonyl azide (XJ-2-113)

¹H NMR (800 MHz, CDCl₃) δ 7.81 (dd, J=8.4, 1.9 Hz, 1H), 7.67 (d, J=1.9 Hz, 1H), 7.30 (d, J=8.4 Hz, 1H). ¹³C NMR (200 MHz, CDCl₃) δ 147.69, 143.69, 133.82, 131.28 (t, J=260.4 Hz), 124.60, 109.85, 108.76. ¹⁹F NMR (564 MHz, CDCl₃) δ −52.23. HRMS (ESI) calcd for C₇H₃F₂N₃O₄S [M+H]⁺: 263.1748, found: 263.1747.

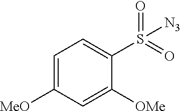 [SMILES: COc1ccc(S(=O)(=O)N=[N+]=[N-])c(OC)c1]

### 2,4-Dimethoxybenzenesulfonyl azide (XJ-2-135)

¹H NMR (800 MHz, CDCl₃) δ 8.01-7.84 (m, 1H), 6.80-6.41 (m, 2H), 4.08 (s, 3H), 3.97 (s, 3H). ¹³C NMR (200 MHz, CDCl₃) δ 165.72, 158.29, 131.79, 119.45, 104.20, 98.91, 76.74, 76.58, 76.42, 55.84, 55.48. HRMS (ESI) calcd for C₈H₉N₃O₄S [M+H]³⁰: 244.0347, found: 244.0346.

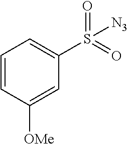 [SMILES: COc1cccc(S(=O)(=O)N=[N+]=[N-])c1]

### 3-Methoxybenzenesulfonyl azide (XJ-2-137)

¹H NMR (800 MHz, CDCl₃) δ 8.02-7.98 (m, 1H), 7.79-7.66 (m, 1H), 7.24-7.16 (m, 2H), 4.13 (s, 3H). ¹³C NMR (200 MHz, CDCl₃) δ 156.56, 135.81, 129.90, 129.89, 127.18, 120.11, 111.84, 55.89. HRMS (ESI) calcd for C₇H₇N₃O₃S [M+H]⁺: 214.0242, found: 214.0241.

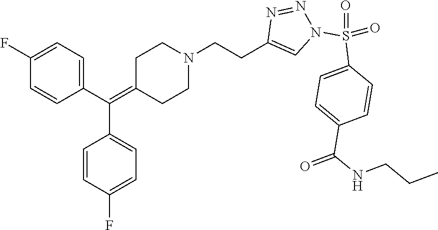 [SMILES: CCCNC(=O)c1ccc(S(=O)(=O)n2cc(CCN3CCC(=C(c4ccc(F)cc4)c4ccc(F)cc4)CC3)nn2)cc1]

### 4-((4-(2-(4-(Bis(4-fluorophenyl)methylene)piperidin-1-yl)ethyl)-1H-1,2,3-triazol-1-yl)sulfonyl)-N-propylbenzamide (TH225)

¹H NMR (800 MHz, CDCl₃) δ 8.14-8.10 (m, 2H), 8.00 (s, 1H), 7.96-7.92 (m, 2H), 7.09-7.01 (m, 4H), 7.00-6.94 (m, 4H), 6.43 (t, J=5.9 Hz, 1H), 3.45-3.35 (m, 2H), 2.92 (t, J=7.5 Hz, 2H), 2.67 (t, J=7.5 Hz, 2H), 2.52 (t, J=5.9 Hz, 4H), 2.36 (t, J=5.6 Hz, 4H), 1.62 (h, J=7.4 Hz, 2H), 0.96 (t, J=7.4 Hz, 3H). ¹³C NMR (200 MHz, CDCl₃) δ 165.28, 162.15, 160.93, 146.53, 141.43, 138.41, 138.08, 135.78, 134.16, 131.47-131.09 (m, 2C), 128.85-128.80 (m, 2C), 128.41-128.23 (m, 2C), 121.32, 121.25, 115.15-114.87 (m, 2C) 56.75, 54.92, 42.11, 31.60, 23.35, 22.86, 22.76, 11.47, 11.34. ¹⁹F NMR (564 MHz, CDCl₃) δ −119.07. HRMS (ESI) calcd for C₃₂H₃₃F₂N₅O₃S [M+H]⁺: 606.2326, found: 606.2327.

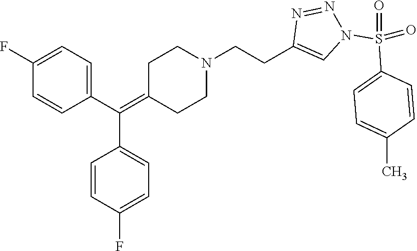 [SMILES: Cc1ccc(S(=O)(=O)n2cc(CCN3CCC(=C(c4ccc(F)cc4)c4ccc(F)cc4)CC3)nn2)cc1]

### 4-(Bis(4-fluorophenyl)methylene)-1-(2-(1-tosyl-1H-1,2,3-triazol-4-yl)ethyl)piperidine (TH207)

¹H NMR (800 MHz, CDCl₃) δ 8.00-7.95 (m, 3H), 7.37 (d, J=8.2 Hz, 2H), 7.10-7.02 (m, 4H), 7.00-6.95 (m, 4H), 2.93 (t, J=7.5 Hz, 2H), 2.68 (t, J=7.5 Hz, 2H), 2.52 (t, J=5.6 Hz, 4H), 2.44 (s, 3H), 2.37 (t, J=5.7 Hz, 4H). ¹³C NMR (201 MHz, CDCl₃) δ 162.14, 160.92, 147.15, 146.20, 138.13, 138.11, 135.97, 134.04, 133.27, 131.41, 131.40, 131.38, 131.36, 131.33, 131.27, 131.25, 131.23, 131.21, 131.19, 130.47, 130.37, 130.32, 128.65, 128.62, 128.59, 121.06, 121.03, 121.00, 115.13, 115.11, 115.08, 115.02, 115.01, 114.98, 114.96, 114.95, 114.89, 114.86, 114.84, 56.89, 54.96, 31.73, 23.53, 21.88. ¹⁹F NMR (564 MHz, CDCl₃) δ −119.10. HRMS (ESI) calcd for C₂₉H₂₈F₂N₄O₂S [M+H]⁺: 535.1968, found: 535.1970.

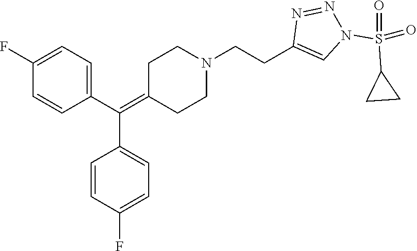 [SMILES: O=S(=O)(C1CC1)n1cc(CCN2CCC(=C(c3ccc(F)cc3)c3ccc(F)cc3)CC2)nn1]

### 4-(bis(4-fluorophenyl)methylene)-1-(2-(1-(cyclopropylsulfonyl)-1H-1,2,3-triazol-4-yl)ethyl)piperidine (TH223)

¹H NMR (800 MHz, CDCl₃) δ 7.92 (s, 1H), 7.12-7.04 (m, 4H), 7.00-6.92 (m, 4H), 2.98 (t, J=7.5 Hz, 2H), 2.86 (tt, J=7.9, 4.7 Hz, 1H), 2.71 (t, J=7.5 Hz, 2H), 2.54 (t, J=5.6 Hz, 4H), 2.44-2.30 (m, 4H), 1.67-1.47 (m, 2H), 1.36-1.16 (m, 2H). ¹³C NMR (200 MHz, CDCl₃) δ 162.12, 160.90, 145.98, 138.13, 138.12, 135.96, 134.04, 131.33, 131.29, 121.42, 115.02, 114.92, 56.90, 54.94, 32.16, 23.38, 7.80. ¹⁹F NMR (564 MHz, CDCl₃) δ −119.07. HRMS (ESI) calcd for C₂₅H₂₆F₂N₄O₂S [M+H]⁺: 485.1805, found: 485.1810.

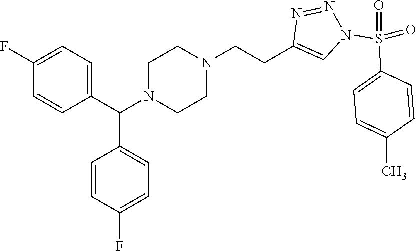 [SMILES: Cc1ccc(S(=O)(=O)n2cc(CCN3CCN(C(c4ccc(F)cc4)c4ccc(F)cc4)CC3)nn2)cc1]

### 1-(Bis(4-fluorophenyl)methyl)-4-(2-(1-tosyl-1H-1,2,3-triazol-4-yl)ethyl)piperazine (TH208)

¹H NMR (800 MHz, CDCl₃) δ 8.00-7.97 (m, 2H), 7.95 (s, 1H), 7.43-7.32 (m, 6H), 7.02-6.93 (m, 4H), 4.22 (s, 1H), 2.96-2.83 (m, 2H), 2.71-2.62 (m, 2H), 2.60-2.21 (m, 1H). ¹³C NMR (200 MHz, CDCl₃) δ 162.44, 161.22, 147.14, 146.09, 138.22, 133.26, 130.46, 130.38, 130.31, 129.32, 129.29, 129.18, 129.14, 128.66, 128.63, 128.60, 121.04, 120.97, 115.56, 115.45, 115.41, 115.30, 56.90, 53.17, 51.74, 23.21, 21.89, 21.86, 21.82. ¹⁹F NMR (564 MHz, CDCl₃) δ −118.83. HRMS (ESI) calcd for C₂₈H₂₉F₂N₅O₂S [M+H]⁺: 538.2072, found: 538.2074.

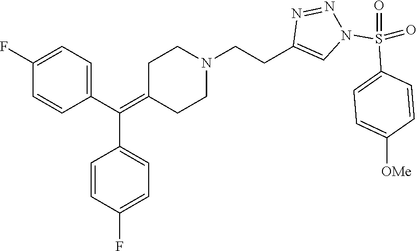 [SMILES: COc1ccc(S(=O)(=O)n2cc(CCN3CCC(=C(c4ccc(F)cc4)c4ccc(F)cc4)CC3)nn2)cc1]

### 4-(Bis(4-fluorophenyl)methylene)-1-(2-(1-tosyl-1H-1,2,3-triazol-4-yl)ethyl)piperidine (TH220)

¹H NMR (800 MHz, CDCl₃) δ 8.08-8.01 (m, 2H), 7.97 (s, 1H), 7.09-7.04 (m, 4H), 7.04-7.01 (m, 2H), 7.00-6.96 (m, 4H), 3.88 (s, 3H), 2.98-2.85 (m, 2H), 2.74-2.63 (m, 2H), 2.57-2.48 (m, 4H), 2.42-2.10 (m, 4H). ¹³C NMR (200 MHz, CDCl₃) δ 165.27, 162.15, 160.93, 146.07, 138.11, 135.98, 134.04, 132.89, 131.5-130.89 (m, 4C), 131.40, 131.36, 131.33, 131.27, 131.23, 131.20, 131.17, 131.14, 131.12, 129.40, 120.91, 120.87, 120.84, 115.30-114.74 (m, 4C), 57.11-56.70 (m, 1C), 55.94 (q, J=49.0 Hz, 1C), 54.94, 31.64, 23.43. ¹⁹F NMR (564 MHz, CDCl₃) δ −119.12. HRMS (ESI) calcd for C₂₉H₂₈F₂N₄O₃S [M+H]⁺: 551.1917, found: 551.1916.

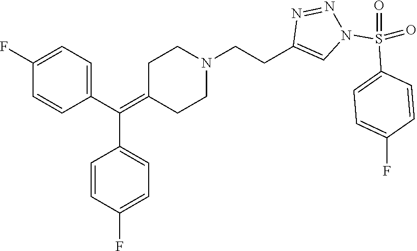 [SMILES: O=S(=O)(c1ccc(F)cc1)n1cc(CCN2CCC(=C(c3ccc(F)cc3)c3ccc(F)cc3)CC2)nn1]

### 4-(Bis(4-fluorophenyl)methylene)-1-(2-(1-((4-fluorophenyl)sulfonyl)-1H-1,2,3-triazol-4-yl)ethyl)piperidine (TH221)

¹H NMR (800 MHz, CDCl₃) δ 8.17-8.13 (m, 2H), 7.99 (s, 1H), 7.30-7.24 (m, 2H), 7.09-7.03 (m, 4H), 7.01-6.94 (m, 4H), 2.95 (t, J=7.5 Hz, 2H), 2.69 (t, J=7.5 Hz, 2H), 2.53 (t, J=5.6 Hz, 4H), 2.37 (t, J=5.6 Hz, 4H). ¹³C NMR (200 MHz, CDCl₃) δ 167.47, 166.18, 162.16, 160.93, 146.34, 138.85, 138.08 (d, J=3.5 Hz), 135.83, 134.13, 132.23 (d, J=3.2 Hz), 131.75 (d, J=10.2 Hz), 131.29 (d, J=7.5 Hz), 121.06, 117.31 (d, J=23.1 Hz), 115.00 (d, J=21.2 Hz), 77.21, 77.05, 76.89, 56.81, 54.94, 31.61, 23.38. ¹⁹F NMR (564 MHz, CDCl₃) δ −102.34. HRMS (ESI) calcd for C₂₈H₂₅F₃N₄O₂S [M+H]⁺: 551.1917, found: 539.1717.

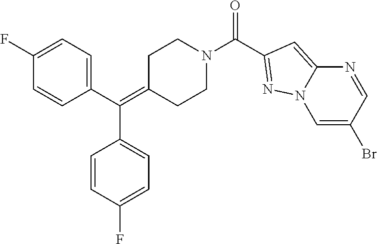 [SMILES: O=C(c1cc2ncc(Br)cn2n1)N1CCC(=C(c2ccc(F)cc2)c2ccc(F)cc2)CC1]

### (4-(Bis(4-fluorophenyl)methylene)piperidin-1-yl)(6-bromopyrazolo[1,5-a]pyrimidin-2-yl)methanone (XJ-2-35)

¹H NMR (800 MHz, DMSO-d₆) δ 9.60 (d, J=2.3, H), 8.69 (d, J=2.2 Hz, 1H), 7.19-7.15 (m, 4H), 7.16-7.10 (m 4, 4H), 7.00 (d, J=0.9 Hz, H), 3.75-3.69 (m, 4H), 2.36 (t, J=5.9 Hz, 2H), 2.29 (t, J=5.8 Hz, 2H). ¹³C NMR (200 MHz, DMSO-d₆) δ 162.29, 162.00, 160.84, 151.84, 151.77, 151.61, 146.64, 138.38, 136.64, 135.13, 134.96, 131.91 (4C), 115.76 (4C), 105.12, 98.31, 47.93, 43.56, 32.41, 31.30. ¹⁹F NMR (564 MHz, CDCl₃) δ −118.42. HRMS (ESI) calcd for C₂₅H₁₉BrF₂N₄O [M+H]⁺: 509.0768, found: 509.0770.

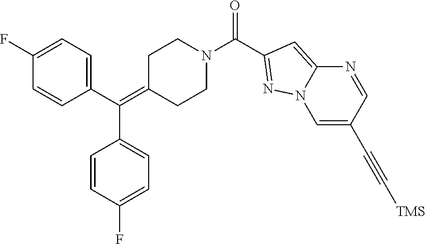 [SMILES: C[Si](C)(C)C#Cc1cnc2cc(C(=O)N3CCC(=C(c4ccc(F)cc4)c4ccc(F)cc4)CC3)nn2c1]

### (4-(Bis(4-fluorophenyl)methylene)piperidin-1-yl)(6-((trimethylsilyl)ethynyl)pyrazolo [1,5-a]pyrimidin-2-yl)methanone (XJ-2-37)

¹H NMR (800 MHz, CDCl₃) δ 8.69 (d, J=1.9 Hz, 1H), 8.52 (s, 1H), 7.11-6.90 (m, 9H), 3.93-3.71 (m, 4H), 2.49 (t, J=5.9 Hz, 2H), 2.41 (t, J=5.8 Hz, 2H), 0.28 (s, 9H). ¹³C NMR (200 MHz, CDCl₃) δ 162.79, 162.60 (d, J=13.2 Hz), 161.38 (d, J=13.0 Hz), 161.41, 161.35, 152.71, 152.35, 138.04, 138.02, 137.86, 137.84, 137.58, 136.32, 134.49, 131.50, 131.46, 115.60, 115.49, 115.38, 101.03, 99.72, 97.27, 48.52, 44.30, 32.70, 31.49, 0.00. ¹⁹F NMR (564 MHz, CDCl₃) δ −114.35. HRMS (ESI) calcd for C₃₀H₂₈F₂N₄OSi [M+H]⁺: 527.2065, found: 527.2070.

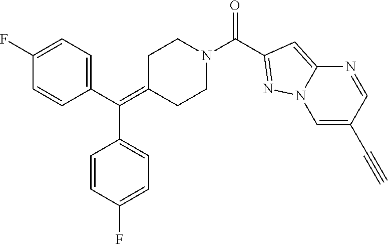 [SMILES: C#Cc1cnc2cc(C(=O)N3CCC(=C(c4ccc(F)cc4)c4ccc(F)cc4)CC3)nn2c1]

### (4-(Bis(4-fluorophenyl)methylene)piperidin-1-yl)(6-ethynylpyrazolo[1,5-a]pyrimidin-2-yl)methanone (XJ-2-39)

¹H NMR (600 MHz, CDCl₃) δ 8.73 (d, J=2.1 Hz, 1H), 8.51 (d, J=2.0 Hz, 1H), 7.14-6.86 (m, 9H), 3.83 (q, J=6.5 Hz, 4H), 3.31 (s, 1H), 2.48 (t, J=5.9 Hz, 2H), 2.41 (t, J=5.8 Hz, 2H). ¹³C NMR (150 MHz, CDCl₃) δ 162.49, 162.43, 162.34, 160.86, 160.80, 152.60, 151.85, 151.81, 147.06, 137.80, 137.72, 137.70, 137.54, 135.99, 134.15, 131.18, 131.13, 115.27, 115.15, 115.03, 106.02, 99.54, 99.53, 82.61, 82.47, 76.36, 76.33, 48.17, 43.98, 32.38, 31.16. ¹⁹F NMR (564 MHz, CDCl₃) δ −118.29. HRMS (ESI) calcd for C₂₇H₂₀F₂N₄O [M+H]⁺: 455.1665, found: 455.1669.

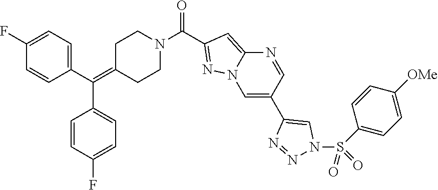 [SMILES: COc1ccc(S(=O)(=O)n2cc(-c3cnc4cc(C(=O)N5CCC(=C(c6ccc(F)cc6)c6ccc(F)cc6)CC5)nn4c3)nn2)cc1]

### (4-(Bis(4-fluorophenyl)methylene)piperidin-1-yl)(6-(1-((4-methoxyphenyl)sulfonyl)-1H-1,2,3-triazol-4-yl)pyrazolo[1,5-a]pyrimidin-2-yl)methanone (XJ-2-47)

¹H NMR (800 MHz, CDCl₃) δ 9.27 (dd, J=2.2, 0.9 Hz, 1H), 8.95 (d, J=2.2 Hz, 1H), 8.54 (s, 1H), 8.24-8.15 (m, 2H), 7.21-7.13 (m, 8H), 7.13-7.09 (m, 2H), 7.09-7.05 (m, 2H), 4.00 (s, 3H), 3.97 (t, J=5.9 Hz, 2H), 3.94 (t, J=5.9 Hz, 2H), 2.59 (t, J=5.9 Hz, 2H), 2.53 (t, J=5.8 Hz, 2H). ¹³C NMR (200 MHz, CDCl₃) δ 165.33, 162.03, 161.87, 161.80, 160.65, 160.58, 152.09, 147.49, 147.40, 140.36, 137.27, 137.25, 137.10, 137.08, 135.56, 133.73, 131.47, 131.07, 130.73, 130.70, 125.80, 118.83, 114.84, 114.83, 114.72, 114.62, 112.17, 98.89, 55.58, 47.78, 43.56, 31.96, 30.74. ¹⁹F NMR (564 MHz, CDCl₃) δ −118.44. HRMS (ESI) calcd for C₃₄H₂₇F₂N₇O₄S [M+H]⁺: 668.1880, found: 668.1884.

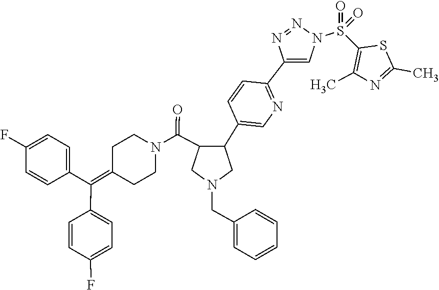

### (1-Benzyl-4-(6-(1-((2,4-dimethylthiazol-5-yl)sulfonyl)-1H-1,2,3-triazol-4-yl)pyridin-3-yl)pyrrolidin-3-yl)(4-(bis(4-fluorophenyl)methylene)piperidin-1-yl)methanone (XJ-2-65)

¹H NMR (800 MHz, CDCl₃) δ 9.35 (s, 1H), 8.86 (d, J=1.2 Hz, 1H), 8.33 (dd, J=9.0, 1.0 Hz, 1H), 7.84 (dd, J=8.9, 1.4 Hz, 1H), 7.37-7.31 (m, 4H), 7.229-7.26 (m, 1H), 7.04-6.90 (m, 8H), 4.19 (ddd, J=7.7, 5.7, 4.0 Hz, 1H), 3.79-3.66 (m, 3H), 3.58 (ddd, J=12.5, 7.0, 4.9 Hz, 1H), 3.40-3.36 (m, 1H), 3.35-3.29 (m, 2H), 3.22 (td, J=8.4, 5.7 Hz, 1H), 3.02-2.90 (m, 2H), 2.70 (s, 3H), 2.69 (s, 3H), 2.53 (t, J=8.9 Hz, 1H), 2.35-2.28 (m, 2H), 2.26-2.18 (m, 2H). ¹³C NMR (200 MHz, CDCl₃) δ 170.31, 170.12, 162.35, 162.31, 161.73, 161.13, 161.08, 158.17, 138.24, 137.47, 137.45, 137.31, 137.30, 136.82, 136.34, 133.37, 133.15, 132.95, 132.91, 131.11, 131.07, 131.04, 128.58, 128.56, 127.61, 127.43, 124.02, 120.05, 115.31, 115.27, 115.20, 60.20, 59.66, 57.98, 50.32, 46.64, 43.74, 43.42, 32.24, 31.13, 19.55, 16.59. ¹⁹F NMR (564 MHz, CDCl₃) δ −118.23. HRMS (ESI) calcd for C₄₂H₃₉F₂N₇O₃S₂ [M+H]⁺: 792.2602, found: 792.2589.

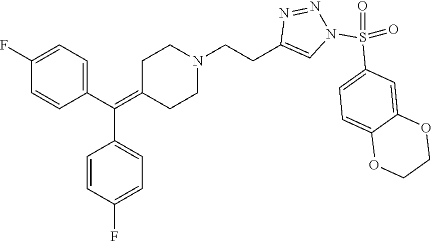 [SMILES: O=S(=O)(c1ccc2c(c1)OCCO2)n1cc(CCN2CCC(=C(c3ccc(F)cc3)c3ccc(F)cc3)CC2)nn1]

### 4-(Bis(4-fluorophenyl)methylene)-1-(2-(1-((2,3-dihydrobenzo[b][1,4]dioxin-6-yl)sulfonyl-)-1H-1,2,3-triazol-4-yl)ethyl)piperidine (XJ-2-77)

¹H NMR (800 MHz, CDCl₃) δ 7.94 (s, 1H), 7.62-7.56 (m, 2H), 7.08-7.03 (m, 4H), 7.01-6.95 (m, 5H), 4.36-4.31 (m, 2H), 4.30-4.25 (m, 2H), 2.93 (t, J=7.5 Hz, 2H), 2.68 (t, J=7.6 Hz, 2H), 2.55-2.49 (m, 4H), 2.37 (t, J=5.7 Hz, 4H). ¹³C NMR (201 MHz, CDCl₃) δ 161.68, 160.46, 149.55, 145.59, 143.53, 137.64, 137.62, 135.44, 133.60, 130.85, 130.81, 127.42, 122.17, 120.50, 117.98, 117.81, 114.57, 114.47, 64.25, 63.57, 56.43, 54.46, 31.13, 22.94. ¹⁹F NMR (564 MHz, CDCl₃) δ −119.13. HRMS (ESI) calcd for C₃₀H₂₈F₂N₄O₄S [M+H]⁺: 579.1872, found: 579.1874.

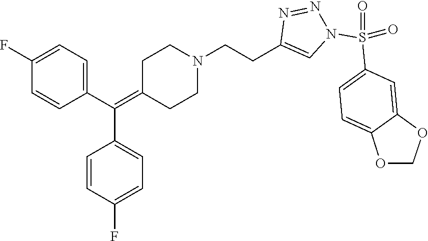 [SMILES: O=S(=O)(c1ccc2c(c1)OCO2)n1cc(CCN2CCC(=C(c3ccc(F)cc3)c3ccc(F)cc3)CC2)nn1]

### 1-(2-(1-(Benzo[d][1,3]dioxol-5-ylsulfonyl)-(H-1,2,3-triazol-4-yl)ethyl)-4-(bis(4-fluorophenyl)methylene)piperidine (XJ-2-87)

¹H NMR (800 MHz, CDCl₃) δ 7.95 (s, 1H), 7.71 (dd, J=8.3, 2.0 Hz, 1H), 7.45 (d, J=2.0 Hz, 1H), 7.07-7.03 (m, 4H), 7.00-6.94 (m, 6.92 (d, J=8.3 Hz, 1H), 6.10 (s, 2H), 2.93 (t, J=7.5 Hz, 2H), 2.68 (t, J=7.5 Hz, 2H), 2.53 (t, J=5.6 Hz, 4H), 2.37 (t, J=5.6 Hz, 4H). ¹³C NMR (201 MHz, CDCl₃) δ 161.60, 160.38, 137.62, 15.44, 133.60, 145.74, 137.69, 137.67, 135.50, 133.51, 130.87, 130.83, 128.24, 124.97, 120.67, 114.53, 114.42, 108.36, 107.69, 102.65, 56.34, 54.39, 31.09, 22.87. ¹⁹F NMR (564 MHz, CDCl₃) δ −119.00. HRMS (ESI) calcd for C₂₉H₂₆F₂N₄O₄S [M+H]⁺: 565.1713, found: 565.1715.

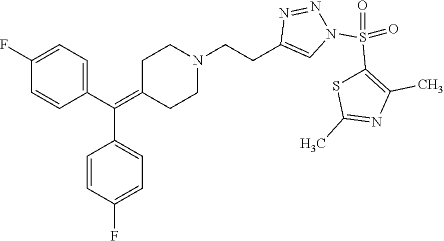 [SMILES: Cc1nc(C)c(S(=O)(=O)n2cc(CCN3CCC(=C(c4ccc(F)cc4)c4ccc(F)cc4)CC3)nn2)s1]

### 5-((4-(2-(4-(Bis(4-fluorophenyl)methylene)piperidin-1-yl)ethyl)-1H-1,2,3-triazol-1-yl)sulfonyl)-2,4-dimethylthiazole (XJ-2-105)

¹H NMR (800 MHz, CDCl₃) δ 8.08 (s, 1H), 7.18-7.12 (m, 4H), 7.10-7.04 (m, 4H), 3.07 (t, J=7.4 Hz, 2H), 2.83 (s, 3H), 2.82-2.80 (m, 2H), 2.80 (s, 3H), 2.65 (t, J=5.7 Hz, 4H), 2.48 (t, J=5.7 Hz, 4H). ¹³C NMR (200 MHz, CDCl₃) δ 173.01, 161.71, 160.48, 145.69, 137.55, 137.54, 130.82, 130.78, 124.42, 120.47, 114.66, 114.61, 114.55, 114.50, 56.30, 54.46, 31.03, 22.84, 19.35, 16.53. ¹⁹F NMR (564 MHz, CDCl₃) δ −119.02. HRMS (ESI) calcd for C₂₇H₂₇F₂N₅O₂S₂ [M+H]⁺: 565.1652, found: 556.1643.

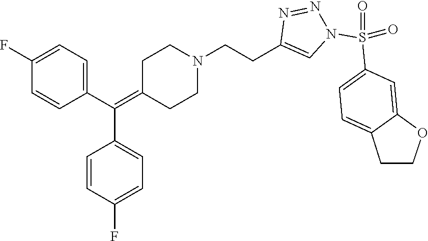 [SMILES: O=S(=O)(c1ccc2c(c1)OCC2)n1cc(CCN2CCC(=C(c3ccc(F)cc3)c3ccc(F)cc3)CC2)nn1]

### 4-(Bis(4-fluorophenyl)methylene)-1-(2-(1-((2,3-dihydrobenzofuran-6-yl)sulfonyl)-1H-1,2,3-triazol-4-yl)ethyl)piperidine (XJ-2-111)

¹H NMR (800 MHz, CDCl₃) δ 7.95 (s, 1H), 7.90 (dt, J=2.4, 1.3 Hz, 1H), 7.86 (dd, J=8.6, 2.2 Hz, 1H), 7.06-7.02 (m, 4H), 6.97-6.93 (m, 4H), 6.84 (d, J=8.6 Hz, 1H), 4.67 (t, J=8.9 Hz, 2H), 3.24 (t, J=8.9 Hz, 2H), 2.91 (t, J=7.6 Hz, 2H), 2.67 (dd, J=8.2, 6.9 Hz, 2H), 2.51 (t, J=5.6 Hz, 4H), 2.36-2.33 (m, 4H). ¹³C NMR (201 MHz, CDCl₃) δ 165.96, 161.64, 160.42, 145.59, 137.67, 137.66, 135.54, 133.51, 130.86, 130.82, 130.43, 129.08, 126.42, 125.63, 120.41, 114.55, 114.44, 109.75, 72.45, 56.45, 54.44, 31.14, 28.25, 22.94. ¹⁹F NMR (564 MHz, CDCl₃) δ −119.09. HRMS (ESI) calcd for C₂₉H₂₆F₂N₄O₄S [M+H]⁺: 563.19235, found: 563.1926.

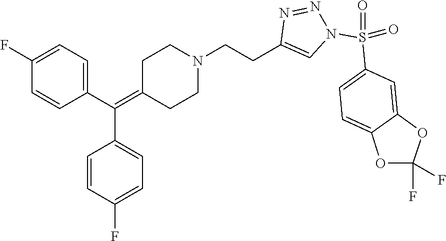 [SMILES: O=S(=O)(c1ccc2c(c1)OC(F)(F)O2)n1cc(CCN2CCC(=C(c3ccc(F)cc3)c3ccc(F)cc3)CC2)nn1]

### 4-(Bis(4-fluorophenyl)methylene)-1-(2-(1-((2,2-difluorobenzo[d][1,3]dioxol-5-yl)sulfonyl)-1H-1,2,3-triazol-4-yl)ethyl)piperidine (XJ-2-115)

¹H NMR (800 MHz, CDCl₃) δ 7.99 (s, 1H), 7.97 (dd, J=8.5, 1.9 Hz, 1H), 7.81 (d, J=1.9 Hz, 1H), 7.28-7.22 (m, 1H), 7.07-7.02 (m, 4H), 7.00-6.91 (m, 4H), 2.95 (t, J=7.5 Hz, 2H), 2.70 (t, J=7.5 Hz, 2H), 2.54 (t, J=5.7 Hz, 4H), 2.37 (t, J=5.7 Hz, 4H). ¹³C NMR (200 MHz, CDCl₃) δ 161.70, 160.48, 148.26, 145.89, 143.64, 137.57, 137.55, 135.58-132.28 (t, J=260.2 Hz). 131.44, 131.25, 130.82, 130.78, 126.04, 120.65, 114.59, 114.49, 109.97, 109.73, 56.28, 54.45, 31.05, 22.83. ¹⁹F NMR (564 MHz, CDCl₃) δ −47.73, −114.60. HRMS (ESI) calcd for C₂₉H₂₄F₄N₄O₄S [M+H]⁺: 601.1530, found: 601.1532.

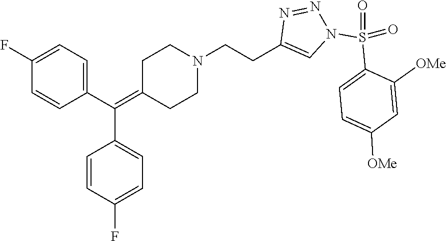 [SMILES: COc1ccc(S(=O)(=O)n2cc(CCN3CCC(=C(c4ccc(F)cc4)c4ccc(F)cc4)CC3)nn2)c(OC)c1]

### 4-(Bis(4-fluorophenyl)methylene)-1-(2-(1-((2,4-dimethoxyphenyl)sulfonyl)-1H-1,2,3-triazol-4-yl)ethyl)piperidine (XJ-2-139)

¹H NMR (800 MHz, CDCl₃) δ 8.15 (d, J=9.1 Hz, 1H), 8.09 (s, 1H), 7.10-7.06 (m, 4H), 7.03-6.96 (m, 4H), 6.66 (dd, J=9.0, 2.2 Hz, 1H), 6.44 (d, J=2.3 Hz, 1H), 3.90 (s, 5H), 3.79 (s, 5H), 2.99 (t, J=7.5 Hz, 2H), 2.74-2.72 (m, 2H), 2.57 (t, J=5.6 Hz, 4H), 2.45-2.37 (m, 4H). ¹³C NMR (200 MHz, CDCl₃) δ 166.90, 161.67, 160.45, 159.05, 144.63, 137.66, 137.64, 135.55, 133.55, 133.44, 130.85, 130.81, 121.83, 115.30, 114.57, 114.46, 104.99, 99.11, 56.79, 55.74, 55.55, 54.53, 31.21, 22.99. ¹⁹F NMR (564 MHz, CDCl₃) δ −118.99. HRMS (ESI) calcd for C₂₉H₂₄F₄N₄O₄S [M+H]⁺: 601.1530, found: 601.1532.

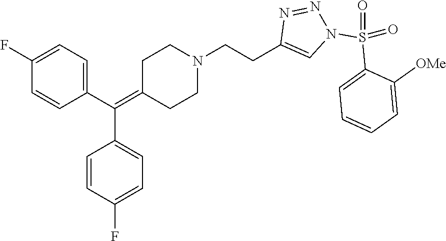 [SMILES: COc1ccccc1S(=O)(=O)n1cc(CCN2CCC(=C(c3ccc(F)cc3)c3ccc(F)cc3)CC2)nn1]

### 4-(Bis(4-fluorophenyl)methylene)-1-(2-(1-((2-methoxyphenyl)sulfonyl)-1H-1,2,3-triazol-4-yl)ethyl)piperidine (XJ-2-141)

¹H NMR (800 MHz, CDCl₃) δ 8.18 (dq, J=7.9, 1.9 Hz, 1H), 8.10 (s, 1H), 7.65 (ddt, J=8.7, 7.3, 1.5 Hz, 1H), 7.18-7.12 (m, 1H), 7.09-7.02 (m, 4H), 7.01-6.92 (m, 4H), 3.79 (s, 3H), 2.97 (t, J=7.5 Hz, 2H), 2.71 (t, J=7.5 Hz, 2H), 2.54 (t, J=5.6 Hz, 4H), 2.37 (t, J=5.6 Hz, 4H). ¹³C NMR (200 MHz, CDCl₃) δ 161.67, 160.45, 157.25, 144.73, 137.66, 137.64, 137.24, 135.51, 133.58, 131.21, 130.85, 130.81, 123.51, 122.14, 120.51, 114.57, 114.46, 112.29, 56.73, 55.78, 54.52, 31.20, 22.95. ¹⁹F NMR (564 MHz, CDCl₃) δ −119.06. HRMS (ESI) calcd for C₂₉H₂₈F₂N₄O₃S [M+H]⁺: 551.1927, found: 551.1929.

### Example 12

### Proteomic Assays with Inhibitors from Example 11

### Gel-Based Chemical Proteomic Assay (In Situ ABPP Analysis):

HEK293T cells were incubated in 10 cm petri dishes with DMEM medium supplemented with 10% fetal bovine serum and 1% L-glutamine until the confluency reached about 90%. Aspirate the medium and then add serum-free-medium (SFM) with vehicle only (DMSO) or SuTEx compounds in DMSO, which afforded final concentrations of 0.1% DMSO. After incubation at 37° C. for 1 hrs in CO₂ incubator, the medium was aspirated and 25 μM of probe TH211 in SFM was added. After incubation at 37° C. for 2 hrs, the medium was aspirated and the cells were washed off in cold PBS. Centrifuge at 400×g for 5 min to pellet and resuspend pellet again in PBS for 2× total washes. The cell pellets were resuspended and lysed by sonication (1 sec pulse, 20% amplitude, 3 times) in PBS in the presence of EDTA-free protease inhibitor cocktail tablet (Pierce Biotechnology, Waltham, Massachusetts, United States of America). The cell lysates were subject to ultracentrifugation (100,000×g, 45 min at 4° C.) to separate the cytosolic fraction in the supernatant and the membrane fraction as a pellet. Protein concentrations in soluble fraction were measured by the Bio-Rad DC protein assay (Bio-Rad Laboratories, Hercules, California, United States of America). Proteome aliquots (2 mg/mL, 50 μL) were conjugated with fluorophore which was accomplished by copper-catalyzed azide-alkyne cycloaddition (CuAAC) with rhodamine-azide (TAMRA-azide, 1.25 mM, 1 μL, final concentration of 25 μM) in the presence of tris(2-carboxyethyl)phosphine (TCEP, 50 mM fresh in water, 1 μL, final concentration of 1 mM), tris[(1-benzyl-1H-1,2,3-triazol-4-yl)methyl]amine (TBTA, 1.7 mM in 4:1 t-butanol/DMSO, 3 μL, final concentration of 100 μM) and CuSO₄ (50 mM, 1 μL, final concentration of 1 mM). After 1 hr incubation at room temperature, reactions were quenched by adding 4×SDS-PAGE loading buffer and beta-mercaptoethanol (17 μL) and samples were resolved by SDS-PAGE followed by in-gel fluorescence scanning.

### SILAC Sample Preparation for Competitive Chemical Proteomic Assay:

The light and heavy proteomes (2.3 mg/mL, 432 μL) prepared from HEK293T cells that were pretreated in situ with vehicle (DMSO) or the inhibitor (2 μM), respectively, at 37° C. for 1 hr followed by probe TH211 treatment (50 μM) for 2 h, were directly were subjected to click reaction with the desthiobiotin-PEG3-azide (10 mM in DMSO, 10 μL, final concentration 200 μM) in the presence of TCEP (50 mM, 10 μL), TBTA (1.7 mM, 33 μL) and CuSO₄ (50 mM, 10 μL) at room temperature for 1 hr. The light and the heavy samples were mixed in the chloroform-methanol extraction step. The subsequent steps including reduction with dithiothreitol, alkylation with iodoacetamide, digestion with Trypsin/Lys-C, enrichment with avidin beads were conducted as previously described²⁶.

### Gel-Based Chemical Proteomic Assay (In Vitro ABPP Analysis):

HEK293T cells at 30-50% confluency were transfected with 2.6 μg of Flag-PFKL, Flag-PFKM or Flag-PFKP plasmid DNA a serum-free media for 48 hrs. After 48 hrs, the media was aspirated and cells washed with cold PBS and harvested. Cells were spun at 400×g for 5 min at 4° C. and supernatant was removed. Pellets were resuspended in 1 mL of cold DPBS and spun at 400×g for 5 min at 4° C. and supernatant was removed. The cells were resuspended and lysed by sonication (1 sec pulse, 20% amplitude, 3 times) in PBS in the presence of EDTA-free protease inhibitor cocktail tablet (Pierce Biotechnology, Waltham, Massachusetts, United States of America) by sonication and fractionated (100,000×g, 45 min, 4° C.). The protein concentration of the lysates in soluble fractions was determined on the Clariostar plate reader (BMG Labtech, Ortenberg, Germany) using the Bio-Rad DC protein assay (Bio-Rad Laboratories, Hercules, California, United States of America). The soluble fraction was diluted to 1 mg/mL in PBS in the presence of EDTA-free protease inhibitor cocktail tablet (Pierce Biotechnology, Waltham, Massachusetts, United States of America) and 48 μL was used for analysis. DMSO or compound was added and the tube gently flicked tube to mix. The tube was incubated for 1 h at 37° C. in an incubator. Then 1 μL of probe TH211 was added (1.25 mM of TH211 stock) to a final concentration of 25 μM and the tube again incubated for 1 h at 37° C. in an incubator. The probe-modified proteomes were conjugated to Rhodamine-azide (1 μl of 1.25 mM stock in DMSO) using TCEP (1 μl of fresh 50 mM stock in water), TBTA ligand (3 μl of a 1.7 mM 4:1 t-butanol/DMSO stock,) and CuSO₄ (1 μl of 50 mM stock) and incubated for 1 hr at room temperature. The reaction was quenched with 17 μL of 4×SDS-PAGE loading buffer+βME and vortexed to mix. The samples were analyzed by SDS-PAGE (30 μL) and imaged by in-gel fluorescence scanning.

### Discussion:

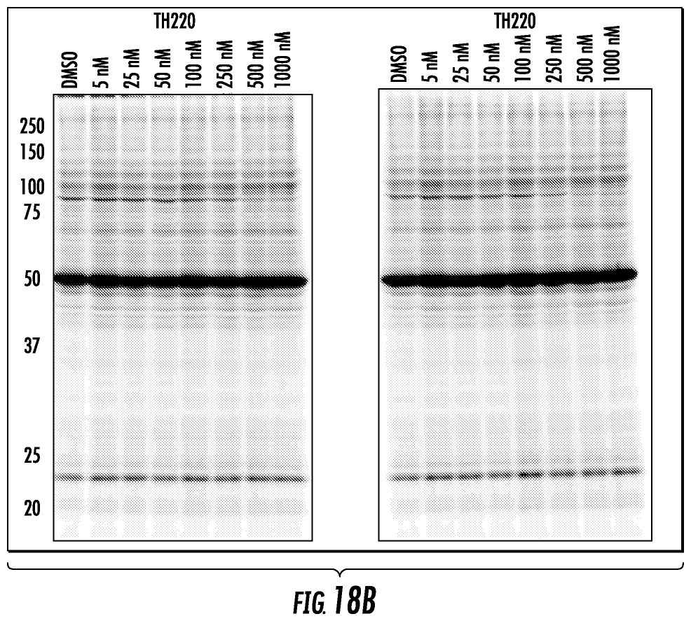

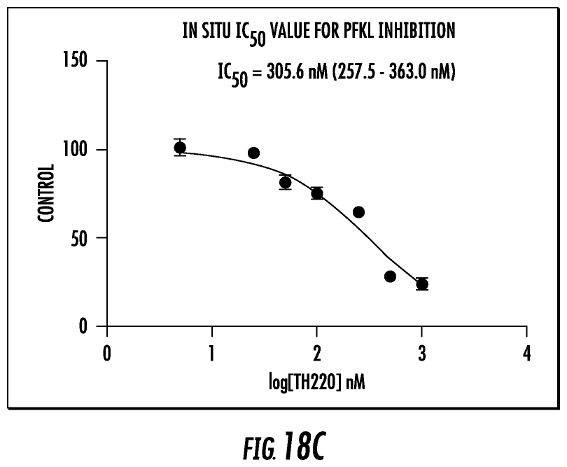

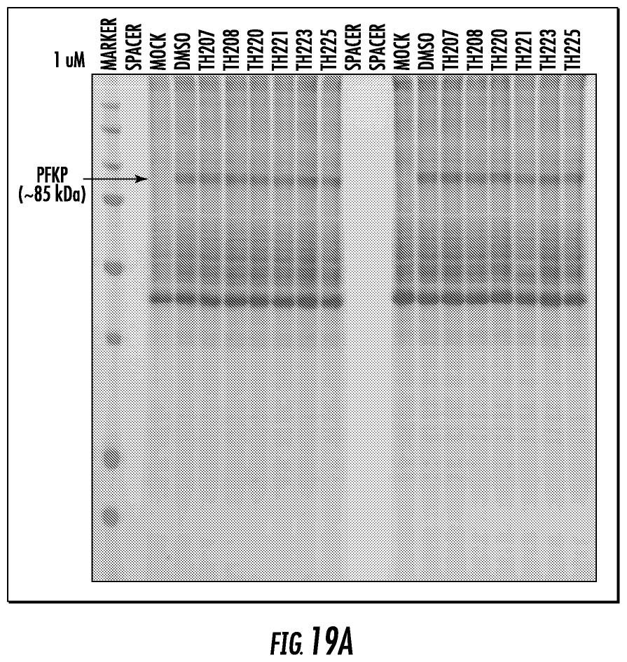

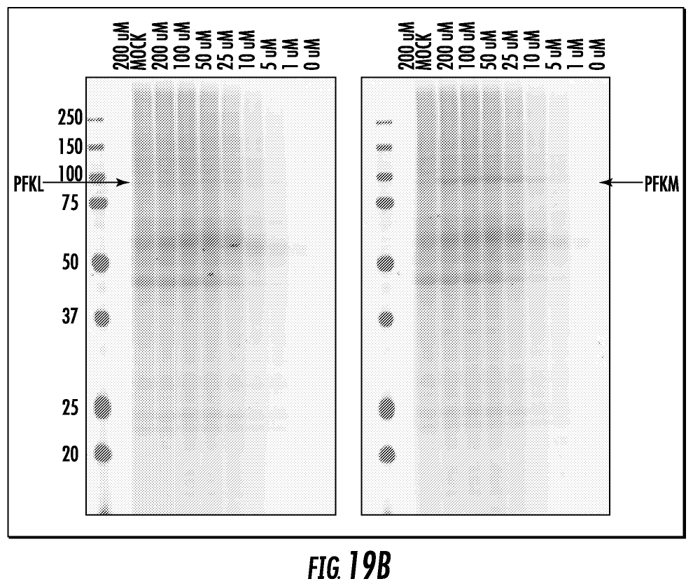

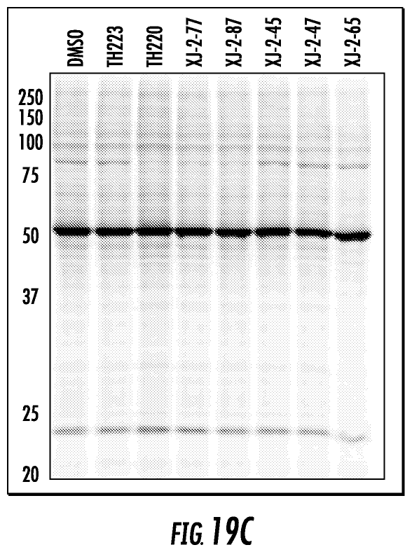

Some SuTEx analogues with RF001 fragment were synthesized and characterized. The in situ competition assay was then conducted, during which HEK293T cells were treated with 1 μM of a SuTEx analogue for 1 h, followed by treatment with 25 μM of TH211 as the probe for 2 h. Some analogues displayed good probinding activity against the protein at ˜80 molecular weight. See FIG. 18A. The analogues with electron-donating groups like methyl or methoxy on the para position of phenyl performed better than those with electron-withdrawing groups. Under the same condition, the IC₅o of TH220 was determined as 305.6 nM. See FIGS. 18B and 18C. The identity of the bound protein was studied by LC-MS/MS. The assay results indicated PFKL as the potential target and that binding sites include Y674 and K677, since the ratio trends are the same as that found in the in situ competition assay. See Tables 4-6, below. To study selective targeting of PFKL by the series of SuTEx analogues, Fag-PFKL, Flag-PFKM, and Flag-PFKP were overexpressed in HEK293T cells and in vitro competition assays were performed for comparison. See FIGS. 19A and 19B. As shown in FIG. 19A, these analogues did not display significant competition of probe binding activity against PFKP. To optimize these SuTEx ligands, another series of derivatives were synthesized and evaluated with TH233 as a negative control and TH220 as positive control, respectively. As shown in FIG. 19C, XJ-2-77, XJ-2-87, XJ-2-115 and XJ-2-141 displayed good probinding activity at 1 μM. Compared to TH220, the probinding activity of XJ-2-103 with trifluoromethyl group at para position of the phenyl ring is not as good, which is consistent with the results shown in FIG. 18A.

TABLE 4
SILAC ratios (SR) from competitive chemical proteomic assay.

| Treatment | SR |
|---|---|
| P/NP | >20 |
| P/P | 0.95 |
| TH207 | 3.41 |
| TH208 | 1.04 |
| TH220 | 8.41 |
| TH221 | 2.48 |
| TH223 | 0.95 |
| TH224 | 0.89 |

TABLE 5
Normalized SILAC ration (SR) for HEK293T soluble
fraction proteins, tyrosine (Y) binding sites
identified by LC-MS/MS, TH207 inhibitor.

| UniProt | | | |
|---|---|---|---|
| ID | residue | SR | protein |
| Q9ULV4.1 | 301 | 20 | Coronin-1C |
| Q99541.2 | 190 | 3.5 | Perilipin-2 |
| Q9H773.1 | 129 | 3 | dCTP pyrophosphatase 1 |
| P17858.6 | 674 | 2.7 | PFKL |
| P24666.3 | 132 | 1.9 | Phosphotyrosine protein phosphatase |
| P48739.2 | 154 | 1.8 | Phosphatidylinositol transfer protein beta |
| | | | isoform |
| Q9NSE4.2 | 260 | 1.8 | Isoleucine tRNA ligase |
| P12268.2 | 509 | 1.6 | Inosine-5′-monophosphate dehydrogenase |
| | | | 2 |
| P07900.5 | 61 | 1.5 | Heat shock protein HSP 90-beta |
| P99999.2 | 49 | 1.5 | Cytochrome c |

TABLE 6
Normalized SILAC ration (SR) for HEK293T soluble
fraction proteins, lysine (K) binding sites
identified by LC-MS/MS, TH207 inhibitor.

| UniProt ID | residue | SR | protein |
|---|---|---|---|
| Q9UNH7.1 | 204 | 6.9 | Sorting nexin-6 |
| P62258.1 | 215 | 3.7 | 14-3-3 protein epsilon |
| P17858.6 | 677 | 2.7 | PFKL |
| P48739.2 | 155 | 1.8 | Phosphatidylinositol transfer protein |
| | | | beta isoform |
| P99999.2 | 54 | 1.8 | Cytochrome c |
| P06733.2 | 80 | 1.6 | Alpha-enolase |
| P99999.2 | 40 | 1.5 | Cytochrome c |
| P99999.2 | 28 | 1.4 | Cytochrome c |
| Q98WD1 | 235 | 1.3 | Major capsid protein VP1 |
| P25787.2 | 92 | 1.2 | Proteasome subunit alpha type-2 |

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
1. Manning, G.; Whyte, D. B.; Martinez, R.; Hunter, T.; Sudarsanam, S., The protein kinase complement of the human genome. Science 2002, 298, 1912-34.
2. Hunter, T., Protein kinases and phosphatases: The Yin and Yang of protein phosphorylation and signaling. Cell 1995, 80, 225-236.
3. Pawson, T.; Scott, J. D., Protein phosphorylation in signaling—50 years and counting. Trends Biochem Sci 2005, 30, 286-90.
4. Cohen, P., The regulation of protein function by multisite phosphorylation—a 25 year update. Trends Biochem Sci 2000, 25, 596-601.
5. Johnson, L. N.; Barford, D., The effects of phosphorylation on the structure and function of proteins. Annu Rev Biophys Biomol Struct 1993, 22, 199-232.
6. Roskoski, R., Jr., Properties of FDA-approved small molecule protein kinase inhibitors: A 2020 update. Pharmacol Res 2020, 152, 104609.
7. Patricelli, M. P.; Nomanbhoy, T. K.; Wu, J.; Brown, H.; Zhou, D.; Zhang, J.; Jagannathan, S.; Aban, A.; Okerberg, E.; Herring, C.; Nordin, B.; Weissig, H.; Yang, Q.; Lee, J. D.; Gray, N. S.; Kozarich, J. W., In situ kinase profiling reveals functionally relevant properties of native kinases. Chem Biol 2011, 18, 699-710.
8. Patricelli, M. P.; Szardenings, A. K.; Liyanage, M.; Nomanbhoy, T. K.; Wu, M.; Weissig, H.; Aban, A.; Chun, D.; Tanner, S.; Kozarich, J. W., Functional interrogation of the kinome using nucleotide acyl phosphates. Biochemistry 2007, 46, 350-8.
9. Shin, M.; Franks, C. E.; Hsu, K.-L., Isoform-selective activity-based profiling of ERK signaling. Chem Sci 2018, 9, 2419-2431.
10. Bantscheff, M.; Eberhard, D.; Abraham, Y.; Bastuck, S.; Boesche, M.; Hobson, S.; Mathieson, T.; Perrin, J.; Raida, M.; Rau, C.; Reader, V.; Sweetman, G.; Bauer, A.; Bouwmeester, T.; Hopf, C.; Kruse, U.; Neubauer, G.; Ramsden, N.; Rick, J.; Kuster, B.; Drewes, G., Quantitative chemical proteomics reveals mechanisms of action of clinical ABL kinase inhibitors. Nat Biotechnol 2007, 25, 1035-44.
11. Klaeger, S.; Heinzlmeir, S.; Wilhelm, M.; Polzer, H.; Vick, B.; Koenig, P. A.; Reinecke, M.; Ruprecht, B.; Petzoldt, S.; Meng, C.; Zecha, J.; Reiter, K.; Qiao, H.; Helm, D.; Koch, H.; Schoof, M.; Canevari, G.; Casale, E.; Depaolini, S. R.; Feuchtinger, A.; Wu, Z.; Schmidt, T.; Rueckert, L.; Becker, W.; Huenges, J.; Garz, A. K.; Gohlke, B. O.; Zolg, D. P.; Kayser, G.; Vooder, T.; Preissner, R.; Hahne, H.; Tonisson, N.; Kramer, K.; Gotze, K.; Bassermann, F.; Schlegl, J.; Ehrlich, H. C.; Aiche, S.; Walch, A.; Greif, P. A.; Schneider, S.; Felder, E. R.; Ruland, J.; Medard, G.; Jeremias, I.; Spiekermann, K.; Kuster, B., The target landscape of clinical kinase drugs. Science 2017, 358, eaan4368.
12. Shi, H.; Zhang, C. J.; Chen, G. Y.; Yao, S. Q., Cell-based proteome profiling of potential dasatinib targets by use of affinity-based probes. J Am Chem Soc 2012, 134, 3001-14.
13. Ranjitkar, P.; Perera, B. G.; Swaney, D. L.; Hari, S. B.; Larson, E. T.; Krishnamurty, R.; Merritt, E. A.; Villen, J.; Maly, D. J., Affinity-based probes based on type II kinase inhibitors. J Am Chem Soc 2012, 134, 19017-25.
14. Zhao, Q.; Ouyang, X.; Wan, X.; Gajiwala, K. S.; Kath, J. C.; Jones, L. H.; Burlingame, A. L.; Taunton, J., Broad-Spectrum Kinase Profiling in Live Cells with Lysine-Targeted Sulfonyl Fluoride Probes. J Am Chem Soc 2017, 139, 680-685.
15. Dong, J.; Krasnova, L.; Finn, M. G.; Sharpless, K. B., Sulfur(VI) fluoride exchange (SuFEx): another good reaction for click chemistry. Angew Chem Int Ed Engl 2014, 53, 9430-48.
16. Liu, Z.; Li, J.; Li, S.; Li, G.; Sharpless, K. B.; Wu, P., SuFEx Click Chemistry Enabled Late-Stage Drug Functionalization. J Am Chem Soc 2018, 140, 2919-2925.
17. Zheng, Q.; Woehl, J. L.; Kitamura, S.; Santos-Martins, D.; Smedley, C. J.; Li, G.; Forli, S.; Moses, J. E.; Wolan, D. W.; Sharpless, K. B., SuFEx-enabled, agnostic discovery of covalent inhibitors of human neutrophil elastase. Proc Natl Acad Sci USA 2019, 116, 18808-18814.
18. Chen, W.; Dong, J.; Plate, L.; Mortenson, D. E.; Brighty, G. J.; Li, S.; Liu, Y.; Galmozzi, A.; Lee, P. S.; Hulce, J. J.; Cravatt, B. F.; Saez, E.; Powers, E. T.; Wilson, I. A.; Sharpless, K. B.; Kelly, J. W., Arylfluorosulfates Inactivate Intracellular Lipid Binding Protein(s) through Chemoselective SuFEx Reaction with a Binding Site Tyr Residue. J Am Chem Soc 2016, 138, 7353-64.
19. Mortenson, D. E.; Brighty, G. J.; Plate, L.; Bare, G.; Chen, W.; Li, S.; Wang, H.; Cravatt, B. F.; Forli, S.; Powers, E. T.; Sharpless, K. B.; Wilson, I. A.; Kelly, J. W., “Inverse Drug Discovery” Strategy To Identify Proteins That Are Targeted by Latent Electrophiles As Exemplified by Aryl Fluorosulfates. J Am Chem Soc 2018, 140, 200-210.
20. Brighty, G. J.; Botham, R. C.; Li, S.; Nelson, L.; Mortenson, D. E.; Li, G.; Morisseau, C.; Wang, H.; Hammock, B. D.; Sharpless, K. B.; Kelly, J. W., Using sulfuramidimidoyl fluorides that undergo sulfur(VI) fluoride exchange for inverse drug discovery. Nat Chem 2020, 12, 906-913.
21. Browne, C. M.; Jiang, B.; Ficarro, S. B.; Doctor, Z. M.; Johnson, J. L.; Card, J. D.; Sivakumaren, S. C.; Alexander, W. M.; Yaron, T. M.; Murphy, C. J.; Kwiatkowski, N. P.; Zhang, T.; Cantley, L. C.; Gray, N. S.; Marto, J. A., A Chemoproteomic Strategy for Direct and Proteome-Wide Covalent Inhibitor Target-Site Identification. J Am Chem Soc 2019, 141, 191-203.
22. Ficarro, S. B.; Browne, C. M.; Card, J. D.; Alexander, W. M.; Zhang, T.; Park, E.; McNally, R.; Dhe-Paganon, S.; Seo, H. S.; Lamberto, I.; Eck, M. J.; Buhrlage, S. J.; Gray, N. S.; Marto, J. A., Leveraging Gas-Phase Fragmentation Pathways for Improved Identification and Selective Detection of Targets Modified by Covalent Probes. Anal Chem 2016, 88, 12248-12254.
23. Borne, A. L.; Brulet, J. W.; Yuan, K.; Hsu, K. L., Development and biological applications of sulfur-triazole exchange (SuTEx) chemistry. RSC Chem Biol 2021, 2, 322-337.
24. Brulet, J. W.; Borne, A. L.; Yuan, K.; Libby, A. H.; Hsu, K. L., Liganding Functional Tyrosine Sites on Proteins Using Sulfur-Triazole Exchange Chemistry. J Am Chem Soc 2020, 142, 8270-8280.
25. Hahm, H. S.; Toroitich, E. K.; Borne, A. L.; Brulet, J. W.; Libby, A. H.; Yuan, K.; Ware, T. B.; McCloud, R. L.; Ciancone, A. M.; Hsu, K. L., Global targeting of functional tyrosines using sulfur-triazole exchange chemistry. Nat Chem Biol 2020, 16, 150-159.
26. Huang, T.; Hosseinibarkooie, S.; Borne, A. L.; Granade, M. E.; Brulet, J. W.; Harris, T. E.; Ferris, H. A.; Hsu, K.-L., Chemoproteomic profiling of kinases in live cells using electrophilic sulfonyl triazole probes. Chem Sci 2021, 12, 3295-3307.
27. Rostovtsev, V. V.; Green, L. G.; Fokin, V. V.; Sharpless, K. B., A Stepwise Huisgen Cycloaddition Process: Copper(I)-Catalyzed Regioselective “Ligation” of Azides and Terminal Alkynes. Angew Chem Int Ed Engl 2002, 41, 2596-2599.
28. Tweeten, K. A.; Tweeten, T. N., Reversed-phase chromatography of proteins on resin-based wide-pore packings. J Chromatogr 1986, 359, 111-119.
29. Lloyd, L. L., Rigid macroporous copolymers as stationary phases in high-performance liquid chromatography. J Chromatogr 1991, 544, 201-217.
30. Masque, N.; Galia, M.; Marcé, R. M.; Borrull, F., Chemically modified polymeric resin used as sorbent in a solid-phase extraction process to determine phenolic compounds in water. J Chromatogr 1997, 771, 55-61.
31. Olsen, J. V.; Macek, B.; Lange, O.; Makarov, A.; Horning, S.; Mann, M., Higher-energy C-trap dissociation for peptide modification analysis. Nat Methods 2007, 4, 709-12.
32. Syka, J. E.; Coon, J. J.; Schroeder, M. J.; Shabanowitz, J.; Hunt, D. F., Peptide and protein sequence analysis by electron transfer dissociation mass spectrometry. Proc Natl Acad Sci USA 2004, 101, 9528-33.
33. Chi, A.; Huttenhower, C.; Geer, L. Y.; Coon, J. J.; Syka, J. E.; Bai, D. L.; Shabanowitz, J.; Burke, D. J.; Troyanskaya, O. G.; Hunt, D. F., Analysis of phosphorylation sites on proteins from Saccharomyces cerevisiae by electron transfer dissociation (ETD) mass spectrometry. Proc Natl Acad Sci USA 2007, 104, 2193-8.
34. Ahmadi, S.; Winter, D., Identification of Poly(ethylene glycol) and Poly(ethylene glycol)-Based Detergents Using Peptide Search Engines. Anal Chem 2018, 90, 6594-6600.
35. Rappsilber, J.; Ishihama, Y.; Mann, M., Stop and go extraction tips for matrix-assisted laser desorption/ionization, nanoelectrospray, and LC/MS sample pretreatment in proteomics. Anal Chem 2003, 75, 663-70.
36. Waas, M.; Pereckas, M.; Jones Lipinski, R. A.; Ashwood, C.; Gundry, R. L., SP2: Rapid and Automatable Contaminant Removal from Peptide Samples for Proteomic Analyses. J Proteome Res 2019, 18, 1644-1656.
37. Alpert, A. J., Hydrophilic-interaction chromatography for the separation of peptides, nucleic acids and other polar compounds. J Chromatogr 1990, 499, 177-196.
38. Swaney, D. L.; Wenger, C. D.; Coon, J. J., Value of using multiple proteases for large-scale mass spectrometry-based proteomics. J Proteome Res 2010, 9, 1323-9.
39. Janes, M. R.; Zhang, J.; Li, L. S.; Hansen, R.; Peters, U.; Guo, X.; Chen, Y.; Babbar, A.; Firdaus, S. J.; Darjania, L.; Feng, J.; Chen, J. H.; Li, S.; Li, S.; Long, Y. O.; Thach, C.; Liu, Y.; Zarieh, A.; Ely, T.; Kucharski, J. M.; Kessler, L. V.; Wu, T.; Yu, K.; Wang, Y.; Yao, Y.; Deng, X.; Zarrinkar, P. P.; Brehmer, D.; Dhanak, D.; Lorenzi, M. V.; Hu-Lowe, D.; Patricelli, M. P.; Ren, P.; Liu, Y., Targeting KRAS Mutant Cancers with a Covalent G12C-Specific Inhibitor. Cell 2018, 172, 578-589 e17.
40. Honigberg, L. A.; Smith, A. M.; Sirisawad, M.; Verner, E.; Loury, D.; Chang, B.; Li, S.; Pan, Z.; Thamm, D. H.; Miller, R. A.; Buggy, J. J., The Bruton tyrosine kinase inhibitor PCI-32765 blocks B-cell activation and is efficacious in models of autoimmune disease and B-cell malignancy. Proc Natl Acad Sci USA 2010, 107, 13075-80.
41. Lanning, B. R.; Whitby, L. R.; Dix, M. M.; Douhan, J.; Gilbert, A. M.; Hett, E. C.; Johnson, T. O.; Joslyn, C.; Kath, J. C.; Niessen, S.; Roberts, L. R.; Schnute, M. E.; Wang, C.; Hulce, J. J.; Wei, B.; Whiteley, L. O.; Hayward, M. M.; Cravatt, B. F., A road map to evaluate the proteome-wide selectivity of covalent kinase inhibitors. Nat Chem Biol 2014, 10, 760-767.
42. Cohen, P.; Cross, D.; Janne, P. A., Kinase drug discovery 20 years after imatinib: progress and future directions. Nat Rev Drug Discov 2021, 20, 551-569.
43. Skoulidis, F.; Li, B. T.; Dy, G. K.; Price, T. J.; Falchook, G. S.; Wolf, J.; Italiano, A.; Schuler, M.; Borghaei, H.; Barlesi, F.; Kato, T.; Curioni-Fontecedro, A.; Sacher, A.; Spira, A.; Ramalingam, S. S.; Takahashi, T.; Besse, B.; Anderson, A.; Ang, A.; Tran, Q.; Mather, O.; Henary, H.; Ngarmchamnanrith, G.; Friberg, G.; Velcheti, V.; Govindan, R., Sotorasib for Lung Cancers with KRAS p.G12C Mutation. N Engl J Med 2021, 384, 2371-2381.
44. Wang, M. L.; Rule, S.; Martin, P.; Goy, A.; Auer, R.; Kahl, B. S.; Jurczak, W.; Advani, R. H.; Romaguera, J. E.; Williams, M. E.; Barrientos, J. C.; Chmielowska, E.; Radford, J.; Stilgenbauer, S.; Dreyling, M.; Jedrzejczak, W. W.; Johnson, P.; Spurgeon, S. E.; Li, L.; Zhang, L.; Newberry, K.; Ou, Z.; Cheng, N.; Fang, B.; McGreivy, J.; Clow, F.; Buggy, J. J.; Chang, B. Y.; Beaupre, D. M.; Kunkel, L. A.; Blum, K. A., Targeting BTK with ibrutinib in relapsed or refractory mantle-cell lymphoma. N Engl J Med 2013, 369, 507-16.
45. Ji, Y.; Leymarie, N.; Haeussler, D. J.; Bachschmid, M. M.; Costello, C. E.; Lin, C., Direct detection of S-palmitoylation by mass spectrometry. Anal Chem 2013, 85, 11952-9.
46. Riley, N. M.; Malaker, S. A.; Driessen, M. D.; Bertozzi, C. R., Optimal Dissociation Methods Differ for N- and O-Glycopeptides. J Proteome Res 2020, 19, 3286-3301.
47. Boutureira, O.; Bernardes, G. J., Advances in chemical protein modification. Chem Rev 2015, 115, 2174-95.
48. Conway, L. P.; Jadhav, A. M.; Homan, R. A.; Li, W.; Rubiano, J. S.; Hawkins, R.; Lawrence, R. M.; Parker, C. G., Evaluation of fully-functionalized diazirine tags for chemical proteomic applications. Chem Sci 2021, 12, 7839-7847.
49. Mahoney, K. E., Improving methods for isolation and identification of MHC-associated peptides. University of Virginia, Unpublished Thesis.
50. Lee, H. J.; Kim, H. J.; Liebler, D. C., Efficient Microscale Basic Reverse Phase Peptide Fractionation for Global and Targeted Proteomics. J Proteome Res 2016, 15, 2346-54.
51. Kim, H.; Dan, K.; Shin, H.; Lee, J.; Wang, J. I.; Han, D., An efficient method for high-pH peptide fractionation based on C18 StageTips for in-depth proteome profiling. Analytical Methods 2019, 11, 4693-4698.
52. Wuhrer, M.; de Boer, A. R.; Deelder, A. M., Structural glycomics using hydrophilic interaction chromatography (HILIC) with mass spectrometry. Mass Spectrom Rev 2009, 28, 192-206.
53. Lin, Y—I.; Lang, S. A.; Lovell, M. F.; Perkinson, N. A., New synthesis of 1,2,4-triazoles and 1,2,4-oxadiazoles. The Journal of Organic Chemistry 1979, 44(23), 4160-4164.
54. Lazreg, F., et. al., Organometallics 2018, 37, 679-683.
55. Yamauchi, M., et. al., Heterocycles 2010, 80(1), 177-181.
56. Dhillon, A. S., Hagan, S., Rath, O. & Kolch, W. MAP kinase signalling pathways in cancer. Oncogene 2007, 26, 3279-90.
57. Newton, A. C. Protein kinase C: structure, function, and regulation. J Biol Chem 1995, 270, 28495-8.
58. Krishna, S. & Zhong, X.-P. Regulation of Lipid Signaling by Diacylglycerol Kinases during T Cell Development and Function. Frontiers in Immunology 2013, 4.
59. Merida, I., Andrada, E., Gharbi, S. I. & Avila-Flores, A. Redundant and specialized roles for diacylglycerol kinases alpha and zeta in the control of T cell functions. Sci Signal 2015, 8, re6.
60. Zhou, P. et al. In vivo discovery of immunotherapy targets in the tumour microenvironment. Nature 2014, 506, 52-7.
61. Jing, W. et al. T Cells Deficient in Diacylglycerol Kinase zeta Are Resistant to PD-1 Inhibition and Help Create Persistent Host Immunity to Leukemia. Cancer Res 2017, 77, 5676-5686.
62. Merida, I. et al. Diacylglycerol kinases in cancer. Adv Biol Regul 2017, 63, 22-31.
63. Sakane, F., Mizuno, S. & Komenoi, S. Diacylglycerol Kinases as Emerging Potential Drug Targets for a Variety of Diseases: An Update. Front Cell Dev Biol 2016, 4, 82.
64. Olenchock, B. A. et al. Disruption of diacylglycerol metabolism impairs the induction of T cell anergy. Nat Immunol 2006, 7, 1174-81.
65. Zha, Y. et al. T cell anergy is reversed by active Ras and is regulated by diacylglycerol kinase-alpha. Nat Immunol 2006, 7, 1166-73.
66. Prinz, P. U. et al. High DGK-alpha and disabled MAPK pathways cause dysfunction of human tumor-infiltrating CD8+ T cells that is reversible by pharmacologic intervention. J Immunol 2012, 188, 5990-6000.
67. Riese, M. J. et al. Enhanced effector responses in activated CD8+ T cells deficient in diacylglycerol kinases. Cancer Res 2013, 73, 3566-77.
68. Chen, S. S., Hu, Z. & Zhong, X. P. Diacylglycerol Kinases in T Cell Tolerance and Effector Function. Front Cell Dev Biol 2016, 4, 130.
69. Riese, M. J., Moon, E. K., Johnson, B. D. & Albelda, S. M. Diacylglycerol Kinases (DGKs): Novel Targets for Improving T Cell Activity in Cancer. Front Cell Dev Biol 2016, 4, 108 (2016).
70. Guo, R. et al. Synergistic control of T cell development and tumor suppression by diacylglycerol kinase alpha and zeta. Proc Natl Acad Sci USA 2008, 105, 11909-14.
71. Jung, I. Y. et al. CRISPR/Cas9-Mediated Knockout of DGK Improves Antitumor Activities of Human T Cells. Cancer Res 2018, 78, 4692-4703.
72. Yang, J. et al. DGK alpha and zeta Activities Control TH1 and TH17 Cell Differentiation. Front Immunol 2019, 10, 3048.
73. Macián, F. et al. Transcriptional Mechanisms Underlying Lymphocyte Tolerance. Cell 2002, 109, 719-731.
74. Baine, I., Abe, B. T. & Macian, F. Regulation of T-cell tolerance by calcium/NFAT signaling. Immunol Rev 2009, 231, 225-40.
75. Arranz-Nicolas, J. et al. Diacylglycerol kinase alpha inhibition cooperates with PD-1-targeted therapies to restore the T cell activation program. Cancer Immunol Immunother 2021, 70, 3277-3289.
76. Fu, L. et al. DGKA Mediates Resistance to PD-1 Blockade. Cancer Immunol Res 2021, 9, 371-385.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A compound having a structure of formula (I), (II), or (III): 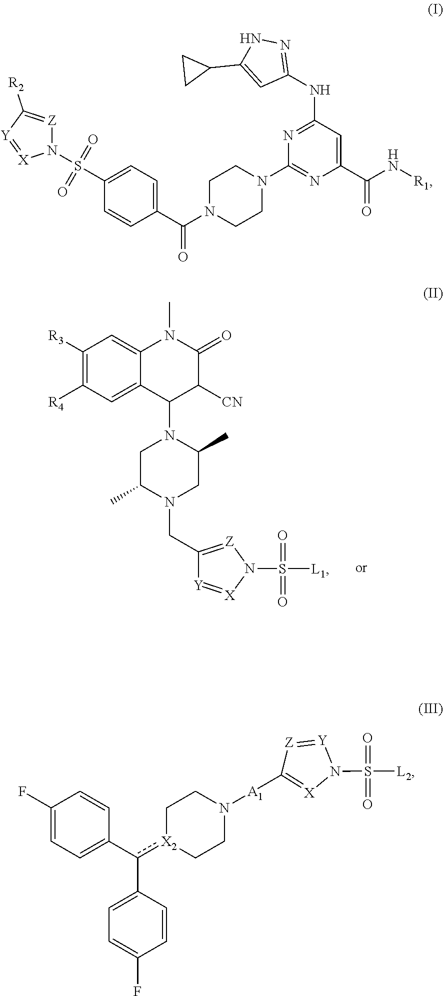
wherein: [symbol]
is a double or single bond;
X, Y, and Z are independently C or N, subject to the proviso that at least one of X, Y, and Z is N;
X₂ is C or N, subject to the proviso that when [symbol] is a single bond, X₂ is N and when [symbol] is a double bond, X₂ is C;
R₁ is alkyl, cycloalkyl, aralkyl, aryl, or heteroaryl, which alkyl, cycloalkyl, aralkyl, aryl or heteroaryl is optionally substituted with one or more alkyl or aryl group substituent selected from the group consisting of halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene, and subject to the proviso that R₁ does not comprise an alkyne group;
R₂ is alkyl, cycloalkyl, aralkyl, or aryl, which alkyl, cycloalkyl, aralkyl, or aryl is optionally substituted with one or more alkyl or aryl group substituent selected from the group consisting of halo, cyano, alkyl, alkoxy, perhaloalkyl, perhaloalkoxy, cycloalkyl, aralkyl, aryl, and amido;
R₃ and R₄ are independently selected from the group consisting of H, halo, alkyl, perhaloalkyl, and alkoxy;
L₁ and L₂ are each alkyl, cycloalkyl, aralkyl, aryl, or heteroaryl, which alkyl, cycloalkyl, aralkyl, aryl or heteroaryl is optionally substituted with one or more alkyl or aryl group substituent selected from the group consisting of halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene, and subject to the proviso that L₁ and L₂ do not comprise an alkyne group; and
A₁ is selected from the group consisting of ethylene, 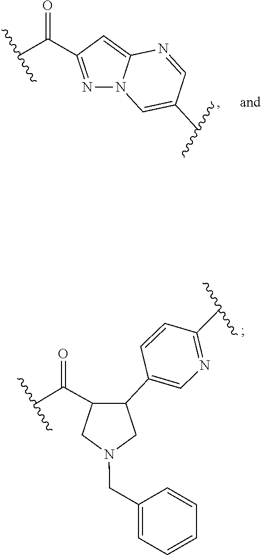 [SMILES: *C(=O)c1cc2ncc(*)cn2n1 ; *C(=O)C1CN(Cc2ccccc2)CC1c1ccc(*)nc1]
or a pharmaceutically acceptable salt or solvate thereof.

2. The compound of claim 1, wherein the compound has a structure of formula (I): 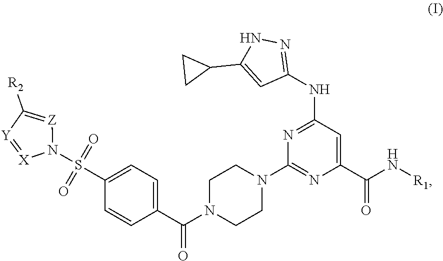
wherein:
X, Y, and Z are independently C or N, subject to the proviso that two of X, Y, and Z are N;
R₁ is alkyl, cycloalkyl, aralkyl, aryl, or heteroaryl, which alkyl, cycloalkyl, aralkyl, aryl or heteroaryl is optionally substituted with one or more alkyl or aryl group substituent selected from the group consisting of halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene, and subject to the proviso that R₁ does not comprise an alkyne group; and
R₂ is alkyl, cycloalkyl, aralkyl, or aryl, which alkyl, cycloalkyl, aralkyl, or aryl is optionally substituted with one or more alkyl or aryl group substituent selected from the group consisting of halo, cyano, alkyl, alkoxy, perhaloalkyl, perhaloalkoxy, cycloalkyl, aralkyl, aryl, and amido;
or a pharmaceutically acceptable salt or solvate thereof.

3. The compound of claim 2, wherein Y and Z are each N and X is C.

4. The compound of claim 2 or claim 3, wherein R₁ is alkyl.

5. The compound of any one of claims 2-4, wherein R₁ is n-propyl.

6. The compound of any one of claims 2-5, wherein R₂ is aryl.

7. The compound of any one of claims 2-6, wherein R₂ is phenyl.

8. The compound of any one of claims 2-7, wherein the compound is 6-((5-cycloproypyl-1H-pyrazol-3-yl)amino)-2-(4-(4-((3-phenyl-1H-1,2,4-triazol-1-yl)sulfonyl)-benzoyl)piperaz-in-1-yl)-N-propylpyrimidine-4-carboxamide) (KY-424), or a pharmaceutically acceptable salt or solvate thereof.

9. The compound of claim 1, wherein the compound has a structure of formula (II): 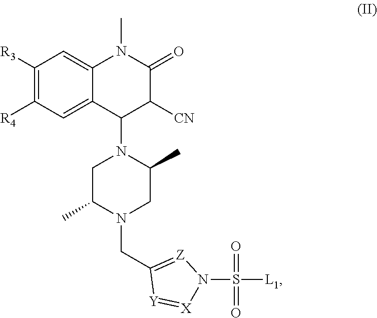
wherein:
X, Y, and Z are independently C or N, subject to the proviso that two of X, Y, and Z are N;
R₃ and R₄ are independently selected from the group consisting of H, halo, alkyl, perhaloalkyl, and alkoxy; and
L₁ is alkyl, cycloalkyl, aralkyl, aryl, or heteroaryl, which alkyl, cycloalkyl, aralkyl, aryl or heteroaryl is optionally substituted with one or more alkyl or aryl group substituent selected from the group consisting halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene, and subject to the proviso that L₁ does not contain an alkyne group;
or a pharmaceutically acceptable salt or solvate thereof.

10. The compound of claim 9, wherein X and Y are N and Z is C.

11. The compound of claim 9 or claim 10, wherein R₃ and R₄ are independently selected from the group consisting of H, halo, and alkoxy.

12. The compound of any one of claims 9-11, wherein R₃ is H or methoxy and wherein R₄ is H, Br, or F.

13. The compound of any one of claims 9-12, wherein L₁ is selected from the group consisting of alkyl, substituted alkyl, cycloalkyl, aralkyl, phenyl, substituted phenyl, thiazole, and substituted thiazole.

14. The compound of any one of claims 9-13, wherein L₁ is selected from the group consisting of isopropyl, isobutyl, cyclopropyl, 2-methoxyethyl, 3,3,3-trifluoropropyl, benzyl, phenyl, p-fluorophenyl, p-bromophenyl, p-cyanophenyl, and dimethylthiazole.

15. The compound of any one of claims 9-14, wherein the compound is selected from the group consisting of:
4-((2S,5R)-2,5-dimethyl-4-((1-phenylsulfonyl)-1H-1,2,3-triazol-4-yl) methyl)piperazin-1-yl)-1-methyl-2-oxo-1,2-dihydroquinoline-3-carbonitrile (SMS-55);
4-((2S,5R)-4-((cyclopropylsulfonyl)-1H-1,2,3-triazol-4-yl) methyl) 2,5-dimethyl piperazin-1-yl)-1-methyl-2-oxo-1,2-dihydroquinoline-3-carbonitrile (SMS-59);
4-((2S,5R)-4-((isopropylsulfonyl)-1H-1,2,3-triazol-4-yl)methyl)-2,5-dimethyl piperazin-1-yl)-1-methyl-2-oxo-1,2-dihydroquinoline-3-carbonitrile (SMS-63);
4-((2S,5R)-4-((1-((4-bromophenyl)sulfonyl)-1H-1,2,3-triazol-4-yl)methyl)-2,5-dimethylpiperazin-1-yl)-1-methyl-2-oxo-1,2-dihydroquinoline-3-carbonitrile (SMS-65);
4-((2S,5R)-4-((1-((4-fluorophenyl)sulfonyl)-1H-1,2,3-triazol-4-yl)methyl)-2,5-dimethylpiperazin-1-yl)-1-methyl-2-oxo-1,2-dihydroquinoline-3-carbonitrile (SMS-67);
4-((2S,5R)-4-((1-((4-cyanophenyl)sulfonyl)-1H-1,2,3-triazol-4-yl)methyl)-2,5-dimethylpiperazin-1-yl)-1-methyl-2-oxo-1,2-dihydroquinoline-3-carbonitrile (SMS-69);
4-((2S,5R)-4-((1-((2,4-dimethylthiazol-5-yl)sulfonyl)-1H-1,2,3-triazol-4-yl) methyl)-2,5-dimethylpiperazin-1-yl)-1-methyl-2-oxo-1,2-dihydroquinoline-3-carbonitrile (SMS-71);
4-((2S,5R)-4-((1-benzylsulfonyl)-1H-1,2,3-triazol-4-yl)methyl)-2,5-dimethyl piperazin-1-yl)-1-methyl-2-oxo-1,2-dihydroquinoline-3-carbonitrile (SMS-73);
4-((2S,5R)-4-((isobutylsulfonyl)-1H-1,2,3-triazol-4-yl)methyl)-2,5-dimethyl piperazin-1-yl)-1-methyl-2-oxo-1,2-dihydroquinoline-3-carbonitrile (SMS-75);
4-((2S,5R)-4-((2-methoxyethyl)sulfonyl)-1H-1,2,3-triazol-4-yl)methyl)-2,5-dimethyl piperazin-1-yl)-1-methyl-2-oxo-1,2-dihydroquinoline-3-carbonitrile (SMS-77);
4-((2S,5R)-2,5-dimethyl-4-((1-((3,3,3-trifluoropropyl)sulfonyl)-1H-1,2,3-triazol-4-yl) methyl)-piperazin-1-yl)-1-methyl-2-oxo-1,2-dihydroquinoline-3-carbonitrile (SMS-79);
6-bromo-4-((2S,5R)-2,5-dimethyl-4-((1-phenylsulfonyl)-1H-1,2,3-triazol-4-yl) methyl)piperazin-1-yl)-1-methyl-2-oxo-1,2-dihydroquinoline-3-carbonitrile (SMS-81);
4-((2S,5R)-2,5-dimethyl-4-((1-phenylsulfonyl)-1H-1,2,3-triazol-4-yl)methyl) piperazin-1-yl)-6-fluoro-1-methyl-2-oxo-1,2-dihydroquinoline-3-carbonitrile (SMS-83);
6-fluoro-4-((2S,5R)-4-((isopropylsulfonyl)-1H-1,2,3-triazol-4-yl)methyl)-2,5-dimethylpiperazin-1-yl)-1-methyl-2-oxo-1,2-dihydroquinoline-3-carbonitrile (SMS-85);
4-((2S,5R)-2,5-dimethyl-4-((1-phenylsulfonyl)-1H-1,2,3-triazol-4-yl)methyl) piperazin-1-yl)-7-methoxy-1-methyl-2-oxo-1,2-dihydroquinoline-3-carbonitrile (SMS-87);
and pharmaceutically acceptable salts or solvates thereof.

16. The compound of claim 1, wherein the compound has a structure of formula (III): 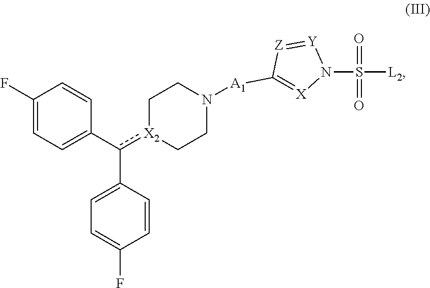
wherein: [symbol]
is a double or single bond;
X, Y, and Z are independently C or N, subject to the proviso that two of X, Y, and Z are N;
X₂ is C or N, subject to the proviso that when [symbol] is a single bond, X₂ is N and when [symbol] is a double bond, X₂ is C;
L₂ is alkyl, cycloalkyl, aralkyl, aryl, or heteroaryl, which alkyl, cycloalkyl, aralkyl, aryl or heteroaryl is optionally substituted with one or more alkyl or aryl group substituent selected from the group consisting of halo, cyano, alkyl, alkoxy, perhaloalkyl, perhaloalkoxy, cycloalkyl, aralkyl, aryl, and amido, or wherein two alkyl or aryl group substituents together form alkylene or substituted alkylene, and subject to the proviso that L₂ does not comprise an alkyne group; and
A₁ is selected from the group consisting of ethylene, 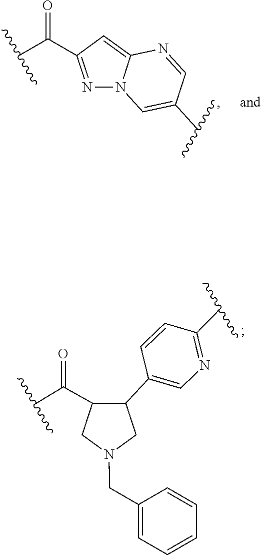 [SMILES: *C(=O)c1cc2ncc(*)cn2n1 ; *C(=O)C1CN(Cc2ccccc2)CC1c1ccc(*)nc1]
or a pharmaceutically acceptable salt or solvate thereof.

17. The compound of claim 16, wherein Y and Z are each N and X is C.

18. The compound of claim 16 or claim 17, wherein L₂ is selected from the group consisting of cyclopropyl, phenyl, substituted phenyl, thiazole, and dimethylthiazole.

19. The compound of any one of claims 16-18, wherein L₂ is phenyl substituted with one or two substituents selected from the group consisting of alkyl, alkoxy, halo, and amido, or wherein L₂ is phenyl substituted with two substituents that together form an alkylene or substituted alkylene.

20. The compound of any one of claims 16-19, wherein A₁ is ethylene.

21. The compound of any one of claims 16-20, wherein the compound is selected from the group consisting of:
4-((4-(2-(4-(Bis(4-fluorophenyl)methylene)piperidin-1-yl)ethyl)-1H-1,2,3-triazol-1-yl)sulfonyl)-N-propylbenzamide (TH225);
4-(Bis(4-fluorophenyl)methylene)-1-(2-(1-tosyl-1H-1,2,3-triazol-4-yl)ethyl)-piperidine (TH207);
4-(bis(4-fluorophenyl)methylene)-1-(2-(1-(cyclopropylsulfonyl)-1H-1,2,3-triazol-4-yl)ethyl)piperidine (TH223);
1-(Bis(4-fluorophenyl)methyl)-4-(2-(1-tosyl-1H-1,2,3-triazol-4-yl)ethyl)piperazine (TH208);
4-(Bis(4-fluorophenyl)methylene)-1-(2-(1-tosyl-1H-1,2,3-triazol-4-yl)ethyl)-piperidine (TH220);
4-(Bis(4-fluorophenyl)methylene)-1-(2-(1-((4-fluorophenyl)sulfonyl)-1H-1,2,3-triazol-4-yl)ethyl)piperidine (TH221);
(4-(Bis(4-fluorophenyl)methylene)piperidin-1-yl)(6-(1-((4-methoxyphenylsulfonyl)-1H-1,2,3-triazol-4-yl)pyrazolo[1,5-a]pyrimidin-2-yl)methanone (XJ-2-47);
(1-Benzyl-4-(6-(1-((2,4-dimethylthiazol-5-yl)sulfonyl)-1H-1,2,3-triazol-4-yl)pyridin-3-yl)pyrrolidin-3-yl)(4-(bis(4-fluorophenyl)methylene)piperidin-1-yl)methanone (XJ-2-65);
4-(Bis(4-fluorophenyl)methylene)-1-(2-(1-((2,3-dihydrobenzo[b][1,4]dioxin-6-yl)sulfonyl)-1H-1,2,3-triazol-4-yl)ethyl)piperidine (XJ-2-77);
1-(2-(1-(Benzo[d][1,3]dioxol-5-ylsulfonyl)-1H-1,2,3-triazol-4-yl)ethyl)-4-(bis(4-fluorophenyl)methylene)piperidine (XJ-2-87);
5-((4-(2-(4-(Bis(4-fluorophenyl)methylene)piperidin-1-yl)ethyl)-1H-1,2,3-triazol-1-yl)sulfonyl)-2,4-dimethylthiazole (XJ-2-105);
4-(Bis(4-fluorophenyl)methylene)-1-(2-(1-((2,3-dihydrobenzofuran-6-yl)sulfonyl)-1H-1,2,3-triazol-4-yl)ethyl)piperidine (XJ-2-111);
4-(Bis(4-fluorophenyl)methylene)-1-(2-(1-((2,2-difluorobenzo[d][1,3]dioxol-5-yl)sulfonyl)-1H-1,2,3-triazol-4-yl)ethyl)piperidine (XJ-2-115);
4-(Bis(4-fluorophenyl)methylene)-1-(2-(1-((2,4-dimethoxyphenyl)sulfonyl)-1H-1,2,3-triazol-4-yl)ethyl)piperidine (XJ-2-139);
4-(Bis(4-fluorophenyl)methylene)-1-(2-(1-((2-methoxyphenyl)sulfonyl)-1H-1,2,3-triazol-4-yl)ethyl)piperidine (XJ-2-141);
and pharmaceutically acceptable salts and solvates thereof.

22. A pharmaceutical composition comprising a compound of any one of claims 1-21 and a pharmaceutically acceptable carrier.

23. A method of inhibiting a kinase, the method comprising contacting a sample comprising the kinase with a compound of any one of claims 1-21 or a pharmaceutical composition of claim 22.

24. The method of claim 23, wherein the sample is selected from the group consisting of a biological fluid, a cell culture, a cell extract, a tissue, a tissue extract, an organ, and an organism.

25. The method of claim 23 or claim 24, wherein the kinase is selected from the group consisting of Cyclin-dependent kinase 1 (CDK1), Cyclin-dependent kinase 2 (CDK2), Cyclin-dependent-like kinase 5 (CDK5), Dual specificity mitogen-activated protein kinase kinase 1, eIF-2-alpha kinase GCN2, Interleukin-1 receptor-associated kinase 4, MAP/microtubule affinity-regulating kinase 4, Mitogen-activated protein kinase kinase kinase kinase 1, Mitogen-activated protein kinase kinase kinase kinase 2, Mitogen-activated protein kinase kinase kinase kinase 5, Phosphatidylinositol 4,5-bisphosphate 3-kinase catalytic subunit delta, Phosphoglycerate kinase 1, Protein-tyrosine kinase 2-beta, Pyruvate kinase PKM, Receptor-interacting serine/threonine-protein kinase 1, Serine/threonine-protein kinase 4, Serine/threonine-protein kinase MARK2, Serine/threonine-protein kinase tousled-like 2, Thymidylate kinase, Tyrosine-protein kinase Fer, Tyrosine-protein kinase Lck, 5′-AMP-activated protein kinase catalytic subunit alpha-1, Cyclin-dependent-like kinase 6, Dual specificity mitogen-activated protein kinase kinase 2, Interferon-induced, double-stranded RNA-activated protein kinase, Nucleoside diphosphate kinase B, Serine/threonine-protein kinase tousled-like 1,Tyrosine-protein kinase CSK, a diacylglycerol kinase (DGK), and phosphofructokinase, liver type (PFKL).


SEQUENCE LISTING (5 sequences)

SEQ ID NO: 1 - protein, 15 aa, synthetic construct
  REGION 1..15: note = synthetic construct
  MOD_RES 1: note = ACETYLATION
  MOD_RES 15: note = amidation
RLNERHYGGLTGLNK

SEQ ID NO: 2 - protein, 298 aa, Homo sapiens
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 3 - protein, 735 aa, Homo sapiens
MAKERGLISPSDFAQLQKYMEYSTKKVSDVLKLFEDGEMAKYVQGDAIGYEGFQQFLKIYLEVDNVPRHLSLALFQSFETGHCLNETNVTKDVVCLNDVSCYFSLLEGGRPEDKLEFTFKLYDTDRNGILDSSEVDKIILQMMRVAEYLDWDVSELRPILQEMMKEIDYDGSGSVSQAEWVRAGATTVPLLVLLGLEMTLKDDGQHMWRPKRFPRPVYCNLCESSIGLGKQGLSCNLCKYTVHDQCAMKALPCEVSTYAKSRKDIGVQSHVWVRGGCESGRCDRCQKKIRIYHSLTGLHCVWCHLEIHDDCLQAVGHECDCGLLRDHILPPSSIYPSVLASGPDRKNSKTSQKTMDDLNLSTSEALRIDPVPNTHPLLVFVNPKSGGKQGQRVLWKFQYILNPRQVFNLLKDGPEIGLRLFKDVPDSRILVCGGDGTVGWILETIDKANLPVLPPVAVLPLGTGNDLARCLRWGGGYEGQNLAKILKDLEMSKVVHMDRWSVEVIPQQTEEKSDPVPFQIINNYFSIGVDASIAHRFHIMREKYPEKFNSRMKNKLWYFEFATSESIFSTCKKLEESLTVEICGKPLDLSNLSLEGIAVLNIPSMHGGSNLWGDTRRPHGDIYGINQALGATAKVITDPDILKTCVPDLSDKRLEVVGLEGAIEMGQIYTKLKNAGRRLAKCSEITFHTTKTLPMQIDGEPWMQTPCTIKITHKNQMPMLMGPPPRSTNFFGFLS

SEQ ID NO: 4 - protein, 928 aa, Homo sapiens
MEPRDGSPEARSSDSESASASSSGSERDAGPEPDKAPRRLNKRRFPGLRLFGHRKAITKSGLQHLAPPPPTPGAPCSESERQIRSTVDWSESATYGEHIWFETNVSGDFCYVGEQYCVARMLKSVSRRKCAACKIVVHTPCIEQLEKINFRCKPSFRESGSRNVREPTFVRHHWVHRRRQDGKCRHCGKGFQQKFTFHSKEIVAISCSWCKQAYHSKVSCFMLQQIEEPCSLGVHAAVVIPPTWILRARRPQNTLKASKKKKRASFKRKSSKKGPEEGRWRPFIIRPTPSPLMKPLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRWDLHAEPNPEAGPEDRDEGATDRLPLDVFNNYFSLGFDAHVTLEFHESREANPEKFNSRFRNKMFYAGTAFSDFLMGSSKDLAKHIRVVCDGMDLTPKIQDLKPQCVVFLNIPRYCAGTMPWGHPGEHHDFEPQRHDDGYLEVIGFTMTSLAALQVGGHGERLTQCREVVLTTSKAIPVQVDGEPCKLAASRIRIALRNQATMVQKAKRRSAAPLHSDQQPVPEQLRIQVSRVSMHDYEALHYDKEQLKEASVPLGTVVVPGDSDLELCRAHIERLQQEPDGAGAKSPTCQKLSPKWCFLDATTASRFYRIDRAQEHLNYVTEIAQDEIYILDPELLGASARPDLPTPTSPLPTSPCSPTPRSLQGDAAPPQGEELIEAAKRNDFCKLQELHRAGGDLMHRDEQSRTLLHHAVSTGSKDVVRYLLDHAPPEILDAVEENGETCLHQAAALGQRTICHYIVEAGASLMKTDQQGDTPRQRAEKAQDTELAAYLENRQHYQMIQREDQETAV

SEQ ID NO: 5 - protein, 780 aa, Homo sapiens
MAAVDLEKLRASGAGKAIGVLTSGGDAQGMNAAVRAVTRMGIYVGAKVFLIYEGYEGLVEGGENIKQANWLSVSNIIQLGGTIIGSARCKAFTTREGRRAAAYNLVQHGITNLCVIGGDGSLTGANIFRSEWGSLLEELVAEGKISETTARTYSHLNIAGLVGSIDNDFCGTDMTIGTDSALHRIMEVIDAITTTAQSHQRTFVLEVMGRHCGYLALVSALASGADWLFIPEAPPEDGWENFMCERLGETRSRGSRLNIIIIAEGAIDRNGKPISSSYVKDLVVQRLGFDTRVTVLGHVQRGGTPSAFDRILSSKMGMEAVMALLEATPDTPACVVTLSGNQSVRLPLMECVQMTKEVQKAMDDKRFDEATQLRGGSFENNWNIYKLLAHQKPPKEKSNFSLAILNVGAPAAGMNAAVRSAVRTGISHGHTVYVVHDGFEGLAKGQVQEVGWHDVAGWLGRGGSMLGTKRTLPKGQLESIVENIRIYGIHALLVVGGFEAYEGVLQLVEARGRYEELCIVMCVIPATISNNVPGTDFSLGSDTAVNAAMESCDRIKQSASGTKRRVFIVETMGGYCGYLATVTGIAVGADAAYVFEDPFNIHDLKVNVEHMTEKMKTDIQRGLVLRNEKCHDYYTTEFLYNLYSSEGKGVFDCRTNVLGHLQQGGAPTPFDRNYGTKLGVKAMLWLSEKLREVYRKGRVFANAPDSACVIGLKKKAVAFSPVTELKKDTDFEHRMPREQWWLSLRLMLKMLAQYRISMAAYVSGELEHVTRRTLSMDKGF